(19) United States
(12) Patent Application Publication
(10) Pub. No.: US 2010/0144722 A1
(43) Pub. Date: Jun. 10, 2010

(54) NOVEL HETEROCYCLIC COMPOUNDS AS GATA MODULATORS

(75) Inventors: Christopher W. Alexander, Atlanta, GA (US); Dibyendu De, Suwanee, GA (US); Ish Kumar Khanna, Alpharetta, GA (US); Sivaram Pillarisetti, Norcross, GA (US)

Correspondence Address: Nelson Mullins Riley & Scarborough LLP;IP Department, 100 North Tryon Street, 42nd Floor, Charlotte, NC, 28202-4000, US

(73) Assignee: Dr. Reddy's Laboratories Ltd., Hyderabad (IN)

(21) Appl. No.: 12/553,623

(22) Filed: Sep. 3, 2009

Related U.S. Application Data
(60) Provisional application No. 61/190,836, filed on Sep. 3, 2008.

(51) Int. Cl. A61K 31/541; A61P 3/10; A61P 9/10; C07D 239/48; A61K 31/505; C07D 413/04; A61K 31/5377; A61K 31/496; C07D 413/14; C07D 417/04

(52) U.S. Cl. 514/227.8; 544/323; 514/275; 544/122; 514/235.8; 514/252.19; 544/357; 544/58.2

(57) ABSTRACT

Novel heterocyclic compounds, sstereoisomers thereof and/or pharmaceutically acceptable salts of formula (I) and its stereoisomers are provided. Additionally, methods of forming novel heterocyclic compounds, stereoisomers thereof and/or pharmaceutically acceptable salts of formula (I) and its stereoisomers are provided.

The present application claims priority to U.S. Provisional Patent Application Ser. No. 61/190,836, filed Sep. 3, 2008, which is incorporated herein by reference in its entirety.

### BACKGROUND OF THE INVENTION

The present invention relates to novel heterocyclic compounds, stereoisomers thereof and/or pharmaceutically acceptable salts of the compounds and stereoisomers.

Atherosclerosis is generally considered an inflammatory disease—with inflammation being the cause of both initiation and progression of the lesion. Without being bound by theory, cholesterol accumulation in macrophages of atherosclerotic lesion (fatty streaks) is believed to be a contributor of localized inflammation and lesion progression. Several types of lipid particles can contribute to the formation of fatty streaks—cholesterol rich low density lipoprotein particles (LDLs) and triglyceride rich very low density lipoproteins (VLDLs) and remnant particles. Cholesterol and other lipids from these particles are typically taken by macrophages in atherosclerotic lesions leading to the formation of lipid-loaded foam cells. Atherosclerotic plaques containing lipid-loaded macrophages are typically inflammatory, are unstable, and are prone to plaque rupture. Acute coronary syndromes (ACS) are often the end manifestations of such plaque rupture, resulting in angina (chest pain), myocardial ischemia (MI, heart attack), fatal MI (sudden death), or stroke. LDL-lowering therapy may allow for remodeling of these plaques, rendering the plaques less prone to rupture. Statin-mediated remodeling processes, however, may take months to years to occur, and hence the benefit of lipid-lowering therapy may not be clinically noticed for several years, as was shown in recent statin prevention trials. Also, although statins are effective at reducing cardiovascular risk, they typically reduce risk by only about one-third over five years. Stabilizing plaque, by reducing cholesterol content or by reducing inflammation, may be important for ACS patients to minimize/prevent reoccurrence of cardiac events.

Cholesterol-loaded macrophages are typically present at all stages of atherosclerosis and are typically abundant in ruptured atherosclerotic plaques. Pathways leading to cholesterol accumulation and egress may determine the physiological (inflammatory/apoptotic) state of the macrophage. Without being bound by theory, macrophage cholesterol is thought to be removed by reverse cholesterol transport (RCT), a process that involves several players including ATP-binding cassette transporters ABCA1 and ABCG1, lecithin cholesterol acyltransferase (LCAT), and scavenger receptor, class B, type I (SR-B1). An increase in the activity of ABCA1, LCAT, and SR-B1, typically results in a boost in the arteries. The ABCA1, LCAT, and SR-B1 genes are, therefore, commonly referred to as reverse cholesterol transport RCT) genes.

Elevated levels of LDL and triglycerides and low levels of HDL are often found in diabetics. This phenotype is referred to as “diabetic dyslipidemia”. This condition may result in cholesterol accumulation, especially in tissues that are important in glucose metabolism. Cholesterol accumulation in tissues may lead tissue dysfunction. For example cholesterol accumulation in the pancreas may result in decreased secretion of insulin, the critical hormone required for glucose uptake. Cholesterol accumulation in other tissues, e.g. adipose and skeletal muscle, may lead to insulin resistance and, thus, defective glucose uptake in response to insulin. Removal of cholesterol from these tissues will typically have a beneficial effect on insulin resistance, pancreatic function and, thus, is useful for prevention and treatment of diabetes.

GATA can modulate the expression of RCT proteins and pharmacological modulation of GATA can serve as a mechanism for the treatment of atherosclerosis, diabetes, and its associated complications (U.S. patent application Ser. No. 12/113,426, incorporated herein by reference in its entirety). GATA transcription factor contains three domains, the C-finger, the N-finger, and the Activation Domain. The C-finger, named for being near the C-terminal, has two highly conserved zinc finger binding domains, which form the Activation Domain that binds the consensus sequence (A/T)GATA(A/G). The N-finger, named for being near the N-terminal also binds DNA and a cofactor named FOG-1. The Activation Domain is responsible for GATA's strong transcriptional activation. The gene for GATA is on the X-chromosome.

### SUMMARY OF THE INVENTION

The present invention relates to novel heterocyclic compounds having the general formula (I),

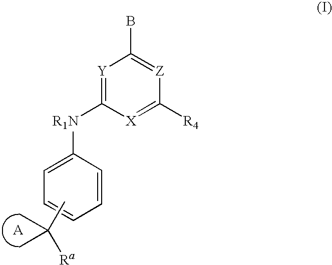

stereoisomers thereof and/or pharmaceutically acceptable salts of compounds of formula (I) and/or pharmaceutically acceptable salts of the stereoisomers of compounds of formula (I); wherein,

X, Y and Z are each independently selected from N or CR^{b}, wherein R^{b} is selected from hydrogen, halogen, or alkyl; with a proviso that when X and Y are each independently N, Z is —CR^{b} and when Y and Z are each independently N, X is —CR^{b};

A is a 3 to 7 membered cycloalkyl ring;

B is an optionally substituted group selected from alkyl, alkoxy, cycloalkyl, aryl, heteroaryl or —NR₂R₃, wherein one or more optional substituents on B are selected from halogen, hydroxy, cyano, alkyl, haloalkyl, alkoxy, cycloalkyl, —CO-alkyl, —COOR^{c}, —CONR^{d}R^{e,}—(CH₂)ₙSO₂R^{f}, —(CH₂)ₙSO₂NR^{c}R^{d}, aryl, heteroaryl or heterocyclyl;

R₁ is selected from hydrogen or an alkyl;

R₂ and R₃ are each independently selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl, wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl; or

R₂ and R₃ optionally combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring are selected from halogen, hydroxy, alkoxy, alkyl, cycloalkyl or —CO-alkyl;

R₄ is hydrogen or an optionally substituted group selected from amino, alkyl, cycloalkyl, aryl, heterocyclyl or heteroaryl, wherein one or more optional substituents on R₄ are selected from halogen, hydroxy, cyano, alkyl, —(CH₂)ₙ—OH, cycloalkyl, alkoxy or —CO-alkyl;

R^{a} is selected from cyano, —(CH₂)ₙ—OH, —(CH₂)ₙ-alkoxy, alkyl, aryl, —NH-aryl, heteroaryl, —(CH₂)ₙCO-alkyl, —(CH₂)ₙCOOR^{c}, —(CH₂)ₙCONR^{d}R^{e}, —(CH₂)ₙSO₂R^{f}, or —(CH₂)ₙSO₂NR^{c}R^{d};

R^{c} is selected from hydrogen, alkyl, cycloalkyl, aryl, heterocyclyl or heteroaryl;

R^{d} and R^{e} are each independently selected from hydrogen or an optionally substituted group selected from alkyl, cycloalkyl, alkoxy, aryl, heteroaryl or heterocyclyl, wherein one or more optional substituents on R^{d} and R^{e} are each independently selected from halogen, hydroxy or alkyl;

R^{f} is selected from alkyl, cycloalkyl, heterocyclyl, aryl or heteroaryl; and

n is an integer from 0 to 2.

In one embodiment, the invention is directed to novel compounds of formula (I), stereoisomers thereof and/or pharmaceutically acceptable salts of compounds of formula (I) and/or pharmaceutically acceptable salts of the stereoisomers of compounds of formula (I) as GATA modulators.

In another embodiment, the invention is directed to novel compounds of foimula (I), stereoisomers thereof and/or pharmaceutically acceptable salts of compounds of formula (I) and/or pharmaceutically acceptable salts of the stereoisomers of compounds of formula (I) as RCT activators.

In another embodiment, the invention is directed to a method for the treatment of atherosclerosis, diabetes and its associated complications, Alzheimer's disease, and cardiovascular disease in a subject, which comprises administering to the subject a therapeutically effective amount of a compound of formula (I), stereoisomers thereof and/or pharmaceutically acceptable salts of compounds of formula (I) and/or pharmaceutically acceptable salts of the stereoisomers of compounds of formula (I).

### DETAILED DESCRIPTION OF THE INVENTION

Reference will now be made in detail to the embodiments of the invention, one or more examples of which are set forth below. Each example is provided by way of explanation of the invention, not limited of the invention. In fact, it will be apparent to those skilled in the art that various modification and variations can be made in the present invention without departing from the scope or spirit of the invention. For instance, features illustrated or described as part of one embodiment can be used on another embodiment to yield a still further embodiment. Thus it is intended that the present invention cover such modifications and variations as come within the scope of the appended claims and their equivalents. Other objects, features, and aspects of the present invention are disclosed in, or are obvious from, the following detailed description. It is to be understood by one of ordinary skill in the art that the present discussion is a description of exemplary embodiments only, and is not intended as limiting the broader aspects of the present invention.

The present invention relates to novel heterocyclic compounds having the general formula (I),

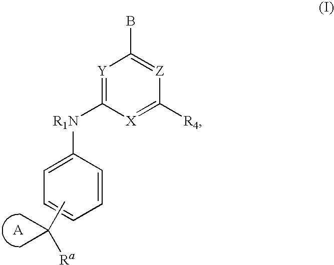

stereoisomers thereof and/or pharmaceutically acceptable salts of compounds of formula (I) and/or pharmaceutically acceptable salts of the stereoisomers of compounds of formula (I); wherein,

X, Y and Z are each independently selected from N or CR^{b}, wherein R^{b} is selected from hydrogen, halogen or alkyl;

A is a 3 to 7 membered cycloalkyl ring;

B is an optionally substituted group selected from alkyl, alkoxy, cycloalkyl, aryl, heteroaryl or —NR₂R₃, wherein one or more optional substituents on B are selected from halogen, hydroxy, cyano, alkyl, haloalkyl, alkoxy, cycloalkyl, —CO-alkyl, —COOR^{c}, —CONR^{d}R^{e,}—(CH₂)ₙSO₂R^{f}, —(CH₂)ₙSO₂NR^{c}R^{d}, aryl, heteroaryl or heterocyclyl;

R₁ is hydrogen or an alkyl;

R₂ and R₃ are each independently selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl, wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl; or

R₂ and R₃ optionally combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring are selected from halogen, hydroxy, alkoxy, alkyl, cycloalkyl or —CO-alkyl;

R₄ is hydrogen or an optionally substituted group selected from amino, alkyl, cycloalkyl, aryl, heterocyclyl or heteroaryl, wherein one or more optional substituents on R₄ are selected from halogen, hydroxy, cyano, alkyl, —(CH₂)ₙ—OH, cycloalkyl, alkoxy or —CO-alkyl;

R^{a} is selected from cyano, —(CH₂)ₙ—OH, —(CH₂)ₙ-alkoxy, alkyl, aryl, —NH-aryl, heteroaryl, —(CH₂)ₙCO-alkyl, —(CH₂)ₙCOOR^{c}, —(CH₂)ₙCONR^{d}R^{e}, —(CH₂)ₙSO₂R^{f}, or —(CH₂)ₙSO₂NR^{c}R^{d};

R^{c} is selected from hydrogen, alkyl, cycloalkyl, aryl, heterocyclyl or heteroaryl;

R^{d} and R^{c} are each independently selected from hydrogen or an optionally substituted group selected from alkyl, cycloalkyl, alkoxy, aryl, heteroaryl or heterocyclyl, wherein one or more optional substituents on R^{d} and R^{e} are each independently selected from halogen, hydroxy or alkyl;

R^{f} is selected from alkyl, cycloalkyl, heterocyclyl, aryl or heteroaryl; and

n is an integer from 0 to 2.

In one embodiment, the invention includes compounds of the formula (I),

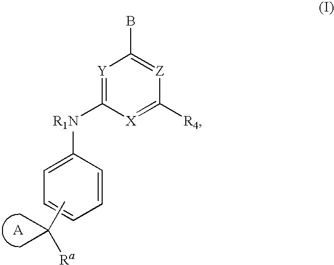

stereoisomers thereof and/or pharmaceutically acceptable salts of compounds of formula (I) and/or pharmaceutically acceptable salts of the stereoisomers of compounds of formula (I); wherein,

X, Y and Z are each independently selected from N or CR^{b}, wherein R^{b} is selected from hydrogen, halogen, or alkyl; with a proviso that when X and Y are each independently N, Z is —CR^{b} and when Y and Z are each independently N, X is —CR^{b};

A is a 3 to 7 membered cycloalkyl ring;

B is an optionally substituted group selected from alkyl, alkoxy, cycloalkyl, aryl, heteroaryl or —NR₂R₃, wherein one or more optional substituents on B are selected from halogen, hydroxy, cyano, alkyl, haloalkyl, alkoxy, cycloalkyl, —CO-alkyl, —COOR^{c}, —CONR^{d}R^{e,}—(CH₂)ₙSO₂R^{f}, —(CH₂)ₙSO₂NR^{c}R^{d}, aryl, heteroaryl, or heterocyclyl;

R₁ is hydrogen or an alkyl;

R₂ and R₃ are each independently selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl, wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂),SO₂R^{f}, —(CH₂)ₙSO₂NR^{c}R^{d}, aryl, heteroaryl or heterocyclyl; or

R₂ and R₃ optionally combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring are selected from halogen, hydroxy, alkoxy, alkyl, cycloalkyl or —CO-alkyl;

R₄ is hydrogen or an optionally substituted group selected from amino, alkyl, cycloalkyl, aryl, heterocyclyl or heteroaryl, wherein one or more optional substituents on R₄ are selected from halogen, hydroxy, cyano, alkyl, —(CH₂)ₙ—OH, cycloalkyl, alkoxy or —CO-alkyl;

R^{a} is selected from cyano, —(CH₂)—OH, —(CH₂)ₙ-alkoxy, alkyl, aryl, —NH-aryl, heteroaryl, —(CH₂)ₙCOOR^{c}, —(CH₂),CONR^{d}R^{e}, —(CH₂)ₙSO₂R^{f}, or —(CH₂)ₙSO₂NR^{c}R^{d};

R^{e} is selected from hydrogen, alkyl, cycloalkyl, aryl, heterocyclyl or heteroaryl;

R^{d} and R^{e} are each independently selected from hydrogen or an optionally substituted group selected from alkyl, cycloalkyl, alkoxy, aryl, heteroaryl or heterocyclyl, wherein one or more optional substituents on R^{d} and R^{e} are each independently selected from halogen, hydroxy or alkyl;

R^{f} is selected from alkyl, cycloalkyl, heterocyclyl, aryl or heteroaryl; and

n is an integer from 0 to 2.

In one embodiment, the invention includes compounds of the formula (II),

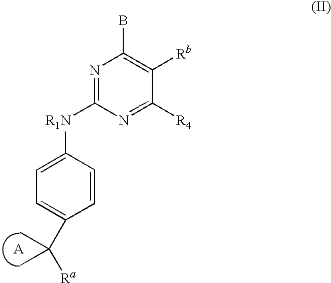

stereoisomers thereof and/or pharmaceutically acceptable salts of compounds of foimula (II) and/or pharmaceutically acceptable salts of the stereoisomers of compounds of formula (II); wherein the groups represented by A, B, R^{a}, R₁ and R₄ are same as that of formula (I).

In another embodiment, the invention includes compounds of the formula (III),

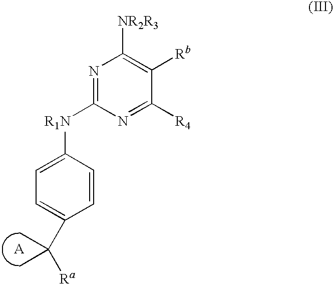

stereoisomers thereof and/or pharmaceutically acceptable salts of compounds of formula (III) and/or pharmaceutically acceptable salts of the stereoisomers of compounds of formula (III); wherein the groups represented by A, R^{a}, R₁, R₂, R₃ and R₄ are same as that of formula (I).

In yet another embodiment, the invention includes compounds of formula (III), wherein

A is selected from cyclopropyl, cyclobutyl or cyclopentyl;

R₁ is hydrogen;

R₂ is selected from hydrogen or —(CH₂)ₙ-cycloalkyl;

R₃ is selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl; wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl;

R₄ is an optionally substituted group selected from amino, alkyl, cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;

R^{a} is selected from cyano, alkyl, —(CH₂)ₙ—OH, —(CH₂)ₙ-alkoxy, —CO-alkyl, heteroaryl, —(CH₂)ₙCOOR^{c}, —(CH₂)ₙCONR^{d}R^{e}, —(CH₂)ₙSO₂R^{f} or —(CH₂)ₙSO₂NR^{c}R^{d};

R^{b} is selected from hydrogen or alkyl;

R^{c} is selected from hydrogen or alkyl;

R^{d} and R^{e} are each independently selected from hydrogen, alkyl, alkoxy, or aryl, which is optionally substituted with halogen;

R^{f} is selected from alkyl, cycloalkyl, heterocyclyl, aryl or heteroaryl; and

n is an integer from 0 to 2.

In still another embodiment, the invention includes compounds of formula (III), wherein

R₃ is selected from hydrogen, alkyl, —(CH₂)ₙ-cycloalkyl, and an optionally substituted groups selected from -(CH₂)ₙ—OH, —(CH₂)ₙ-heterocyclyl, —(CH₂)ₙ-aryl, —(CH₂)ₙ-heteroaryl, wherein the optional substituents on R₃ are selected from halogen, —CH₂—OH, alkyl, aryl, —(CH₂)ₙSO₂NR^{c}R^{d} or —(CH₂)ₙSO₂R^{f};

R₄ is selected from an optionally substituted group selected from amino, alkyl, cycloalkyl, aryl, heterocyclyl or heteroaryl, wherein one or more optional substituents on R₄ are selected from halogen, alkyl or alkoxy;

R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂),SO₂NR^{c}R^{d};

R^{c} is selected from hydrogen or alkyl, and

R^{d} and R^{e} are each independently selected from hydrogen, alkyl, alkoxy or aryl, which is optionally substituted with halogen.

In still another embodiment, the invention includes compounds of formula (III), wherein

R₂ and R₃ combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring formed may be selected from halogen, hydroxyl, alkoxy, alkyl, cycloalkyl or —CO-alkyl;

R₄ is an optionally substituted group selected from cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;

R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂)ₙSO₂NR^{c}R^{d};

R^{c} is selected from hydrogen or alkyl;

R^{d} and R^{e} are each independently selected from hydrogen, alkyl or alkoxy;

R^{f} is selected from alkyl, cycloalkyl, heterocyclyl, aryl or heteroaryl; and

n is an integer from 0 to 2.

Another embodiment of the invention comprises compounds of formula (III), wherein

R₂ and R₃ combine with the nitrogen atom, to which they are attached, to form an optionally substituted morpholinyl, piperidinyl, pyrrolidinyl, piperazinyl, thiomorpholinyl, dioxo-thiomorpholinyl, wherein one or more optional substituents on the heterocyclic ring formed may be selected from halogen, hydroxy, alkoxy, alkyl or —CO-alkyl;

R₄ is an optionally substituted group selected from amino, alkyl, cycloalkyl, aryl, heterocyclyl or heteroaryl, wherein one or more optional substituents on R₄ are selected from halogen, alkyl or alkoxy;

R^{a} is selected from —COOR^{c} or —CONR^{d}R^{e}

R^{e} is selected from hydrogen or alkyl; and

R^{d} and R^{e} are independently selected from hydrogen, alkyl, alkoxy or aryl, which is optionally substituted with halogen.

In yet another embodiment, the invention includes compounds of the formula (IV),

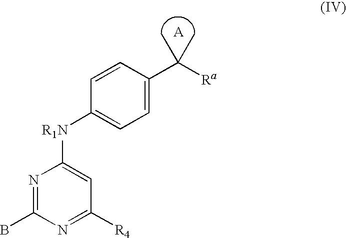

stereoisomers thereof and/or pharmaceutically acceptable salts of compounds of formula (IV) and/or pharmaceutically acceptable salts of the stereoisomers of compounds of formula (IV); wherein the groups represented by A, B, R^{a} and R₄ are same as that of formula (I).

In yet another embodiment, the invention includes compounds of the formula (IV), wherein

R₁ is hydrogen;

A is selected from cyclopropyl, cyclobutyl or cyclopentyl;

B is an optionally substituted group selected from alkyl, cycloalkyl, aryl, heteroaryl or —NR₂R₃, wherein one or more optional substituents on B are selected from halogen, hydroxy, alkyl, haloalkyl, alkoxy, —CO-alkyl, —(CH₂)ₙSO₂R^{f} or —(CH₂)ₙSO₂NR^{c}R^{d};

R₂ is selected from hydrogen or —(CH₂)ₙ-cycloalkyl;

R₃ is selected from hydrogen, —(CH₂)ₙ—OH, —(CH₂)ₙ-cycloalkyl, an optionally substituted groups selected from alkyl, —(CH₂)ₙ-heterocyclyl, —(CH₂)ₙ-aryl, —(CH₂)ₙ-heteroaryl, wherein one or more optional substituents on R₃ are selected from halogen, —CH₂—OH, alkyl, aryl, —SO₂NR^{c}R^{d} or —SO₂R^{f};

R₄ is an optionally substituted group selected from alkyl, cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;

R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂)ₙSO₂NR^{c}R^{d};

R^{e} is hydrogen;

R^{d} and R^{e} are each independently selected from hydrogen, alkyl, alkoxy or aryl, which is optionally substituted with halogen;

R^{f} is selected from alkyl, cycloalkyl, heterocyclyl, aryl or heteroaryl; and

n is an integer from 0 to 2.

In a different embodiment, the invention includes compounds of the formula (V),

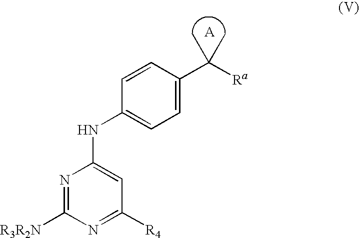

stereoisomers thereof and/or pharmaceutically acceptable salts of compounds of formula (V) and/or pharmaceutically acceptable salts of the stereoisomers of compounds of formula (V); wherein

R₂ and R₃ combine with the nitrogen atom, to which they are attached, to form an optionally substituted 5- to 6-membered cyclic ring optionally having 1 to 3 additional hetero atoms or groups selected from nitrogen, oxygen, sulfur, SO₂ or CO; wherein one or more optional substituents on the heterocyclic ring formed may be selected from halogen, hydroxy, alkoxy, alkyl, cycloalkyl or —CO-alkyl;

R₄ is an optionally substituted group selected from cycloalkyl, aryl, heterocyclyl, or heteroaryl, wherein one or more optional substituents on R₄ are selected from halogen, hydroxy, cyano, alkyl, haloalkyl, cycloalkyl, alkoxy or —CO-alkyl;

Ra is selected from —(CH₂)ₙ—OH, —(CH₂)ₙCOOR^{c}, —(CH₂)ₙCONR^{d}R^{e}, —(CH₂)ₙSO₂R^{f} or —(CH₂)ₙSO₂NR^{c}R^{d};

R^{c} is hydrogen;

R^{d} and R^{e} are each independently selected from hydrogen, alkyl or alkoxy;

R^{f} is selected from alkyl, cycloalkyl, heterocyclyl, aryl or heteroaryl; and

n is an integer from 0 to 2.

In some embodiments, the invention includes one or more compounds of formula (I), which are represented by
1-[4-(4-tert-Butylamino-6-phenyl-pyrimidin-2-ylamino)-phenyl]-cyclobutane carboxylic acid,
1-[4-(4-tert-Butylamino-6-phenyl-pyrimidin-2-ylamino)-phenyl]-cyclopropane carboxylic acid,
1-[4-(4-tert-Butylamino-6-phenyl-pyrimidin-2-ylamino)-phenyl]-cyclopentane carboxylic acid,
1-[4-(4-tert-Butyl-6-tert-butylamino-pyrimidin-2-ylamino)-phenyl]-cyclobutane carboxylic acid,
1-(4-{4-[(1-Ethyl-pyrrolidin-2-ylmethyl)-amino]-6-phenyl-pyrimidin-2-ylamino}-phenyl)-cyclobutanecarboxylic acid methyl ester,
1-[4-(4-tert-Butylamino-5-fluoro-6-phenyl-pyrimidin-2-ylamino)-phenyl]-cyclobutanecarboxylic acid,
1-{4-[4-(Bis-cyclopropylmethyl-amino)-6-(4-fluoro-phenyl)-pyrimidin-2-ylamino]-phenyl}-cyclobutanecarboxylic acid methyl ester,
1-{4-[4-(Cyclopropylmethyl-amino)-6-(4-fluoro-phenyl)-pyrimidin-2-ylamino]-phenyl}-cyclopentanecarboxylic acid,
1-{4-[4-(Cyclopropylmethyl-amino)-6-(4-fluoro-phenyl)-pyrimidin-2-ylamino]-phenyl}-cyclobutanecarboxylic acid,
1-[4-(4-Methylamino-6-phenyl-pyrimidin-2-ylamino)-phenyl]-cyclobutane carboxylic acid,
1-{4-[4-(tert-Butyl-methyl-amino)-6-phenyl-pyrimidin-2-ylamino]-phenyl}-cyclobutanecarboxylic acid,
1-{4-[4-(Bicyclo[2.2.1]hept-2-ylamino)-6-phenyl-pyrimidin-2-ylamino}-phenyl cyclobutanecarboxylic acid,
1-{4-[4-(4-Fluoro-phenyl)-6-morpholin-4-yl-pyrimidin-2-ylamino]-phenyl}-cyclobutanecarboxylic acid,
1-{4-[4-(4-Fluoro-phenyl)-6-(4-hydroxy-piperidin-1-yl)-pyrimidin-2-ylamino]-phenyl}-cyclobutanecarboxylic acid,
1-[4-(4-Cyclobutylamino-6-phenyl-pyrimidin-2-ylamino)-phenyl]-cyclobutane carboxylic acid,
1-[4-(4-Cyclopentylamino-6-phenyl-pyrimidin-2-ylamino)-phenyl]-cyclobutane carboxylic acid,
1-{4-[4-(1,1-Dimethyl-propylamino)-6-phenyl-pyrimidin-2-ylamino]-phenyl}-cyclobutanecarboxylic acid,
1-{4-[4-(4-Fluoro-phenylamino)-6-phenyl-pyrimidin-2-ylamino]-phenyl}-cyclobutanecarboxylic acid,
1-{4-[4-(4-Methanesulfonyl-phenylamino)-6-phenyl-pyrimidin-2-ylamino]-phenyl}-cyclobutanecarboxylic acid,
1-{4-[4-Phenyl-6-(1-phenyl-ethylamino)-pyrimidin-2-ylamino]-phenyl}-cyclobutanecarboxylic acid,
1-{4-[4-Phenyl-6-(1-phenyl-ethylamino)-pyrimidin-2-ylamino]-phenyl}-cyclobutanecarboxylic acid,
1-{4-[4-(2-Hydroxy-1-phenyl-ethylamino)-6-phenyl-pyrimidin-2-ylamino]-phenyl}-cyclobutanecarboxylic acid,
1-[4-(4-Amino-6-phenyl-pyrimidin-2-ylamino)-phenyl]-cyclobutanecarboxylic acid,
1-{4-[4-(5-Methyl-isoxazol-3-ylamino)-6-phenyl-pyrimidin-2-ylamino]-phenyl}-cyclobutanecarboxylic acid,
1-[4-(4-Cyclopentyl-6-cyclopentylamino-pyrimidin-2-ylamino)-phenyl]-cyclobutanecarboxylic acid,
1-[4-(4-tert-Butylamino-6-cyclopentyl-pyrimidin-2-ylamino)-phenyl]-cyclobutane carboxylic acid,
1-{4-[4-(1-Methyl-1H-pyrazol-4-yl)-6-morpholin-4-yl-pyrimidin-2-ylamino]-phenyl}-cyclobutanecarboxylic acid,
1-{4-[4-(1-Methyl-1H-pyrazol-4-yl)-6-pyrrolidin-1-yl -pyrimidin-2-ylamino]-phenyl}-cyclobutanecarboxylic acid,
1-{4-[4-(4-Hydroxy-piperidin-1-yl)-6-(1-methyl-1H-pyrazol-4-yl)-pyrimidin-2-ylamino]-phenyl}-cyclobutanecarboxylic acid,
1-{4-[4-(3,5-Dimethyl-isoxazol-4-yl)-6-morpholin-4-yl-pyrimidin-2-ylamino]-phenyl}-cyclobutanecarboxylic acid,
1-{3-[4-(3,5-Dimethyl-isoxazol-4-yl)-6-morpholin-4-yl-pyrimidin-2-ylamino]-phenyl}-cyclobutanecarboxylic acid,
1-{4-[4-(3,5-Dimethyl-isoxazol-4-yl)-5-methyl-6-morpholin-4-yl-pyrimidin-2-ylamino]-phenyl}-cyclobutanecarboxylic acid,
1-{4-[4-(3,5-Dimethyl-isoxazol-4-yl)-6-piperazin-1-yl-pyrimidin-2-ylamino]-phenyl}-cyclobutanecarboxylic acid,
1-{4-[4-(4-Acetyl-piperazin-1-yl)-6-(3,5-dimethyl-isoxazol-4-yl)-pyrimidin-2-ylamino]-phenyl}-cyclobutanecarboxylic acid,
1-{4-[4-(6-Methoxy-pyridin-3-yl)-6-morpholin-4-yl-pyrimidin-2-ylamino]-phenyl}-cyclobutanecarboxylic acid,
1-{4-[4-(4,4-Difluoro-piperidin-1-yl)-6-(3,5-dimethyl-isoxazol-4-yl)-pyrimidin-2-ylamino]-phenyl}-cyclobutanecarboxylic acid,
1-{4-[4-Butylamino-6-(3,5-dimethyl-isoxazol-4-yl)-pyrimidin-2-ylamino]-phenyl}-cyclobutanecarboxylic acid,
1-{4-[4-tert-Butylamino-6-(3,5-dimethyl-isoxazol-4-yl)-pyrimidin-2-ylamino]-phenyl}-cyclobutanecarboxylic acid,
1-{4-[4-Cyclopentylamino-6-(3,5-dimethyl-isoxazol-4-yl)-pyrimidin-2-ylamino]-phenyl}-cyclobutanecarboxylic acid,
1-[4-(4-Cyclopentylamino-6-phenyl-pyrimidin-2-ylamino)-phenyl]-cyclobutanecarbonitrile,
Sodium 1-[4-(4-tert-butylamino-6-phenyl-pyrimidin-2-ylamino)-phenyl]-cyclobutane carboxylate,
1-{4-(4-tert-Butylamino-6-phenyl-pyrimidin-2-ylamino)-phenyl]-cyclobutyl}-methanol,
1-[4-(4-tert-Butylamino-6-phenyl-pyrimidin-2-ylamino)-phenyl}-cyclobutane carboxylic acid ethylamide,
1-[4-(4-tert-Butylamino-6-phenyl-pyrimidin-2-ylamino)-phenyl]-cyclobutane carboxylic acid dimethylamide,
1-[4-(4-tert-Butylamino-6-phenyl-pyrimidin-2-ylamino)-phenyl}-cyclobutane carboxylic acid tert-butylamide,
1-[4-(4-tert-Butylamino-6-phenyl-pyrimidin-2-ylamino)-phenyl)-cyclobutane carboxylic acid amide,
1-[4-(4-tert-Butylamino-6-phenyl-pyrimidin-2-ylamino)-phenyl]-cyclobutane carboxylic acid methoxy-methyl-amide,
1-{1-[4-(4-tert-Butylamino-6-phenyl-pyrimidin-2-ylamino)-phenyl)-cyclobutyl}-ethanone,
N⁴-tert-Butyl-N²-[4-(1-oxazol-2-yl-cyclobutyl)-phenyl]-6-phenyl-pyrimidine-2,4-diamine,
1-[4-(4-tert-Butyl amino-6-phenyl-pyrimidin-2-ylamino)-phenyl]-cyclobutane carboxylic acid (4-fluoro-phenyl)-amide,
(1-{4-[4-(3,5-Dimethyl-isoxazol-4-yl)-6-morpholin-4-yl-pyrimidin-2-ylamino]-phenyl}-cyclobutyl)-methanol,
N⁴-tert-Butyl-N²-[4-(1-methoxymethyl-cyclobutyl)-phenyl]-6-phenyl-pyrimidine-2,4-diamine,
N⁴-tert-Butyl-N²-[4-(1-methyl-cyclobutyl)-phenyl]-6-phenyl-pyrimidine-2,4-diamine,
[4-(3,5-Dimethyl-isoxazol-4-yl)-6-morpholin-4-yl-pyrimidin-2-yl]-[4-(1-methanesulfonylmethyl-cyclobutyl)-phenyl]-amine,
1-[4-(2-tert-Butylamino-6-phenyl-pyrimidin-4-ylamino)-phenyl)-cyclobutane carboxylic acid,
1-{4-[2-(tert-Butyl-methyl-amino)-6-phenyl-pyrimidin-4-ylamino]-phenyl}-cyclobutanecarboxylic acid,
1-[4-(6-tert-Butylamino-2-phenyl-pyrimidin-4-ylamino)-phenyl]-cyclobutane carboxylic acid,
1-{4-[6-(3,5-Dimethyl-isoxazol-4-yl)-2-methyl-pyrimidin-4-ylamino]-phenyl}-cyclobutanecarboxylic acid,
1-{4-[2-(4-Fluoro-phenyl)-6-phenyl-pyrimidin-4-ylamino]-phenyl}-cyclobutanecarboxylic acid,
1-{4-[2-(4-Acetyl-phenyl)-6-phenyl-pyrimidin-4-ylamino]-phenyl}-cyclobutanecarboxylic acid,
1-{4-[6-(3,5-Dimethyl-isoxazol-4-yl)-2-(4-fluoro-phenyl)-pyrimidin-4-ylamino]-phenyl}-cyclobutanecarboxylic acid,
1-{4-[2-(6-Methoxy-pyridin-3-yl)-6-phenyl-pyrimidin-4-ylamino]-phenyl}-cyclobutanecarboxylic acid,
1-{4-[2-(4-Methanesulfonyl-phenyl)-6-phenyl-pyrimidin-4-ylamino]-phenyl}-cyclobutanecarboxylic acid,
1-{4-[2-(6-Methyl-pyridin-3-yl)-6-phenyl-pyrimidin-4-yl amino]-phenyl}-cyclobutanecarboxylic acid,
1-{4-[2-(1,1-Dioxo-1λ⁶-thiomorpholin-4-yl)-6-phenyl-pyrimidin-4-ylamino]-phenyl}-cyclobutanecarboxylic acid,
1-{4-[6-(3,5-Dimethyl-isoxazol-4-yl)-2-morpholin-4-yl-pyrimidin-4-ylamino]-phenyl}-cyclobutanecarboxylic acid,
1-[4-(2-Morpholin-4-yl-6-phenyl-pyrimidin-4-ylamino)-phenyl]-cyclobutane carboxylic acid,
1-[4-(4-Isopropoxy-6-phenyl-pyrimidin-2-ylamino)-phenyl]-cyclobutane carboxylic acid,
stereoisomers thereof and/or pharmaceutically acceptable salts of the compounds and/or pharmaceutically acceptable salts of the stereoisomers of the compounds.

In some embodiments, the invention includes one or more compounds of formula (III), which may be represented by:
1-[4-(4-tert-Butylamino-6-phenyl-pyrimidin-2-ylamino)-phenyl]-cyclobutanecarboxylic acid,
1-[4-(4-tert-Butylamino-6-phenyl-pyrimidin-2-yl-amino)-phenyl]-cyclopropanecarboxylic acid,
1-[4-(4-tert-Butylamino-6-phenyl-pyrimidin-2-ylamino)-phenyl]-cyclopentanecarboxylic acid,
1-[4-(4-tert-Butyl-6-tert-butylamino-pyrimidin-2-ylamino1-phenyl]-cyclobutanecarboxylic acid,
1-(4-{4-[(1-Ethyl-pyrrolidin-2-ylmethyl)-amino]-6-phenyl-pyrimidin-2-ylamino}-phenyl)-cyclobutanecarboxylic acid methyl ester,
1-[4-(4-tert-Butylamino-5-fluoro-6-phenyl-pyrimidin-2-ylamino)-phenyl]-cyclobutanecarboxylic acid,
1-{4-[4-(bis-Cyclopropylmethyl-amino)-6-(4-fluoro-phenyl)-pyrimidin-2-ylamino]-phenyl}-cyclobutanecarboxylic acid methyl ester,
1-{4-[4-(Cyclopropylmethyl-amino)-6-(4-fluoro-phenyl)-pyrimidin-2-ylamino]-phenyl}-cyclopentane-carboxylic acid,
1-{4-[4-(Cyclopropylmethyl-amino)-6-(4-fluoro-phenyl)-pyrimidin-2-ylamino]-phenyl}-cyclobutane-carboxylic acid,
1-[4-(4-Methylamino-6-phenyl-pyrimidin-2-ylamino)-phenyl]-cyclobutanecarboxylic acid,
1-{4-[4-(tert-Butyl-methyl-amino)-6-phenyl-pyrimidin-2-ylamino]-phenyl}-cyclobutane carboxylic acid,
1-{4-[4-(Bicyclo[2.2.1]hept-2-ylamino)-6-phenyl-pyrimidin-2-ylamino]-phenyl}-cyclobutane carboxylic acid,
1-[4-(4-Cyclobutylamino-6-phenyl-pyrimidin-2-ylamino)-phenyl]-cyclobutanecarboxylic acid,
1-[4-(4-Cyclopentylamino-6-phenyl-pyrimidin-2-ylamino)-phenyl]-cyclobutane-carboxylic acid,
1-{4-[4-(1,1-Dimethyl-propylamino)-6-phenyl-pyrimidin-2-ylamino]-phenyl}-cyclobutanecarboxylic acid,
1-{4-[4-(4-Fluoro-phenylamino)-6-phenyl-pyrimidin-2-ylamino]-phenyl}-cyclobutanecarboxylic acid,
1-{4-[4-(4-Methanesulfonyl-phenylamino)-6-phenyl-pyrimidin-2-ylamino]-phenyl}-cyclobutanecarboxylic acid,
(1-{4-[4-Phenyl-6-((R)-1-phenyl-ethylamino)-pyrimidin-2-ylamino]-phenyl}-cyclobutyl)-acetic acid,

(1-{4-[4-Phenyl-6-((S)-1-phenyl-ethylamino)-pyrimidin-2-ylamino]-phenyl}-cyclobutyl)-acetic acid,
1-{4-[4-((S)-2-Hydroxy-1-phenyl-ethylamino)-6-phenyl-pyrimidin-2-ylamino]-phenyl}-cyclobutanecarboxylic acid,
1-[4-(4-Amino-6-phenyl-pyrimidin-2-ylamino)-phenyl]-cyclobutane-carboxylic acid,
1-{4-[4-(5-Methyl-isoxazol-3-ylamino)-6-phenyl-pyrimidin-2-ylamino]-phenyl}-cyclobutanecarboxylic acid,
1-[4-(4-Cyclopentyl-6-cyclopentylamino-pyrimidin-2-ylamino)-phenyl]-cyclobutanecarboxylic acid,
1-[4-(4-tert-Butylamino-6-cyclopentyl-pyrimidin-2-ylamino)-phenyl]-cyclobutanecarboxylic acid,
1-{4-[4-Butylamino-6-(3,5-dimethyl-isoxazol-4-yl)-pyrimidin-2-ylamino]-phenyl}-cyclobutane carboxylic acid,
1-{4-[4-tert-Butylamino-6-(3,5-dimethyl-isoxazol-4-yl)-pyrimidin-2-ylamino]-phenyl}-cyclobutanecarboxylic acid,
1-{4-[4-Cyclopentylamino-6-(3,5-dimethyl-isoxazol-4-yl)-pyrimidin-2-yl-amino]-phenyl}-cyclobutanecarboxylic acid,
1-[4-(4-Cyclopentylamino-6-phenyl-pyrimidin-2-ylamino)-phenyl]-cyclobutanecarbonitrile,
Sodium 1-[4-(4-tert-butylamino-6-phenyl-pyrimidin-2-ylamino)-phenyl)-cyclobutane carboxylate,
{1-[4-(4-tert-Butylamino-6-phenyl-pyrimidin-2-ylmethyl)-phenyl]-cyclobutyl}-methanol,
1-[4-(4-tert-Butylamino-6-phenyl-pyrimidin-2-ylamino)-phenyl]-cyclobutanecarboxylic acid ethylamide,
1-[4-(4-tert-Butylamino-6-phenyl-pyrimidin-2-ylamino)-phenyl]-cyclobutanecarboxylic acid dimethylamide,
1-[4-(4-tert-Butylamino-6-phenyl-pyrimidin-2-ylamino)-phenyl]-cyclobutanecarboxylic acid tert-butylamide,
1-{4-(4-tert-Butylamino-6-phenyl-pyrimidin-2-ylamino)-phenyl]-cyclobutanecarboxylic acid methoxy-methyl-amide,
1-{1-[4-(4-tert-Butylamino-6-phenyl-pyrimidin-2-ylamino)-phenyl]-cyclobutyl}-ethanone,
N⁴-tert-Butyl-N²-[4-(1-oxazol-2-yl-cyclobutyl)-phenyl]-6-phenyl-pyrimidine-2,4-diamine,
1-[4-(4-tert-Butylamino-6-phenyl-pyrimidin-2-ylamino)-phenyl]-cyclobutanecarboxylic acid (4-fluoro-phenyl)-amide,
N⁴-tert-Butyl-N²-[4-(1-methoxymethyl-cyclobutyl)-phenyl]-6-phenyl-pyrimidine-2,4-diamine,
N⁴-tert-Butyl-N²-[4(1-methyl-cyclobutyl)-phenyl]-6-phenyl-pyrimidine-2,4-diamine,
stereoisomers thereof and/or pharmaceutically acceptable salts of the compounds and/or pharmaceutically acceptable salts of the stereoisomers of the compounds.

In some embodiments, the invention includes one or more compounds of formula (III), which may be represented by:
1-{4-[4-(4-fluoro-phenyl)-6-morpholin-4-yl-pyrimidin-2-ylamino]-phenyl}-cyclobutane-carboxylic acid,
1-{4-[4-(4-fluoro-phenyl)-6-(4-hydroxy-piperidin-1-yl)-pyrimidin-2-ylamino]-phenyl}-cyclobutane-carboxylic acid,
1-{4-[4-(1-methyl-1H-pyrazol-4-yl)-6-morpholin-4-yl-pyrimidin-2-ylamino]-phenyl}-cyclobutanecarboxylic acid,
1-{4-[4-(1-methyl-1H-pyrazol-4-yl)-6-pyrrolidin-1-yl-pyrimidin-2-ylamino]-phenyl}-cyclobutanecarboxylic acid,
1-{4-[4-(4-hydroxy-piperidin-1-yl)-6-(1-methyl-1H-pyrazol-4-yl)-pyrimidin-2-ylamino]-phenyl}-cyclobutanecarboxylic acid,
1-{4-[4-(3,5-dimethyl-isoxazol-4-yl)-6-morpholin-4-yl-pyrimidin-2-ylamino]-phenyl}-cyclobutanecarboxylic acid,
1-{3-[4-(3,5-dimethyl-isoxazol-4-yl)-6-morpholin-4-yl-pyrimidin-2-ylamino]-phenyl}-cyclobutanecarboxylic acid,
1-{4-[4-(3,5-dimethyl-isoxazol-4-yl)-5-methyl-6-morpholin-4-yl-pyrimidin-2-ylaminokphenyl}-cyclobutanecarboxylic acid,
1-{4-[4-(3,5-dimethyl-isoxazol-4-yl)-6-piperazin-1-yl-pyrimidin-2-ylamino]-phenyl}-cyclobutanecarboxylic acid,
1-{4-[4-(4-acetyl-piperazin-1-yl)-6-(3,5-dimethyl-isoxazol-4-yl)-pyrimidin-2-ylamino]-phenyl}-cyclobutanecarboxylic acid,
1-{4-[4-(6-methoxy-pyridin-3-yl)-6-morpholin-4-yl-pyrimidin-2-ylamino]-phenyl}-cyclobutanecarboxylic acid,
1-{4-[4-(4,4-difluoro-piperidin-1-yl)-6-(3,5-dimethyl-isoxazol-4-yl)-pyrimidin-2-ylamino]-phenyl}-cyclobutanecarboxylic acid,
(1-{4-[4-(3,5-dimethyl-isoxazol-4-yl)-6-morpholin-4-yl-pyrimidin-2-ylamino]-phenyl}-cyclobutyl)-methanol,
[4-(3,5-dimethyl-isoxazol-4-yl)-6-morpholin-4-yl-pyrimidin-2-yl]-[4-(1-methanesulfonyl methyl-cyclobutyl)-phenyl]-amine,
stereoisomers thereof and/or pharmaceutically acceptable salts of the compounds and/or pharmaceutically acceptable salts of the stereoisomers of the compounds.

In some embodiments, the invention includes one or more compounds of formula (IV), which may be represented by:
1-[4-(2-tert-butylamino-6-phenyl-pyrimidin-4-ylamino)-phenyl]-cyclobutane-carboxylic acid,
1-{4-[2-(tert-butyl-methyl-amino)-6-phenyl-pyrimidin-4-ylamino]-phenyl}-cyclobutane carboxylic acid,
1-[4-(6-tert-butylamino-2-phenyl-pyrimidin-4-ylamino)-phenyl]-cyclobutanecarboxylic acid,
1-{4-[6-(3,5-dimethyl-isoxazol-4-yl)-2-methyl-pyrimidin-4-ylamino]-phenyl}-cyclobutane carboxylic acid,
1-{4-[2-(4-fluoro-phenyl)-6-phenyl-pyrimidin-4-ylamino]-phenyl}-cyclobutanecarboxylic acid,
1-{4-[2-(4-acetyl-phenyl)-6-phenyl-pyrimidin-4-ylamino]-phenyl}-cyclobutanecarboxylic acid,
1-{4-[6-3,5-dimethyl-isoxazol-4-yl)-2-(4-fluoro-phenyl)-pyrimidin-4-ylamino]-phenyl}-cyclobutanecarboxylic acid,
1-{4-[2-(6-methoxy-pyridin-3-yl)-6-phenyl-pyrimidin-4-ylamino]-phenyl}-cyclobutane carboxylic acid,
1-{4-[2-(4-methanesulfonyl-phenyl)-6-phenyl-pyrimidin-4-ylamino]-phenyl}-cyclobutane carboxylic acid,
1-{4-[2-(6-methyl-pyridin-3-yl)-6-phenyl-pyrimidin-4-ylamino]-phenyl}-cyclobutanecarboxylic acid,
1-{4-[2-(1,1-dioxo-1λ⁶-thiomorpholin-4-yl)-6-phenyl-pyrimidin-4-ylamino]-phenyl}-cyclobutanecarboxylic acid,
1-{4-[6-(3,5-dimethyl-isoxazol-4-yl)-2-morpholin-4-yl-pyrimidin-4-ylamino]-phenyl}-cyclobutanecarboxylic acid,
1-[4-(2-morpholin-4-yl-6-phenyl-pyrimidin-4-ylamino)-phenyl]-cyclobutanecarboxylic acid,
stereoisomers thereof and/or pharmaceutically acceptable salts of the compounds and/or pharmaceutically acceptable salts of the stereoisomers of the compounds.

In one embodiment, the invention is directed to novel compounds of formula (I), stereoisomers thereof and/or pharmaceutically acceptable salts of formula (I) and/or pharmaceutically acceptable salts of the stereoisomers of formula (I) as GATA modulators.

In another embodiment, the invention is directed to novel compounds of formula (I), stereoisomers thereof and/or pharmaceutically acceptable salts of formula (I) and/or pharmaceutically acceptable salts of the stereoisomers of formula (I) as RCT enhancers.

In another embodiment, the invention is directed to a method for the treatment of atherosclerosis, diabetes and its associated complications in a subject, which comprises administering to the subject a therapeutically effective amount of a compound of formula (I), stereoisomers thereof and/or pharmaceutically acceptable salts of formula (I) and/or pharmaceutically acceptable salts of the stereoisomers of formula (I).

In another embodiment, the invention is directed to a method for the treatment of diabetic dyslipidemia, Alzheimer's disease and cardiovascular disease in a subject, which comprises administering to the subject a therapeutically effective amount of a compound of formula (I), stereoisomers thereof and/or pharmaceutically acceptable salts of formula (I) and/or pharmaceutically acceptable salts of the stereoisomers of formula (I).

In another embodiment, the diabetic-associated complications are those complications known in the art as likely resulting from the diabetic condition. Additionally, diabetic-associated complications are those complications known in the art that are exacerbated by the diabetic condition.

In one embodiment compounds of formula (I), stereoisomers and/or pharmaceutically acceptable salt thereof are useful for the treatment of hyperlipidemia, hypercholesterolemia, coronary heart disease, atherosclerosis, diabetes and its associated complications and Alzheimer's disease. The compounds of formula (I) may influence one or more lipid parameters such as increasing the HDL levels, lowering plasma levels of LDL, lowering plasma glucose, and/or lowering triglycerides.

As used hereinthroughout, the following definitions apply.

The groups defined for various symbols of the compounds of formulae (I)-(V) and optional substituents defined on those groups are defined in the detailed manner as follows:

‘Halogen or Halo’ represents one or more of fluorine, chlorine, bromine, or iodine.

‘Alkyl’ group refers to linear or branched alkyl groups. Exemplary alkyl groups include one or more of, but are not limited to, methyl, ethyl, n-propyl, iso-propyl, n-butyl, iso-butyl, t-butyl, n-pentyl, iso-pentyl, hexyl, heptyl, octyl and the like. Unless otherwise specified, an alkyl group typically has from 1 to 10 carbon atoms, but the invention is not limited in that respect.

‘Haloalky’ means at least one halogen atom is substituted on an alkyl group. Both halogen and alkyl have the meaning as defined above. Representative examples of haloalkyl groups include one or more of, but are not limited to, fluoromethyl, chloromethyl, fluoroethyl, chloroethyl, difluoromethyl, trifluoromethyl, dichloroethyl, dichloroethyl and the like. Unless otherwise specified, a halolkyl group typically has from 1 to 10 carbon atoms, but the invention is not limited in that respect.

‘Cycloalky’ group refers to a cyclic alkyl group which may be mono, bicyclic, polycyclic, or fused/bridged ring systems. Exemplary cycloalkyl groups include one or more of, but are not limited to, cyclopropyl, cyclobutyl, cyclopentyl, cyclohexyl, cycloheptyl, cyclooctyl, and the like. Unless otherwise specified, a cycloalkyl group typically has from 3 to about 10 carbon atoms. Typical bridged cycloalkyls include, but are not limited to, adamantyl, noradamantyl, bicyclo[1.1.0]butanyl, norboranyl(bicyclo[2.2.1]heptanyl), norbornenyl (bicyclo[2.2.1]heptanyl), norbornadienyl(bicyclo[2.2.1]heptadienyl), bicyclo[2.2.1]heptanyl, bicyclo[3.2.1]octanyl, bicyclo[3.2.1]octanyl, bicyclo[3.2.1]octadienyl, bicyclo[2.2.2]octanyl, bicyclo[2.2.2]octenyl, bicycl0[2.2.2]octadienyl, bicyclo[5.2.0]nonanyl, bicyclo[4.3.2]undecanyl, tricyclo[5.3.1.1]dodecanyl, and the like.

‘3 to 7 membered cycloalkyl’ group refers to a monocyclic alkyl group having 3 to 7 carbon atoms but the invention is not limited in that respect. ‘3 to 7 membered cycloalkyl’ groups include one or more of, but are not limited to, cyclopropyl, cyclobutyl, cyclopentyl, cyclohexyl and cycloheptyl.

‘Alkoxy’ represents an —O-alkyl group, where alkyl is as defined above. Exemplary alkoxy groups include one or more of, but are not limited to, methoxy, ethoxy, propoxy, butoxy, iso-propoxy, iso-butoxy, and the like. Unless otherwise specified, an alkoxy group typically has from 1 to about 10 carbon atoms but the invention is not limited in that respect.

‘Aryl’ is an optionally substituted monocyclic or polycyclic aromatic ring system. Exemplary aryl groups include one or more of, but are not limited to, phenyl, naphthyl, and the like. Unless otherwise specified, an aryl group typically has from 6 to about 14 carbon atoms but the invention is not limited in that respect.

‘Heteroaryl’ is an aromatic monocyclic or polycyclic ring system, having at least one heteroatom or heterogroup selected from —O—, —N—, —S—, —SO₂, or —CO. Exemplary heteroaryl groups include one or more of, but are not limited to, pyrazinyl, isothiazolyl, oxazolyl, isooxazolyl, pyrazolyl, pyrrolyl, pyridazinyl, thienopyrimidyl, furanyl, indolyl, isoindolyl, benzo[1,3]dioxolyl, 1,3-benzoxathiole, pyrrolidine 2,4-dione, quinazolinyl, pyridyl, thiophenyl, and the like. Unless otherwise specified, a heteroaryl group typically has from 4 to about 10 carbon atoms but the invention is not limited in that respect.

‘Heterocycly’ is a non-aromatic saturated monocyclic or polycyclic ring system of 3 to 10 members having at least one heteroatom or heterogroup selected from one or more of —O—, —N—, —S—, —SO₂, or —CO. Exemplary heterocyclyl groups include one or more of, but not limited to, pyrrolidinyl, piperidinyl, piperazinyl, morpholinyl, thiomorpholinyl, thiomorpholine 1,1-dioxide, thiazolidinyl, 1,3-dioxolanyl, 1,4-dioxanyl, and the like. Unless otherwise specified, a heterocyclyl group typically has from 3 to about 10 carbon atoms but the invention is not limited in that respect.

‘5- to 6-Membered heterocyclyl’ is a saturated monocyclic ring system of 5 or 6 ring atoms but the invention is not limited in that respect, having at least one heteroatom that is a nitrogen atom. The rings may optionally contain an additional 1 to 2 heteroatoms or heterogroups selected from one or more of —O—, —N—, —S—, —SO₂, or —CO. Exemplary ‘5- to 6-membered heterocyclyl’ groups include one or more of, but are not limited to, pyrrolidinyl, piperdinyl, piperazinyl, morpholinyl, thiomorpholinyl, thiomorpholine 1,1-dioxide and the like.

‘Optionally substituted’ means that substitution is optional and therefore it is possible for the designated atom or molecule to be unsubstituted. In the event a substitution is desired, then such substitution means that any number of hydrogens on the designated atom is replaced with a selection from the indicated group, provided that the normal valence of the designated atom is not exceeded, and that the substitution results in a stable compound. For example, in formula (I) when a substituent is keto (i.e., ═O), then 2 hydrogens on the atom are replaced and when the substitution is fluoro, then 1 hydrogen on the atom is replaced and the like.

The one or more compounds of formula (I) can be supplied in the form of a novel therapeutic composition that is within the scope of the present invention.

‘Salts’ refer to any acid or base salt, pharmaceutically acceptable solvates, or any complex of the compound that, when administered to a recipient, is capable of providing (directly or indirectly) a compound as described herein. It should be appreciated, however, that salts that are not pharmaceutically acceptable also lie within the scope of the invention. The preparation of salts can be carried out using known methods.

For example, pharmaceutically acceptable salts of compounds contemplated herein may be synthesized by conventional chemical methods using a parent compound containing a base or an acid residue. Generally, such salts may be prepared, for example, by making free acid or base forms of the compounds and reacting with a stoichiometric quantity of the appropriate base or acid in water or in an organic solvent, or in a mixture of the two. Generally, non-aqueous media such as one or more of ether, ethyl acetate, ethanol, isopropanol or acetonitrile may be utilized. Examples of acid addition salts include one or more of, but are not limited to, mineral acid addition salts such as hydrochloride, hydrobromide, hydroiodide, sulphate, nitrate, phosphate, and organic acid addition salts such as acetate, maleate, fumarate, citrate, oxalate, succinate, tartrate, malate, mandelate, methanesulphonate and p-toluenesulphonate. Examples of base addition salts include one or more of, but are not limited to, inorganic salts such as sodium, potassium, calcium, ammonium, magnesium, and lithium salts, and organic base salts such as ethylenediamine, ethanolamine, N,N-dialkyl-ethanolamine, triethanolamine, glucamine and basic amino acid salts.

Also included in present invention are the isomeric forms and tautomers and the pharmaceutically-acceptable salts of compounds of formula (I). Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acids.

Suitable pharmaceutically-acceptable base addition salts of compounds of the present invention include metallic ion salts and organic ion salts. Commonly used metallic ion salts include one or more of, but are not limited to, appropriate alkali metal (Group IA) salts, alkaline earth metal (Group IIA) salts, and other physiological acceptable metal ions. Such salts can be made from one or more of the ions of aluminum, calcium, lithium, magnesium, potassium, sodium, and zinc. Exemplary organic salts can be made from one or more of tertiary amines and quaternary ammonium salts, including in part, trimethylamine, diethylamine, N,N′-dibenzylethylenediamine, chloroprocaine, choline, diethanolamine, ethylenediamine, meglumine (N- methylglucamine) and procaine. All of the above salts may be prepared by those skilled in the art by conventional means from the corresponding compound of the present invention.

For any particular compound disclosed herein, any general structure presented also encompasses all confoimational isomers, regioisomers, stereoisomers and tautomers that may arise from a particular set of substituents.

Compounds of formula (I) contain more than one asymmetric carbons. It is to be understood accordingly that the stereoisomers arising from such asymmetry (e.g., all enantiomers and diastereomers) are included within the scope of this invention, unless explicitly indicated otherwise. Such isomers can be obtained in substantially pure form by classical separation techniques and by stereochemically controlled synthesis.

Contemplated derivatives are those that increase the bioavailability of the compounds of this invention when such compounds are administered to a subject (e.g., by making an orally administered compound more easily absorbed). Formulae (I)-(V) compounds can be amorphous, semi-crystalline, or crystalline and may be given as parent compounds, its salts, and/or in solvated form. The solvate may be part of a crystalline lattice or superficially associated. It is intended that all of these forms should be within the scope of the present invention. Methods of solvation are generally known within the art. Suitable solvates are pharmaceutically acceptable solvates. In one embodiment, the solvate is a hydrate.

The GATA family of transcription factors is known to include GATA1, GATA2, GATA3, GATA4, GATA5, and GATA6. As used herein, the term GATA shall be interpreted as including one or more of the GATA family of transcription factors unless explicitly stated otherwise.

As used herein, the term ‘subject’ means mammals, such as humans and other animals, including horses, dogs, cats, rats, mice, sheep, pigs, etc. In exemplary embodiments, the subject may include subjects for which treatment and/or prevention of the conditions described herein would be beneficial.

For ease of reference, the present invention will be described in terms of administration to human subjects. It will be understood, however, that such descriptions are not limited to administration to humans, but will also include administration to other animals unless explicitly stated otherwise.

The phrase ‘therapeutically effective’ indicates the capability of an agent to prevent, or improve the severity of, the disorder, while avoiding adverse side effects typically associated with alternative therapies.

The terms ‘treating’ or ‘to treat’ means to alleviate symptoms, eliminate the causation either on a temporary or permanent basis, or to prevent or slow the appearance of symptoms. The term ‘treatment’ includes alleviation, elimination of causation of or prevention of any of the diseases or disorders described above. Besides being useful for human treatment, these combinations are also useful for treatment of other mammals, including horses, dogs, cats, rats, mice, sheep, pigs, etc.

The compounds described herein are typically administered in admixture with one or more pharmaceutically acceptable excipients or carriers in the form of a pharmaceutical composition. A ‘composition’ may contain one compound or a mixture of compounds. A ‘pharmaceutical composition’ is any composition useful or potentially useful in producing at least one physiological response in a subject to which such pharmaceutical composition is administered.

The pharmaceutical compositions of compounds of formulae (I)-(V) may be administered enterally and/or parenterally. Parenteral administration includes subcutaneous, intramuscular, intradermal, intramammary, intravenous, and other administrative methods known in the art. Enteral administration includes solution, tablets, sustained release capsules, enteric coated capsules, syrups, beverages, foods, and other nutritional supplements. When administered, the present pharmaceutical compositions may be at or near body temperature. In some embodiments, the present pharmaceutical compositions may be below body temperatures. In other embodiments, the present pharmaceutical compositions may be above body temperatures.

The compounds of the present invention may be administered in a wide variety of different dosage forms. For example, they may be combined with various pharmaceutically acceptable inert carriers in the form of one or more of, but not limited to, tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers may include one or more of solid diluents or fillers, sterile aqueous media, and various nontoxic organic solvents, etc. Moreover, oral pharmaceutical compositions may be sweetened and/or flavored. In general, the compounds of the invention may be present in such dosage forms at concentration levels ranging from about 0.1% to about 90% by weight.

For oral administration, tablets may contain various excipients such as one or more of microcrystalline cellulose, sodium citrate, calcium carbonate, dicalcium phosphate, and glycine, along with various disintegrants such as starch (such as corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulphate and talc may be employed. Solid compositions of a similar type may also be employed as fillers in gelatin capsules; exemplary materials in this connection may also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents, together with diluents such as water, ethanol, propylene glycol, glycerin, and various combinations thereof

For parenteral administration (including intraperitoneal subcutaneous, intravenous, intradennal or intramuscular injection), solutions of compounds of the present invention in, for example, either sesame or peanut oil or in aqueous propylene glycol may be employed. The aqueous solutions may be buffered, if necessary or desirable, and the liquid diluent first rendered isotonic. These aqueous solutions may be suitable for intravenous injection purposes. The oily solutions may be suitable for intraarticular, intramuscular, and/or subcutaneous injection purposes. The preparation of such solutions under sterile conditions may be accomplished by standard pharmaceutical techniques known to those having ordinary skill in the art. For parenteral administration, examples of suitable preparations may include solutions, such as oily or aqueous or non-aqueous solutions, as well as suspensions, emulsions, and/or implants, including suppositories. Compounds of the present invention may be formulated in sterile form in multiple or single dose formats. For example, the compounds of the present invention may be dispersed in a fluid carrier such as sterile saline and/or 5% saline dextrose solutions commonly used with injectables.

In another embodiment, the compounds of the present invention may be administered topically. For example, it may be desirable to administer the compounds of the present invention topically when treating inflammatory conditions of the skin. Non-limiting examples of methods of topical administration include transdermal, buccal, or sublingual application. For topical applications, therapeutic compounds may be suitably admixed in a pharmacologically inert topical carrier such as a gel, an ointment, a lotion, and/or a cream. Such topical carriers may include water, glycerol, alcohol, propylene glycol, fatty alcohols, triglycerides, fatty acid esters, and/or mineral oils. Other possible topical carriers may include liquid petrolatum, isopropylpalmitate, polyethylene glycol, ethanol 95%, polyoxyethylene monolaurate 5% in water, sodium lauryl sulphate 5% in water, and the like, and combinations thereof. In addition, materials such as surfactants, anti-oxidants, humectants, viscosity stabilizers, and the like, and combinations thereof, also may be added if desired.

It will be appreciated by those having ordinary skill in the art that the exemplary amounts of active compounds used in a given therapy will vary according to the specific compound being utilized, the particular compositions formulated, the mode of application, the particular site of administration, etc. Optimal administration rates for a given protocol of administration may be ascertained by those having ordinary skill in the art using conventional dosage determination tests conducted with regard to the foregoing guidelines.

In general, compounds of the present invention for treatment may be administered to a subject in a suitable effective dose of one or more compounds of the present invention may be in the range of from about 0.01 to about 100 milligrams per kilogram of body weight of recipient per day, in some embodiments, in the range of from about 0.5 to about 50 milligrams per kilogram body weight of recipient per day, in still other embodiments, in the range of from about 0.1 to about 20 milligrams per kilogram body weight of recipient per day. The exemplary dose may be suitably administered once daily, or several sub-doses, e.g. 2 to 5 sub-doses, may be administered at appropriate intervals through the day, or on other appropriate schedules.

An embodiment of the present invention provides preparation of the novel compounds of formulae (I)-(V) according to the procedures of the following examples, using appropriate materials. Those skilled in the art will understand that known variations of the conditions and processes of the following preparative procedures can be used to prepare these compounds. Moreover, by utilizing the procedures described in detail, one of ordinary skill in the art can prepare additional compounds of the present invention claimed herein. All temperatures are in degrees Celsius unless otherwise noted.

### Examples

The following acronyms, abbreviations, teinis and definitions have been used throughout the reaction scheme and experimental section.

### Acronyms or Abbreviations:

THF (tetrahydrofuran), TBME (tert-butyl methyl ether), MeOH (methanol), Ether (diethyl ether), EtOAc (ethyl acetate), DMF (N,N-dimethylformamide), DMSO (dimethyl sulfoxide), NMM (N-methyl morpholine), DIEA [(N,N-diisopropylethylamine) (Hünig's base)], LiHMDS (lithium hexamethyldisilazine), Pd₂dba₃ [tris(dibenzylideneacetone)dipalladium(0)], dppp [1,3-bis(diphenylphosphino)propane], TMS (tetramethylsilane), TFA (trifluoracetic acid), DDQ (2,3-dichloro-5,6-dicyano-p-benzoquinone), HATU [O-(-7-azabenzotriazol-1-yl)-N,N,N′,N′-tetramethyluronium hexafluorophosphate], EDAC (N-(3-dimethylaminopropyl)-N′-ethylcarbodiimide hydrochloride), TBAF (tetrabutylammmonium fluoride), HOBt (1-hydroxybenzotriazole), h (hour), min (minute), TLC (thin layer chromatography), LCMS (liquid chromatographic mass spectroscopy), MS (mass spectroscopy), NMR (nuclear magnetic resonance), HPLC (high performance liquid chromatography), HPFC (high performance flash chromatography), Mp/mp (melting point), aq (aqueous).

NMR abbreviations: br (broad), apt (apparent), s (singlet), d (doublet), t (triplet), q (quartet), dd (doublet of doublets), m (multiplet).

The following general schemes describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

The compound of formula (I) can be synthesized by following any of the process explained in following General Scheme A to E, wherein all symbols/variables are as defined earlier:

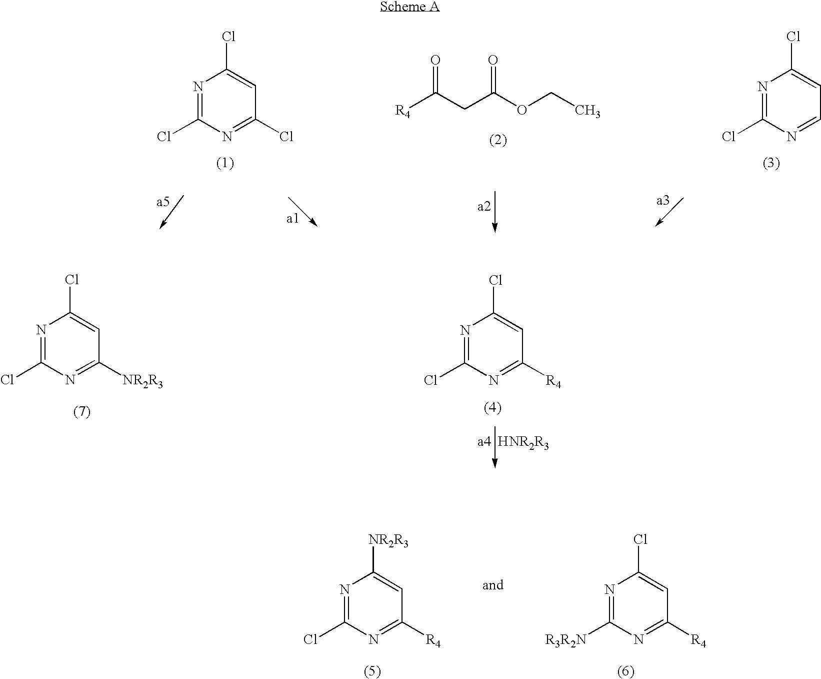

Pathway a1: Reacting 2,4,6-trichloropyrimidine (1), with R₄B(OH)₂, wherein R₄ is same as in the description of compound of general formula (I), using a Pd metal catalyst such as palladium acetate, tetrakis(triphenylphosphine)palladium and the like, in the presence of a solvent such as THF, dioxane and the like, and a ligand such as triphenylphosphine under thermal conditions at about 60° C. to obtain a compound of general formula (4). Or, compound (4) could be prepared by using a Pd catalyzed coupling methodology such as a Suzuki-type reaction.

Pathway a2: Alternatively, a compound of general formula (4) can be prepared in two step process by condensing a compound of general formula (2), wherein R₄ is the same as defined in compound of general formula (I), with urea under thermal heating conditions followed by reaction with phosphorus oxychloride.

Pathway a3: Alternatively, a compound of general formula (4) can be prepared by reacting 2,4-dichloropyrimidine (3) with R₄Li, wherein R₄ is the same as defined in compound of general formula (I), using aprotic solvent such as diethyl ether, THF, dioxane and the like, followed by oxidation reaction using solvents like DDQ.

Pathway a4: Reacting a compound of general formula (4) with HNR₂R₃, wherein R₂ and R₃ are the same as defined in compound of general formula (I), in the presence of a Pd metal catalyst such as palladium acetate, Pd₂dba₃ and the like, using a solvent such as THF, dioxane and the like a ligand such as triphenylphosphine, and a base such as LiHMDS, KHMDS and the like, to obtain a compound of general formula (5) and potentially (6). Or, compound (5) and potentially compound (6) could be prepared by using a Pd catalyzed amination methodology.

Alternatively, reacting a compound of general formula (4) with HNR₂R₃, wherein R₂ and R₃ are the same as defined in compound of general formula (I), in the absence or presence of a base such as K₂CO₃, DIEA and the like, using a solvent such as DMF, methanol, 1-butanol and the like, at temperature ranging from about 0° C. to reflux temperature to obtain a compound of general formula (5) and (6) which could be isolated and separated using trituration, recrystallization, or chromatography conditions.

Pathway a5: Reacting a compound of general formula (1) with HNR₂R₃, wherein R₂ and R₃ are the same as defined in compound of general formula (I), in the presence or absence of a base such as K₂CO₃, DIEA and the like, using a solvent such as DMF, methanol or 1-butanol and the like, at thermal conditions ranging from about 0° C. to the reflux temperature of the solvent used, to obtain a compound of general formula (7).

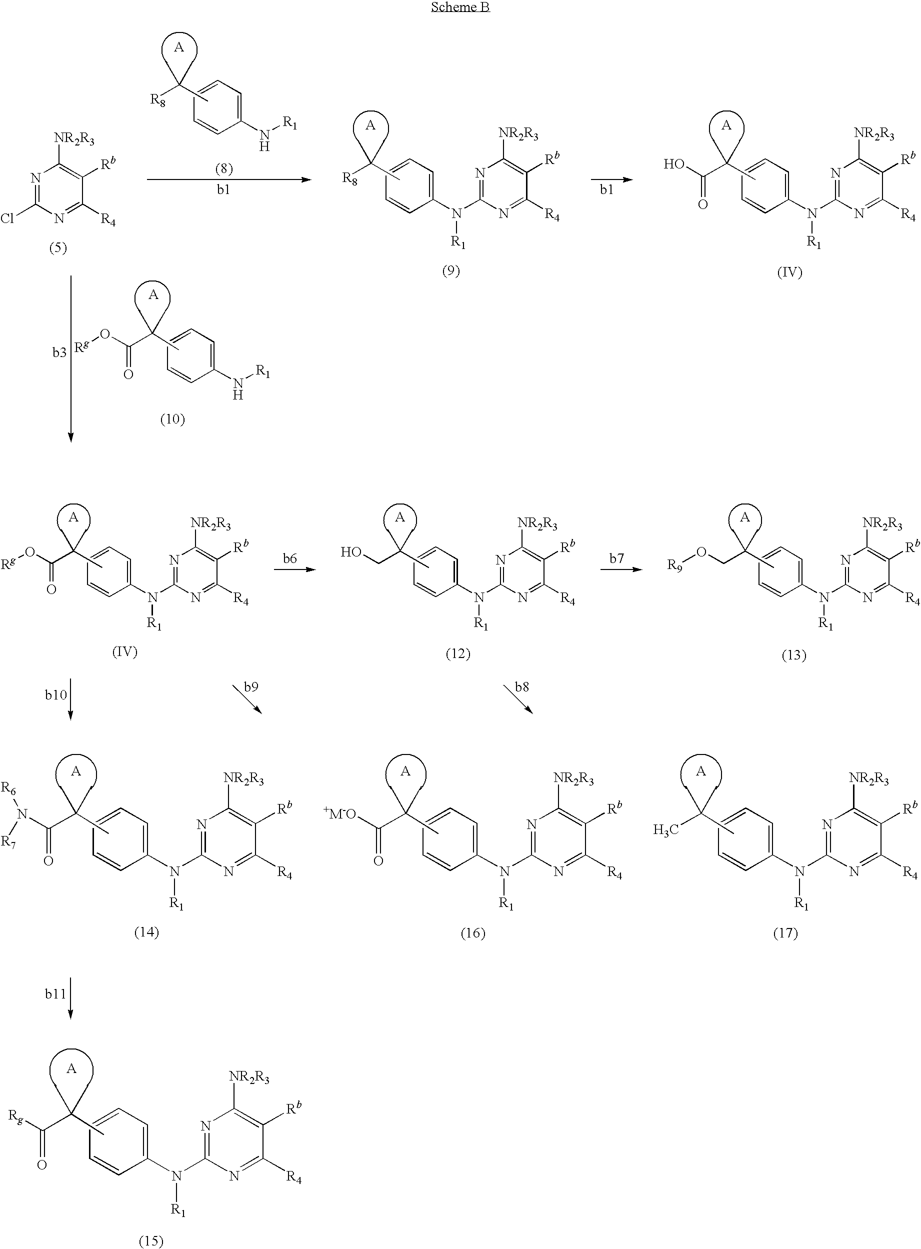

Pathway b1: Reacting a compound of general formula (5), wherein R^{b}, R₂, R₃ and R₄, are the same as defined in compound of general formula (I) with a compound of formula (8), wherein A and Rᵢ are the same as defined in compound of general formula (I) and R₈ is cyano, in the presence of a solvent such as 1-butanol, 2-propanol and the like under thermal heating conditions to obtain a compound of formula (9).

Pathway b2: Hydrolyzing a compound of general formula (9) wherein R^{b}, A, R₁, R₂, R₃ and R₄ are the same as defined in compound of general formula (I) and R₈ is cyano, using aqueous acid such as aq HCl and the like or base such as NaOH, in the presence of a solvent such as 2-propanol/water, to obtain a compound of formula (IV).

Pathway b3: Reacting a compound of general formula (5), wherein R^{b}, R₂, R₃ and R₄ are the same as defined in compound of general formula (I) with a compound of general formula (10), wherein A and R₁ are the same as defined in compound of general formula (I) and R^{g} is an alkyl, cycloalkyl, aryl, heterocyclyl or heteroaryl group, using a Pd catalyst such as Pd₂dba₃, tetrakis(triphenylphosphine)palladium and the like, in the presence of a solvent such as toluene and the like, a ligand such as dppp and the like, and a base such as NaOtBu, KOtBu and the like, at a temperature at about 60° C., to obtain a compound of general formula (11).

Pathway b4: Hydrolyzing a compound of general formula (11) wherein A, R^{b}, R₁, R₂, R₃ and R₄ are the same as defined in compound of general formula (I) and R^{g} is an alkyl, cycloalkyl, aryl, heterocyclyl or heteroaryl group, by following the procedure as described for pathway b2, to obtain a compound of general formula (IV).

Pathway b5: A compound of general formula (12) can be prepared by reacting a compound of general formula (11), wherein A, R^{b}, R₁, R₂, R₃ and R₄ are the same as defined in compound of general formula (I) and R^{g} is an alkyl, cycloalkyl, aryl, heterocyclyl or heteroaryl group, with a reducing agent such as lithium aluminum hydride and the like, at an appropriate temperature such as ambient to reflux temperature, in the presence of a solvent such as THF and the like.

Pathway b6: Reacting a compound of general formula (IV) wherein A, R^{b}, R₁, R₂, R₃ and R₄ are the same as defined in compound of general formula (I), with a reducing reagent such as lithium aluminum hydride and the like, in the presence of a solvent such as THF and the like, to obtain a compound of general formula (12).

Pathway b7: Reacting a compound of general formula (12) wherein A, R^{b}, R₁, R₂, R₃ and R₄ are the same as defined in description of compound of general formula (I), with methanesulfonyl chloride in the presence of a base such as triethylamine and the like, in a solvent such as dichloromethane and the like, at an appropriate temperature to form a mesylated intermediate. The mesylate can be reacted with sodium methoxide in a solvent such as MeOH and the like, at ambient temperature to yield a compound of general formula (13), where R₉ is an alkyl.

Pathway b8: Reacting a compound of general formula (12) wherein A, R^{b}, R₁, R₂, R₃ and R₄ are the same as defined in compound of general formula (I), with methanesulfonyl chloride in the presence of a base such as triethylamine, DIEA and the like, in a solvent such as dichloromethane and the like, at an appropriate temperature to form a mesylated intermediate which can be isolated and subsequently treated with a reducing reagent such as lithium aluminum hydride and the like, in the presence of a solvent such as THF and the like, to obtain a compound of general formula (17).

Pathway b9: Reacting a compound of general formula (IV), wherein A, R^{b}, R₁, R₂, R₃ and R₄ are the same as defined in compound of general formula (I), with a base such as potassium tert-butoxide, sodium tert-butoxide and the like, in a solvent such as MeOH and the like, at an appropriate temperature such as ambient to reflux temperature to obtain a compound of general formula (16).

Pathway b 10: Reacting compound of general formula (IV) with NHR₆R₇, wherein R₆ and R₇ independently selected from alkyl or alkoxy group, in the presence of coupling reagent such as EDAC and the like, and reagents such as HOBt, NMM and the like, in a solvent such as dichloromethane and the like, or a coupling reagent such as HATU and base such as DIEA and the like, in the presence of a solvent such as DMF and the like, to obtain a compound of general formula (14).

Pathway b11: Reacting a compound of general formula (14), wherein A, R^{b}, R₁, R₂, R₃ and R₄ are the same as defined in compound of general formula (I) and R₆ and R₇ are independently selected from an alkyl or alkoxy group, with R₉Li, wherein R₉ is alkyl group, in the presence of a solvent such as THF and under thermal conditions such as at about reflux temperature to prepare a compound of general formula (15).

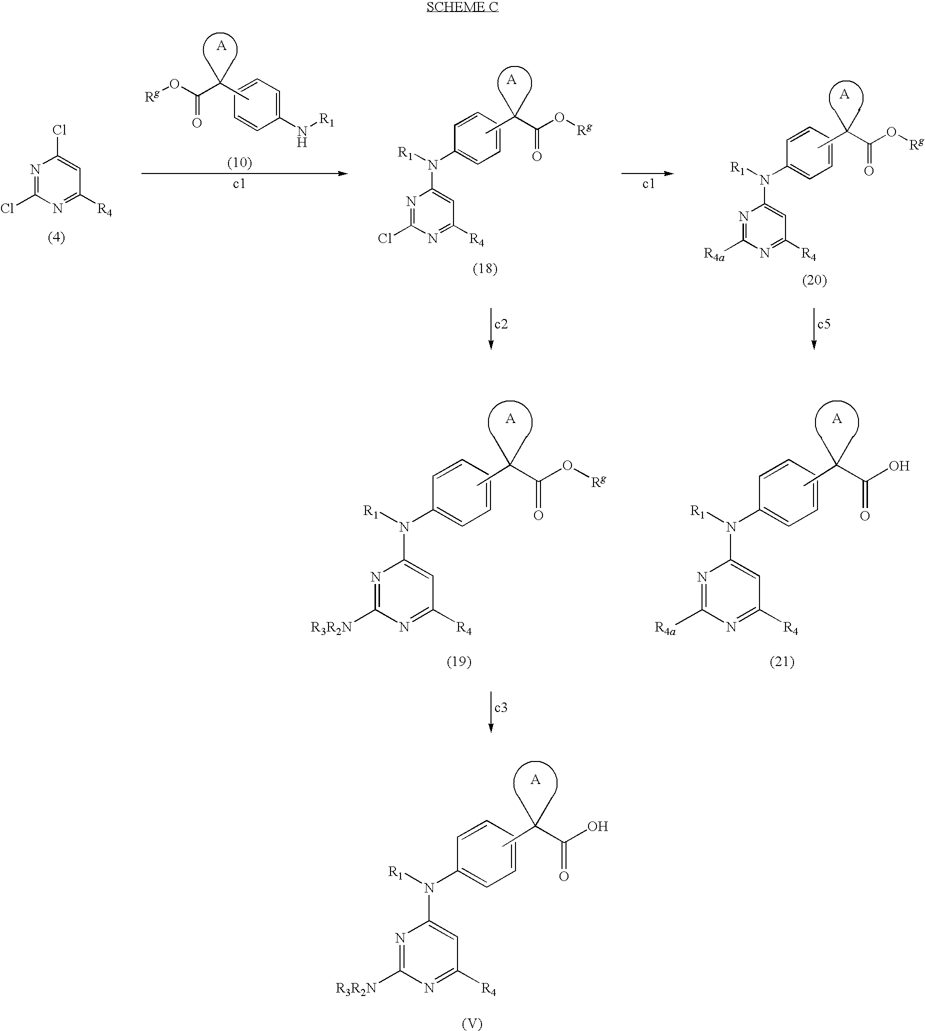

Pathway c1: Reacting a compound of general formula (4), wherein R₄ is the same as defined in compound of general formula (I), with a compound of general formula (10), wherein A and R₁ are the same as defined in description of compound of general formula (I) and R^{g} is an alkyl, cycloalkyl, aryl, heterocyclyl or heteroaryl group, using a base such as LiHMDS and the like, in the presence of a solvent such as THF and the like, at a temperature ranging from about −78° C. to ambient temperature to obtain a compound of general formula (18) and possibly its regioisomer which could be isolated and separated using trituration, recrystallization, or chromatography.

Pathway c2: Reacting a compound of general formula (18), wherein A, R₁ and R₄ are the same as defined in compound of general formula (I) and R^{g} is an alkyl, cycloalkyl, aryl, heterocyclyl or heteroaryl group, with HNR₂R₃, wherein R₂ and R₃ are the same as defined in compound of general formula (I), in the presence or absence of a base such as K₂CO₃, DIEA and the like, in the presence of a solvent such as DMF, methanol, 1-butanol and the like, at a temperature ranging from about 0° C. to reflux temperature to obtain a compound of general formula (19).

Alternatively, reacting a compound of general formula (18) with HNR₂R₃, wherein R₂ and R₃ are the same as defined in compound of general formula (I), in the presence of an excess of HNR₂R₃ such as morpholine as a solvent, at thermal condition such as at about the reflux temperature to obtain a compound of general formula (19) or general formula (V) upon pH adjustment and isolation.

Pathway c3: Reacting a compound of general formula (19) wherein A, R₁, R₂, R₃ and R₄ are the same as defined in compound of general formula (I) and R^{g} is an alkyl, cycloalkyl, aryl, heterocyclyl or heteroaryl group, with a base such as NaOH and the like, in the presence of a solvent such as methanol, water and the like, under thermal heating conditions such as at about the reflux temperature followed by acidification with HCl (aq) to obtain a compound of general formula (V).

Pathway c4: Reacting a compound of general formula (18), wherein A, R₁ and R₄ are the same as defined in compound of general formula (I) and R^{g} is an alkyl, cycloalkyl, aryl, heterocyclyl or heteroaryl group, with R₄ₐB(OH)₂, wherein R₄ₐ is selected from aryl, heteroaryl, with Pd metal catalyst such as Pd(PPh₃)₄] and the like, in the presence of a solvent such as THF, diglyme and the like, and a base such as sodium carbonate and the like, under thermal conditions such as at about the reflux temperature to obtain a compound of general formula (20). Or, a compound of formula (20) could be prepared by using a Pd catalyzed coupling methodology such as a Suzuki-type reaction.

Pathway c5: Reacting a compound of general formula (20), wherein A, R₁ and R₄ are the same as defined in compound of general formula (I), R₄ₐ is selected from aryl, heteroaryl; in the presence of a base such as NaOH and the like, in the presence of a solvent such as methanol, water and the like, under thermal heating conditions such as at about the reflux temperature, followed by acidification with HCl (aq) to obtain a compound of general formula (21).

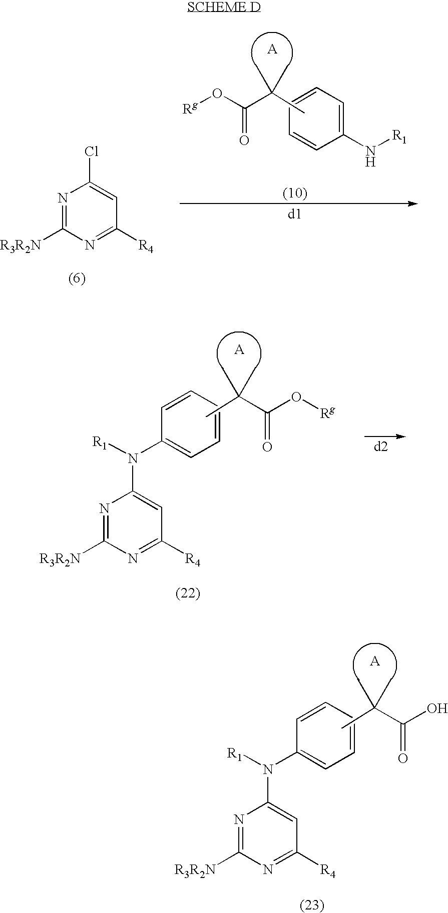

Pathway d1: Reacting a compound of general formula (6) wherein R₂, R₃ and R₄ are the same as defined in compound of general formula (I), with a compound of general formula (10), wherein A and R₁ are the same as defined in compound of general formula (I) and R^{g} is an alkyl, cycloalkyl, aryl, heterocyclyl or heteroaryl group, in the presence of a solvent such as 1-butanol and the like, under thermal conditions such as at about reflux temperature, to obtain a compound of general formula (22).

Alternatively, reacting a compound of general formula (6) wherein R₂, R₃ and R₄ are the same as defined in compound of general formula (I), with a compound of general formula (10), wherein A and R₁ are the same as defined in compound of general formula (I) and R^{g} is an alkyl, cycloalkyl, aryl, heterocyclyl or heteroaryl group, in the presence of a Pd metal catalyst such as Pd₂dba₃ and the like, a solvent such as toluene and the like, a ligand such as dppp and the like, and a base such as NaOtBu and the like, at a temperature at about 60° C. to obtain a compound of general formula (22). Or, a compound of formula (22) could be prepared by using a Pd catalyzed amination reaction.

Pathway d2: Reacting a compound of general foimula (22) wherein A, R₁, R₂, R₃ and R₄ are the same as defined in compound of general formula (I), and R^{g} is an alkyl, cycloalkyl, aryl, heterocyclyl or heteroaryl group, with a base such as KOH and the like, in the presence of a solvent such as 2-propanol, water and the like, under thermal conditions such as at about reflux temperature, followed by acidification with HCl (aq) to obtain a compound of general formula (23).

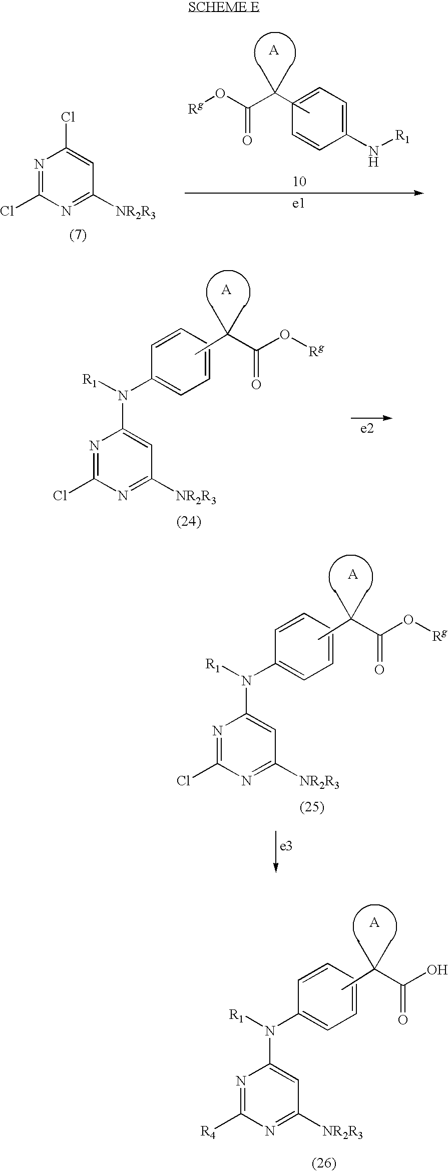

Pathway e1: Reacting a compound of general formula (7), wherein R₂ and R₃ are the same as defined in compound of general formula (I), with a compound of general formula (10), wherein A and R₁ are the same as defined in compound of general formula (I) and R^{g} is selected from an alkyl, cycloalkyl, aryl, heterocyclyl or heteroaryl group, in the presence of a Pd metal catalyst such as Pd₂dba₃ and the like, a solvent such as toluene and the like, a ligand such as dppp and the like, and a base such as NaOtBu and the like, at a temperature at about 60° C. to obtain a compound of general formula (24). Or, compound of formula (24) could be prepared by using a Pd catalyzed amination reaction.

Pathway e2: Reacting a compound of general formula (24), wherein A, R₁, R₂ and R₃ are the same as defined in compound of general formula (I) and R^{g} is selected from an alkyl, cycloalkyl, aryl, heterocyclyl or heteroaryl group, with R₄B(OH)₂, wherein R₄ is same as in the description of compound of general formula (I), with a Pd catalyst such as Pd(PPh₃)₄ and the like, in the presence of a solvent such as THF, diglyme and the like, in the presence of a base such as sodium carbonate and the like, under thermal conditions such as the reflux temperature to obtain a compound of general formula (25). Or, compound of formula (25) could be prepared by using a Pd catalyzed coupling methodology such as a Suzuki-type reaction.

Pathway e3: Reacting a compound of general formula (25), wherein A, R₁, R₂, R₃ and R₄ are the same as defined in compound of general formula (I) and R^{g} is an alkyl, cycloalkyl, aryl, heterocyclyl or heteroaryl group, with a base such as NaOH and the like, in the presence of a solvent such as 2-propanol, water and the like, under thermal heating conditions such as at about the reflux temperature, followed by acidification with HCl (aq) to obtain a compound of general formula (26).

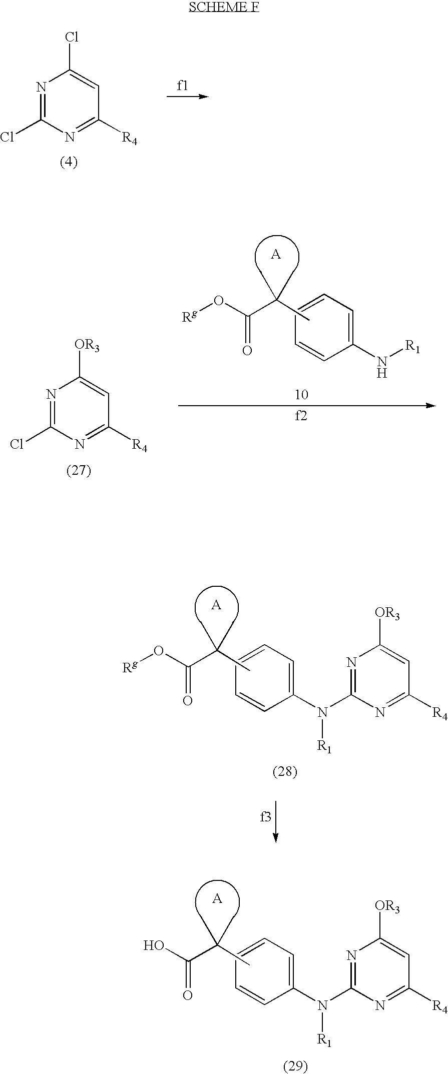

Pathway f1: Reacting a compound of general formula (4), wherein R₄ is same as in the description of compound of general formula (I), with R₃OH, wherein R₃ is an alkyl group, in the presence of a base such as K₂CO₃, Cs₂CO₃ and the like, and a solvent such as DMF and the like, at thermal conditions ranging from about 60 to 100° C. to obtain a compound of general formula (27) and could be isolated and separated using titration, recrystallization, or chromatography conditions.

Pathway f2 Reacting a compound of general formula (27) wherein R₄ is same as in the description of compound of general formula (I) and R₃ is an alkyl group, with a compound of general formula (10), wherein A and R₁ are the same as defined in compound of general formula (I) and R^{g} is selected from an alkyl, cycloalkyl, aryl, heterocyclyl or heteroaryl group, in the presence of a solvent such as 1-butanol and the like, under thermal conditions such as at about 100° C. or at the reflux temperature to obtain a compound of general formula (28).

Pathway f3: Reacting a compound of general formula (28), wherein A, R₁ and R₄ are the same as defined in compound of general formula (I) and R₃ and R^{g} is selected from an alkyl, cycloalkyl, aryl, heterocyclyl or heteroaryl group, in the presence of a base such as NaOH and the like, in the presence of a solvent such as MeOH, water and the like, under thermal heating conditions at about 100° C., followed by acidification with HCl (aq) to obtain a compound of general formula (29).

The novel compounds of the present invention were prepared according to the procedure of the following schemes and examples, using appropriate materials and are further exemplified by the following specific examples. Exemplary compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention.

Those skilled in the art will readily understand that known variations of the conditions and processes of the following preparative procedures can be used to prepare these compounds.

### Example 1

### Synthesis of 1-[4-(4-tert-butylamino-6-phenyl-pyrimidin-2-ylamino)-phenyl]-cyclobutanecarboxylic acid

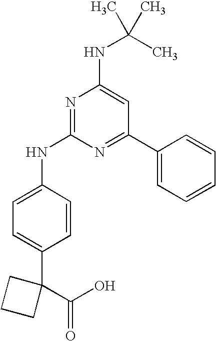

### Step (i): Synthesis of the intermediate, 1-(4-amino-phenyl)-cyclobutanecarboxylic acid methyl ester

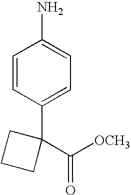

### (a) Synthesis of (4-nitro-phenyl)-acetic acid methyl ester

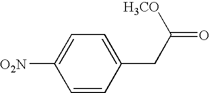

To 4-nitrophenyl acetic acid (50.12 g, 276 mmol) dissolved in methanol (300 mL) was added concentrated sulfuric acid (8 mL). The reaction mixture was stirred at reflux for 18 h under nitrogen atmosphere. The methanol was removed in vacuo and the resulting residue was diluted with H₂O. The reaction mixture was extracted two times with CH₂Cl₂, and then washed with 0.5% NaHCO₃. The organic phase was dried over Na₂SO₄, filtered, and concentrated by rotary evaporation. The resulting solid was dried under vacuum for about 10-19 h. The title compound as pale yellow solid was used as such with no further purification (52.2 g, 97% yield).

¹H NMR (300 MHz, CDCl₃, TMS): δ 3.72 (s, 3H), 3.74 (s, 2H), 7.43 to 7.48 (m, 2H), 8.17-8.21 (m, 2H).

LC-MSD (ES+): (m/z) 196 [(M+H)⁺, 100].

### (b) Synthesis of 1-(4-nitro-phenyl)-cyclobutanecarboxylic acid methyl ester

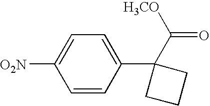

(4-Nitro-phenyl)-acetic acid methyl ester (15.21 g, 76.9 mmol) was dissolved in anhydrous DMF (80 mL) and allowed to stir until all solid dissolved. The solution was cooled to 0° C. with an ice bath. Sodium hydride (6.32 g, 153.8 mmol; 60% dispersion in oil) was added slowly and cautiously. The resulting mixture was allowed to warm to about 20-35° C. and was stirred for approximately 15 min. The solution was cooled again to 0° C. with an ice bath. 1,3-Diiodopropane (17.7 mL, 153.8 mmol) was added slowly and drop-wise, and the resulting solution was allowed to stir at 0° C. for about 30 min. The solution was warmed to about 20-35° C. and stirred at about 20-35° C. for about 1 h. The solution was cooled to 0° C. with an ice bath and quenched with H₂O, maintaining 0° C. throughout the quenching process. The reaction mixture was extracted 3 times with dichloromethane. The combined organic layers were washed with water and brine; dried over sodium sulfate; filtered; and then concentrated under vacuum to give product. The material was purified by column chromatography (SiO₂, 80:20 hexane: EtOAc) and gave the title compound as a yellow solid material (7.5 g, 41% yield).

¹H NMR (300 MHz, CDCl₃, TMS) δ 1.87-2.19 (m, 2H), 2.48-2.58 (m, 2H), 2.86-2.95 (m, 2H), 3.67 (s, 3H), 7.43 to 7.48 (m, 2H), 8.17-8.21 (m, 2H).

LC-MSD (ES+): (m/z) 236 [(M+H)⁺, 100].

### (c) Synthesis of 1-(4-amino-phenyl)-cyclobutanecarboxylic acid methyl ester

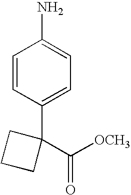

To 1-(4-nitro-phenyl)-cyclobutanecarboxylic acid methyl ester (7.3 g, 31 mmol) dissolved in ethanol (70 mL) was added tin (II) chloride dihydrate (28.02 g, 124 mmol). The resulting solution was stirred at 85° C. for approximately 4 h. After cooling to about 20-35° C., the solution was adjusted to a pH ˜10 with concentrated NaOH (aq). The mixture was extracted 3 times with EtOAc. The combined organic layers were washed with water and brine; dried over sodium sulfate; filtered; and then concentrated by rotary evaporation. The title compound, brown solid, was dried under vacuum for about 10-19 h and was used without further purification (5.7 g, 90% yield).

¹H NMR (300 MHz, CDCl₃, TMS): δ 1.79-2.05 (m, 2H), 2.38-2.49 (m, 2H), 2.72-2.82 (m, 2H), 3.62 (s, 3H), 3.71 (s, 2H), 6.63-6.68 (m, 2H), 7.07-7.12 (m, 2H).

LC-MSD (ES+): (m/z) 206 [(M+H)⁺, 100].

### Step (ii): Synthesis of 2,4-dichloro-6-phenyl-pyrimidine

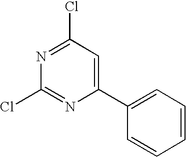

The title compound was prepared by following procedures reported in (a) Schomaker, J. M.; Delia, T. J. J. Org. Chem. 2001, 66, 7125-7128, and (b) Peng, Z-H.; Journet, M.; Humphrey, G. Org. Lett. 2006, 8, 395-398.

To 2,4,6-trichloropyrimidine (25.01 g, 136 mmol) dissolved in THF was added phenylboronic acid (13.97 g, 114 mmol), palladium acetate (II) (516.2 g, 2.3 mmol), triphenylphosphine (1.193 g, 4.5 mmol) and sodium carbonate (114 mL, 228 mmol, 2 M) sequentially. The resulting mixture was stirred at reflux in a 60° C. oil bath under a nitrogen atmosphere for 3.5 h. The mixture was diluted with TBME, and was washed two times with water and one time with brine. The organic phase was dried over sodium sulfate, filtered, and concentrated by rotary evaporation. The solid was triturated with ether and hexanes, filtered, and dried under vacuum to give title compound as a white solid (13.7 g, 45% yield).

HPLC: Inertsil ODS-3V C18, 30:70 [KH₂PO₄ (0.01M, pH 3.2): CH₃CN], 264 nm, Rₜ 11.7 min, 92% purity.

¹H NMR (300 MHz, CDCl₃, TMS): δ 8.24-8.21 (m, 2H), 8.07-8.03 (m, 2H), 7.64 (s, 1H), 7.54-7.50 (m, 1H).

LC-MSD (ES+): m/z [226 (M+H)⁺, 100].

### Step (iii): Synthesis of tert-butyl-(2-chloro-6-phenyl-pyrimidin-4-yl)-amine

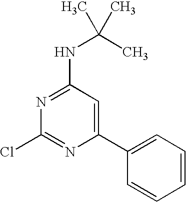

2,4-Dichloro-6-phenyl-pyrimidine (10.9 g, 45 mmol) was dissolved in tert-butylamine (40 mL, 379 mmol). The resulting mixture was stirred at reflux for 1.5 h under nitrogen atmosphere. The mixture was concentrated by rotary evaporation. The solid was triturated with ether and hexanes, filter, and dried under vacuum to give title compound as white solid (6.8 g, 58% yield).

HPLC: Inertsil ODS-3V C18, 30:70 [KH₂PO₄ (0.01M, pH 3.2): CH₃CN], 264 nm, Rₜ 14.7 min, 98.6% purity.

¹H NMR (300 MHz, CDCl₃, TMS): δ 7.94-7.90 (m, 2H), 7.46-7.44 (m, 2H), 6.62 (s, 1H), 1.48 (s, 9H).

LC-MSD (ES+): m/z [262 (M+H)⁺, 100].

### Step (iv): Synthesis of 1-[4-(4-tert-butylamino-6-phenyl-pyrimidin-2-ylamino)-phenyl]-cyclobutanecarboxylic acid methyl ester

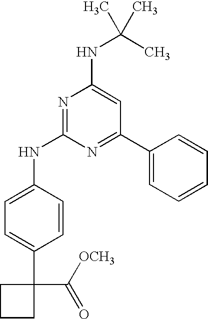

tert-Butyl-(2-chloro-6-phenyl-pyrimidin-4-yl)-amine (5.89 g, 22.2 mmol) was dissolved in n-butanol (50 mL) and 1-(4-aminophenyl)-cyclobutane-carboxylic acid methyl ester (5.46 g, 26.6 mmol) was added. The resulting mixture was stirred at reflux for about 10-19 h under nitrogen atmosphere. The mixture was diluted with dichloromethane, and was washed two times with water and one time with brine. The organic phase was dried over sodium sulfate, filtered, and concentrated by rotary evaporation. The resulting sample was dried for about 10-19 h under vacuum. Flash column chromatography (SiO₂, 80:20 hexane: EtOAc) purification gave the title compound as a pale yellow solid (4.96 g, 51% yield).

HPLC: Inertsil ODS-3V C18, 30:70 [KH₂PO₄ (0.01M, pH 3.2): CH₃CN], 264 nm, Rₜ 8.5 min, 97% purity.

¹H NMR (300 MHz, CDCl₃, TMS): δ 7.85 (br s, 2H), 7.70 (d, J=4.2 Hz, 2H), 7.45-7.25 (m, 6H), 6.74 (s, 1H), 3.64 (s, 3H), 2.87-2.78 (m, 2H), 2.54-2.44 (m, 2H), 2.05-1.84 (m, 2H), 1.47 (s, 9H).

LC-MSD (ES+): m/z [431 (M+H)⁺, 100].

### Step (v): Synthesis of 1-[4-(4-tert-butylamino-6-phenyl-pyrimidin-2-ylamino)-phenyl}-cyclobutanecarboxylic acid:

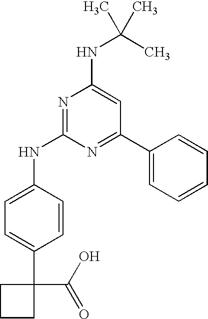

1-[4-(4-tert-Butylamino-6-phenyl-pyrimidin-2-ylamino)-phenyl]-cyclobutanecarboxylic acid methyl ester (4.88 g, 4.4 mmol) was dissolved in methanol (50 mL) then a solution of NaOH (1.597 g, 39.6 mmol) in 20 mL water was added. The resulting mixture was stirred at reflux for about 10-19 h under nitrogen atmosphere. The mixture was concentrated by rotary evaporation to remove most of the methanol and was adjusted to pH 3-4 with dilute HCl (aq). The precipitate was then filtered and washed with pentane. The resulting white solid was dried under vacuum to give the title compound (3.8 g, 80% yield).

HPLC: Inertsil ODS-3V C18, [KH₂PO₄ (0.01M, pH 3.2): CH₃CN], gradient, 264 nm, Rₜ 13.9 min, 97.6% purity.

¹H NMR (300 MHz, DMSO-d₆): δ 12.23 (b s, 1H), 9.23 (s, 1H), 7.92 (d, J=6.3 Hz, 2H), 7.78 (d, J—8.4 Hz, 2H), 7.52 (d, J=6.3 Hz, 2H), 7.24-7.18 (m, 3H), 6.51 (s, 1H), 2.74-2.68 (m, 2H), 2.40-2.33 (m, 2H), 1.94-1.85 (m, 2H), 1.45 (s, 9H).

LC-MSD (ES+): m/z [417 (M+H)⁺, 100].

### Example 2

### Synthesis of 1-[4-(4-tert-butylamino-6-phenyl-pyrimidin-2-yl-amino)-phenyl]-cyclopropanecarboxylic acid

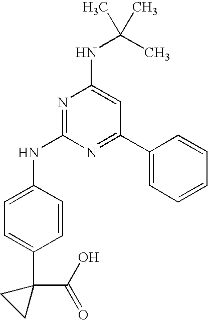

### Step (i): Synthesis of 1-(4-amino-phenyl)-cyclopropanecarboxylic acid methyl ester

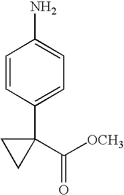

### (a) Synthesis of 1-(4-nitro-phenyl)-cyclopropanecarboxylic acid methyl ester

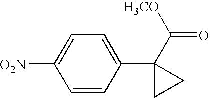

(4-Nitro-phenyl)-acetic acid methyl ester (2.50 g, 12.8 mmol) was dissolved in anhydrous DMF (20 mL) and stirred until the entire solid dissolved. The solution was cooled to 0° C. with an ice bath. Sodium hydride (1.02 g, 25.6 mmol; 60% dispersion in oil) was added slowly and cautiously. The resulting mixture was allowed to warm to about 20-35° C. and was stirred at about 20-35° C. for approximately 20 min. The solution was cooled again to 0° C. with an ice bath. 1,2-Dibromoethane (2.2 mL, 25.6 mmol) was added drop wise, and the resulting solution was allowed to stir at 0° C. for about 30 min. The solution was warmed to about 20-35° C. and stirred at about 20-35° C. for about 1 h. The solution was cooled to 0° C. with an ice bath and quenched with H₂O, maintaining 0° C. throughout the quenching process. The reaction mixture was extracted 2 times with dichloromethane. The combined organic layers were washed with water and brine; dried over sodium sulfate; filtered; and then concentrated under vacuum to give product. The material was purified by column chromatography (SiO₂, 70:30 hexane: EtOAc) and gave the title compound as a white solid material (1.26 g, 44% yield).

¹H NMR (300 MHz, CDCl₃, TMS) δ 8.15-8.19 (m, 2H), 7.54-7.49 (m, 2H), 3.64 (s, 3H), 1.79-1.68 (m, 2H), 1.26-1.22 (m, 2H).

LC-MSD (ES+): (m/z) 222 [(M+H)⁺, 100].

### (b) Synthesis of 1-(4-amino-phenyl)-cyclopropanecarboxylic acid methyl ester

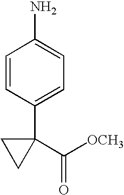

A similar method was used as in Example 1, step (ic) 1-(4-nitro-phenyl)-cyclopropanecarboxylic acid methyl ester was used instead of 1-(4-nitro-phenyl)-cyclobutanecarboxylic acid methyl ester. The title compound pale yellow solid, was dried under vacuum for about 10-19 h and was used without further purification (1.1 g, 96% yield).

¹H NMR (300 MHz, CDCl₃, TMS): δ 7.13 to 7.08 (m, 2H), 6.58-6.64 (m, 2H), 3.60 (s, 3H), 1.55-1.52 (m, 2H), 1.13-1.10 (m, 2H).

LC-MSD (ES+): (m/z) 192 [(M+H)⁺, 100].

### Step (ii): Synthesis of 1-[4-(4-tert-butylamino-6-phenyl-pyrimidin-2-ylamino)-phenyl]-cyclopropanecarboxylic acid methyl ester

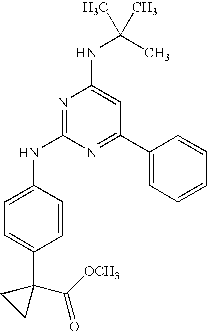

A similar method was used as in Example 1, step (iv) by using 1-(4-amino-phenyl)-cyclopropane-carboxylic acid methyl ester instead of 1-(4-amino-phenyl)-cyclobutane-carboxylic acid methyl ester. Purification (Biotage Horizon HPFC chromatography system, SiO₂, 70:30 hexanes: EtOAc) gave the title compound as a light yellow solid (240 mg, 16% yield).

HPLC: Betasil ODS-3V C18, 30:70 [KH₂PO₄ (0.01M, pH 3.2): CH₃CN], 264 nm, Rₜ 5.9 min, 96% purity.

¹H NMR (300 MHz, CDCl₃, TMS): δ 7.95-7.92 (m, 2H), 7.63 to 7.61 (m, 2H), 7.46-7.41(m, 3H), 7.29-7.26 (m, 2H), 7.03 (br s, 1H), 6.26 (s, 1H), 4.81(s, 1H), 3.62 (s, 3H), 1.59-1.55 (m, 2H), 1.48 (s, 9H), 1.19-1.15 (m, 2H).

LC-MSD (ES+): m/z [417 (M+H)⁺, 100].

### Step (iii): Synthesis of 1-[4-(4-tert-butylamino-6-phenyl-pyrimidin-2-ylamino)-phenyl]-cyclopropanecarboxylic acid

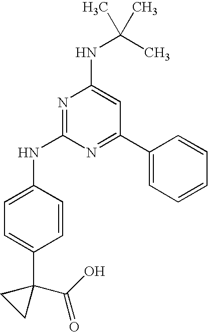

A similar method was used as in Example 1, step (v) by using 1-[4-(4-tert-butylamino-6-phenyl-pyrimidin-2-ylamino)-phenyl]-cyclopropane carboxylic acid methyl ester instead of 1-[4-(4-tert-butylamino-6-phenyl-pyrimidin-2-ylamino)-phenyl]-cyclobutanecarboxylic acid methyl ester. The title compound (220 mg, 95% yield) was isolated as a pale yellow solid.

HPLC: Betasil ODS-3V C18, [KH₂PO₄ (0.01M, pH 3.2): CH₃CN], gradient, 264 nm, Rₜ 12.6 min, 97.6% purity.

¹H NMR (300 MHz, DMSO-d₆): δ 12.24 (br s, 1H), 9.59 (br s, 1H), 7.91-7.88 (m, 2H), 7.67-7.54 (m, 5H), 7.26 (d, J=8.4 Hz, 3H), 6.53 (s, 1H), 1.44 (s, 9H), 1.1 (s, 4H).

LC-MSD (ES+): m/z [403 (M+H)⁺, 100].

### Example 3

### Synthesis of 1-[4-(4-tert-butylamino-6-phenyl-pyrimidin-2-ylamino)-phenyl]-cyclopentanecarboxylic acid

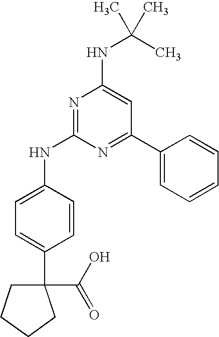

### Step (i): Synthesis of 1-[4-(4-tert-butylamino-6-phenyl-pyrimidin-2-ylamino)-phenyl]-cyclopentanecarboxylic acid methyl ester

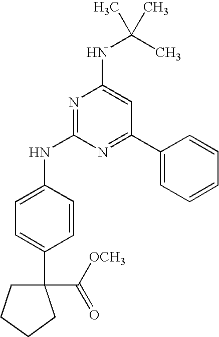

A similar method was used as in Example 1, step (iv) by using 1-(4-aminophenyl)-cyclopentane-carboxylic acid methyl ester instead of 1-(4-aminophenyl)-cyclobutane-carboxylic acid methyl ester. Purification (Biotage Horizon HPFC chromatography system, SiO₂, 70:30 hexanes: EtOAc) gave the title compound as a light yellow solid (310 mg, 18% yield).

HPLC: Betasil ODS-3V C18, 30:70 [KH₂PO₄ (0.01M, pH 3.2): CH₃CN], 264 nm, Rₜ 9.6 min, 97% purity.

¹H NMR (300 MHz, CDCl₃, TMS): δ 7.95-7.92 (m, 2H), 7.64-7.61 (m, 2H), 7.47-7.42 (m, 3H), 7.32-7.29 (m, 2H), 6.91 (s, 1H), 6.26 (s, 1H), 4.77 (s, 1H), 3.61 (s, 3H), 2.67-2.59 (m, 2H), 1.94-1.87 (m, 3H), 1.76-1.71 (m, 3H), 1.49 (s, 9H).

LC-MSD (ES+): m/z [445 (M+H)⁺, 100].

### Step (ii): Synthesis of 1-[4-(4-tert-butylamino-6-phenyl-pyrimidin-2-ylamino)-phenyl]-cyclopentanecarboxylic acid

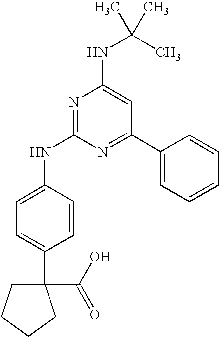

A similar method was used as in Example 1, step (v) by using 1-[4-(4-tert-butylamino-6-phenyl-pyrimidin-2-ylamino)-phenyl]-cyclopentane carboxylic acid methyl ester instead of 1-[4-(4-tert-butylamino-6-phenyl-pyrimidin-2-ylamino)-phenyl]-cyclobutanecarboxylic acid methyl ester. The resulting light yellow solid was dried under vacuum to give the title compound (295 mg, 98% yield).

HPLC: Betasil ODS-3V C18, [KH₂PO₄ (0.01M, pH 3.2): CH₃CN], gradient, 264 mu, Rₜ 14.9 min, 98.8% purity.

¹H NMR (300 MHz, DMSO-d₆): δ 12.26 (br s, 1H), 10.38 (br s, 1H), 8.47 (br s, 1H), 7.87-7.85 (m, 2H), 7.60-7.54 (m, 5H), 7.34 (d, J=8.7 Hz, 2H), 6.59 (s, 1H), 2.53-2.48 (m, 5H), 1.80-1.40 (m, 12H).

LC-MSD (ES+): m/z [431 (M+H)⁺, 100].

### Example 4

### Synthesis of 1-[4-(4-tert-butyl-6-tert-butylamino-pyrimidin-2-ylamino]-phenyl]-cyclobutanecarboxylic acid

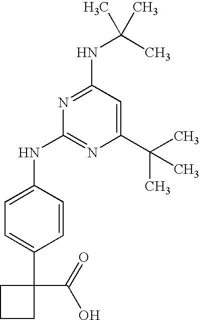

### Step (i): Synthesis of 4-tert-butyl-2,6-dichloro-pyrimidine

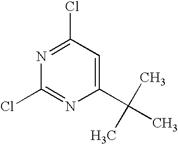

The title compound was prepared by adapting a procedure described by Harden, D. B.; Mokrosz, M. J.; Stekowski, L. J. Org. Chem. 1988, 53, 4137-4140.

To 2,4-dichloropyrimidine (2.509 g, 16.8 mmol) was dissolved in ether (anhydrous, 40 mL), cooled to −30° C., was added to tert-butyl lithium (10.3 mL, 17.5 mmol, 1.7 M). The mixture was stirred for 30 min under nitrogen atmosphere. The solution was allowed to warm to 0° C. and stirred for another 30 min. The mixture was quenched with⁻a solution of acetic acid (1 mL, 17.5 mmol) and water (0.15 mL, 9.4 mmol) in THF (3 mL). After stirring for few min, a solution of DDQ (3.976 g, 17.5 mmol) in THF (15 mL) was added. The resulting mixture was stirred for another 5 min at about 20-35° C. The solution is cooled to 0° C., and slowly a cool solution of 3M NaOH (6.7 mL, 20.16 mmol) was added. The solution was stirred at 0° C. for 5 min, filtered though Celite, and rinsed with dichloromethane. The filtrate was washed one time with water and one time with brine. The organic layer was dried over sodium sulfate and concentrated by rotary evaporation. The resulting solid was dried under vacuum. Flash chromatography purification (SiO₂, 80:20 hexane: EtOAc) gave the title compound as a pale yellow solid (2.2 g, 63% yield).

HPLC: Inertsil ODS-3V C18, 30:70 [KH₂PO₄ (0.01M, pH 3.2): CH₃CN], 264 nm, Rₜ 12.2 min, 99.6% purity.

¹H NMR (300 MHz, CDCl₃, TMS): δ 7.27 (s, 1H), 1.34 (s, 9H).

LC-MSD (ES+): m/z [205 (M+H)⁺, 100].

### Step (ii): Synthesis of tert-butyl-(6-tert-butyl-2-chloro-pyrimidin-4-yl)-amine

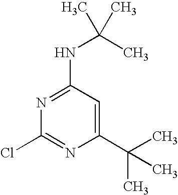

4-tert-Butyl-2,6-dichloro-pyrimidine (2.02 g, 45 mmol) was dissolved in tert-butylamine (8.5 mL, 80.51 mmol). The resulting mixture was stirred and refluxed for 1.5 h under nitrogen atmosphere. The mixture was concentrated by rotary evaporation. The resulting solid was dried under vacuum. Purification (flash chromatography system, SiO₂, 90:10 hexane: EtOAc) gave title compound as a white solid (430 mg, 18% yield).

HPLC: Inertsil ODS-3V C18, 30:70 [KH₂PO₄ (0.01M, pH 3.2): CH₃CN], 264 nm, Rₜ 15.3 min, 99.7% purity.

¹H NMR (300 MHz, CDCl₃, TMS): δ 6.19 (s, 1H), 4.96 (s, 1H), 1.43 (s, 9H), 1.26 (s, 9H).

LC-MSD (ES+): m/z [242 (M+H)⁺, 100].

### Step (iii): Synthesis of 1-[4-(4-tert-butyl-6-tert-butylamino-pyrimidin-2-ylamino)-phenyl}-cyclobutanecarboxylic acid methyl ester

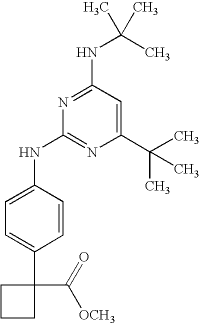

tert-Butyl-(6-tert-butyl-2-chloro-pyrimidin-4-yl)-amine (430 mg, 1.8 mmol) was dissolved in n-butanol (10 mL) followed by addition of 1-(4-aminophenyl)-cyclobutanecarboxylic acid methyl ester (0.449 g, 2.16 mmol). The resulting mixture was stirred at reflux for about 10-19 h under nitrogen atmosphere. The mixture was diluted with dichloromethane, and was washed two times with water and one time with brine. The organic phase was dried over sodium sulfate and concentrated by rotary evaporation. The resulting sample was dried for about 10-19 h under vacuum. Purification (flash chromatography system, SiO₂, 70:30 hexane: EtOAc) gave the title compound as a pale yellow solid (143 mg, 19% yield).

HPLC: Inertsil ODS-3V C18, [KH₂PO₄ (0.01M, pH 3.2): CH₃CN], gradient, 264 nm, Rₜ 14.7 min, 95.5% purity.

¹H NMR (300 MHz, CDCl₃, TMS): δ 7.64 (d, J=8.7 Hz, 2H), 7.22 (d, J=9.0 Hz, 2H), 6.92 (s, 1H), 5.81 (s, 1H), 4.58 (s, 1H), 3.64 (s, 3H), 2.85-2.76 (m, 2H), 2.54-2.44 (m, 2H), 2.04-1.83 (m, 2H), 1.44 (s, 9H), 1.27 (s, 9H).

LC-MSD (ES+): m/z [411 (M+H)⁺, 100].

### Step (iv): Synthesis of 1-[4-(4-tert-butyl-6-tert-butylamino-pyrimidin-2-ylamino)-phenyl]-cyclobutanecarboxylic acid

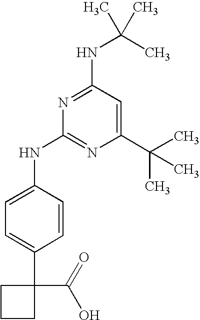

1-[4-(4-tert-butyl-6-tert-butylamino-primidin-2-ylamino)-phenyl}-cyclobutane-carboxylic acid methyl ester (140 mg, 0.34 mmol) was dissolved in methanol (7 mL) followed by solution of NaOH (0.118 g, 2.95 mmol) in 5 mL water. The resulting mixture was stirred at reflux for about 10-19 h under nitrogen atmosphere. The mixture was concentrated by rotary evaporation to remove most of the methanol then adjusted to pH 3-4 with 10% HCl (aq). The precipitate was then filtered and washed with ether. The resulting solid was dried under vacuum. Purification (flash column chromatography, SiO₂, 90:10 dichloromethane: methanol) gave the title compound as a white solid (90 mg, 60% yield).

HPLC: Inertsil ODS-3V C18, [KH₂PO₄ (0.01M, pH 3.2): CH₃CN], gradient, 264 nm, Rₜ 12.6 min, 96% purity.

¹H NMR (300 MHz, DMSO-d₆): δ 12.33-12.251 (m, 1H), 8.63 (s, 1H), 7.79-7.10 (m, 4H), 6.65 (s, 1H), [6.16 (s), 5.95 (s), 1H, possible rotamers or internal protonation], 2.67-2.66 (m, 2H), 2.45-2.32 (m, 2H), 1.89-1.77 (m, 2H), 1.39 (s, 9H), 1.29-1.11 (m, 9H).

LC-MSD (ES+): m/z [397 (M+H)⁺, 100].

### Example 5

### Synthesis of 1-(4-{4-[(1-ethyl-pyrrolidin-2-ylmethyl)-amino]-6-phenyl-pyrimidin-2-ylamino}-phenyl)-cyclobutanecarboxylic acid methyl ester

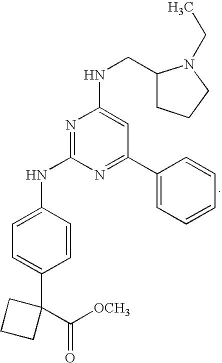

### Step (i): Synthesis of (2-chloro-6-phenyl-pyrimidin-4-yl)-(1-ethyl-pyrrolidin-2ylmethyl)-amine

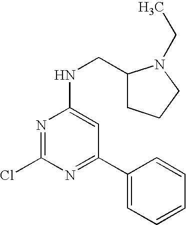

To 2,4-dichloro-6-phenyl-pyrimidine (1.508 g, 6.6 mmol), dissolved in THF (10 mL), was added 2-(aminomethyl)-1-ethylpyrrolidine (0.96 mL, 6.6 mmol). The resulting mixture was cooled to −60° C. and then treated with lithium bis (trimethylsilyl) amide (13.2 mL, 13.2 mmol, 1M). The resulting solution was allowed to stir at −60° C. for 10 min and was quenched with water slowly. The mixture was diluted with ethylacetate, and washed two times with water and one time with brine. The organic phase was dried over sodium sulfate, filtered, and concentrated by rotary evaporation. Purification (Biotage Horizon HPFC chromatography system, SiO₂, 95:5 dichloromethane: MeOH) gave the title compound as a solid (1.1 g, 52% yield) in addition to C-2 regioisomer (not fully characterized).

HPLC: Betasil ODS-3V C18, [KH₂PO₄ (0.01M, pH 3.2): CH₃CN], gradient, 264 nm, Rₜ 10.1 & 10.7 min, 52.8, 43.8% purity respectively.

¹H NMR (300 MHz, CDC1₃, TMS): δ 7.98-7.94 (m, 2H), 7.48-7.44 (m, 3H), 6.96 (s, 1H), 6.02-5.87 (m, 1H), 3.75 (b s, 1H), 3.39-3.19 (m, 2H), 2.91-2.65 (m, 2H), 2.29-2.12 (m, 2H), 1.97-1.60 (m, 4H), 1.12 (t, J=7.5 Hz, 3H).

LC-MSD (ES+): m/z [317 (M+H)⁺, 100].

### Step (ii): Synthesis of 1-(4-{4-[(1-ethyl-pyrrolidin-2-ylmethyl)-amino]-6-phenyl-pyrimidin-2-ylamino}-phenyl)-cyclobutanecarboxylic acid methy ester

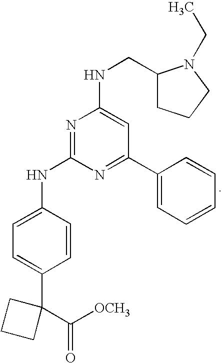

A similar method was used as in Example 1, step (iv) by using (2-chloro-6-phenyl-pyrimidin-4-yl)-(1-ethyl-pyrrolidin-2-ylmethyl)-amine instead of tert-butyl-(2-chloro-6-phenyl-pyrimidin-4-yl)-amine. Flash column chromatography (SiO₂, 93:6:1 dichloromethane: MeOH: NH₄OH) purification gave the title compound as a light brown solid (200 mg, 18% yield).

HPLC: Betasil ODS-3V C18, [KH₂PO₄ (0.01M, pH 3.2): CH₃CN], gradient, 264 nm, Rₜ 11.4 min, 95% purity.

¹H NMR (300 MHz, CDCl₃, TMS): δ 7.93 (b s, 2H), 7.43 to 7.26 (m, 7H), 6.61 (s, 1H), 6.46 (s, 1H), 5.47 (s, 1H), 3.82-3.71 (m, 1H), 3.67 (s, 3H), 3.40-3.19 (m, 2H), 2.99-2.46 (m, 6H), 2.41-1.70 (m, 8H), 1.14 (t, J=7.5 Hz, 3H).

LC-MSD (ES+): m/z [486 (M+H)⁺, 100].

### Example 6

### Synthesis of 1-[4-(4-tert-butylamino-5-fluoro-6-phenyl-pyrimidin-2-ylamino)-phenyl]-cyclobutanecarboxylic acid

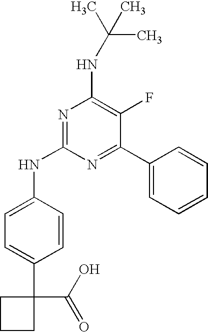

### Step (1): Synthesis of 2,4-dichloro-5-fluoro-6-phenyl-pyrimidine

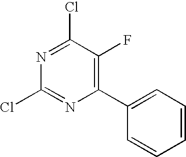

The title compound was prepared following the procedure as described in Example 4 step (i), by using 2,4-dichloro-5-fluoro pyrimidine and phenyl}itium instead of 2,4-dichloropyrimidine and tert-butyllithium. Purification (Biotage Horizon HPFC chromatography system, SiO₂, 90:10 hexane: EtOAc) gave the title compound as a light yellow solid (1.7 g, 69% yield).

HPLC: Inertsil ODS-3V C18, 30:70 [KH₂PO₄ (0.01M, pH 3.2): CH₃CN], 264 nm, Rₜ 14.0 min, 96.4% purity.

¹H NMR (300 MHz, CDCl₃, TMS): δ 8.12-8.09 (m, 2H), 7.59-7.51 (m, 3H).

LC-MSD (ES+): m/z [242 (M+H)⁺, 100].

### Step (ii): Synthesis of tert-butyl-(2-chloro-5-fluoro-6-phenyl-pyrimidin-4-yl)-amine

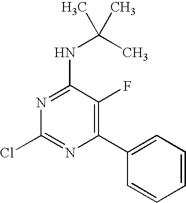

A similar method was used as in Example 5, step (i) by using 2,4-dichloro-5-fluoro-6-phenyl-pyrimidine and tert-butylamine instead of 2,4-dichloro-6-phenyl-pyrimidine and 2-(aminomethyl)-1-ethylpyrrolidine. Flash column chromatography (SiO₂, 100% dichloromethane) purification gave the title compound as a white solid (0.34 g, 41% yield).

HPLC: Inertsil ODS-3V C18, 30:70 [KH₂PO₄ (0.01M, pH 3.2): CH₃CN], 240 nm, Rₜ 21.4 min, 97% purity.

¹H NMR (300 MHz, CDCl₃, TMS): δ 7.96-7.92 (m, 2H), 7.48-7.43 (m, 3H), 5.18 (s, 1H), 1.53 (s, 9H).

LC-MSD (ES+): m/z [280 (M+H)⁺, 100].

### Step (iii): Synthesis of 1-[4-(4-tert-butylamino-5-fluoro-6-phenyl-pyrimidin-2-ylamino)-phenyl]-cyclobutanecarboxylic acid methyl ester

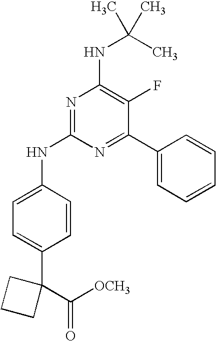

A similar method was used as in Example 1, step (iv) by using tert-butyl-(2-chloro-5-fluoro-6-phenyl-pyrimidin-4-yl)-amine instead of tert-butyl-(2-chloro-6-phenyl-pyrimidine-4-yl)-amine and 1-(4-aminophenyl)-cyclobutane-carboxylic acid methyl ester. Purification (Biotage Horizon HPFC chromatography system, SiO₂, 80:20 hexane: EtOAc) gave the title compound as a pale yellow solid (300 mg, 48% yield).

HPLC: Inertsil ODS-3V C18, 30:70 [KH₂PO₄ (0.01M, pH 3.2): CH₃CN], 264 nm, Rₜ 45.44 min, 99% purity.

¹H NMR (300 MHz, CDCl₃, TMS): δ 7.96 (d, J=8.1 Hz, 2H), 7.62 (d, J=8.7 Hz, 2H), 7.50-7.43 (m, 2H), 7.26-7.24 (m, 3H), 6.84 (s, 1H), 5.00 (s, 1H), 3.64 (s, 3H), 2.86-2.77 (m, 2H), 2.55-2.45 (m, 2H), 2.05-1.80 (m, 2H), 1.54 (s, 9H).

LC-MSD (ES+): m/z [449 (M+H)⁴, 100].

### Step (iv): Synthesis of 1-[4-(4-tert-butylamino-5-fluoro-6-phenyl-pyrimidin-2-ylamino)-phenyl]-cyclobutanecarboxylic acid

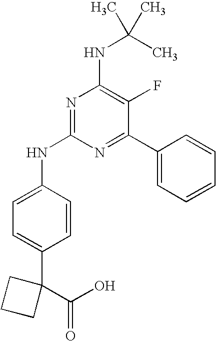

A similar method was used as in Example 1, step (v) by using 1-[4-(4-tert-butylamino-5-fluoro-6-phenyl-pyrimidin-2-ylamino)-phenyl]-cyclobutanecarboxylic acid methyl ester instead of 1-[4-(4-tert-butylamino-6-phenyl-pyrimidin-2-ylamino)-phenyl]-cyclobutanecarboxylic acid methyl ester. The resulting pale yellow solid was dried under vacuum to give the title compound (230 mg, 95% yield).

HPLC: Inertsil ODS-3V C18, 30:70 [KH₂PO₄ (0.01M, pH 3.2): CH₃CN], 264 nm, Rₜ 18.0 min, 99% purity.

¹H NMR (300 MHz, DMSO-d₆): δ 10.0 (br s, 1H), 8.1 (s, 1H), 7.86-7.85 (m, 2H), 7.61-7.54 (m, 5H), 7.28 (d, J=8.7 Hz, 2H), 2.78-2.72 (m, 2H), 2.47-2.37 (m, 2H), 2.04-1.75 (m, 2H), 1.48 (s, 9H).

LC-MSD (ES+): m/z [435 (M+H)⁺, 100].

### Example 7

### Synthesis of 1-[4-[4-(bis-cyclopropylmethyl-amino)-6-(4-fluoro-phenyl)-pyrimidin-2-ylamino]-phenyl}-cyclobutanecarboxylic acid methyl ester

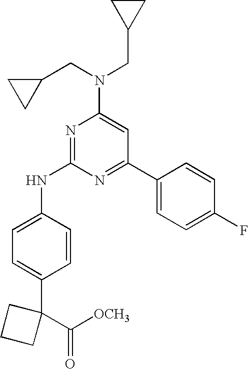

### Step (i): Synthesis of 2,4-dichloro-6-(4-fluoro-phenyl)-pyrimidine

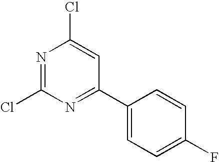

The title compound was prepared using the literature procedures referenced in the synthesis of 2,4-dichloro-6-phenyl-pyrimidine.

A flask was charged with 2,4,6-trichloropyrimidine (5.54 g, 30 mmol), 4-fluorophenylboronic acid (2.8 g, 20 mmol), palladium acetate (0.1 g, 0.4 mmol), triphenyiphosphine (0.23 g, 0.88 mmol), THF (20 mL) and alternately purged and backfilled with nitrogen atmosphere. After addition of 1M sodium carbonate (40 mL) the mixture was heated at 60° C. under stirring for 3 h. The resulting mixture was cooled to about 20-35° C. and diluted with EtOAc (80 mL). The organic layer was separated, washed with brine, and concentrated. The material was purified by flash chromatography over silica gel to afford title compound as a light yellow solid (4.2 g, 57% yield).

¹H NMR (CDCl₃, 300 MHz): δ 8.12-8.07 (m, 2H), 7.63 (s, 1H), 7.24-7.18 (m, 2H).

LC-MSD (ES+): m/z [243 (M+H)⁺, 100].

### Step (ii): Synthesis of [2-chloro-6-(4-fluoro-phenyl)-pyrimidin-4-yl]-bis-cyclopropylmethyl-amine

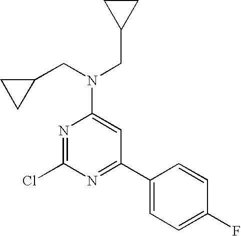

A flask was charged with 2,4-dichloro-6-(4-fluoro-phenyl)-pyrimidine (1.21 g, 5 mmol), bis-cyclopropylmethyl-amine [(0.65 g, 5.1 mmol); prepared following a method disclosed in U.S. Pat. No. 3,546,295], palladium acetate (0.025 g, 0.1 mmol), 1,3-bis(diphenylphosphino)propane (0.045 g, 0.1 mmol), anhydrous THF (20 mL), and then alternately purged and backfilled with nitrogen atmosphere. After cooling the mixture to −30° C., a solution of LiHMDS (7.5 mmol) in THF was added slowly under nitrogen atmosphere. After stirring for 3 h the mixture was warmed to about 20-35° C. and stirred additionally for an hour. EtOAc (50 mL) followed by water (40 mL) was added to the reaction mixture and stirred for 0.5 h. The organic layer was separated, washed with brine, and concentrated. The crude material was purified by flash chromatography over silica gel to afford the title compound as a light yellow viscous liquid (1.4 g, 85% yield).

¹H NMR (CDCl₃, 300 MHz): δ 7.97-7.93 (m, 2H), 7.25-7.10 (m, 2H), 6.70 (s, 1H), 3.52 (d, J=5.4 Hz, 4H), 1.11-1.06 (m, 2H), 0.60-0.54 (m, 4H), 0.35-0.30 (m, 4H).

LC-MSD (ES+): m/z [322 (M+H)⁺, 100].

### Step (iii): Synthesis of 1-{4-[4-(bis-cyclopropylmethyl-amino)-6-(4-fluoro-phenyl)-pyrimidin-2-ylamino]-phenyl}-cyclobutanecarboxylic acid methyl ester

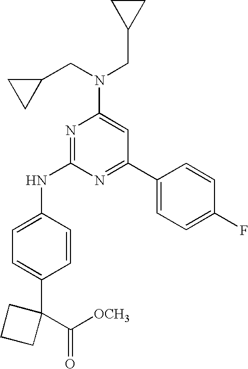

A mixture of [2-chloro-6-(4-fluoro-phenyl)-pyrimidin-4-yl]-bis-cyclopropylmethyl-amine (0.4 g, 1.2 mmol) and 1-(4-amino-phenyl)-cyclobutanecarboxylic acid methyl ester (0.37 g, 1.6 mmol) in anhydrous 2-propanol (5 mL) was heated at reflux for 18 h under nitrogen atmosphere. After evaporation of volatiles, the mixture was cooled to about 20-35° C. and diluted with EtOAc. The organic solution was washed with 5% sodium bicarbonate, brine, dried over sodium sulfate, filtered, and concentrated under reduced pressure. The material was purified by chromatography over silica gel to afford the title compound as a thick liquid, which solidified on standing at about 20-35° C. (0.36 g, 60% yield).

HPLC: Inertsil ODS-3V C18, 30:70 [0.01M KH₂PO₄ (0.01M, pH 3.2):CH₃CN], 264 nm, Rₜ 33.7 min, 95.69% purity.

¹H NMR (CDCl₃, 300 MHz): δ 7.98-7.93 (m, 2H), 7.64 (d, J=6.6 Hz, 2H), 7.26 (d, J=6.6 Hz, 2H), 7.17-7.11 (m, 2H) 6.92 (br s, 1H), 6.39 (s, 1H), 3.65 (s, 3H), 3.54 (d, J=6.3 Hz, 4H), 2.87-2.78 (m, 2H), 2.55-2.46 (m, 2H), 2.03-1.88 (m, 2H), 1.17-1.12 (m, 2H), 0.58-0.52 (m, 4H), 0.33-0.28 (m, 4H).

LC-MSD (ES+): m/z [501 (M+H)⁺, 100].

### Example 8

### Synthesis of 1-{4-[4-(cyclopropylmethyl-amino)-6-(4-fluoro-phenyl)-pyrimidin-2-ylamino]-phenyl}-cyclopentane-carboxylic acid

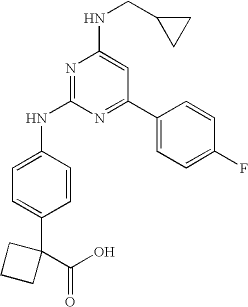

### Intermediate: Preparation of 1-(4-amino-phenyl)cyclopentanecarboxylic acid methyl ester

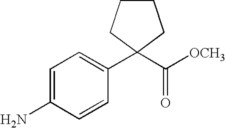

### Step (a): Synthesis of 1-(4-amino-phenyl)cyclopentanecarboxylic acid methyl ester

Sodium hydride (2.8 g, 60% dispersion in mineral oil) was added slowly to an ice-cold solution of (4-nitro-phenyl)-acetic acid methyl ester (6.0 g, 30.7 mmol) in anhydrous DMF (30 mL) under nitrogen atmosphere. After stirring at 0° C. for 20 min, 1,4-diiodobutane (7.86 mL, 61 mmol) was added drop wise under stirring. After complete addition, the reaction mixture was warmed to about 20-35° C. and stirring continued for an additional 3 h. The reaction was stopped by adding water drop wise followed by EtOAc. The organic layer was separated, washed with brine, dried over sodium sulfate, and concentrated under reduced pressure. The material was purified by chromatography over silica gel to afford a pale yellow viscous liquid that solidified on standing at about 20-35° C. (4.1 g, 54% yield). The solid (3.4 g, 13.6 mmol) was dissolved in anhydrous ethanol and tin (II) chloride dihydrate (13.86 g, 61 mmol) was added slowly under nitrogen atmosphere. Thereafter, the mixture was heated at 90° C. for 3 h. After evaporation of volatiles under reduced pressure the resultant mixture was diluted with ice water, and the solution was adjusted to pH ˜11 by adding NaOH (aq). The mixture was extracted with EtOAc (3×50 mL) and the combined organic extracts were washed with water, brine, dried over sodium sulfate, and concentrated under reduced pressure to afford the title compound as a granular light yellow solid (2.67 g, 88% yield).

¹H NMR (CDCl₃, 300 MHz): δ 7.16 (dd, J=8.4, 1.8 Hz, 2H), 6.63 (dd, J=8.4, 1.8 Hz, 2H), 3.59 (s, 3H), 2.60-2.55 (m, 2H), 1.87-1.67 (m, 6H).

LC-MSD (ES+): m/z [220 (M+H)⁺, 100].

### Step (i): Synthesis of 1-{4-[4-(cyclopropylmethyl-amino)-6-(4-fluoro-phenyl)-pyrimidin-2-ylamino]-phenyl}-cyclopentanecarboxylic acid methyl ester

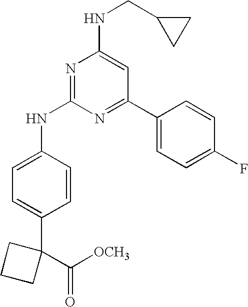

The title compound (98% yield) was afforded following the procedure as described in Example 7, steps (ii) and (iii), by using [2-chloro-6-(4-fluoro-phenyl)-pyrimidin-4-yl]-cyclopropylmethyl-amine instead of [2-chloro-6-(4-fluoro-phenyl)-pyrimidin-4-yl]-bis-cyclopropylmethyl-amine.

HPLC: Inertsil ODS-3V C18, 30:70 [0.01M KH₂PO₄ (0.01M, pH 3.2): CH₃CN], 264 nm, Rₜ 15.38 min, 94.0% purity.

¹H NMR (CDCl₃, 300 MHz): δ 7.98-7.94 (m, 2H), 7.64 (d, J=8.7 Hz, 2H), 7.33 (d, J=8.7 Hz, 2H), 7.16-7.10 (m, 2H), 6.92 (br s, 1H), 6.21 (s, 1H), 4.94 (br s, 1H), 3.61 (s, 3H), 3.27 (t, J=6.0 Hz, 2H), 2.67-2.61 (m, 2H), 1.94-1.87 (m, 2H), 1.74-1.71 (m, 4H), 1.14-1.09 (m, 1H), 0.62-0.56 (m, 2H), 0.31-0.26 (m, 2H).

LC-MSD (ES+): m/z [461 (M+H)⁺, 100].

### Step (ii): Synthesis of 4-(cyclopropylmethyl-amino)-6-(4-fluoro-phenyl)-pyrimidin-2-ylamino]-phenyl}-cyclopentane-carboxylic acid

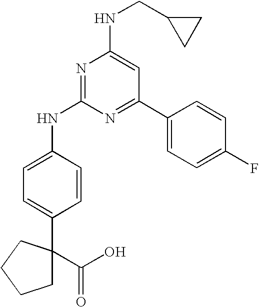

A solution of water (12 mL) and NaOH (0.26 g, 6.5 mmol) was added to a solution of 1-{4-[4-(cyclopropylmethyl-amino)-6-(4-fluoro-phenyl)-pyrimidin-2-ylamino]-phenyl}-cyclopentanecarboxylic acid methyl ester (0.6 g, 1.3 mmol) in methanol (15 mL) and the mixture was heated at 80° C. for 8 h. After evaporation of methanol the aqueous mixture was cooled to about 20-35° C., and acidified with concentrated hydrochloric acid. The precipitate, which fowled after standing at about 20-35° C., was filtered and washed several times with water. Recrystallization from methanol afforded the title compound as a white powder (0.31 g, 54% yield).

HPLC: Inertsil ODS-3V C18, 30:70 [0.01M KH₂PO₄ (0.01M, pH 3.2): CH₃CN], 264 nm, Rₜ 6.75 min, 97.16% purity.

¹H NMR (CDCl₃+CD₃OD, 300 MHz): δ 7.85-7.80 (m, 2H), 7.56 (d, J=8.7 Hz, 2H), 7.36 (d, J=8.7 Hz, 2H), 7.12-7.07 (m, 2H), 6.22 (s, 1H), 3.23 (d, J=7.2 Hz, 2H), 2.60-2.53 (m, 2H), 1.87-1.77 (m, 2H), 1.74-1.64 (m, 4H), 1.08-0.96 (m, 1H), 0.53-0.48 (m, 2H), 0.28-0.19 (m, 2H).

LC-MSD (ES+): m/z [447 (M+H)⁺, 100].

### Example 9

### Synthesis of 1-{4-[4-(cyclopropylmethyl-amino)-6-(4-fluoro-phenyl)-pyrimidin-2-ylamino]-phenyl}-cyclobutane-carboxylic acid

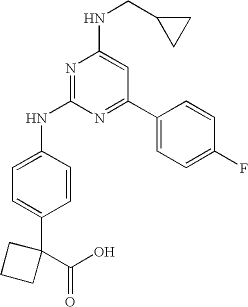

### Step (i): Preparation of [2-chloro-6-(4-fluoro-phenyl)-pyrimidin-4-yl]-cyclopropylmethyl-amine

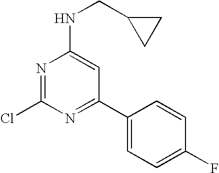

The title compound was afforded as a pale yellow liquid (58% yield) following the procedure as described in Example 7, step (ii), by using commercially available aminomethyl-cyclopropane instead of bis-cyclopropylmethyl-amine.

¹H NMR (CDCl₃, 300 MHz) δ 7.98-7.94 (m, 2H), 7.16-7.10 (m, 2H), 6.53 (s, 1H), 5.32 (br s, 1H), 3.24 (m, 2H), 1.10-1.06 (m, 1H), 0.62-0.59 (m, 2H), 0.31-0.28 (m, 2H).

LC-MSD (ES+): m/z [278 (M+H)⁺, 100].

### Step (ii): Synthesis of 1-{4-[4-(cyclopropylmethyl-amino)-6-(4-fluoro-phenyl)-pyrimidin-2-ylamino]-phenyl}-cyclobutanecarboxylic acid methyl ester

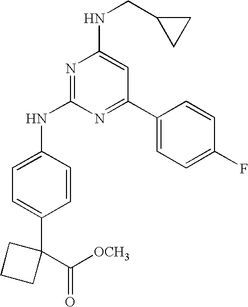

A mixture of [2-chloro-6-(4-fluoro-phenyl)-pyrimidin-4-yl]-cyclopropylmethyl-amine (4.0 g, 14.4 mmol) and 1-(4-amino-phenyl)-cyclobutanecarboxylic acid methyl ester (4.0 g, 19.5 mmol) in anhydrous 1-butanol (30 mL) under nitrogen atmosphere was heated with stirring at 100° C. for 16 h. Thereafter, the reaction was cooled to about 20-35° C. The precipitated solid was collected in a Buchner funnel and washed with ether. The solid was re-dissolved in EtOAc, washed with 5% aqueous sodium bicarbonate, water, brine, and dried over sodium sulfate. After concentration under reduced pressure the title compound was afforded as a white powder (5.5 g, 86% yield).

HPLC: Inertsil ODS-3V C18, 30:70 [0.01M KH₂PO₄ (0.01M, pH 3.2): CH₃CN], 264 nm, Rₜ 11.98 min, 93.82% purity.

¹H NMR (CDCl₃, 300 MHz): δ 7.99-7.94 (m, 2H), 7.66 (d, J=8.7 Hz, 2H), 7.28-7.25 (m, 2H), 7.16-7.10 (m, 2H), 6.93 (br s, 1H), 6.21 (s, 1H), 4.94 (br s, 1H), 3.64 (s, 3H), 3.27 (t, J=6.0 Hz, 2H), 2.86-2.77 (m, 2H), 2.55-2.45 (m, 2H), 1.99-1.85 (m, 2H), 1.14-1.09 (m, 1H), 0.62-0.56 (m, 2H), 0.31-0.26 (m, 2H).

LC-MSD (ES+): m/z [447 (M+H)⁺, 100].

### Step (iii): Synthesis of 1-{4-[4-(cyclopropylmethyl-amino)-6-(4-fluoro-phenyl)-pyrimidin-2-ylamino]-phenyl}-cyclobutane-carboxylic acid

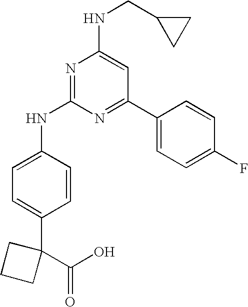

The title compound was afforded as a white powder (76% yield) following the procedure as described in Example 8, step (ii) by using appropriate starting material.

HPLC: Inertsil ODS-3V C18, 30:70 [0.01M KH₂PO₄ (0.01M, pH 3.2): CH₃CN], 264 nm, Rₜ 15.38 min, 98.66% purity.

¹H NMR (CDCl₃+CD₃OD, 300 MHz): δ 7.85-7.80 (m, 2H), 7.56 (d, J=8.7 Hz, 2H), 7.23 (d, J=8.7 Hz, 2H), 7.12-7.07 (m, 2H), 6.18 (s, 1H), 3.24 (t, J=7.5 Hz, 2H), 2.77-2.69 (m, 2H), 2.44-2.35 (m, 2H), 2.02-1.77 (m, 2H), 1.08-0.98 (m, 1H), 0.52-0.46 (m, 2H), 0.23-0.18 (m, 2H).

LC-MSD (ES+): m/z [433 (M+H)⁺, 100].

### Example 10

### Synthesis of 1-[4-(4-methylamino-6-phenyl-pyrimidin-2-ylamino)-phenyl]-cyclobutanecarboxylic acid

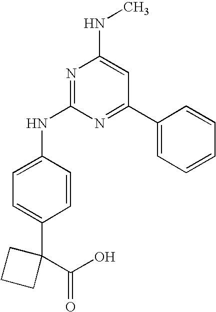

### Step (i): Synthesis of tert-butyl-(2-chloro-6-phenyl-pyrimidin-4yl)-methylamine and tert-butyl-(4-chloro-6-phenyl-pyrimidin-2yl)-methylamine

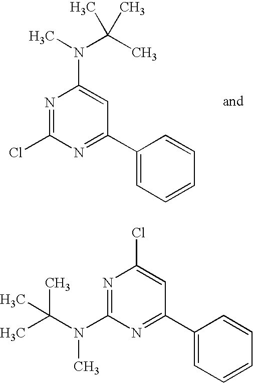

2,4-Dichloro-6-phenyl-pyrimidine (2.52 g, 11.1 mmol) was dissolved in N-methyl-tert-butylamine (2.6 mL, 17.58 mmol); then DMF (7 mL) was added. The resulting mixture was stirred at 60° C. in a sealed tube for about 10-19 h. The mixture was diluted with dichloromethane, and was washed two times with water and one time with brine. The organic phase was dried over sodium sulfate and concentrated by rotary evaporation. The resulting sample was dried for about 10-19 h under vacuum. Both regioisomers were isolated. Purification (Biotage Horizon HPFC system chromatography, SiO₂, 80:20 hexane: EtOAc) gave the title compounds as colorless gummy solids

tert-Butyl-(2-chloro-6-phenyl-pyrimidin-4yl)-methylamine (1.7 g, 55% yield):

HPLC: Betasil ODS-3V C18, 30:70 [KH₂PO₄ (0.01M, pH 3.2): CH₃CN], 264 nm, Rₜ 19.2 min, 93.1% purity.

¹H NMR (300 MHz, CDCl₃, TMS): δ 7.93 to 7.90 (m, 2H), 7.44-7.42 (m, 3H), 6.74 (s, 1H), 3.09 (s, 3H), 1.57 (s, 9H).

LC-MSD (ES+): m/z [276 (M+H)⁺, 100].

tert-Butyl-(4-chloro-6-phenyl-pyrimidin-2yl)-methylamine (600 mg, 19% yield):

HPLC: Betasil ODS-3V C18, 30:70 [KH₂PO₄ (0.01M, pH 3.2): CH₃CN], 264 nm, Rₜ 53.3 min, 97.4% purity.

¹H NMR (300 MHz, CDCl₃, TMS): δ 8.00-7.97 (m, 2H), 7.44-7.41 (m, 3H), 6.9 (s, 1H), 3.28 (s, 3H), 1.58 (s, 9H).

LC-MSD (ES+): m/z [276 (M+H)⁺, 100].

### Step (ii): Synthesis of 1-[4-(4-methylamino-6-phenyl-pyrimidin-2-ylamino)-phenyl}-cyclobutanecarboxylic acid methyl ester

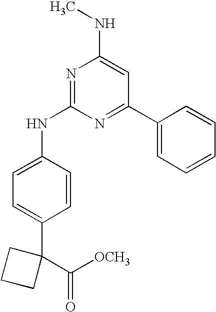

tert-Butyl-(2-chloro-6-phenyl-pyrimidin-4yl)-methylamine (1.8 g, 6.54 mmol) was dissolved in n-butanol (10 mL) followed by the addition of 1-(4-aminophenyl)-cyclobutanecarboxylic acid methyl ester (1.598 g, 7.84 mmol). The resulting mixture was stirred at reflux for about 10-19 h under nitrogen atmosphere. The mixture was diluted with dichloromethane, and was washed two times with water and one time with brine. The organic phase was dried over sodium sulfate, filtered, concentrated by rotary evaporation, and dried under vacuum. Purification (Biotage Horizon HPFC system chromatography, SiO₂, 50:50 hexane: EtOAc) gave the title compound as a pale yellow solid (800 mg, 31% yield).

HPLC: Inertsil ODS-3V C18, 30:70 [KH₂PO₄ (0.01M, pH 3.2): CH₃CN], 264 nm, Rₜ 6.2 min, 96.5% purity.

¹H NMR (300 MHz, CDCl₃, TMS): δ 7.97-7.94 (m, 2H), 7.66 (d, J=8.4 Hz, 2H), 7.42-7.48 (m, 3H), 7.28-7.23 (m, 3H), 6.22 (s, 1H), 5.04 (s, 1H), 3.63 (s, 3H), 2.98 (d, J=5.4 Hz, 3H), 2.86-2.77 (m, 2H), 2.54-2.44 (m, 2H), 2.07-1.84 (m, 2H).

LC-MSD (ES+): m/z [389 (M+H)⁺, 100].

### Step (iii): Synthesis of 1-[4-(4-methylamino-6-phenyl-pyrimidin-2-ylamino)-phenyl]-cyclobutanecarboxylic acid

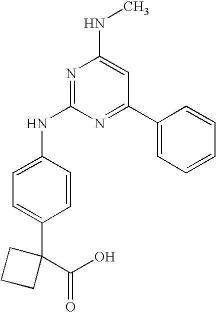

1-[4-(4-Methylamino-6-phenyl-pyrimidin-2-ylamino)-phenyl]-cyclobutanecarboxylic acid methyl ester (800 mg, 2.06 mmol) was dissolved in methanol (10 mL) followed by solution of NaOH (0.670 g, 16.48 mmol) in 10 mL water. The resulting mixture was stirred at reflux for about 10-19 h under nitrogen atmosphere. The mixture was concentrated by rotary evaporation to remove most of the methanol then adjusted to pH 3-4 with 10% HCl (aq). The precipitate was then filtered and washed with pentane. The resulting solid was dried under vacuum which gave the title compound as a white solid (710 mg, 92% yield).

HPLC: Inertsil ODS-3V C18, [KH₂PO₄ (0.01M, pH 3.2): CH₃CN], gradient, 264 nm, Rₜ 18.9 min, 96.8% purity.

¹H NMR (300 MHz, DMSO-d₆): δ 12.24 (s, 1H), 9.37 (s, 1H), 7.98 (s, 2H), 7.80 (d, J=8.4 Hz, 2H), 7.53 to 7.51 (m, 4H), 7.17 (d, J=6.3 Hz, 2H), 6.45 (s, 1H), 2.92 (d, J=4.2 Hz, 3H), 2.72-2.62 (m, 2H), 2.43-2.34 (m, 2H), 1.94-1.75 (m, 2H).

LC-MSD (ES+): m/z [375 (M+H)⁺, 100].

### Example 11

### Synthesis of 1-{4-[4-(tert-butyl-methyl-amino)-6-phenyl-pyrimidin-2-ylamino]-phenyl}-cyclobutanecarboxylic acid

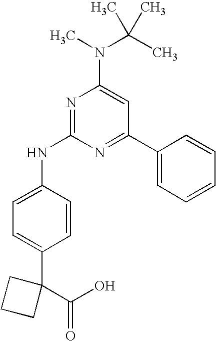

### Step (1): Synthesis of 1-{4-[4-(tert-butyl-methyl-amino)-6-phenyl-pyrimidin-2-ylamino]-phenyl}-cyclobutanecarboxylic acid methyl ester

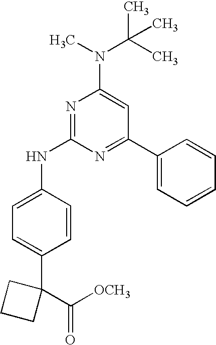

tert-Butyl-(2-chloro-6-phenyl-pyrimidin-4-yl)-methylamine (1.5 g, 5.43 mmol) was dissolved in dry toluene (15 mL) and followed by adding 1-(4-aminophenyl)-cyclobutanecarboxylic acid methyl ester (1.671 g, 8.14 mmol), tris(dibenzylideneacetone) dipalladium (0) (0.101 g, 0.108 mmol), 1,3-bis(diphenylphospino)propane (0.099 g, 0.217 mmol), sodium-tert-butoxide (0.7359 g, 7.6 mmol). The reaction mixture was stirred at reflux for about 10-19 h under nitrogen atmosphere. The reaction mixture was diluted with dichloromethane and filtered though Celite, then rinsed with dichloromethane. The filtrate was washed two times with water and one time with brine. The organic phase was dried over sodium sulfate and concentrated by rotary evaporation, and the material was dried under vacuum. Purification (Biotage Horizon HPFC system chromatography, SiO₂, 90:10 hexane: EtOAc) gave the title compound as a pale yellow solid (1.2 g, 50% yield).

HPLC: Betasil C18, 30:70 [KH₂PO₄ (0.01M, pH 3.2): CH₃CN], 264 nm, Rₜ 15.1 min, 95.9% purity.

¹H NMR (300 MHz, CDCl₃, TMS): δ 7.98-7.95 (m, 2H), 7.68-7.65 (m, 2H), 7.47-7.45 (m, 3H), 7.28-7.25 (m, 2H), 6.88 (s, 1H), 6.50 (s, 1H), 3.65 (s, 3H), 3.51 (s, 3H), 2.87-2.78 (m, 2H), 2.56-2.47 (m, 2H), 2.06-1.79 (m, 2H), 1.58 (s, 9H).

LC-MSD (ES+): m/z [445 (M+H)⁺, 100].

### Step (ii): Synthesis of 1-{4-[4-(tert-butyl-methyl-amino)-6-phenyl-pyrimidin-2-ylamino]-phenyl}-cyclobutanecarboxylic acid

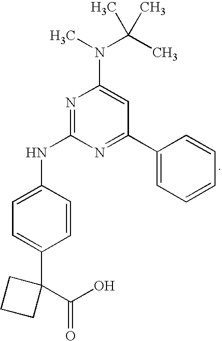

1-{4-[4-(tert-Butyl-methyl-amino)-6-phenyl-pyrimidin-2-ylamino]-phenyl}-cyclobutanecarboxylic acid methyl ester (1.1 g, 2.47 mmol) was dissolved in methanol (10 mL) and THF (2 mL) followed by solution of NaOH (0.3958 g, 9.88 mmol) in 10 mL water. The resulting mixture was stirred at reflux for about 10-19 h under nitrogen atmosphere. The mixture was concentrated by rotary evaporation to remove most of the methanol then adjusted pH 3-4 with 10% HCl (aq). The precipitate was then filtered with ether. The resulting solid was dried under vacuum which gave the title compound as a pale yellow solid (950 mg, 89% yield).

HPLC: Betasil C18, 30:70 [KH₂PO₄ (0.01M, pH 3.2): CH₃CN], 264 nm, Rₜ 6.4 min, 96.1% purity.

¹H NMR (300 MHz, CDCl₃, TMS): δ 8.77 (br s, 1), 7.85-7.82 (m, 2H), 7.47-7.42 (m, 514), 7.26 (d, J=8.7 Hz, 2H), 6.32 (s, 1H), 3.10(s, 3H), 2.87-2.79 (m, 2H), 2.50-2.41 (m, 2H), 2.04-1.80 (m, 2H), 1.49 (s, 9H).

LC-MSD (ES+): m/z [431 (M+H)⁺, 100].

### Example 12

### Synthesis of 1-{4-[4-(bicyclo[2.2.1]hept-2-ylamino)-6-phenyl-pyrimidin-2-ylamino]-phenyl}-cyclobutanecarboxylic acid

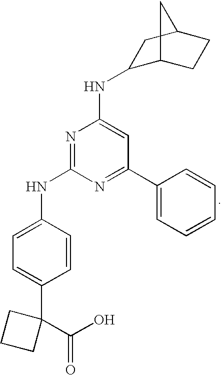

### Step (i): Synthesis of bicyclo[2.2.1]hept-2-yl-(2-chloro-6-phenyl-pyrimidin-4-yl)-amine

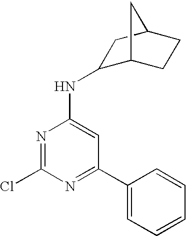

To 2,4-dichloro-6-phenyl-pyrimidine (2.50 g, 11.3 mmol) dissolved in DMF (15 mL) was added exo-2-aminonorbornane (1.3 mL, 11.3 mmol) followed by addition of K₂CO₃ (4.70 g, 33.8 mmol). After stirring at about 20-35° C. for about 10-19 h, the reaction mixture was extracted 3 times with dichloromethane. The combined organic layers were washed with water and brine, dried over sodium sulfate, filtered and then concentrated under vacuum to give product. Purification (Biotage Horizon HPFC chromatography system, SiO₂, 80:20 hexane: EtOAc) gave the title compound as a white solid (1.672 mg, 49% yield).

HPLC: Inertsil ODS-3V C18, 30:70 [KH₂PO₄ (0.01M, pH 3.2): CH₃CN], 264 nm, Rₜ 21.3 min, 99.3% purity.

¹H NMR (CDCl₃, 300 MHz): δ 7.98-7.94 (m, 2H), 7.56-7.44 (m, 3H), 6.55 (s, 1H), 5.23 (b s, 1H), 3.50 (b s, 1H), 1.66-1.05 (m, 10H).

LC-MSD (ES+): m/z [300 (M+H)⁺, 100].

### Step (ii): Synthesis of 1-{4-[4-(bicyclo[2.2.1]hept-2-ylamino)-6-phenyl-pyrimidin-2-ylamino]-phenyl}-cyclobutanecarboxylic acid methyl ester

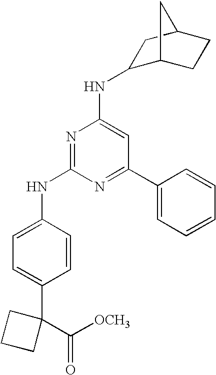

To bicyclo[2.2.1]hept-2-yl-(2-chloro-6-phenyl-pyrimidin-4-yl)-amine (1.5 g, 5.0 mmol) dissolved in n-butanol (25 mL) was added 1-(4-aminophenyl)-cyclobutanecarboxylic acid methyl ester (1.033 g, 5.0 mmol). The resulting mixture was stirred at reflux for about 10-19 h under nitrogen atmosphere. The mixture was diluted with dichloromethane and was washed two times with water and one time with brine. The organic phase was dried over sodium sulfate and concentrated by rotary evaporation. The resulting sample was dried for about 10-19 h under vacuum to give the title compound as a white solid (1.76 g, 75% yield).

HPLC: Inertsil ODS-3V C18, 30:70 [KH₂PO₄ (0.01M, pH 3.2): CH₃CN], 264 nm, Rₜ 14.9 min, 99.65% purity.

¹H NMR (CDCl₃, 300 MHz): δ 11.08 (s, 1H), 7.86-7.83 (m, 2H), 7.73 to 7.70 (m, 2H), 7.60-7.54 (m, 2H), 7.46-7.34 (m, 3H), 7.02-7.00 (m, 1H), 6.45 (s, 1H), 3.66 (s, 3H), 2.86-2.35 (m, 6H), 1.56-1.17 (m, 11H).

LC-MSD (ES+): m/z [469 (M+H)⁴, 100].

### Step (iii): Synthesis of 1-{4-[4-(bicyclo[2.2.1]hept-2-ylamino)-6-phenyl-pyrimidin-2-ylamino]-phenyl}-cyclobutanecarboxylic acid

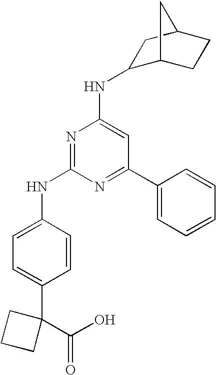

To 1-{4-[4-(bicyclo[2.2.1]hept-2-ylamino)-6-phenyl-pyrimidin-2-ylamino]-phenyl}-cyclobutanecarboxylic acid methyl ester (1.5 g, 3.2 mmol) dissolved in methanol (40 mL) was added a solution of NaOH (468.5 mg, 11.2 mmol) in 20 mL of water. The resulting mixture was stirred at reflux for about 10-19 h under nitrogen atmosphere. The mixture was concentrated by rotary evaporation to remove most of the methanol and then adjusted to a pH 4-5 with dilute HCl (aq). The precipitate that formed was collected by vacuum filtration and rinsed with ether. The resulting sample was dried under vacuum to give the title compound as a white solid (1.266 g, 87% yield).

HPLC: Inertsil ODS-3V C18, 30:70 [KH₂PO₄ (0.01M, pH 3.2): CH₃CN], 264 nm, Rₜ 6.6 min, 99.63% purity.

¹H NMR (300 MHz, DMSO-d₆): δ 10.75 (s, 1H), 8.99 (s, 1H), 7.89 (br s, 3H), 7.68-7.60 (m, 5H), 7.27-7.25 (m, 2H), 6.52 (s, 1H), 3.80 (br s, 1H), 2.42-2.30 (m, 6H), 1.95-1.06 (m, 10H).

LC-MSD (ES+): m/z [455 (M+H)⁺, 100].

### Example 13

### Synthesis of 1-{4-[4-(4-fluoro-phenyl)-6-morpholin-4-yl-pyrimidin-2-ylamino]-phenyl}-cyclobutane-carboxylic acid

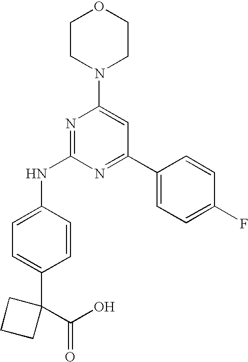

### Step (i): Synthesis of {4-[2-chloro-6-(4-fluoro-phenyl)-pyrimidin-4-yl]-morpholine

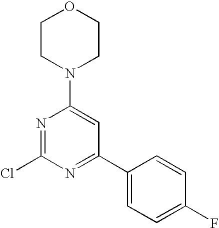

Morpholine (2.6 g, 29 mmol) was added slowly to a solution of 2,4-dichloro-6-(4-fluoro-phenyl)-pyrimidine (7.0 g, 29 mmol) and DIEA (11.24 g, 87 mmol) in anhydrous methanol (70 mL) at 0-5° C. under stirring. After addition the mixture was slowly warmed to about 20-35° C. and stirring continued for 6 h. Thereafter, volatiles were evaporated under reduced pressure and the residue was diluted with EtOAc. The organic mixture was washed with brine, dried over sodium sulfate, and solvent was evaporated under reduced pressure. The material was purified by chromatography over silica gel to afford the title compound (5.95 g, 71% yield).

HPLC: Inertsil ODS-3V C18, gradient, [KH₂PO₄ (0.01M, pH 3.2): CH₃CN], 264 nm, Rₜ 18.6 min, 97.42% purity.

¹H NMR (CDCl₃, 300 MHz): δ 7.99-7.94 (m, 2H), 7.08-7.20 (m, 2H), 6.64 (s, 1H), 3.64-3.84 (m, 8H).

LC-MSD (ES+): m/z [294 (M+H)⁺, 100].

### Step (ii): Synthesis of 1-{4-[4-(4-fluoro-phenyl)-6-morpholin-4-yl-pyrimidin-2-ylamino]-phenyl}-cyclobutanecarboxylic acid methyl ester

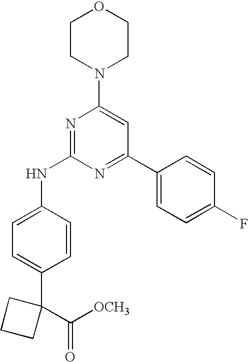

1-(4-Amino-phenyl)-cyclobutanecarboxylic acid methyl ester (4.4 g, 21.5 mmol) was added to a solution of 4-[2-chloro-6-(4-fluoro-phenyl)-pyrimidin-4-yl]-morpholine (5.93 g, 20.2 mmol) in anhydrous 1-butanol (25 mL) and heated at 110° C. for 16 h. After cooling to about 20-35° C. the precipitated solid was filtered, washed with ether, and then re-dissolved in EtOAc. The organic solution was washed successively with 10% aqueous sodium bicarbonate, brine, and dried over sodium sulfate. Evaporation of solvent afforded the title compound as a light brown solid (7.6 g, 82% yield).

HPLC: Inertsil ODS-3V C18, [KH₂PO₄ (0.01M, pH 3.2): CH₃CN], gradient, 264 nm, Rₜ 22.2 min, 94.21% purity.

¹H NMR (CDCl₃, 300 MHz): δ 7.99-7.94 (m, 2H), 7.63 to 7.58 (m, 2H), 7.29-7.24 (m, 2H), 7.17-7.11 (m, 2H), 6.37 (s, 1H), 3.84-3.80 (m, 4H), 3.70-3.65 (m, 4H), 3.63 (s, 3H), 2.87-2.78 (m, 2H), 2.55-2.45 (m, 2H), 2.03-1.85 (m, 2H).

LC-MSD (ES+): m/z [463 (M+H)⁺, 100].

### Step (iii): Synthesis of 1-{4-[4-(4-fluoro-phenyl)-6-morpholin-4-yl-pyrimidin-2-ylamino]-phenyl}-cyclobutaneearboxylic acid

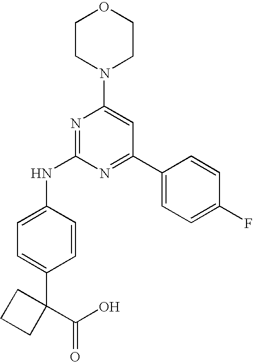

An aqueous solution (100 mL) of NaOH (4.0 g, 100 mmol) was added to a solution of 1-{4-[4-(4-fluoro-phenyl)-6-morpholin-4-yl-pyrimidin-2-ylamino]-phenyl}-cyclobutane-carboxylic acid methyl ester (7.6 g, 16.5 mmol) in methanol (100 mL) and heated at reflux for 4 h. Thereafter, the mixture was cooled to about 20-35° C., diluted with 50% methanol (aq, 200 mL), and then heated at reflux for an additional 10 h. Thereafter, methanol was evaporated under reduced pressure and the aqueous solution was adjusted to pH 4. The precipitated solid was filtered, washed with water, and dried in vacuo. Recrystallization from methanol afforded the title compound as a solid (6.3 g, 86% yield).

HPLC: Inertsil ODS-3V C18, [KH₂PO₄ (0.01M, pH 3.2): CH₃CN], gradient, 280 nm, Rₜ 16.94 min, 99.07% purity.

¹H NMR (DMSO-d₆, 300 MHz): δ 8.21-8.16 (m, 2H), 7.59 (d, J=8.4 Hz , 2H), 7.45 (t, J=8.4 Hz, 2H), 7.29 (d, J=8.4 Hz, 2H), 6.99 (s, 1H), 3.86-3.83 (m, 4H), 3.75-3.71 (m, 4H), 2.69-2.64 (m, 2H), 2.42-2.32 (m, 2H), 1.93-1.77 (m, 2H).

LC-MSD (ES+): m/z [449 (M+H)⁺, 100).

### Example 14

### Synthesis of 1-{4-[4-(4-fluoro-phenyl)-6-(4-hydroxy-piperidin-1-yl)-pyrimidin-2-ylamino]-phenyl}-cyclobutane-carboxylic acid

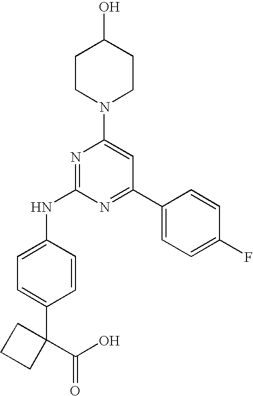

The title compound was afforded as a white solid (94% yield) following the procedure as described in Example 13 by using 4-hydroxy-piperidine instead of morpholine in step (i) .

HPLC: Inertsil ODS-3V C18, 30:70 [KH₂PO₄ (0.01M, pH 3.2): CH₃CN], gradient, 264 nm, Rₜ 12.63 min, 99.55% purity.

¹H NMR (CDCl₃+CD₃OD, 300 MHz): δ 7.81-7.65 (m, 2H), 7.40 (d, J=8.4 Hz, 2H), 7.17 (d, J=8.4 Hz, 2H), 7.11-7.05 (m, 2H), 6.34 (s, 1H), 4.12-4.03 (m, 1H), 3.90-3.74 (m, 2H), 3.64-3.52 (m, 1H), 3.44-3.36 (m, 1H), 2.72-2.61 (m, 2H), 2.41-2.24 (m, 2H), 1.98-1.68 (m, 4H), 1.60-1.42 (m, 2H).

LC-MSD (ES+): m/z [463 (M+H)⁺, 100].

### Example 15

### Synthesis of 1-[4-(4-cyclobutylamino-6-phenyl-pyrimidin-2-ylamino)-phenyl]-cyclobutanecarboxylic acid

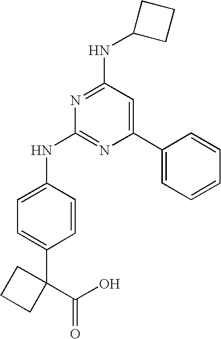

### Step (i): Synthesis of intermediate (2-chloro-6-phenyl-pyrimidin-4-yl)-cyclobutyl-amine

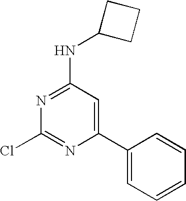

To 2,4-dichloro-6-phenylpyrimidine (630 mg, 2.8 mmol), dissolved in 30 mL MeOH, was added DIEA (0.7 mL, 4 mmol) and was stirred for ˜25 min at about 20-35° C. under a nitrogen atmosphere. To this mixture was added cyclobutylamine hydrochloride (323 mg, 3 mmol) and stirred ˜22 h. The mixture was concentrated by rotary evaporation and purification (Biotage Horizon HPFC chromatography system, SiO₂, 30:70 EtOAc: hexane) gave the title compound as a thick light yellow oil (222 mg, 30.6% yield).

¹H NMR (300 MHz, CDCl₃, TMS): δ 7.96-7.93 (m, 2H), 7.49-7.42 (m 3H), 6.51 (s, 1H), 5.44 (br s, 1H), 2.53-2.43 (m, 2H), 2.01-1.76 (m, 5H).

LC-MSD (ES+): m/z [260 (M+H)⁺, 100].

### Step (ii): Synthesis of intermediate 1-[4-(4-cyclobutylamino-6-phenyl-pyrimidin-2-ylamino)-phenyl]-cyclobutanecarboxylic acid methyl ester

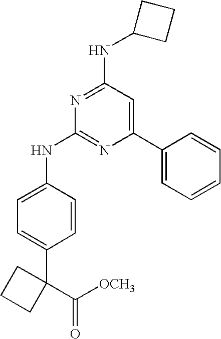

(2-Chloro-6-phenyl-pyrimidin-4-yl)-cyclobutyl-amine (220 mg, 0.85 mmol) and 1-(4-aminophenyl)-cyclobutanecarboxylic acid methyl ester (190 mg, 0.93 mmol) were mixed in n-butanol (8 mL) and heated at reflux under a nitrogen atmosphere for ˜18 h. The reaction was cooled to about 20-35° C. The precipitate was collected by filtration, rinsed with diethyl ether, and dried under vacuum to give the title compound as a white solid (247 mg, 67.8% yield).

¹H NMR (300 MHz, CDCl₃, TMS): δ 11.07 (s, 1H), 8.00 (b s, 1H), 7.58 (d, J=7.8 Hz, 1H), 7.69 (d, J=8.4 Hz), 7.57 (b s, 1H), 7.43 to 7.27 (m, 6H), 6.39 (s, 1H), 4.54-4.46 (m, 1H), 3.66 (s, 3H), 2.84 (b s, 2H), 2.56-2.36 (m, 4H), 2.17-1.79 (6H).

LC-MSD (ES+): m/z [429 (M+H)⁺, 100].

### Step (iii): Synthesis of 1-[4-(4-cyclobutylamino-6-phenyl-pyrimidin-2-ylamino)-phenyl]-cyclobutanecarboxylic acid

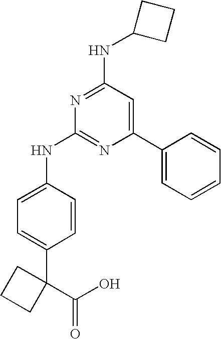

1-[4-(4-Cyclobutylamino-6-phenyl-pyrimidin-2-ylamino)-phenyl]-cyclobutane-carboxylic acid methyl ester (204 mg, 0.47 mmol) was dissolved in 8 mL MeOH, and a NaOH solution (67.3 mg, 1.7 mmol dissolved in 3 mL water) was added. The mixture was heated at reflux for ˜18 h and then cooled to ambient temperature. The reaction mixture was concentrated by rotary evaporation to remove the MeOH and then diluted with water. The supernatant was made acidic by slowly adding 6M HCl(aq) to a pH-5-6 (adjusted with 10% NaOH). The precipitate that formed was collected by vacuum filtration and rinsed with excess water. The collected solid was dried under vacuum to give the title compound as a white solid (168 mg, 85% yield).

HPLC: Betasil C-18, gradient, 30:70 [KH₂PO₄ (0.01M, pH 3.0): CH₃CN], 246 nm, Rₜ 18.4 min, 99% purity).

¹H NMR (300 MHz, DMSO-d₆): δ 12.23 (s, 1H), 8.15 (s, 1H), 7.96 (b s, 2H), 7.80 (d, J=8.7 Hz), 7.51-7.49 (m, 3H), 7.19 (d, J=8.7 Hz, 2H), 4.49 (b s, 1H), 2.73-2.64 (m, 2H), 2.43-2.34 (m, 5H), 2.00-1.66 (m, 6H).

LC-MSD (ES+): m/z [415 (M+H)⁺, 100].

### Example 16

### Synthesis of 1-[4-(4-cyclopentylamino-6-phenyl-pyrimidin-2-ylamino)-phenyl]-cyclobutane-carboxylic acid

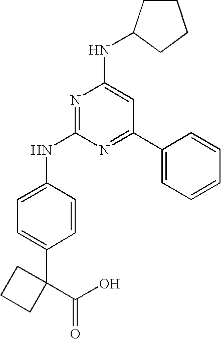

### Step (i): Synthesis of (2-chloro-6-phenyl-pyrimidin-4-yl)-cyclopentyl-amine

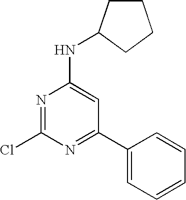

Cyclopentylamine (1.6 mL, 16 mmol) was added to a solution of 2,4-dichloro-6-phenyl-pyrimidine (3.0 g, 13.3 mmol) and DIEA (2.7 mL, 16 mmol) in anhydrous methanol (20 mL) under nitrogen atmosphere. The mixture was heated at reflux under stirring for 16 h. After evaporation of the volatiles the concentrate was diluted with EtOAc, washed with water, brine, and dried over sodium sulfate. After concentrating under reduced pressure the material was purified by chromatography over silica gel to afford the title compound as a white powder (2.1 g, 58% yield).

¹H NMR (CDCl₃, 300 MHz): δ 7.97-7.94 (m, 2H), 7.48-7.44 (m, 3H), 6.60 (s, 1H), 5.35 (b s, 1H), 4.10-4.02 (m, 1H), 2.11-2.04 (m, 2H), 1.80-1.68 (m, 4H), 1.56-1.52 (m, 2H).

LC-MSD (ES+): m/z [274 (M+H)⁺, 100].

### Step (ii): Synthesis of 1-[4-(4-cyclopentylamino-6-phenyl-pyrimidin-2-ylamino)-phenyl]-cyclobutane-carboxylic acid

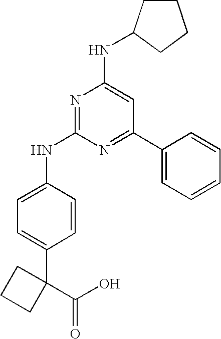

The title compound was afforded as a white powder (69% yield) following the method described in Example 9, steps (ii and iii).

HPLC: Inertsil ODS-3V C18, gradient, [KH₂PO₄ (0.01M, pH 3.2): CH₃CN], 264 nm, Rₜ 7.65 min, 97.7% purity.

¹H NMR (DMSO-d₆, 400 MHz): δ 10.40 (b s, 1H), 8.70 (b s, 1H), 8.14-7.20 (m, 9H), 6.52 (s, 1H), 4.36-4.29 (m, 1H), 2.78-2.40 (m, 4H), 2.15-1.55 (m, 10H).

LC-MSD (ES+): m/z [449 (M+H)⁴, 100].

### Example 17

### Synthesis of 1-{4-[4-(1,1-dimethyl-propylamino)-6-phenyl-pyrimidin-2-ylamino]-phenyl}-cyclobutanecarboxylic acid

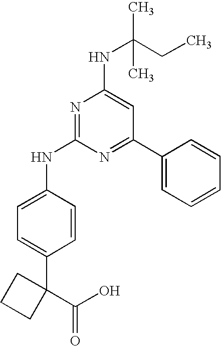

The title compound was afforded as a white powder (87% yield) following the method described in Example 9.

HPLC: Inertsil ODS-3V C18, 30:70 [KH₂PO₄ (0.01M, pH 3.2): CH₃CN], gradient, 264 nm, Rₜ 14.61 min, 94.46% purity.

¹H NMR (CDCl₃+CD₃OD, 300 MHz): δ 7.81-7.77 (m, 2H), 7.49-7.41 (m, 5H), 7.27-7.21 (m, 2H), 6.31 (s, 1H), 2.79-2.72 (m, 2H), 2.46-2.37 (m, 2H), 2.04-1.74 (m, 4H), 1.31 (s, 6H), 0.78 (t, J=7.2 Hz, 3H).

LC-MSD (ES+): m/z [431 (M+H)⁺, 100].

### Example 18

### Synthesis of 1-{4-[4-(4-fluoro-phenylamino)-6-phenyl-pyrimidin-2-ylamino]-phenyl}-cyclobutanecarboxylic acid

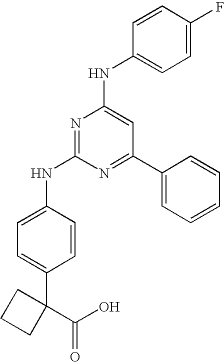

### Step (i): Synthesis of (2-chloro-6-phenyl-pyrimidin-4-yl)-(4-fluoro-phenyl)-amine

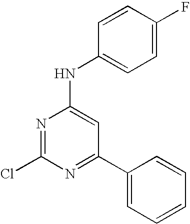

A similar procedure was used as in Example 5, step (i) by using 4-fluoro-aniline instead of 2-(aminomethyl)-1-ethylpyrrolidine. The solid was triturated with dichloromethane and hexane, filter, and dried under vacuum to give title compound as white solid (960 mg, 48% yield).

HPLC: Betasil ODS-3V C18, 30:70 [KH₂PO₄ (0.01M, pH 3.2): CH₃CN], 264 nm, Rₜ 11.3 min, 99% purity.

¹H NMR (300 MHz, DMSO-d₆): δ 10.10 (s, 1H), 7.98-7.95 (m, 2H), 7.66-7.52 (m, 5H), 7.25-7.20 (m, 2H), 7.12 (s, 1H).

LC-MSD (ES+): m/z [300 (M+H)⁺, 100].

### Step (ii): Synthesis of 1-{4-[4-(4-fluoro-phenylamino)-6-phenyl-pyrimidin-2-ylamino]-phenyl}-cyclobutanecarboxylic acid methyl ester

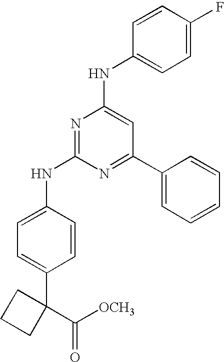

A similar method was used as in Example 1, step (iv) by using (2-chloro-6-phenyl-pyrimidin-4-yl)-(4-fluoro-phenyl)-amine instead of tert-butyl-(2-chloro-6-phenyl-pyrimdin-4-yl)-amine. The resulting solid was triturated with dichloromethane and hexane, filter, and dried under vacuum to give title compound as white solid (790 mg, 98% yield).

HPLC: Betasil ODS-3V C18, 30:70 [KH₂PO₄ (0.01M, pH 3.2): CH₃CN], 264 nm, Rₜ 11.3 min, 99% purity.

LC-MSD (ES+): m/z [469 (M+H)⁺, 100].

### Step (iii): Synthesis of 1-{4-[4-(4-fluoro-phenylamino)-6-phenyl-pyrimidin-2-ylamino]-phenyl}-cyclobutanecarboxylic acid

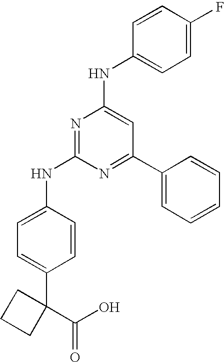

A similar method was used as in Example 1, step (v) by using 1-{4-[4-(4-fluoro-phenylamino)-6-phenyl-p yrimidin-2-ylamino]-phenyl}-cyclo butanecarboxylic acid methyl ester was used instead of 1-[4-(4-tert-butylamino-6-phenyl-pyrimidin-2-ylamino)-phenyl}-cyclobutanecarboxylic acid methyl ester. The resulting white solid was dried under vacuum to give the title compound (520 mg, 65% yield).

HPLC: Betasil ODS-3V C18, 30:70 [KH₂PO₄ (0.01M, pH 3.2): CH₃CN], 264 nm, Rₜ 8.2 min, 99% purity.

¹H NMR (300 MHz, DMSO-d₆): δ 11.08 (br s, 1H), 10.71 (br s, 1H), 8.14-7.19 (m, 13H), 6.85 (s, 1H), 2.89-2.40 (m, 4H), 2.00-1.82 (m, 2H).

LC-MSD (ES+): m/z [455 (M+H)⁺, 100].

### Example 19

### Synthesis of 1-{4-[4-(4-methanesulfonyl-phenylamino)-6-phenyl-pyrimidin-2-ylamino]-phenyl}-cyclobutanecarboxylic acid

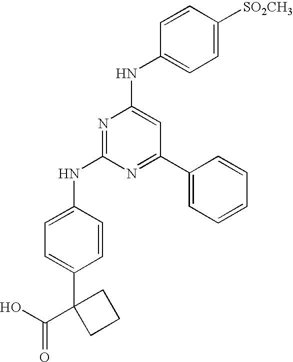

The title compound was afforded as a yellow solid (74% yield) following the procedure as described in Example 1, by using 4-methylthioaniline. The methylthio-group was oxidized to the methylsulfonyl group using the procedure: (2-chloro-6-phenyl-pyrimidin-4-yl)-(4-methanesulfonyl-phenyl)-amine (1.186 g, 3.6 mmol) was dissolved and stirred in acetone (30 mL). Oxone (6.673 g, 10.9 mmol) dissolved in water (15 mL) was added and the resulting solution was allowed to stir at about 20-35° C. for about 1 h. The mixture was diluted with dichloromethane, and washed two times with water and one time with brine. The organic phase was dried over sodium sulfate, filtered, and concentrated by rotary evaporation. The resulting sample was dried under vacuum and purified by Biotage Horizon HPFC chromatography system (SiO₂, 50:50 hexanes: EtOAc). HPLC: Betasil ODS-3V C18, 30:70 [KH₂PO₄ (0.01M, pH 3.2): CH₃CN], 264 nm, Rₜ 5.69 min, 99.7% purity.

¹H NMR (300 MHz, DMSO-d₆): δ 10.01 (s, 1H), 9.40 (s, 1H), 8.07-8.01 (m, 5H), 7.81-7.76 (m, 4H), 7.57-7.50 (m, 4H), 7.22 (d, J=8.7 Hz, 1H), 6.79 (s, 1H), 3.21 (s, 3H), 2.70-2.65 (m, 2H), 2.43-2.34 (m, 2H), 1.90-1.79 (m, 2H).

LC-MSD (ES+): m/z [515 (M+H)⁺, 100].

### Example 20

### Synthesis of (1-{4-[4-phenyl-6-((R)-1-phenyl-ethylamino)-pyrimidin-2-ylamino]-phenyl}-cyclobutyl)-acetic acid

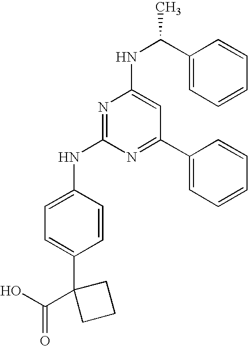

### Step (i): Synthesis of (2-chloro-6-phenyl-pyrimidin-4-yl)-((R)-1-phenyl-ethyl)-amine

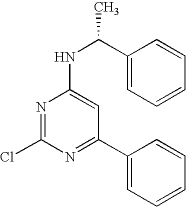

To 2,4-dichloro-6-phenylpyrimidine (1.343 g, 6 mmol), dissolved in 30 mL methanol, was added DIEA (1.1 mL, 6 mmol) and was stirred for ˜23 min at about 20-35° C. under a nitrogen atmosphere. To this mixture was added (R)-(+)-α-methyl-benzylamine (0.76 mL, 6 mmol) and stirred for about 10-19 h. The mixture was concentrated by rotary evaporation and purification (Biotage Horizon HPFC system chromatography, SiO₂, 20:80 EtOAc: hexane) gave the title compound as a white solid (570 mg, 30.8% yield).

¹H NMR (300 MHz, CDCl₃, TMS): δ 7.81-7.90 (m, 2H), 7.46-7.25 (m, 8H), 6.43 (s, 1H), 5.57 (s, 1H), 4.88 (s, 1H), 1.60 (d, J=6.6 Hz, 3H).LC-MSD (ES+): m/z [310 (M+H)⁺, 100].

### Step (ii): Synthesis of 1-{4-[4-phenyl-6-((R)-1-phenyl-ethylamino)-pyrimidin-2-ylamino]phenyl}-cyclobutanecarboxylic acid methyl ester

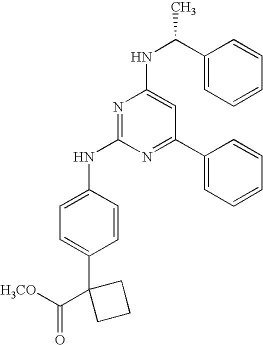

2-Chloro-6-phenyl-pyrimidin-4-yl)-((R)-1-phenyl-ethyl)-amine (490 mg, 1.31 mmol) and 1-(4-aminophenyl)-cyclobutanecarboxylic acid methyl ester (271.6 mg, 1.32 mmol) were mixed in n-butanol (15 mL) and heated at reflux under nitrogen atmosphere for ˜10 h. The mixture was diluted with dichloromethane (˜75 mL) and washed three times with water. The organic layer was separated, dried over anhydrous K₂CO₃, filtered, and concentrated. Purification (Biotage Horizon HPFC system chromatography, SiO₂, 1:1 hexane: EtOAc) gave the title compound as a white solid (466 mg, 74% yield).

¹H NMR (300 MHz, CDCl₃, TMS): δ 7.87-7.84 (m, 2H), 7.57 (d, J=8.7 Hz, 2H), 7.43 to 7.22 (m, 10H), 6.93 (s, 1H), 6.17 (s, 1H), 5.14 (b s, 1H), 5.02 (b s, 1H), 3.64 (s, 3H), 2.86-2.78 (m, 2H), 2.55-2.45 (m, 2H), 2.05-1.84 (m, 2H), 1.60 (d, J=6.6 Hz, 3H).

LC-MSD (ES+): m/z [479 (M+H)⁺, 100].

### Step (iii): Synthesis of (1-{4-[4-phenyl-6-((R)-1-phenyl-ethylamino)-pyrimidin-2-ylamino]phenyl}-cyclobutyl)-acetic acid

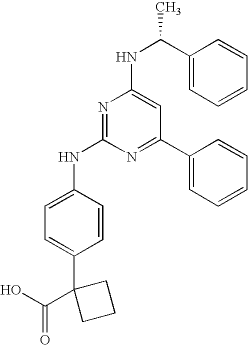

1-{4-[4-Phenyl-6-((R)-1-phenyl-ethylamino)-pyrimidin-2-ylamino]-phenyl}-cyclo-butane-carboxylic acid methyl ester (436 mg, 0.91 mmol) was dissolved in 12 mL MeOH, and a NaOH solution (131 mg, 3.2 mmol dissolved in 3 mL water) was added. The mixture was heated at reflux for ˜21 h and then cooled to ambient temperature. The reaction mixture was concentrated by rotary evaporation to remove the MeOH and then filtered by vacuum. The supernatant was made acidic by slowly adding 6M HCl (aq) to a pH˜2. The precipitate that formed was collected by vacuum filtration, rinsed with excess water, diethyl ether, and 2-propanol followed by another diethyl ether rinse. The collected solid was dried for about 10-19 h under vacuum to give the title compound as a white solid (403 mg, 95% yield).

HPLC: Inertsil ODS-3V C18, 30:70 [KH₂PO₄ (0.01M, pH 3.2): CH₃CN], 264 nm, Rₜ 5.0 min, 99.5% purity.

HPLC (chiral): Chiralpak AD-H, 60:40 hexane: ethanol, 275 nm, Rₜ 22.8 min, 99.5% purity.

[α]_{D}²⁵+172, MeOH.

¹H NMR (300 MHz, DMSO-d₆): δ 12.35 (s, 1H), 10.21 (s, 1H), 9.19 (s, 1H), 7.92-7.90 (m, 2H), 7.60-7.21 (m, 12H), 5.21 (apt b s, 1H), 2.76-2.68 (m, 2H), 2.41 (apt b s, 2H), 2.00-1.76 (m, 2H), 1.51 (d, J=6.9 Hz, 3H).

LC-MSD (ES+): m/z [465 (M+H)⁺, 100].

HRMS (TOF): m/z calcd for C₂₉H₂₉N₄O₂[M+H]⁺: 465.2291, found 465.2295 (A 0.9 ppm).

### Example 21

### Synthesis of (1-[4-[4-phenyl-6-((S)-1-phenyl-ethylamino)-pyrimidin-2-ylamino]-phenyl}-cyclobutyl)-acetic acid

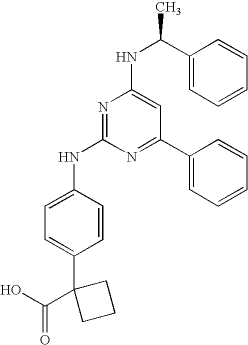

### Step (i): Synthesis of (2-chloro-6-phenyl-pyrimidin-4-yl)-((S)-1-phenyl-ethyl)-amine

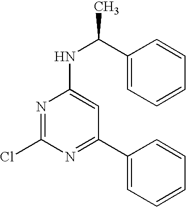

To 2,4-dichloro-6-phenylpyrimidine (450.3 mg, 2 mmol), dissolved in 30 mL methanol, was added DIEA (0.35 mL, 2 mmol) and was stirred for ˜30 min at about 20-35° C. under a nitrogen atmosphere. To this mixture was added (S)-(−)-a-methyl-benzylamine (0.25 mL, 2 mmol) and stirred ˜8 h. An additional DIEA (0.12 mL, 0.68 mmol) was added, and the mixture was stirred for ˜16.5 h. The mixture was concentrated by rotary evaporation and purification (Biotage Horizon HPFC system chromatography, SiO₂, 20: 80 EtOAc : hexane) gave the title compound as a white solid (260 mg, 42% yield).

¹H NMR (300 MHz, CDCl₃, TMS): δ 7.81-7.79 (m, 2H), 7.42-7.27 (m, 8H), 6.43 (s, 1H), 5.56 (b s, 1H), 4.88 (b s, 1H), 1.60 (d, J=6.9 Hz, 3H).

LC-MSD (ES+): m/z [310 (M+H)⁺, 100].

### Step (ii): Synthesis of 1-{4-[4-phenyl-6-((S)-1-phenyl-ethylamino)-pyrimidin-2-ylamino]-phenyl}-cyclobutanecarboxylic acid methyl ester

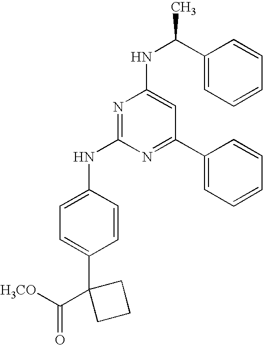

A similar procedure was followed as in Example 20, step (ii) by using 2-chloro-6-phenyl-pyrimidin-4-yl)-((S)-1-phenyl-ethyl)-amine (260 mg, 0.84 mmol), 1-(4-aminophenyl)-cyclobutanecarboxylic acid methyl ester (172.7 mg, 0.84 mmol) and n-butanol (4 mL). The title compound was isolated as a thick oil (400 mg) and was used without further purification.

¹H NMR (300 MHz, CDCl₃, TMS): δ 7.87-7.84 (m, 2H), 7.57 (d, J=8.7 Hz, 2H), 7.43 to 7.22 (m, 10H), 6.93 (s, 1H), 6.17 (s, 1H), 5.13 (b s, 1H), 5.02 (b s, 1H), 3.65 (s, 3H), 2.86-2.78 (m, 2H), 2.55-2.45 (m, 2H), 2.06-1.86 (m, 2H), 1.61 (signal overlap with H₂O, 3H).

### Step (iii): Synthesis of (1-{4-[4-phenyl-6-((S)-1-phenyl-ethylamino)-pyrimidin-2-ylamino]-phenyl}-cyclobutyl)-acetic acid

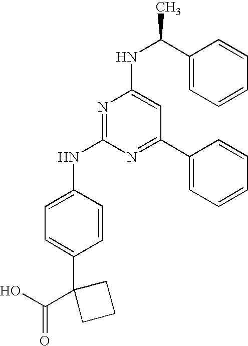

A similar procedure was followed as in Example 20, step (iii) by using 1-{4-[4-phenyl-6-((S)-1-phenyl-ethylamino)-pyrimidin-2-ylamino]-phenyl{-cyclobutane-carboxylic acid methyl ester (380 mg, 0.79 mmol), 10 mL MeOH, and NaOH solution (115.6 mg, 2.78 mmol dissolved in 2 mL water). The title compound was isolated as a white solid (340 mg, 92.7% yield).

HPLC: Inertsil ODS-3V C18, 30:70 [KH₂PO₄ (0.01M, pH 3.2): CH₃CN], 264 nm, Rₜ 5.2 min, 95.3% purity.

HPLC (chiral): Chiralpak AD-H, 60:40 Hexanes: ethanol, 275 nm, Rₜ 22.8 min, 99.69% purity.

[α]_{D}²⁵-182, MeOH.

¹H NMR (300 MHz, DMSO-d₆): δ 12.81 (b s, 1H), 10.51 (b s, 1H), 9.52 (b s, 1H), 7.91-7.89 (m, 2H), 7.63 to 7.25 (m, 12H), 6.62 (s, 1H), 5.26-5.16 (m, 1H), 2.77-2.68 (m, 2H), 2.41 (m, 2H), 2.01-1.74 (m, 2H), 1.53 (d, J=6.9 Hz, 3H).

LC-MSD (ES+): m/z [464 (M+H)⁺, 100].

HRMS (TOF): m/z calcd for C₂₉H₂₉N₄O_{2 [M+H]}⁺: 465.2291, found 465.2307 (Δ3.4 ppm).

### Example 22

### Synthesis of 1-{4-[4-((S)-2-hydroxy-1-phenyl-ethylamino)-6-phenyl-pyrimidin-2-ylamino]-phenyl}-cyclobutanecarboxylic acid

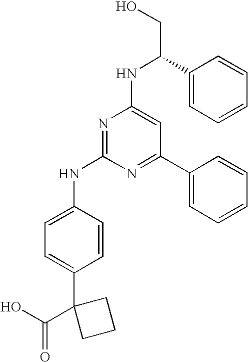

### Step (i): Synthesis of (S)-2-(2-chloro-6-phenyl-pyrimidin-4-ylamino)-2-phenyl-ethanol

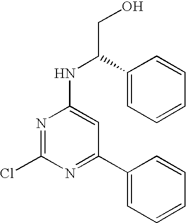

To 2,4-dichloro-6-phenylpyrimidine (642 mg, 2.8 mmol), dissolved in 30 mL methanol, was added DIEA (0.41 mL, 3 mmol) and was stirred for ˜25 min at about 20-35° C. under a nitrogen atmosphere. To this mixture was added (S)-(+)-2-phenyl glycinol (411 mg, 3 mmol) and stirred for ˜22 h. An additional DIEA (0.41 mL, 3 mmol) was added, and the mixture was stirred for about 10-19 h. The mixture after concentrating by rotary evaporation and purification (Biotage Horizon HPFC system chromatography, SiO₂, 35:65 EtOAc:hexane) gave the title compound as a white solid (367 mg, 39% yield).

¹H NMR (300 MHz, DMSO-d₆): δ 8.43 (apt d, J=7.8 Hz, 1H), 7.92 (b s, 2H), 7.50-7.22 (m, 8H), 7.08 (s, 1H), 5.18 (b s, 1H), 5.03 (b s, 1H), 3.68 (b s, 2H).

LC-MSD (ES+): m/z [326 (M+H)⁺, 100].

### Step (ii): Synthesis of 1-{4-[4-((S)-2-hydroxy-1-phenyl-ethylamino)-6-phenyl-pyrimidin-2-ylamino]-phenyl}-cyclobutanecarboxylic acid methyl ester

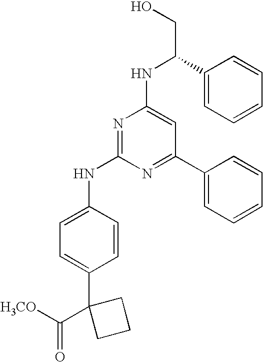

A similar method was followed as in Example 20, step (ii) by using (S)-2-(2-chloro-6-phenyl-pyrimidin-4-ylamino)-2-phenyl-ethanol (283 mg, 0.87 mmol), 1-(4-aminophenyl)-cyclo-butanecarboxylic acid methyl ester (180 mg, 0.87 mmol) and n-butanol (10 mL). Purification (Biotage Horizon HPFC system chromatography, SiO₂, 40:60 EtOAc: hexane) gave the title compound as a pale yellow solid (324 mg, 75% yield).

¹H NMR (300 MHz, DMSO-d₆): δ 8.99 (s, 1H), 7.95 (apt d, J=7.5 Hz, 2H), 7.68-7.11 (m, 13H), 6.60 (b s, 1H), 5.20 (b s, 1H), 5.01-4.98 (m, 1H), 3.69 (m, 2H), 3.58 (s, 2H), 2.75-2.66 (m, 2H), 2.44-2.38 (m, 2H), 1.98-1.81 (m 2H).

LC-MSD (ES+): m/z [479 (M+H)⁺, 100].

### Step (iii): Synthesis of 1-{4-[4(S)-2-hydroxy-1-phenyl-ethylamino)-6-phenyl-pyrimidin-2-ylamino]-phenyl}-cyclobutanecarboxylic acid

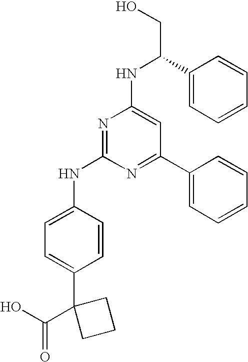

A similar method was followed as in Example 20, step (iii) by using 1-{4-[4-((S)-2-hydroxy-1-phenyl-ethylamino)-6-phenyl-pyrimidin-2-ylamino]-phenyl}-cyclobutanecarboxylic acid methyl ester (296 mg, 0.598 mmol), 8 mL Me0H and a NaOH solution (86 mg, 2.1 mmol dissolved in 2 mL water). The collected solid was dried for about 10-19 h under vacuum and gave the title compound as a white solid (241 mg, 83.7% yield).

HPLC: Inertsil ODS-3V C18, 30:70 [KH₂PO₄ (0.01M, pH 3.2): CH₃CN], 264 nm, Rₜ 3.4 min, 98.2% purity.

¹H NMR (300 MHz, DMSO-d₆): δ 12.38 (b s, 1H), 10.56 (s, 1H), 9.59 (s, 1H), 7.92-7.24 (m, 14H), 6.75 (s, 1H), 5.19-5.11 (m, 2H), 2.77-2.69 (m, 2H), 2.41 (m, 2H), 2.00-1.74 (m, 2H).

LC-MSD (ES+): m/z [481 (M+H)⁺, 100].

### Example 23

### Synthesis of 1-[4-(4-amino-6-phenyl-pyrimidin-2-ylamino)-phenyl]-cyclobutane-carboxylic acid

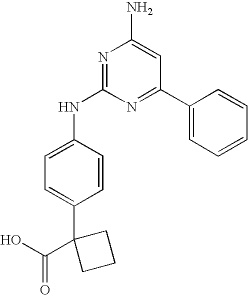

### Step (i): Synthesis of (2-chloro-6-phenyl-pyrimidin-4-yl)-(1-cyclopropyl-1-methyl-ethyl)-amine

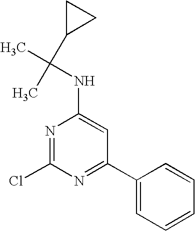

To 2,4-dichloro-6-phenylpyrimidine (674 mg, 3 mmol), dissolved in 3 mL DMF, was added 2-cyclopropyl-2-propylamine-p-toluenesulfonate salt (1 g, 3.68 mmol) followed by K₂CO₃ (1.026 g, 7.4 mmol). The sealed tube was heated in a ˜60° C. oil bath for ˜17 h. The mixture was cooled and diluted with EtOAc and water. The organic layer was separated and the aqueous layer was back extracted with EtOAc. The combined organic layers were washed two times with water, one time with brine, dried over anhydrous K₂CO₃, filtered, and concentrated by rotary evaporation. Purification (Biotage Horizon HPFC system chromatography, SiO₂, 1:9 EtOAc: hexanes) gave a white solid as the title compound (572 mg, 66% yield).

¹H NMR (300 MHz, CDCl₃, TMS): δ 7.95-7.92 (m, 2H), 7.47-7.45 (m, 3H), 6.81 (s, 1H), 5.34 (b s, 1H), 1.33-1.20 (m, 7H), 0.60-0.43 (m, 4H).

LC-MSD (ES+): m/z [288 (M+H)⁺, 100].

### Step (ii): Synthesis of 1-[4-(4-amino-6-phenyl-pyrimidin-2-ylamino)-phenyl]-cyclobutanecarboxylic acid methyl ester

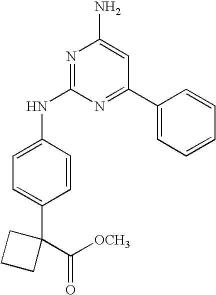

(2-Chloro-6-phenyl-pyrimidin-4-yl)-(1-cyclopropyl-1-methyl-ethyl)-amine (545 mg, 1.895 mmol) and 1-(4-aminophenyl)-cyclobutanecarboxylic acid methyl ester (426 mg, 2.08 mmol) were mixed in n-butanol (15 mL) and heated at reflux under nitrogen atmosphere for ˜17.5 h. After cooling to ambient temperature, a precipitate formed which was collected by vacuum filtration. The solid was rinsed with diethyl ether and dried under vacuum to give the title compound as a white solid (420 mg, 59% yield).

¹H NMR (300 MHz, CDCl₃, TMS): δ 10.76 (s, 1H), 8.51 (b s, 2H), 7.96-9.93 (m, 2H), 7.64-7.62 (m, 5H), 7.29 (apt d, J=8.7 Hz, 2H), 6.53 (s, 1H), 3.58 (s, 3H), 2.77-2.69 (m, 2H), 2.50-2.39 (m, 2H), 1.94-1.81 (m, 2H).

LC-MSD (ES+): m/z [375 (M+H)⁺, 100].

### Step (iii): Synthesis of 1-[4-(4-amino-6-phenyl-pyrimidin-2-ylamino)-phenyl]-cyclobutane-carboxylic acid

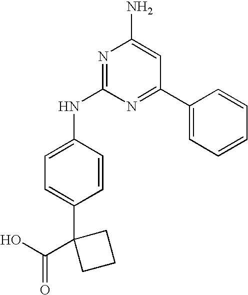

A similar method was used as in Example 20, step (iii) by using 1-[4-(4-amino-6-phenyl-pyrimidin-2-ylamino)-phenyl]-cyclobutanecarboxylic acid methyl ester (293 mg, 0.78 mmol), 3 mL MeOH and a NaOH solution (109 mg, 2.74 mmol dissolved in 5 mL water). The collected solid was dried for about 10-19 h under vacuum to give the title compound as a solid (221 mg, 78.7% yield).

HPLC: Betasil C18, 80:20 [KH₂PO₄ (0.01M, pH 3.2): CH₃CN], 264 nm, Rₜ 11.5 min, 98.6% purity.

¹H NMR (300 MHz, DMSO-d₆): δ 12.23 (s, 1H), 9.25 (s, 1H), 7.76 (d, J=6.3 Hz, 2H), 7.53 to 7.51 (m, 3H), 7.19 (d, J=8.4 Hz, 2H), 7.01 (b s, 2H), 2.64-2.74 (m, 2H), 2.42-2.33 (m, 2H), 2.00-1.72 (m, 2H).

LC-MSD (ES+): m/z 361 [(M+H)⁺, 100].

### Example 24

### Synthesis of 1-{4-[4-(5-methyl-isoxazol-3-ylamino)-6-phenyl-pyrimidin-2-ylamino]-phenyl}-cyclobutanecarboxylic acid

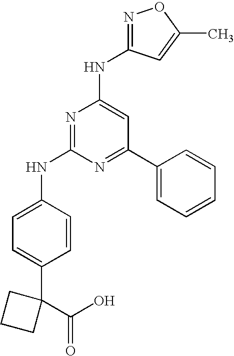

### Step (i): Synthesis of (2-chloro-6-phenyl-pyrimidin-4-yl)-(5-methyl-isoxazole-3yl)-amine:

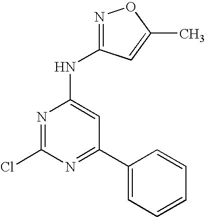

A similar procedure was used as in Example 5, step (i) by using 3-amino-5-methyl isoxazole instead of 2-(aminomethyl)-1-ethylpyrrolidine. The resulting solid was triturated with dichloromethane and ether, filter, and dried under vacuum to give title compound as pale yellow solid (1.68 g, 66% yield).

HPLC: Betasil ODS-3V C18, 30: 70 [KH₂PO₄ (0.01M, pH 3.2): CH₃CN], 264 nm, Rₜ 7.4 min, 95% purity.

¹H NMR (300 MHz, DMSO-d₆): δ 10.81 (s, 1H), 8.04-8.00 (m, 2H), 7.81 (s, 1H), 7.53 to 7.51 (m, 3H), 6.38 (s, 1H), 2.41 (s, 3H).

LC-MSD (ES+): m/z [287 (M+H)⁺, 100].

### Step(ii): Synthesis of 1-{4-[4-(5-methyl-isoxazol-3-ylamino)-6-phenyl-pyrimidin-2-ylamino]-phenyl}-cyclobutanecarboxylic acid methyl ester

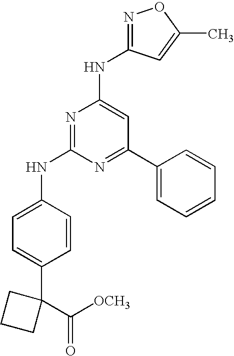

A similar method was used as in Example 1, step (iv) by using (2-chloro-6-phenyl-pyrimidin-4-yl)-(5-methyl-isoxazole-3yl)-amine instead of tert-butyl-(2-chloro-6-phenyl-pyrimdin-4-yl)-amine. Purification (Biotage Horizon HPFC chromatography system, SiO₂, 70:30 hexane: ethyl acetate) gave the title compound as a pale yellow solid (380 mg, 48% yield)

HPLC: Betasil ODS-3V C18, 30:70 [KH₂PO₄ (0.01M, pH 3.2): CH₃CN], 264 nm, Rₜ 13.2 min, 89% purity.

¹H NMR (300 MHz, DMSO-d₆): δ 10.23 (s, 1H), 9.37 (s, 1H), 8.03-8.00 (m, 2H), 7.81 (d, J=8.4 Hz, 2H), 7.55-7.53 (m, 3H), 7.23 (d, J=8.4 Hz, 2H), 7.09 (s, 1H), 6.82 (s, 1H), 3.59 (s, 3H), 2.27-2.30 (m, 7H), 1.97-1.71 (m, 2H).

LC-MSD (ES+): m/z [456 (M+H)⁺, 100].

### Step(iii): Synthesis of 1-{4-[4-(5-methyl-isoxazol-3-ylamino)-6-phenyl-pyrimidin-2-ylamino]-phenyl}-cyclobutanecarboxylic acid

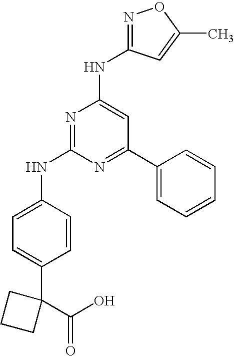

A similar method was used as in Example 1, step (v) by using 1-{4-[4-(5-methyl-isoxazol-3-ylamino)-6-phenyl-pyrimidin-2-ylamino]-phenyl}-cyclobutane carboxylic acid methyl ester instead of 1-[4-(4-tert-butylamino-6-phenyl-pyrimidin-2-ylamino)-phenyl]-cyclobutanecarboxylic acid methyl ester. The resulting pale yellow solid was dried under vacuum to give the title compound (80 mg, 27% yield).

HPLC: Betasil ODS-3V C18, 30:70 [KH₂PO₄ (0.01M, pH 3.2): CH₃CN], 264 nm, Rₜ 4.6 min, 95% purity.

¹H NMR (300 MHz, DMSO-d₆): δ 12.36 (s, 1H), 10.28 (s, 1H), 8.17 (d, J=6.9 Hz, 2H), 7.98 (d, J=8.4 Hz, 2H), 7.77 (s, 1H), 7.55-7.47 (m, 4H), 7.33 (d, J=8.4 Hz, 3H), 2.83-2.39 (m, 4H), 2.27 (s, 3H), 2.06-1.79 (m, 2H).

LC-MSD (ES+): m/z [442 (M+H)⁺, 100].

### Example 25

### Synthesis of 1-[4-(4-cyclopentyl-6-cyclopentylamino-pyrimidin-2-ylamino)-phenyl]-cyclobutanecarboxylic acid

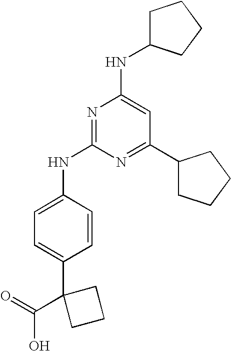

### Step (i): Synthesis of 2,4-dichloro-6-cyclopentyl-pyrimidine

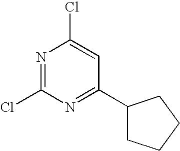

The starting compound 6-cyclopentyl-pyrimidine-2,4-diol was synthesized by following procedures reported: (a) (Li, A-H.; Moro, S.; Melman, N.; Ji, X.; Jacobson, K. A. J. Med. Chem. 1998, 41, 3186-3201 and (b) Jackman, M.; Bergman, A. J.; Archer, S. J. Am. Chem. Soc. 1948, 70, 497-499. Spectral data for 6-cyclopentyl-pyrimidine-2,4-diol: ¹H NMR (CDCl₃, 300 MHz) δ 5.46 (s, 1H), 3.06 (b s, 2H), 2.67-2.62 (m, 1H), 1.97-1.54 (m, 8H).

6-Cyclopentyl-pyrimidine-2,4-diol (2.3 g, 12.7 mmol) was mixed with phosphorus oxychloride (10 mL) and heated at 80° C. for 4 h. After evaporation of volatiles the crude residue was mixed with ice-water and adjusted to pH˜10. Dichloromethane (75 mL) was added and organic layer was separated, washed with brine, dried, and concentrated under reduced pressure to afford the title compound as a pale yellow liquid (2.2 g, 80% yield).

¹NMR (CDCl₃, 300 MHz): δ 7.15 (s, 1H), 3.17-3.05 (m, 1H), 2.09-1.99 (m, 2H), 1.85-1.68 (m, 6H).

LC-MSD (ES+): m/z [217 (M+H)⁺, 100].

### Step (ii): Synthesis of (2-chloro-6-cyclopentyl-pyrimidin-4-yl)-cyclopentyl-amine

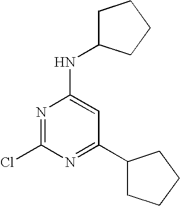

Cyclopentylamine (0.59 mL, 5.99 mmol) and DIEA (2 mL, 11.98 mmol) were added to a solution of 2,4-dichloro-6-cyclopentyl-pyrimidine (1.3 g, 5.99 mmol) in anhydrous methanol (5 mL) at 0° C. under nitrogen atmosphere. After 1 h the mixture was warmed to about 20-35° C. and stirred for 48 h. Thereafter, volatiles were evaporated under reduced pressure, and the crude material was purified by chromatography over silica gel to afford the title compound as a pale yellow viscous liquid (1.10 g, 64% yield).

¹H NMR (CDCl₃, 400 MHz): δ 6.09 (s, 1H), 5.12 (b s, 1H), 4.08-3.96 (m, 1H), 3.0-2.92 (m, 1H), 2.18-1.98 (m, 4H), 1.86-1.60 (m, 10H), 1.56-1.42 (m, 2H).

LC-MSD (ES+): m/z [266 (M+H)⁺, 100].

### Step (iii): Synthesis of 1-[4-(4-cyclopentyl-6-cyclopentylamino-pyrimidin-2-ylamino)-phenyl]-cyclobutanecarboxylic acid

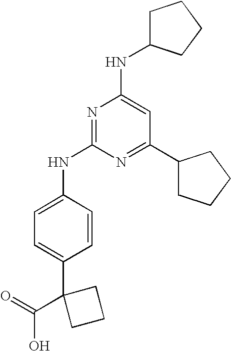

The title compound was afforded in 84% yield following the method as described in Example 3, step (ii) and step (iii) by using an appropriate starting material.

HPLC: Symmetry Shield C18, gradient, 0.01% TFA: CH₃CN, 210 nm, Rₜ 7.07 min, 99.51% purity.

¹H NMR (DMSO-d₆, 400 MHz): δ 12.4 (b s, 1H), 10.51 (b s, 1H), 8.82 (b s, 1H), 7.63 (d, J=7.8 Hz, 2H), 7.28 (d, J=7.8 Hz, 2H), 6.08 (s, 1H), 4.38-4.22 (m, 1H), 2.97-2.89 (m, 1H), 2.79-2.65 (m, 2H), 2.42-2.35 (m, 2H), 2.08-1.88 (m, 5H), 1.82-1.48 (m, 13H).

LC-MSD (ES+): m/z [421 (M+H)⁺, 100].

### Example 26

### Synthesis of 1-[4-(4-tert-butylamino-6-cyclopentyl-pyrimidin-2-ylamino)-phenyl]-cyclobutanecarboxylic acid

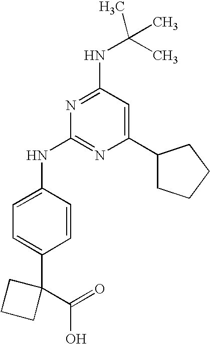

### Step (i): Synthesis of 1-[4-(4-tert-butylamino-6-cyclopentyl-pyrimidin-2-ylamino)-phenyl]-cyclobutanecarboxylic acid methyl ester

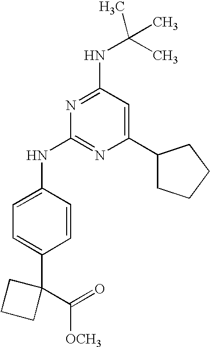

The title compound was prepared as a light yellow powder (74% yield) following the procedure as described in Example 9, steps i and ii.

HPLC: Symmetry Shield C18, gradient, [KH₂PO₄ (0.01M, pH 3.0): CH₃CN], 256 nm, Rₜ 7.8 min, 98.83% purity.

¹H NMR (CDCl₃, 400 MHz): δ 7.60 (d, J=8.0 Hz, 2H), 7.25 (d, J=8.0 Hz, 2H), 6.80 (b s, 1H), 5.78 (s, 1H), 4.60 (b s, 1H), 3.61 (s, 3H), 2.92-2.78 (m, 3H), 2.52-2.44 (m, 2H), 2.12-1.60 (m, 10H), 1.42 (s, 9H).

LC-MSD (ES+): m/z [423 (M+H)⁺, 100].

### Step (ii): Synthesis of 1-[4-(4-tert-butylamino-6-cyclopentyl-pyrimidin-2-ylamino)-phenyl]-cyclobutanecarboxylic acid

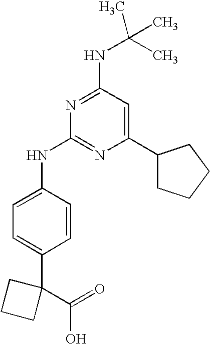

The title compound was afforded as a white solid (81% yield) following a procedure described in the synthesis of Example 9, step (iii) by using an appropriate starting material.

HPLC: Symmetry Shield C18, gradient, [KH₂PO₄ (0.01M, pH 3.0): CH₃CN], 256 nm, Rₜ 7.8 min, 98.83% purity.

¹H NMR (DMSO-d₆, 400 MHz): δ 12.6 (b s, 1H), 10.24 (b s, 1H), 8.31 (b s, 1H), 7.58 (d, J=8.0 Hz, 2H), 7.26 (d, J=8.0 Hz, 2H), 6.10 (s, 1H), 2.92-2.85 (m, 1H), 2.76-2.67 (m, 2H), 2.37-2.29 (m, 2H), 2.06-1.86 (m, 3H), 1.80-1.58 (m, 7H), 1.38 (s, 9H).

LC-MSD (ES+): m/z [409 (M+H)⁺, 100].

### Example 27

### Synthesis of 1-{4-[4-(1-methyl-1H-pyrazol-4-yl)-6-morpholin-4-yl-pyrimidin-2-ylamino]-phenyl}-cyclobutanecarboxylic acid

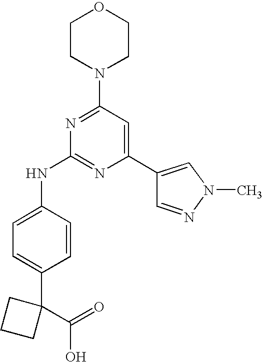

### Step (i): Synthesis of 2,4-dichloro-6-(1-methyl-1H-pyrazol-4-yl)-pyrimidine

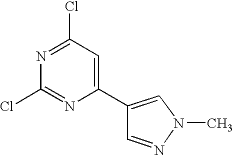

To 2,4,6-trichloropyrimidine (12.0 mL, 104.2 mmol), dissolved in THF (100 mL), was added 1-methyl-1H-pyrazole-4-boronic acid pinacol ester (18.01 g, 86.5 mmol), palladium acetate (II) (0.372 g, 1.65 mmol), triphenylphosphine (0.8975 g, 3.41 mmol), and sodium carbonate (86 mL, 173.54 mmol, 2M) sequentially. The resulting mixture was stirred at 60° C. in oil bath under nitrogen atmosphere for 3.5 h. The mixture was diluted with TBME, and was washed two times with water and one time with brine. The organic phase was dried over sodium sulfate and concentrated by rotary evaporation. The resulting solid was recrystallized with ether, hexane, and 2-propanol. The solid was dried under vacuum which gave the title compound as an off-white solid (13.9 g, 58% yield).

HPLC: Inertsil ODS-3V C18, 30:70 [KH₂PO₄ (0.01M, pH 3.2): CH₃CN], 264 nm, Rₜ 4.8 min, 86% purity.

¹H NMR (300 MHz, CDCl₃, TMS): δ 8.10 (s, 1H), 8.01 (s, 1H), 7.32 (s, 1H), 3.98 (s, 3H).

LC-MSD (ES+): m/z [229 (M+H)⁺, 100].

### Step (ii): Synthesis of 4-[2-chloro-6-(1-methyl-1H-pyrazol-4-yl)-pyrimidin-4-yl]-morpholine

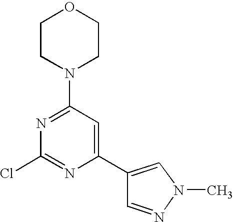

2,4-Dichloro-6-(1-methyl-1H-pyrazol-4-yl)-pyrimidine (8.2 g, 36 mmol) was dissolved in methanol (90 mL) which was followed by DIEA (18.8 mL, 108 mmol) addition. After stirring under nitrogen atmosphere for 30 min, morpholine (3.2 mL, 36 mmol) was added. The reaction mixture was stirred for about 10-19 h at about 20-35° C. The reaction mixture was extracted with dichloromethane. The combined organic layers were washed with water and brine, dried over sodium sulfate, filtered, and then concentrated by rotary evaporation. The resulting solid was dried under vacuum. Flash column chromatography (SiO₂, 96:4 dichloromethane:methanol) gave the title compound as a white solid (3.2 g, 32% yield).

HPLC: Inertsil ODS-3V C18, [KH₂PO₄ (0.01M, pH 3.2): CH₃CN], gradient, 264 nm, Rₜ 12.1 min, 97.6% purity.

¹H NMR (300 MHz, CDCl₃, TMS): δ 8.04 (s, 1H), 7.90 (s, 1H), 6.47 (s, 1H), 3.94 (s, 3H), 3.81-3.78 (m, 4H), 3.71-3.65 (m, 4H).

LC-MSD (ES+): m/z [280 (M+H)⁺, 100].

### Step Synthesis of 1-{4-[4-(1-methyl-1H-pyrazol-4-yl)-6-morpholin-4-yl-pyrimidin-2-ylamino]-phenyl}-cyclobutanecarboxylic acid methyl ester

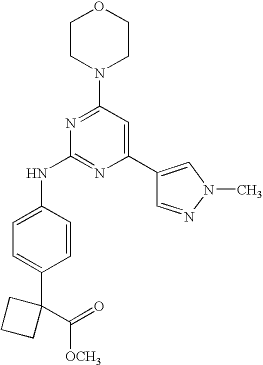

4-[2-Chloro-6-(1-methyl-1H-pyrazol-4-yl)-pyrimidin-4-yl]-morpholine (3.2 g, 11.5 mmol) was dissolved in n-butanol (60 mL) followed by addition of 1-(4-aminophenyl)-cyclobutanecarboxylic acid methyl ester (3.53 g, 17.2 mmol). The resulting mixture was stirred at reflux for about 10-19 h at 120° C. under nitrogen atmosphere. When the solution was cooled to about 20-35° C., the compound precipitated out. The precipitate was then filtered with isopropanol and ether. The resulting solid was dried under vacuum. Purification (flash chromatography system, SiO₂, 96:4 dichloromethane:methanol) gave the title compound as a white solid (4.5 g, 88% yield).

HPLC: Inertsil ODS-3V C18, [KH₂PO₄ (0.01M, pH 3.2): CH₃CN], gradient, 264 nm, Rₜ 12.8 min, 97.6% purity.

¹NMR (300 MHz, CDCl₃, TMS): δ 10.49 (b s, 1H), 8.75 (s, 1H), 7.97 (s, 1H), 7.56 (apt dd, J=2.1, 6.9 Hz, 2H), 7.28 (apt dd, J=1.8, 7.2 Hz, 2H), 6.24 (s, 1H), 3.93 (s, 3H), 3.80 (s, 8H), 3.66 (s, 3H), 2.88-2.79 (m, 2H), 2.55-2.45 (m, 2H), 2.09-1.83 (m, 2H).

LC-MSD (ES+): m/z [449 (M+H)⁺, 100].

### Step (iv): Synthesis of 1-{4-[4-(1-methyl-1H-pyrazol-4-yl)-6-morpholin-4-yl-pyrimidin-2-ylamino]-phenyl}-cyclobutanecarboxylic acid

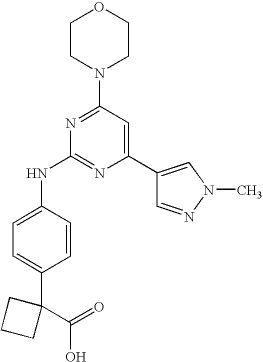

1-{4-[4-(1-Methyl-1H-pyrazol-4-yl)-6-morpholin-4-yl-pyrimidin-2-ylamino]-phenyl}-cyclobutanecarboxylic acid methyl ester (4.5 g, 10 mmol) was dissolved in methanol (100 mL) followed by a solution of NaOH in 300 mL water (1.4 g, 35 mmol). The resulting mixture was stirred at reflux for about 10-19 h under nitrogen atmosphere. The mixture was concentrated by rotary evaporation to remove most of the methanol then adjusted to pH 2-4 with 6M HCl. The precipitate was then filtered with pentane. The resulting solid was dried under vacuum which gave the title compound as a white solid (4.1 g, 95% yield).

HPLC: Inertsil ODS-3V C18, [KH₂PO₄ (0.01M, pH 3.2): CH₃CN], gradient, 264 nm, Rₜ 10.5 min, 97.9% purity.

¹H NMR (300 MHz, DMSO-d₆): δ 12.6 (b s, 1H), 10.59 (br s, 1H), 8.64 (s, 1H), 8.37 (s, 1H), 7.55 (apt d, J=7.2 Hz, 2H), 7.30 (apt d, J=7.5 Hz, 2H), 6.91 (s, 1H), 3.93 (s, 3H), 3.82-3.74 (m, 8H), 2.69-2.67 (m, 2H), 2.43-2.26 (m, 2H), 1.99-1.73 (m, 2H).

LC-MSD (ES+): m/z [435 (M+H)⁺, 100].

### Example 28

### Synthesis of 1-{4-[4-(1-methyl-1H-pyrazol-4-yl)-6-pyrrolidin-1-yl-pyrimidin-2-ylamino]-phenyl}-cyclobutanecarboxylic acid

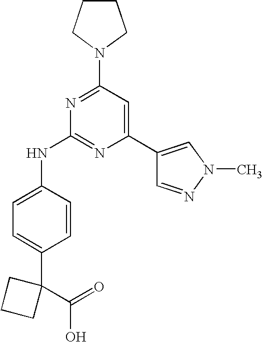

### Step (i): Synthesis of 2-chloro-4-(1-methyl-1H-pyrazol-4-yl)-6-pyrrolidin-1-yl-pyrimidine

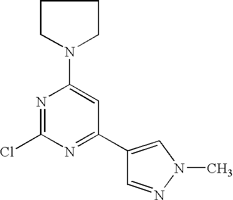

To 2,4-dichloro-6-(1-methyl-1H-pyrazol-4-yl)-pyrimidine (755 mg, 3.3 mmol), dissolved in 15 mL methanol, was added DIEA (0.61 mL, 3.5 mmol) and was stirred for ˜13 min at about 20-35° C. under a nitrogen atmosphere. To this mixture was added pyrrolidine (0.29 mL, 3.5 mmol) and stirred for ˜16 h. The mixture was concentrated by rotary evaporation to give a solid. Purification (Biotage Horizon HPFC system chromatography, SiO₂, 100% EtOAc) gave the title compound as a white solid (440 mg, 50.6% yield).

HPLC: Inertsil ODS-3V C18, 30:70 [KH₂PO₄ (0.01M, pH 3.2): CH₃CN], 264 nm, Rₜ 4.4 min, 99.8% purity.

¹H NMR (300 MHz, CDCl₃, TMS): δ 8.00 (s, 1H), 7.90 (s, 1H), 6.26 (s, 1H), 3.93 (s, 3H), 3.64 (b apt s, 2H), 3.40 (b apt s, 2H), 2.03 (b apt s, 4H).

Mass: LC-MSD (ES+): m/z [264 (M+H)⁺, 100].

### Step (ii): Synthesis of 1-{4-[4-(1-methyl-1H-pyrazol-4-yl)-6-pyrrolidin-1-yl-pyrimidin-2-ylamino]-phenyl}-cyclobutanecarboxylic acid methyl ester

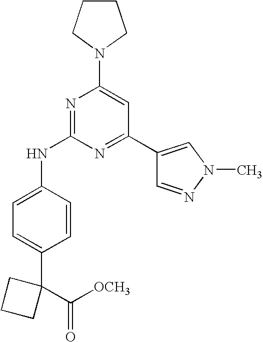

2-Chloro-4-(1-methyl-1H-pyrazol-4-yl)-6-pyrrolidin-1-yl-pyrimidine (400 mg, 1.52 mmol) and 1-(4-aminophenyl)-cyclobutanecarboxylic acid methyl ester (310 mg, 1.52 mmol) were mixed in n-butanol (20 mL) and heated at reflux under a nitrogen atmosphere for ˜15 h. After cooling to ambient temperature, a precipitate formed and was collected by vacuum filtration. The solid was rinsed with diethyl ether, 2-propanol, then diethyl ether, and dried under vacuum to give the title compound as a solid (524 mg, 79.8% yield).

HPLC: Inertsil ODS-3V C18, 30:70 [KH₂PO₄ (0.01M, pH 3.2): CH₃CN], 264 nm, Rₜ 3.7 min, 98.9% purity.

¹H NMR (300 MHz, CDCl₃, TMS): δ 10.5 (s, 1H), 8.84 (s, 1H), 7.93 (s, 1H), 7.70 (d, J=8.7 Hz, 2H), 7.29 (overlap with CHCl₃ solvent), 6.02 (s, 1H), 3.97 (s, 3H), 3.74-3.58 (m, 7H), 2.88-2.80 (m, 2H), 2.55-2.45 (m, 2H), 2.16-1.83 (m, 6H).

Mass: LC-MSD (ES+): m/z [433 (M+H)⁺, 100].

### Step (iii): Synthesis of 1-{4-[4-(1-methyl-1H-pyrazol-4-yl)-6-pyrrolidin-1-yl-pyrimidin-2-ylamino]-phenyl}-cyclobutanecarboxylic acid

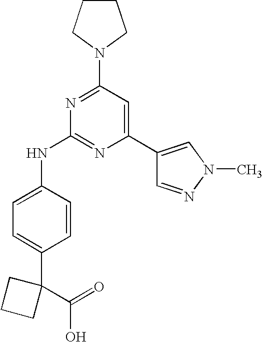

1-{4-[4-(1-Methyl-1H-pyrazol-4-yl)-6-pyrrolidin-1-yl-pyrimidin-2-ylamino]-phenyl}-cyclobutanecarboxylic acid methyl ester (394 mg, 0.9 mmol) was dissolved in 10 mL MeOH, and a NaOH solution (107 mg, 2.7 mmol dissolved in 3 mL water) was added. The mixture was heated at reflux for ˜1.5 h and was followed by addition of NaOH (114 mg, 2.85 mmol dissolved in 3 mL water) and 3 mL MeOH sequentially. The mixture was heated at reflux for ˜17-19 h and then cooled to ambient temperature. The reaction mixture was concentrated by rotary evaporation to remove the MeOH and then filtered by vacuum. The supernatant was made acidic by slowly adding 6M HCl (aq) to a pH˜2-4 resulting in a precipitate. The solid was re-dissolved in 10% NaOH (aq), filtered, and then acidified with 6M HCl (aq). The solid was collected by vacuum filtration and rinsed with excess water. The collected solid was dried for about 10-19 h under vacuum to give the title compound as a solid (56 mg, 14.8% yield).

HPLC: Inertsil ODS-3V C18, [KH₂PO₄ (0.01M, pH 3.2): CH₃CN], gradient, 264 nm, Rₜ 11.3 min, 99.4% purity.

¹H NMR (300 MHz, DMSO-d₆): δ 12.69 (b s, 1H), 10.66 (b s, 1H), 8.67 (s, 1H), 8.39 (s, 1H), 7.64 (d, J=7.8 Hz, 2H), 7.30 (d, J=8.4 Hz, 2H), 6.60 (s, 1H), 3.93 (s, 3H), 3.60-3.68 (m, 4H), 2.68-2.35 (m, 6H), 2.00-1.80 (m, 4H).

Mass: LC-MSD (ES+): m/z [419 (M+H)⁺, 100].

### Example 29

### Synthesis of 1-{4-[4-(4-hydroxy-piperidin-1-yl)-6-(1-methyl-1H-pyrazol-4-yl)-pyrimidin-2-ylamino]-phenyl}-cyclobutanecarboxylic acid

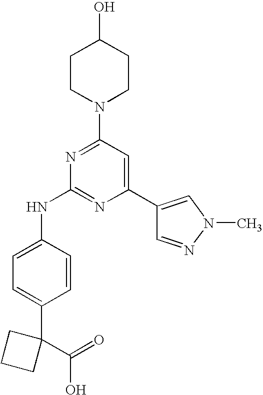

### Step (i): Synthesis of 1-[2-chloro-6-(1-methyl-1H-pyrazol-4-yl)-pyrimidin-4-yl]-piperidin-4-ol hydrochloride

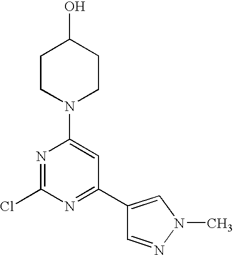

To 2,4-dichloro-6-(1-methyl-1H-pyrazol-4-yl)-pyrimidine (753 mg, 3.3 mmol), dissolved in 15 mL methanol, was added DIEA (0.61 mL, 3.5 mmol) and was stirred for ˜13 min at about 20-35° C. under a nitrogen atmosphere. To this mixture was added 4-hydroxypiperidine (354 mg, 3.5 mmol) and stirred ˜16 h. After concentrating in vacuo, a solid was collected and swirled with 15-20 mL dichloromethane. The solid was collected by vacuum filtration and dried under vacuum to give title compound as a white solid (478 mg, 49.4% yield).

HPLC: Inertsil ODS-3V C18, 30:70 [KH₂PO₄ (0.01M, pH 3.2): CH₃CN], 264 nm, Rₜ 2.9 min, 94% purity.

¹NMR (300 MHz, DMSO-d₆): δ 8.67 (b s, 1H), 8.35 (s, 1H), 8.08 (s, 1H), 7.04 (s, 1H), 5.00-4.81 (m, 1H), 4.05-4.01 (m, 1H), 3.86 (s, 3H), 3.77-3.75 (m, 1H), 3.16 (br s, 1H), 2.92 (b s, 1H), 1.88-1.77 (m, 2H), 1.61-1.50 (m, 1H), 1.40-1.24 (m, 2H).

Mass: LC-MSD (ES+): m/z [294 (M+H)⁺, 100].

### Step (ii): Synthesis of 1-{4-[4-(4-hydroxy-piperidin-1-yl)-6-(1-methyl-1H-pyrazol-4-yl)-pyrimidin-2-ylamino]-phenyl -cyclobutanecarboxylic acid methyl ester

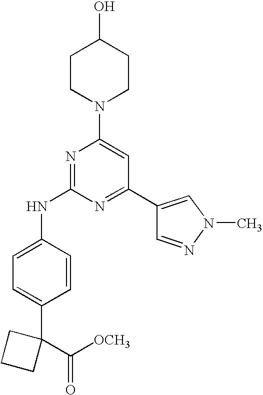

A similar method was followed as in Example 28, step (ii) by using 1-[2-chloro-6-(1-methyl-1H-pyrazol-4-yl)-pyrimidin-4-yl]-piperidin-4-ol hydrochloride (425 mg, 1.45 mmol), and 1-(4-aminophenyl)-cyclobutanecarboxylic acid methyl ester (297 mg, 1.45 mmol). The title compound was isolated as a solid (456 mg, 68% yield).

HPLC: Inertsil ODS-3V C18, [KH₂PO₄ (0.01M, pH 3.2): CH₃CN], gradient, 264 nm, Rₜ 10.7 min, 95% purity.

¹H NMR (300 MHz, CDCl₃, TMS): δ 10.32 (s, 1H), 8.62 (s, 1H), 8.34 (s, 1H), 7.56 (d, J=8.2 Hz, 2H), 7.31 (d, J=8.7, 2H), 6.92 (s, 1H), 4.20 (b s, 1H), 3.94 (s, 3H), 3.85 (b apt s, 1H), 3.58 (s, 3H), 2.77-2.68 (m, 2H), 2.46-2.39 (m, DMSO overlap, 2H), 1.99-1.77 (m, 4H), 1.49 (b apt s, 2H).

Mass: LC-MSD (ES+): m/z [463 (M+H)⁺, 100].

### Step (iii): Synthesis of 1-{4-[4-(4-hydroxy-piperidin-1-yl)-6-(1-methyl-1H-pyrazol-4-yl)-pyrimidin-2-ylamino]-phenyl}-cyclobutanecarboxylic acid

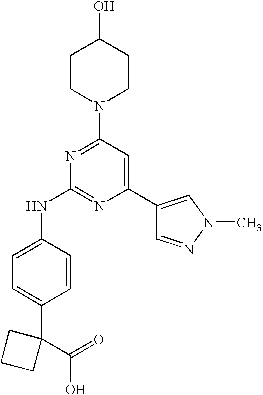

1-{4-[4-(4-Hydroxy-piperidin-1-yl)-6-(1-methyl-1H-pyrazol-4-yl)-pyrimidin-2-ylamino]-phenyl}-cyclobutanecarboxylic acid methyl ester (391 mg, 0.85 mmol) was dissolved in 10 mL MeOH, and NaOH solution (123 mg, 3 mmol dissolved in 3 mL water) was added. The mixture was heated at reflux for about 10-19 h and then cooled to ambient temperature. The reaction mixture was concentrated by rotary evaporation to remove the MeOH and then filtered by vacuum. The supernatant was made acidic by slowly adding 6M HCl (aq) forming a precipitate. The solid was collected by vacuum filtration and rinsed with excess water, diethyl ether, 2-propanol, and then diethyl ether. The collected solid was dried for about 10-19 h under vacuum to give the title compound as a solid (274 mg, 71.9% yield).

HPLC: Inertsil ODS-3V C18, [KH₂PO₄ (0.01M, pH 3.2): CH₃CN], gradient, 264 nm, Rₜ 9.2 min, 98.8% purity.

¹H NMR (300 MHz, DMSO-d₆): δ 12.33 (s, 1H), 10.41 (s, 1H), 8.64 (s, 1H), 8.37 (s, 1H), 7.54 (d, J=8.7 Hz, 2H), 7.31 (d, J=8.7 Hz, 2H), 6.92 (s, 1H), 4.17-3.57 (m, 6H), 3.94 (s, 3H), 2.75-2.66 (m, 2H), 2.44-2.35 (m, 2H), 2.00-1.72 (m, 4H), 1.49-1.46 (m, 2H).

Mass: LC-MSD (ES+): m/z [449 (M+H)⁺, 100].

### Example 30

### Synthesis of 1-{4-[4-(3,5-dimethyl-isoxazol-4-yl)-6-morpholin-4-yl-pyrimidin-2-ylamino]-phenyl}-cyclobutanecarboxylic acid

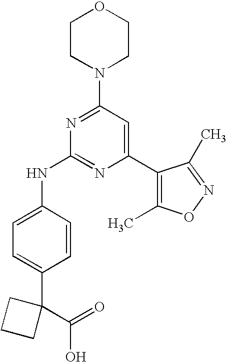

### Step (i): Synthesis of 2,4-dichloro-6-(3,5-dimethyl-isoxazol-4-yl)-pyrimidine

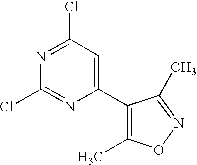

To 2,4,6-trichloropyrimidine (25 g, 170 mmol) dissolved in THF (200 mL) was added 3,5-dimethylisoxazole boronic acid (20 g, 142 mmol), palladium acetate (II) (640.4 g, 2.84 mmol), triphenylphosphine (1.49 g, 5.68 mmol) and sodium carbonate (30.036 g, 284 mmol) in water (125 mL). The resulting mixture was stirred at 60° C. in an oil bath under nitrogen atmosphere for 5 h. The mixture was diluted with TBME, and was washed two times with water and one time with brine. The organic phase was dried over magnesium sulfate and concentrated by rotary evaporation. Purification by flash column chromatography (SiO₂, 80:20 hexane:EtOAc) gave the title compound as a yellow solid (7.46 g, 22% yield).

HPLC: Inertsil ODS-3V C18, 30:70 [KH₂PO₄ (0.01M, pH 3.2): CH₃CN], 264 nm, Rₜ 7.4 min, 95.98% purity.

¹H NMR (CDCl₃, 300 MHz): δ 7.30 (s, 1H), 2.73 (s, 3H), 2.51 (s, 3H).

LC-MSD (ES+): m/z [244 (M+H)⁺, 100].

### Step (ii): Synthesis of 4-[2-chloro-6-(3,5-dimethyl-isoxazol-4-yl)-pyrimidin-4-yl]-morpholine

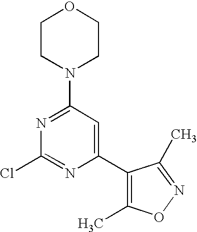

To 2,4-dichloro-6-(3,5-dimethyl-isoxazol-4-yl)-pyrimidine (7.06 g, 28.7 mmol) dissolved in methanol (60 mL) was added DIEA (5 mL, 28.7 mmol). The resulting solution was allowed to stir at about 20-35° C. for about 15 min. Morpholine (2.50 mL, 28.7 mmol) was added to the reaction mixture. After stirring at about 20-35° C. for about 10-19 h, the reaction mixture was extracted 3 times with EtOAc. The combined organic layers were washed with water and brine, dried over sodium sulfate, filtered, and then concentrated under vacuum to give product. Purification by flash column chromatography (SiO₂, 50:50 hexane:EtOAc) gave the title compound as a light yellow solid (6.38 g, 75% yield).

HPLC: Inertsil ODS-3V C18, 30:70 [KH₂PO₄ (0.01M, pH 3.2): CH₃CN], 264 nm, Rₜ 5.7 min, 99.3% purity.

¹HNMR (CDCl₃, 300 MHz): δ 6.34 (s, 1H), 3.80-3.77 (m, 4H), 3.67-3.64 (m, 4H), 2.60 (s, 3H), 2.41 (s, 3H).

LC-MSD (ES+): m/z [295 (M+H)⁺, 100].

### Step (iii): Synthesis of 1-{4-[4-(3,5-dimethyl-isoxazol-4-yl)-6-morpholin-4-yl-pyrimidin-2-ylamino]-phenyl}-cyclobutanecarboxylic acid methyl ester

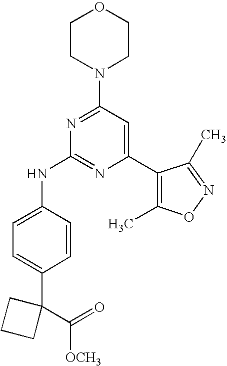

To 4-[2-chloro-6-(3,5-dimethyl-isoxazol-4-yl)-pyrimidin-4-yl]-morpholine (6 g, 20.4 mmol) dissolved in n-butanol (100 mL) was added 1-(4-aminophenyl)-yclobutanecarboxylic acid methyl ester (4.26 g, 20.4 mmol). The resulting mixture was stirred and refluxed for about 10-19 h under nitrogen atmosphere. The mixture was washed one time with saturated sodium carbonate, then extracted two times with EtOAc, and finally washed two times with water and one time with brine. The organic phase was dried over sodium sulfate, filtered, concentrated by rotary evaporation, and dried for about 10-19 h under vacuum. Purification by flash column chromatography (SiO₂, 50:50 hexane:EtOAc) gave the title compound as a white solid (6.37 g, 67% yield).

HPLC: Inertsil ODS-3V C18, 30:70 [KH₂PO₄ (0.01M, pH 3.2): CH₃CN], 264 nm, Rₜ 9.1 min, 96.03% purity.

¹H NMR (CDCl₃, 300 MHz): δ 7.60-7.49 (m, 2H), 7.35-7.13 (m, 2H), 7.08 (br s, 1H), 6.00 (s, 1H), 3.83-3.80 (m, 4H), 3.66-3.63 (m, 7H), 2.86-2.78 (m, 2H), 2.60 (s, 3H), 2.54-2.43 (m, 5H), 2.07-1.82 (m, 2H).

LC-MSD (ES+): m/z [464 (M+H)⁺, 100].

### Step (iv): Synthesis of 1-{4-[4-(3,5-dimethyl-isoxazol-4-yl)-6-morpholin-4-yl-pyrimidin-2-ylamino]-phenyl}-cyclobutanecarboxylic acid

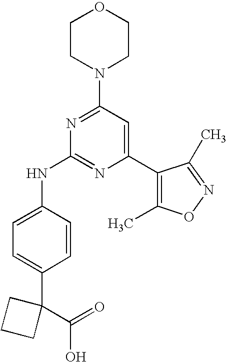

To 1-{4-[4-(3,5-dimethyl-isoxazol-4-yl)-6-morpholin-4-yl-pyrimidin-2-ylamino]-phenyl}-cyclobutanecarboxylic acid methyl ester (5.01 g, 10.8 mmol) dissolved in methanol (75 mL) was added a solution of NaOH (1.54 g, 37.8 mmol) in water (10 mL). The resulting mixture was stirred at reflux for about 10-19 h under nitrogen atmosphere. The mixture was concentrated by rotary evaporation to remove most of the methanol and adjusted to pH 2-3 with dilute HCl (aq). The precipitate was collected by vacuum filtration and rinsed with ether and water. The resulting sample was dried under vacuum to give the title compound as a white solid (4.09 g, 84% yield).

HPLC: Inertsil ODS-3V C18, 30:70 [KH₂PO₄ (0.01M, pH 3.2): CH₃CN], 264 nm, Rₜ 4.9 min, 99.07% purity.

hu 1H NMR (300 MHz, DMSO-d₆): δ 12.23 (br s, 1H), 9.27 (br s, 1H), 7.64 (d, J=8.7 Hz, 2H), 7.17 (d, J=8.4 Hz, 3H), 6.35 (s, 1H), 3.67-3.65 (m, 8H), 2.70-2.61 (m, 2H), 2.57 (s, 3H), 2.47-2.30 (m, 5H), 1.95-1.67 (m, 2H).

LC-MSD (ES+): m/z [450 (M+H)⁺, 100].

### Example 31

### Synthesis of 1-{3-[4-(3,5-dimethyl-isoxazol-4-yl)-6-morpholin-4-yl-pyrimidin-2-ylamino]-phenyl}-cyclobutanecarboxylic acid

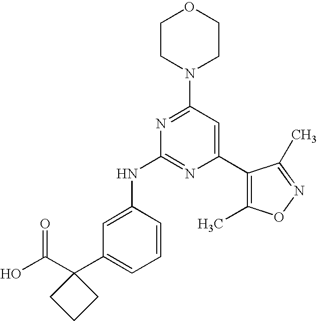

### Step (i): Synthesis of the intermediate 1-(3-amino-phenyl)-cyclobutanecarbonitrile

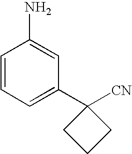

### (a): Synthesis of 1-(3-nitro-phenyl)-cyclobutanecarbonitrile

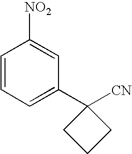

3-Nitro-phenyl acetonitrile (1.02 g, 6.2 mmol) was dissolved in anhydrous acetonitrile (15 mL); and 1,3-dibromopropane (1.4 mL, 13.6 mmol), tetra-butyl-ammonium hydrogen sulfate (1.044 g, 3.1 mmol), and potassium carbonate (3.429 g, 24.8 mmol) were added sequentially. The resulting mixture was heated at reflux for 48 h under nitrogen atmosphere. The reaction mixture was filtered through a Celite plug, rinsed with ethyl acetate, and concentrated by rotary evaporation. The mixture was again diluted with ethyl acetate, and washed two times with water and one time with brine. The organic phase was dried over sodium sulfate, filtered, and concentrated by rotary evaporation. The resulting sample was dried under vacuum. Purification (Biotage Horizon HPFC chromatography system, SiO₂, (70:30 hexane:ethyl acetate) gave the title compound as a brown solid (0.866 g, 68% yield).

¹H NMR (300 MHz, CDCl₃, TMS) δ 8.30-8.18 (m, 2H), 7.81-7.77 (m, 1H), 7.63 to 7.60 (m, 1H), 2.96-2.87 (m, 2H), 2.73-2.46 (m, 3H), 2.20-2.11 (m, 1H).

LC-MSD (ES+): (m/z) [203 (M+H)⁺, 100].

### (b) Synthesis of the intermediate 1-(3-amino-phenyl)-cyclobutanecarbonitrile

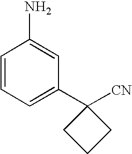

A similar method was used as in Example 1, step i(c) by using 1-(3-nitro-phenyl)-cyclobutanecarbonitrile (0.866 g, 4.2 mmol), ethanol (10 mL) and tin (II) chloride dihydrate (3.789 g, 16.8 mmol). The title compound, thick gummy yellow solid, was dried under vacuum for about 10-19 h and was used without further purification (0.61 g, 73% yield).

¹H NMR (300 MHz, CDCl₃, TMS) δ 7.15 (t, J=7.8 Hz, 1H), 6.78-6.75 (m, 1H), 6.70 (t, J=1.8 Hz, 1H), 6.62-6.59 (m, 1H), 3.81 (s, 2H), 2.79-2.70 (m, 2H), 2.63-2.52 (m, 2H), 2.45-2.29 (m, 1H), 2.09-1.96 (m, 1H).

LC-MSD (ES+): (m/z) [173 (M+H)⁺, 100].

### Step (ii): Synthesis 1-{3-[4-(3,5-dimethyl-isoxazol-4-yl)-6-morpholin-4-yl-pyrimidin-2-ylamino]-phenyl}-cyclobutanecarbonitrile

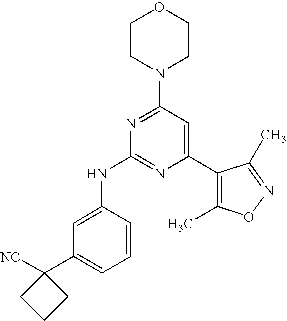

A similar method was used as in Example 1, step (iv) by using 4-[2-chloro-6-(3,5-dimethyl-isoxazol-4-yl)-pyrimidin-4-yl]-morpholine (1.002 g, 3.4 mmol) and 1-(3-amino-phenyl)-cyclobutane-carbonitrile instead of tert-butyl-(2-chloro-6-phenyl-pyrimidin-4-yl)-amine and 1-(4-aminophenyl)-cyclobutane-carboxylic acid methyl ester. Purification (Biotage Horizon HPFC chromatography system, SiO₂, 95:5 dichloromethane:MeOH) gave a white solid (0.762 g, 54% yield).

HPLC: Betasil ODS-3V C18, 30:70 [KH₂PO₄ (0.O1M, pH 3.2): CH₃CN], 264 nm, Rₜ 6.8 min, 95% purity.

¹H NMR (300 MHz, DMSO-d₆) δ 9.19 (s, 1H), 7.96 (s, 1H), 7.58 (d, J=7.8 Hz, 1H), 7.30 (t, J=7.8 Hz, 1H), 6.96 (d, J=1.8 Hz, 1H), 6.30 (s, 1H), 3.70-3.66 (m, 8H), 2.71-2.60 (m, 6H), 2.41-2.21 (m, 5H), 2.19-1.98 (m, 1H).

LC-MSD (ES+): (m/z) [431 (M+H)⁺, 100].

### Step (iii): Synthesis 1-{3-[4-(3,5-dimethyl-isoxazol-4-yl)-6-morpholin-4-yl-pyrimidin-ylamino]-phenyl}-cyclobutanecarboxylic acid

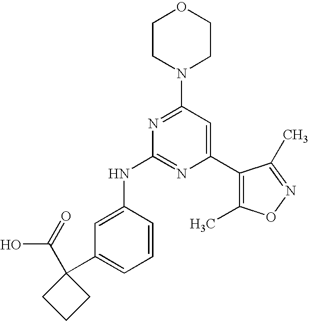

To 1-{3-[4-(3,5-dimethyl-isoxazol-4-yl)-6-morpholin-4-yl-pyrimidin-2-ylamino]-phenyl}-cyclobutanecarbonitrile (0.252 g, 0.58 mmol) was dissolved in water (5 mL) and 1-propanol (2 ml), and concentrated HCl (2 mL, 24.2 mmol) was added. The resulting mixture was refluxed for 72 h under nitrogen atmosphere. The solution was cooled, basified with 10% NaOH and filtered. The filtrate was then re-adjusted to pH 4 with 20% HCl. The precipitate was then filtered and washed with water. Purification (Biotage Horizon HPFC chromatography system, SiO₂, 95:5 dichloromethane:MeOH) gave a white solid (0.065 g, 24% yield) as the title compound.

HPLC: Betasil ODS-3V C18, 30:70 [KH₂PO₄ (0.01M, pH 3.2): CH₃CN], 264 nm, Rₜ 4.5min, 95% purity.

¹H NMR (300 MHz, CDCl₃, TMS) δ 9.34 (s, 1H), 7.65 (d, J=7.5 Hz, 1H), 7.53 (s, 1H), 7.24 (t, J=7.8 Hz, 1H), 6.95 (d, J=7.2 Hz, 1H), 5.86 (s, 1H), 3.78-3.65 (m, 8H), 2.82 (b s, 2H), 2.52-2.31 (m, 8H), 2.11-1.89 (m, 2H).

LC-MSD (ES+): (m/z) [450 (M+H)⁺, 100].

### Example 32

### Synthesis of 1-{4-[4-(3,5-dimethyl-isoxazol-4-yl)-5-methyl-6-morpholin-4-yl-pyrimidin-2-ylamino]-phenyl}-cyclobutanecarboxylic acid

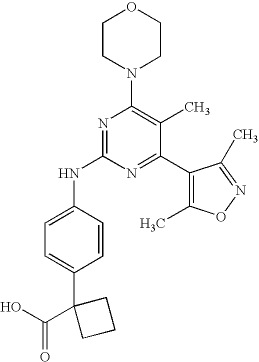

The title compound was afforded as an off white solid following the procedure as described in Example 30, by using 2,4,6-trichloro-5-methyl pyrimidine instead of 2,4,6-trichloropyrimidine. (22% yield).

HPLC: Betasil ODS-3V C18, 30:70 [KH₂PO₄ (0.01M, pH 3.2): CH₃CN], 264 nm, Rₜ 4.21 min, 99.04% purity.

¹H NMR (300 MHz, DMSO-d₆): δ 12.27 (b s, 1H), 9.32 (s, 1H), 7.67 (d, J=8.7 Hz, 2H), 7.13 (d, J=9.0 Hz, 2H), 3.73 (s, 1H), 3.39-3.32 (m, 8H), 2.68-1.63 (m, 12H, including DMSO).

LC-MSD (ES+): m/z [463 (M+H)⁺, 100].

### Example 33

### Synthesis of 1-{4-[4-(3,5-dimethyl-isoxazol-4-yl)-6-piperazin-1-yl-pyrimidin-2-ylamino]-phenyl}-cyclobutanecarboxylic acid

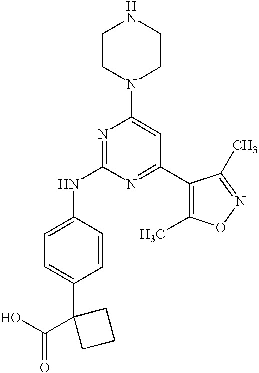

A similar method was used as in example 1, step (v) by using 1-{4-[4-(4-acetyl-piperazin-1-yl)-6-(3,5-dimethyl-isoxazol-4-yl)-pyrimidin-2-ylamino]-phenyl}-cyclobutanecarboxylic acid methyl ester instead of 1-[4-(4-tert-butylamino-6-phenyl-pyrimidin-2-ylamino)-phenyl]-cyclobutanecarboxylic acid methyl ester. The precipitate was allowed to dry under vacuum leaving the product as an off white solid (90 mg, 31% yield).

HPLC: Betasil ODS-3V C18, 30:70 [KH₂PO₄ (0.01M, pH 3.2): CH₃CN], 264 nm, Rₜ 9.64 min, 99.5% purity.

¹H NMR (300 MHz, DMSO-d₆): δ 8.99 (b s, 1H), 7.83 to 7.62 (m, 2H), 7.13 to 7.04 (m, 2H), 6.23 (s, 1H), 3.57 (s, 8H), 2.78-2.35 (m, 10H, including DMSO), 1.86-1.77 (m, 2H).

LC-MSD (ES+): m/z [449 (M+H)⁺, 100].

### Example 34

### Synthesis of 1-{4-[4-(4-acetyl-piperazin-1-yl)-6-(3,5-dimethyl-isoxazol-4-yl)-pyrimidin-2-ylamino]-phenyl}-cyclobutanecarboxylic acid

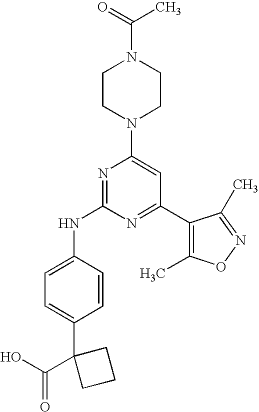

### Step (i): Synthesis of 1-{4-[4-(4-acetyl-piperazin-1-yl)-6-(3,5-dimethyl-isoxazol-4-yl)-pyrimidin-2-ylamino]-phenyl}-cyclobutanecarboxylic acid methyl ester

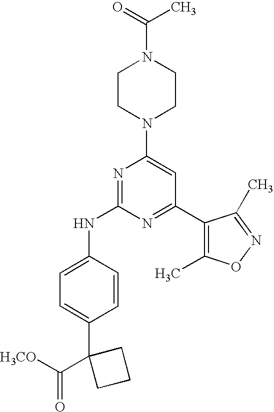

A similar method was used as in Example 1, step (iv) by using 1-{4-[2-chloro-6-(3,5-dimethyl-isoxazol-4-yl)-pyrimidin-4-yl]-piperazin-1-yl}-ethanone instead of tert-butyl-(2-chloro-6-phenyl-pyrimidin-4-yl)-amine. Flash column chromatography (SiO₂, 95:5 dichloromethane:MeOH) purification gave the product as an off-white solid (323 mg, 29% yield).

HPLC: Betasil ODS-3V C18, 30:70 [KH₂PO₄ (0.01M, pH 3.2): CH₃CN], 264 nm, Rₜ 5.43 min, 91.4% purity.

¹H NMR (300 MHz, CDCl₃, TMS): δ 7.79-7.74 (m, 2H), 7.28-7.25 (m, 2H), 6.00 (s, 1H), 3.65 (s, 8H), 2.87-2.78 (m, 2H), 2.61 (s, 3H), 2.43 (s, 3H), 2.16 (s, 3H), 2.07-1.81 (m, 4H), 1.60-1.25 (m, 4H).

LC-MSD (ES+): m/z [505 (M+H)⁺, 100].

### Step (ii): Synthesis of 1-{4-[4-(4-acetyl-piperazin-1-yl)-6-(3,5-dimethyl-isoxazol-4-yl)-pyrimidin-2-ylamino]-phenyl}-cyclobutanecarboxylic acid

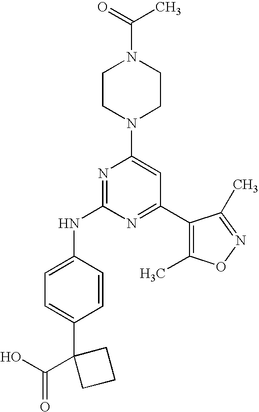

To 1-{4-[4-(4-acetyl-piperazin-1-yl)-6-(3,5-dimethyl-isoxazol-4-yl)-pyrimidin-2-ylamino]-phenyl}-cyclobutanecarboxylic acid methyl ester (1.69 g, 3.33 mmol), dissolved in THF (15 mL), was added lithium hydroxide (1.123 g, 26.8 mmol) dissolved in water (10 mL). The resulting solution was allowed to stir at about 20-35° C. for about 40 h. The solution was concentrated to remove most of the THF and remaining residue was taken up in water. The solution was adjusted to pH 4-5 with 15% hydrochloric acid and the precipitate that formed was dissolved in dichloromethane. The organic phase was dried over sodium sulfate, filtered, and concentrated by rotary evaporation. The resulting sample was dried under vacuum and purified. Purification (Biotage Horizon HPFC chromatography system, SiO₂, 95:5 dichloromethane:methanol) purification gave the title compound as a light yellow solid (352 mg, 22% yield).

HPLC: Betasil ODS-3V C18, 30:70 [KH₂PO₄ (0.01M, pH 3.2): CH₃CN], 264 nm, Rₜ 3.13 min, 98.9% purity.

¹H NMR (300 MHz, CDCl₃, TMS): δ 8.93 (br s, 1H),7.62 (d, J=7.62 Hz, 2H), 7.27 (d, J=8.4 Hz, 2H), 5.93 (s, 1H), 3.82-3.64 (m, 8H), 2.87-2.77 (m, 2H), 2.54-2.37 (m, 4H), 2.34 (s, 3H), 2.16 (s, 3H), 2.12-1.80 (m, 4H).

LC-MSD (ES+): m/z [491 (M+H)⁺, 100].

### Example 35

### Synthesis of 1-{4-[4-(6-methoxy-pyridin-3-yl)-6-morpholin-4-yl-pyrimidin-2-ylamino]-phenyl}-cyclobutanecarboxylic acid

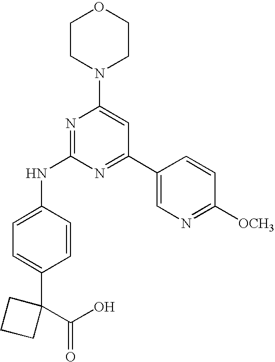

### Step (i): Synthesis of 2,4-dichloro-6-(6-methoxy-pyridin-3-yl)-pyrimidine

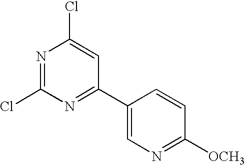

The title compound was prepared following the procedure as described in Example 30, step (i), by using 2-methoxy-3-pyridineboronic acid instead of 3,5-dimethylisoxazole boronic acid as a light yellow solid (40% yield).

¹H NMR (300 MHz, CDCl₃, TMS): δ 8.86 (d, J=1.8 Hz, 1H), 8.31 (dd, J=8.7, 2.7 Hz, 1H), 7.59 (s, 1H), 6.88 (d, J=8.7 Hz, 1H), 4.02 (s, 3H).

LC-MSD (ES+): m/z 256 [(M+H)⁺, 100].

### Step (ii): Synthesis of 4-[2-chloro-6-(6-methoxy-pyridin-3-yl)-pyrimidin-4-yl]-morpholine

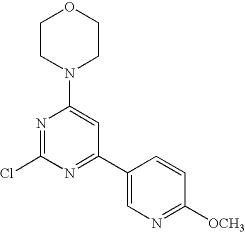

The title compound was prepared following the procedure as described in Example 30, step (ii), by using 2,4-dichloro-6-(6-methoxy-pyridin-3-yl)-pyrimidine instead of 2,4-dichloro-6-(3,5-dimethyl-isoxazol-4-yl)-pyrimidine as a white solid (70% yield).

¹H NMR (300 MHz, CDCl₃): δ 8.71 (d, J=1.8 Hz, 1H), 8.23 (dd, J=8.7, 2.7 Hz, 1H), 6.84 (d, J=8.7 Hz, 1H), 6.67 (s, 1H), 4.01 (s, 3H), 3.82-3.70 (m, 8H).

LC-MSD (ES+): m/z 307 [(M+H)⁺, 100].

### Step (iii): Synthesis of 1-{4-[4-(6-methoxy-pyridin-3-yl)-6-morpholin-4-yl-pyrimidin-2-ylamino]-phenyl}-cyclobutanecarboxylic acid methyl ester

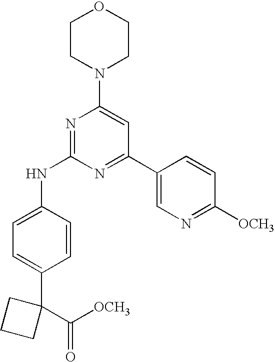

The title compound was prepared following the procedure as described in Example 30, step (iii), by using 4-[2-chloro-6-(6-methoxy-pyridin-3-yl)-pyrimidin-4-yl]-morpholine instead of 4-[2-chloro-6-(3,5-dimethyl-isoxazol-4-yl)-pyrimidin-4-yl]-morpholine as a white solid (67% yield).

HPLC: Betasil ODS-3V C18, 30:70 [KH₂PO₄ (0.01M, pH 3.2): CH₃CN], 280 nm, Rₜ 11.01 min, 93.9% purity.

¹H NMR (300 MHz, CDCl₃): δ 11.0 (s, 1H), 8.75 (d, J=2.4 Hz, 1H), 8.40 (dd, J=8.7, 2.4 Hz, 1H), 7.58 (d, J=8.7 Hz, 2H), 7.30 (d, J=8.7 Hz, 2H), 6.87 (d, J=8.7 Hz, 1H), 6.33 (s, 1H), 3.94 (s, 3H), 3.82-3.67 (m, 8H), 3.65 (s, 3H), 2.86-2.54 (m, 2H), 2.51-2.54 (m, 2H), 2.05-1.91 (m, 2H).

LC-MSD (ES+): m/z [476 (M+H)⁺, 100].

### Step (iv): Synthesis of 1-{4-[4-(6-methoxy-pyridin-3-yl)-6-morpholin-4-yl-pyrimidin-2-ylamino]-phenyl -cyclobutanecarboxylic acid

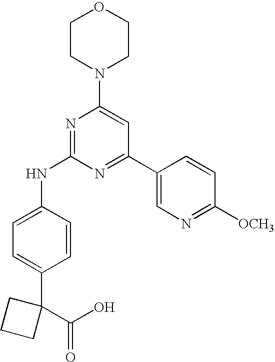

The title compound was prepared following the procedure as described in Example 30, step (iv), by using 1-{4-[4-(6-methoxy-pyridin-3-yl)-6-morpholin-4-yl-pyrimidin-2-ylamino]-phenyl}-cyclobutanecarboxylic acid methyl ester instead of 1-{4-[4-(3,5-dimethyl-isoxazol-4-yl)-6-morpholin-4-yl-pyrimidin-2-ylamino]-phenyl}-cyclobutanecarboxylic acid methyl ester as a white solid (67% yield).

HPLC: Betasil ODS-3V C18, 30:70 [KH₂PO₄ (0.01M, pH 3.2): CH₃CN], 264 nm, Rₜ 5.58 min, 95.42% purity.

¹H NMR (300 MHz, CDCl₃+CD₃OD): δ 8.47 (d, J=2.7 Hz, 1H), 8.40 (dd, J=9.0, 2.4 Hz, 1H), 7.28 (d, J=8.7 Hz, 2H), 7.11 (d, J=8.7 Hz, 2H), 6.71 (d, J=9.0 Hz, 1H), 6.37 (s, 1H), 3.67-3.57 (m, 8H), 3.07 (s, 3H), 2.58-2.54 (m, 2H), 2.27-2.21 (m, 2H), 1.90-1.78 (m, 2H).

LC-MSD (ES+): m/z [462 (M+H)⁺, 100].

### Example 36

### Synthesis of 1-{4-[4-(4,4-difluoro-piperidin-1-yl)-6-(3,5-dimethyl-isoxazol-4-yl)-pyrimidin-2-ylamino]-phenyl}-cyclobutanecarboxylic acid

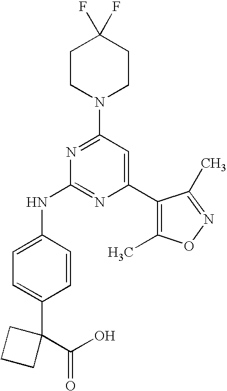

### Step (i): Synthesis of intermediate 2-chloro-4-(4,4-difluoro-piperidin-1-yl)-6-(3,5-dimethyl-isoxazol-4-yl)-pyrimidine

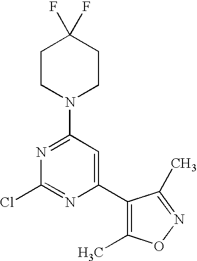

A similar procedure as Example 15 step (i) by using 2,4-dichloro-6-(3,5-dimethyl-isoxazol-4-yl)-pyrimidine and 4,4-difluoropiperidine hydrochloride instead of 2,4-dichloro-6-phenylpyrimidine and cyclobutylamine hydrochloride. Purification (Biotage Horizon HPFC chromatography system, SiO₂, 20:80 EtOAc:hexane) gave the title compound as a white solid (1.03 g, 76.6% yield).

¹H NMR (300 MHz, CDCl₃, TMS): δ 6.42 (s, 1H), 3.83 (apt t, J=3.8, 4H), 2.62 (s, 3H), 2.43 (s, 3H), 2.14-2.01 (m, 4H).

LC-MSD (ES+): m/z [329 (M+H)⁺, 100].

### Step (ii): Synthesis of intermediate 1-{4-[4-(4,4-difluoro-piperidin-1-yl)-6-(3,5-dimethyl-isoxazol-4-yl)-pyrimidin-2-ylamino]-phenyl}-cyclobutanecarboxylic acid methyl ester

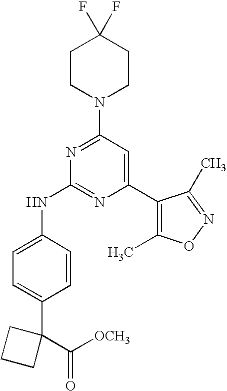

2-Chloro-4-(4,4-difluoro-piperidin-1-yl)-6-(3,5-dimethyl-isoxazol-4-yl)-pyrimidine (220 mg, 0.85 mmol) and 1-(4-aminophenyl)-cyclobutanecarboxylic acid methyl ester (527 mg, 0.1.6 mmol) were mixed in n-butanol (12 mL) and heated at reflux under a nitrogen atmosphere for ˜17 h. The reaction was cooled to about 20-35° C. The precipitate was collected by filtration, rinsed with diethyl ether, 2-propanol, 10% NaHCO₃, water, 2-propanol, and diethyl ether. The material was dried under vacuum to give the title compound as a white solid (460 mg, 57.8% yield).

¹H NMR (300 MHz, CDCl₃, TMS): δ 11.17 (s, 1H), 7.55 (d, J=8.7 Hz, 2H), 7.30 (d, J=8.4 Hz, 2H), 6.04 (s, 1H), 4.12-3.79 (b m, 4H), 3.66 (s, 3H), 2.88-2.81 (m, 2H), 2.79 (s, 3H), 2.70-2.46 (m, 5H), 2.23-1.92 (b m, 6H).

LC-MSD (ES+): m/z [498 (M+H)⁺, 100].

### Step (iii): Synthesis of 1-{4-[4-(4,4-difluoro-piperidin-1-yl)-6-(3,5-dimethyl-isoxazol-4-yl)-pyrimidin-2-ylamino]-phenyl}-cyclobutanecarboxylic acid

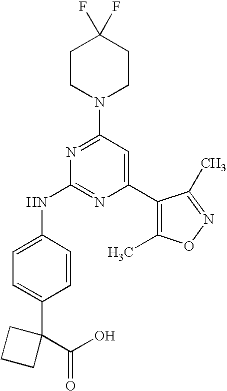

A similar procedure was followed as in Example 12 step (iii). The title compound was isolated as a white solid (287 mg, 74% yield).

HPLC: Betasil C-18, 30:70 [KH₂PO₄ (0.01M, pH 3.0): CH₃CN], 276 nm, Rₜ 6.1 min, 100% purity).

¹H NMR (300 MHz, DMSO-d₆): δ 12.22 (s, 1H), 9.15 (s, 1H), 7.67 (d, J=8.7 Hz, 2H), 7.17 (d, J=8.7 Hz, 2H), 6.43 (s, 1H), 3.81 (br s, 4H), 2.71-2.59 (m, 5H), 2.38-2.32 (m, 5H), 2.14-1.74 (m, 6H).

LC-MSD (ES+): m/z [484 (M+H)⁺, 100].

### Example 37

### Synthesis of 1-{4-[4-butylamino-6-(3,5-dimethyl-isoxazol-4-yl)-pyrimidin-2-ylamino]-phenyl}-cyclobutanecarboxylic acid

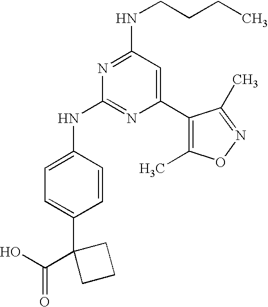

The title compound was afforded as an off white solid following the procedure as described in Example 30, by using n-butylamine instead of morpholine (78% yield).

HPLC: Betasil ODS-3V C18, 30:70 [KH₂PO₄ (0.01M, pH 3.2): CH₃CN], 264 nm, Rₜ 4.62 min, 99.71% purity.

¹H NMR (300 MHz, DMSO-d₆): δ 12.23 (br s, 1H), 9.33 (br s, 1H), 7.68 (d, J=8.1 Hz, 2H), 7.15 (d, J=8.7 Hz, 2H), 6.10 (s, 1H), 3.35 (br s, 6H), 2.70-2.30 (m, 8H, including DMSO), 1.95-1.73 (m, 2H), 1.56-1.30 (m, 4H), 0.898 (t, J=7.2 Hz, 2H).

LC-MSD (ES+): m/z [435 (M+H)⁺, 100].

### Example 38

### Synthesis of 1-{4-[4-tert-butylamino-6-(3,5-dimethyl-isoxazol-4-yl)-pyrimidin-2-ylamino]-phenyl}-cyclobutanecarboxylic acid

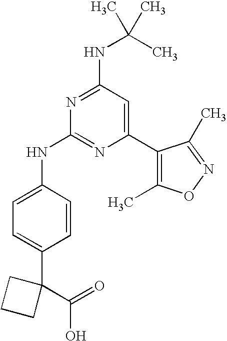

### Step (i): Synthesis of tert-butyl-[2-chloro-6-(3,5-dimethyl-isoxazol-4-yl)-pyrimidin-4-yl-amine

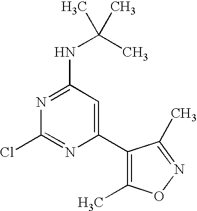

2,4-Dichloro-6-(3,5-dimethyl-isoxazol-4-yl)-pyrimidine (2.5 g, 10.2 mmol) was dissolved in tert-butylamine (9 mL, 84.66 mmol). The resulting mixture was stirred and refluxed for 1.5 h under nitrogen atmosphere. The mixture was concentrated by rotary evaporation and dried for about 10-19 h under vacuum. The resulting solid was dried under vacuum. Purification (Biotage Horizon HPFC system chromatography, SiO₂, 70:30 hexane:EtOAc) gave title compound as a white solid (1.2 g, 41% yield).

HPLC: Inertsil ODS-3V C18, 30:70 [KH₂PO₄ (0.01M, pH 3.2): CH₃CN], 264 nm, Rₜ 10.5 min, 98.2% purity.

¹H NMR (300 MHz, CDCl₃, TMS): δ 6.24 (s, 1H), 5.19 (b s, 1H), 2.62 (s, 3H), 2.43 (s, 3H), 1.47 (s, 9H).

LC-MSD (ES+): m/z [281 (M+H)⁺, 100].

### Step (ii): Synthesis of 1-{4-[4-tert-butylamino-6-(3,5-dimethyl-isoxazol-4-yl)-pyrimidin-2-ylamino]-phenyl}-cyclobutanecarboxylic acid methyl ester

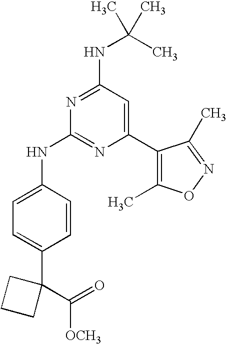

tert-Butyl-[2-chloro-6-(3,5-dimethyl-isoxazol-4-yl)-pyrimidin-4-yl]-amine (1.07 g, 3.8 mmol) was dissolved in n-butanol (10 mL) followed by addition of 1-(4-aminophenyl)-cyclobutanecarboxylic acid methyl ester (0.892 g, 4.18 mmol). The resulting mixture was stirred at reflux for about 10-19 h under nitrogen atmosphere. The mixture was diluted with dichloromethane, and was washed two times with water and one time with brine. The organic phase was dried over sodium sulfate and concentrated by rotary evaporation. The resulting sample was dried under vacuum. Purification (Biotage Horizon HPFC system chromatography, SiO₂, 50:50 hexanes:EtOAc) gave title compound as a pale yellow solid (980 mg, 57% yield).

HPLC: Inertsil ODS-3V C18, 30:70 [KH₂PO₄ (0.01M, pH 3.2): CH₃CN], 264 nm, Rₜ 10.8 min, 96.1% purity.

¹H NMR (300 MHz, CDCl₃, TMS): δ 7.57-7.53 (m, 2H), 7.26-7.20 (m, 2H), 6.92 (s, 1H), 5.85 (s, 1H), 4.80 (s, 1H), 3.64 (s, 3H), 2.86-2.77 (m, 2H), 2.58 (s, 3H), 2.51-2.40 (m, 2H), 2.36 (s, 3H), 2.04-1.81 (m, 2H), 1.48 (s, 9H).

LC-MSD (ES+): m/z [450 (M+H)⁺, 100].

### Step (iii): Synthesis of 1-{4-[4-tert-butylamino-6-(3,5-dimethyl-isoxazol-4-yl]-pyrimidin-2-ylamino]-phenyl}-cyclobutanecarboxylic acid

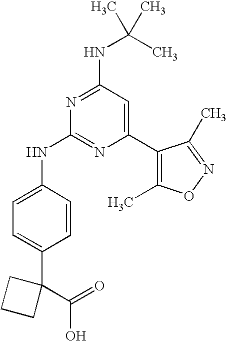

A similar method was used as in Example 4, step (iv) by using 1-{4-[4-tert-butylamino-6-(3,5-dimethyl-isoxazol-4-yl)-pyrimidin-2-ylamino]-phenyl}-cyclobutanecarboxylic acid methyl ester (0.980 g, 2.18 mmol), methanol (10 mL), THF (5 mL) and NaOH (0.352 g, 8.72 mmol) in 15 mL water. The title compound was isolated as a pale yellow solid (600 mg, 63% yield).

HPLC: Inertsil ODS-3V C18, [KH₂PO₄ (0.01M, pH 3.2): CH₃CN], gradient, 264 nm, Rₜ 14.5 min, 96.4% purity.

¹H NMR (300 MHz, CDCl₃, TMS): δ 11.52 (b s, 1H), 8.71 (b s, 1H), 7.54 (d, J=8.4 Hz, 2H), 7.24 (d, J=8.7 Hz, 2H), 5.76 (s, 1H), 5.21 (b s, 1H), 2.85-2.76 (m, 2H), 2.53-2.48 (m, 2H), 2.43 (s, 3H), 2.32 (s, 3H), 2.04-1.78 (m, 2H), 1.44 (s, 9H).

LC-MSD (ES+): m/z [436 (M+H)⁺, 100].

### Example 39

### Synthesis of 1-{4-[4-cyclopentylamino-6-(3,5-dimethyl-isoxazol-4-yl)-pyrimidin-2-yl-amino]-phenyl}-cyclobutanecarboxylic acid

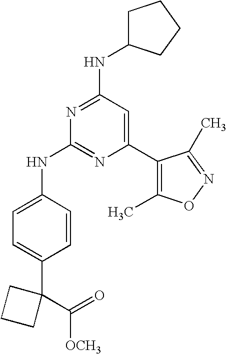

### Step (i): Synthesis of [2-chloro-6-(3,5-dimethyl-isoxazol-4-yl)-pyrimidin-4-yl]-cyclopentyl-amine

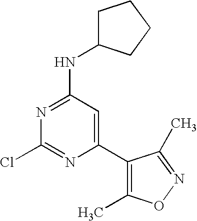

Cyclopentylamine (0.35 mL, 3.5 mmol) and DIEA (0.58 mL, 3.5 mmol) were added to a solution of 2,4-dichloro-6-(3,5-dimethyl-isoxazol-4-yl)-pyrimidine (0.8 g, 3.2 mmol) in anhydrous methanol (10 mL) under nitrogen atmosphere. The mixture was stirred at about 20-35° C. for 18 h. Thereafter, volatiles were evaporated under reduced pressure and the crude was dissolved in EtOAc. After washing with brine the organic solution was dried over sodium sulfate and concentrated under reduced pressure. The crude material was purified by chromatography over silica gel to afford title compound as a white solid (0.42 g, 44% yield).

¹H NMR (CDCl₃, 300 MHz): δ 6.24 (s, 1H), 5.40 (b s, 1H), 4.13-3.99 (m, 1H), 2.62 (s, 3H), 2.42 (s, 3H), 2.09-2.03 (m, 2H), 1.76-1.52 (m, 6H).

LC-MSD (ES+): m/z [293 (M+H)⁺, 100].

### Step (ii): Synthesis of 1-{4-[4-cyclopentylamino-6-(3,5-dimethyl-isoxazol-4-yl)-pyrimidin-2-ylamino]-phenyl}-cyclobutanecarboxylic acid methyl ester

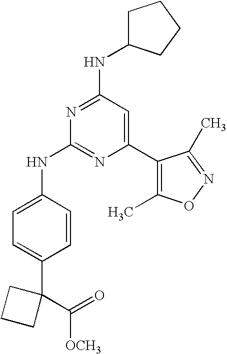

The title compound was afforded as a light brown solid (98% yield) following the procedure described in Example 9, step (ii).

HPLC: Inertsil ODS-3V C18, [KH₂PO₄ (0.01M, pH 3.2): CH₃CN], 264 nm, Rₜ 13.66 min, 95.68% purity.

¹H NMR (CDCl₃, 300 MHz): δ 12.60 (b s, 1H), 10.72 (b s, 1H), 7.65 (d, J=8.4 Hz, 2H), 7.30 (d, J=8.4 Hz, 2H), 6.30 (s, 1H), 4.46-4.39 (m, 1H), 3.65 (s, 3H), 2.82-2.78 (m, 2H), 2.54 (s, 3H), 2.55-2.48 (m, 2H), 2.40 (s, 3H), 2.20-2.02 (m, 2H), 1.99-1.67 (m, 8H).

LC-MSD (ES+): m/z [462 (M+H)⁺, 100].

### Step (iii): Synthesis of 1-{4-[4-cyclopentylamino-6-(3,5-dimethyl-isoxazol-4-yl)-pyrimidin-2-yl-amino]-phenyl}-cyclobutanecarboxylic acid

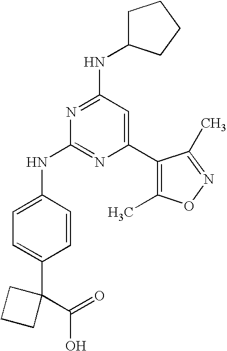

The title compound was afforded as a white solid following the procedure described in Example 9, step (iii).

HPLC: Inertsil-ODS 3V C18, [KH₂PO₄ (0.01M): CH₃CN], gradient, 264 nm, Rₜ 18.62 min, 99.16% purity.

¹H NMR (CDCl₃+CD₃OD, 300 MHz): δ 7.58 (d, J=8.7 Hz, 2H), 7.23 (d, J=8.7 Hz, 2H), 5.93 (s, 1H), 4.36-4.28 (m, 1H), 2.77-2.70 (m, 2H), 2.53 (s, 3H), 2.45-2.35 (m, 2H), 2.32 (s, 3H), 2.0-1.92 (m, 2H), 1.78-1.53 (m, 8H).

LC-MSD (ES+): m/z [448 (M+H)⁺, 100].

### Example 40

### Synthesis of 1-[4-(4-cyclopentylamino-6-phenyl-pyrimidin-2-ylamino)-phenyl]-cyclobutanecarbonitrile

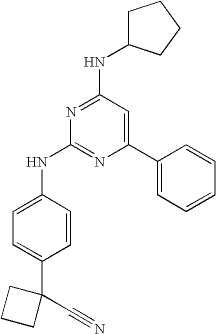

A solution of (2-chloro-6-phenyl-pyrimidin-4-yl)-cyclopentyl-amine (0.35 g, 1.3 mmol) and 1-(4-amino-phenyl)-cyclobutanecarbonitrile (0.23 g, 1.3 mmol) [prepared following a procedure as described in Example 31 step (i) by using 4-nitrophenyl acetonitrile instead of 3-nitrophenyl acetonitrile], in anhydrous 1-butanol (4 mL) was heated at 90° C. to 100° C. under stirring for 18 h. After cooling to about 20-35° C. the mixture was diluted with water and then extracted with EtOAc. The combined organic extracts was washed with brine, dried over Na₂SO₄, filtered, and concentrated under reduced pressure. The crude residue was purified by column chromatography (silica gel) to afford the title compound as a light yellow solid (0.47 g, 90% yield).

HPLC: Betasil ODS-3V C18, 20:80 [KH₂PO₄ (0.01M, pH 3.2): CH₃CN], 274 nm, Rₜ 18.32 min, 97.33% purity.

¹H NMR (300 MHz, CDCl₃): δ 7.99-7.95 (m, 2H), 7.73-7.70 (d, J=8.4 Hz, 2H), 7.48-7.25 (m, 5H), 7.08 (s, 1H), 6.29 (s, 1H), 4.90 (b s, 1H), 4.28-4.24 (m, 1H), 2.85-2.77 (m, 2H), 2.66-2.56 (m, 2H), 2.48-2.03 (m, 4H), 1.76-1.53 (m, 6H).

LC-MSD (ES+): m/z [410 (M+H)⁺, 100].

### Example 41

### Synthesis of sodium 1-[4-(4-tert-butylamino-6-phenyl-pyrimidin-2-ylamino)-phenyl]-cyclobutanecarboxylate

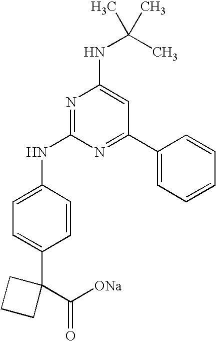

1-[4-(4-tert-Butylamino-6-phenyl-pyrimidin-2-ylamino)-phenyl]-cyclobutane-carboxylic acid (25.00 g, 60.09 mmol), taken in MeOH (250 mL), was refluxed for 30 min, and sodium tert-butoxide (5.70 g, 60.09 mmol), dissolved in MeOH (150 mL), was added drop wise at this temperature. The reaction mixture was refluxed further for about 10-19 h and brought to 60-65° C. The content was filtered though Celite bed, and the solvent was evaporated giving a solid that was stirred with acetonitrile (500-700 mL) for 4-5 h. The solid was filtered, washed with acetonitrile, and dried in vacuum oven at 70° C. for 12-14 h to obtain the title compound as a white solid (26.6 g, 98% yield).

HPLC: Symmetry Shield RP18, [KH₂PO₄ (0.01M, pH 6.5): CH₃CN], gradient, 210 nm, Rₜ 11.82 min, 99.45% purity.

¹H NMR (400 MHz, DMSO-d₆):δ 7.92 (dd, J=1.6 and 8.4 Hz, 2H), 7.62 (d, J=8.4 Hz, 2H), 7.49-7.40 (m, 3H), 7.10 (d, J=8.8 Hz, 2H), 6.36 (b s, 1H, D₂O exchangeable), 2.68-2.62 (m, 2H), 2.18-2.11 (m, 2H), 1.79-1.72 (m, 1H), 1.67-1.59 (m, 1H), 1.45 (s, 9H).

MS (ES): m/z 417 (M−Na)⁺, 360, 315, 249, 216, 116.

### Example 42

### Synthesis of {1-[4-(4-tert-butylamino-6-phenyl-pyrimidin-2-ylmethyl)-phenyl]-cyclobutyl}-methanol

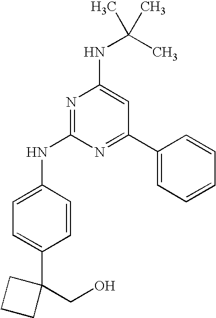

To 1-[4-(4-tert-butylamino-6-phenyl-pyrimidin-2-ylamino)-phenyl]-cyclo-butane carboxylic acid (417.1 mg, 1 mmol) dissolved in THF (10 mL) and cooled to 0° C. in an ice bath was added lithium aluminum hydride in THF (2 mL, 4 mmol, 2M). The resulting mixture was allowed to warm to about 20-35° C. with stirring for about 10-19 h. The mixture was quenched with 6 mL of water and diluted with EtOAc. The mixture was washed two times with water and one time with brine. The organic phase was dried over potassium carbonate and concentrated by rotary evaporation. The resulting solid dried under vacuum gave title compound as a pale yellow solid (336 mg, 83% yield).

HPLC: Inertsil ODS-3V C18, 30:70 [KH₂PO₄ (0.01M, pH 3.2): CH₃CN], 264 nm, Rₜ 5.0 min, 98.1% purity.

^{H}NMR (CDCl₃, 300 MHz): δ 7.96-7.93 (m, 2H), 7.68-7.63 (m, 3H), 7.50-7.44 (m, 4H), 7.14-7.10 (m, 2H), 6.26 (s, 1H), 4.91 (s, 1H), 3.74 (d, J=6.6 Hz, 2H), 2.36-2.04 (m, 6H), 1.50 (s, 9H).

LC-MSD (ES+): m/z [403 (M+H)⁺, 100].

### Example 43

### Synthesis of 1-[4-(4-tert-butylamino-6-phenyl-pyrimidin-2-ylamino)-phenyl]-cyclobutanecarboxylic acid ethylamide

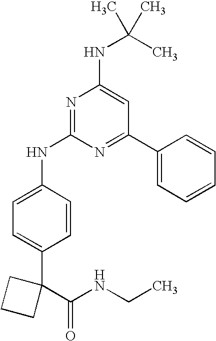

1-[4-(4-tert-Butylamino-6-phenyl-pyrimidin-2-ylamino)-phenyl]-cyclobutane carboxylic acid (302 mg, 0.72 mmol) was suspended in dichloromethane (10 mL) followed by adding HOBt hydrate (97.5 mg, 0.72 mmol) and ethylamine hydrochloride (89.0 mg, 1.08 mmol). The solution was cooled to ˜0° C. in ice bath. EDAC (152.0 mg, 0.79 mmol) and NMM (0.24 mL, 2.16 mmol) were added sequentially. The resulting mixture was stirred at about 20-35° C. under nitrogen atmosphere for 18 h. The mixture was diluted with dichloromethane, and was washed one time with water and one time with 15% citric acid (aq), one time with 10% sodium bicarbonate (aq), and one time with brine. The organic phase was dried over sodium sulfate, filtered, and concentrated by rotary evaporation. The resulting solid was passed though a plug of silica gel using 1:1 hexane:EtOAc as eluent and gave the title compound as a pale yellow solid (220 mg, 62% yield).

HPLC: Inertsil ODS-3V C18, [KH₂PO₄ (0.01M, pH 3.2): CH₃CN], gradient, 264 nm, Rₜ 4.6 min, 97.6% purity.

¹H NMR (300 MHz, CDCl₃, TMS): δ 7.95-7.92 (m, 2H), 7.70 (d, J=9.0 Hz, 2H), 7.49-7.44 (m, 3H), 7.26-7.12 (m, 4H), 6.28 (s, 1H), 5.09 (br s, 1H), 4.85 (s, 1H), 3.22-3.13 (m, 2H), 2.86-2.77 (m, 2H), 2.48-2.38 (m, 2H), 2.21-1.83 (m, 2H), 1.50 (s, 9H), 0.98 (t, J=7.5 Hz, 3H).

LC-MSD (ES+): m/z [444 (M+H)⁺, 100].

### Example 44

### Synthesis of 1-[4-(4-tert-butylamino-6-phenyl-pyrimidin-2-ylamino)-phenyl]-cyclobutanecarboxylic acid dimethylamide

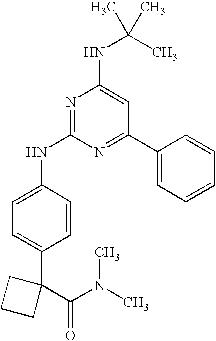

1-[4-(4-tert-Butylamino-6-phenyl-pyrimidin-2-ylamino)-phenyl]-cyclo-butanecarboxylic acid dimethylamide was prepared by a similar procedure described in Example 43 by using dimethylamine hydrochloride instead of ethylamine hydrochloride to give title compound as a white solid (151 mg, 68% yield).

HPLC: Inertsil ODS-3V C18, [KH₂PO₄ (0.01M, pH 3.2): CH₃CN], gradient, 264 nm, Rₜ 14.3 min, 97.2% purity.

¹H NMR (300 MHz, CDCl₃, TMS): δ 7.95-7.91 (m, 2H), 7.68 (d, J=8.4 Hz, 2H), 7.03 (s, 1H), 6.26 (s, 1H), 4.79 (s, 1H), 2.93 (s, 3H), 2.88-2.78 (m, 2H), 2.58 (s, 3H), 2.41-2.34 (m, 2H), 2.07-1.95 (m, 1H), 1.87 (m, 1H), 1.46 (s, 9H).

Mass: LC-MSD (ES+): m/z [444 (M+H)⁺, 100].

### Example 45

### Synthesis of 1-[4-(4-tert-butylamino-6-phenyl-pyrimidin-2-ylamino)-phenyl]-cyclobutanecarboxylic acid tert-butylamide

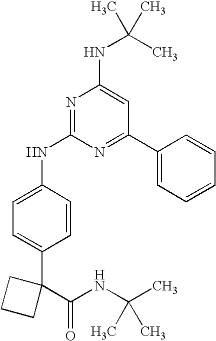

1-[4-(4-tert-Butylamino-6-phenyl-pyrimidin-2-ylamino)-phenyl]-cyclobutane-carboxylic acid tert-butylamide was prepared by a similar procedure described in Example 43 by using tert-butylamine instead of ethylamine hydrochloride to give a white solid (763 mg, 53.7% yield).

HPLC: Inertsil ODS-3V C18, 30:70 [KH₂PO₄ (0.01M, pH 3.2): CH₃CN], 264 nm, Rₜ 8.0 min, 99.3% purity.

¹H NMR (300 MHz, DMSO-d₆): δ 8.87 (s, 1H), 7.91-7.94 (m, 2 H), 7.78 (d, J=8.7 Hz, 2H), 7.44-7.51 (m, 3H), 7.24 (d, J=8.7 Hz, 2H), 6.86 (s, 1H), 6.47-6.48 (m, 2H), 2.58-2.67 (m, 2H), 2.24-2.33 (m, 2H), 1.74-1.75 (m, 2H), 1.45 (s, 9H), 1.18 (s, 9H).

Mass: LC-MSD (ES+): m/z [472 (M+H)⁺, 100].

### Example 46

### Synthesis of 1-[4-(4-tert-butylamino-6-phenyl-pyrimidin-2-ylamino)-phenyl]cyclobutanecarboxylic acid amide

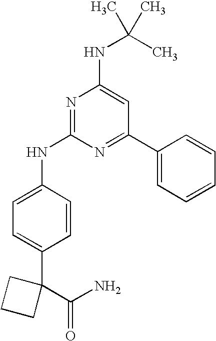

1-[4-(4-tert-Butylamino-6-phenyl-pyrimidin-2-ylamino)-phenyl]-cyclobutane-carboxylic acid amide was prepared by a similar procedure described in Example 43 by using hexamethyldisilizane instead of ethylamine hydrochloride and the following modification. After stirring for ˜24 h, a TBAF (783 mg, 3 mmol) solution in 5 mL H₂O was added and allowed to stir for ˜70 min at about 20-35° C. The general procedure was followed for isolation of the product except EtOAc was the extraction solvent. The impure product was dissolved in EtOAc, washed with 20% KOH (aq) one time with brine wash, dried over anhydrous K₂CO₃, filtered, and concentrated. Purification [Biotage Horizon HPFC system chromatography, SiO₂, 96:3:1 CH₂Cl₂:CH₃OH; NH₄OH (aq); followed by drying the fractions over Na₂SO₄] gave a white solid (276 mg, 66.4% yield) as the title compound.

HPLC: Betasil C18, [KH₂PO₄ (0.01M, pH 3.2): CH₃CN], gradient, 264 nm, Rₜ 11.9 min, 99.4% purity.

¹H NMR (300 MHz, CDCl₃, TMS): δ 7.95-7.92 (m, 2H), 7.71 (d, J=8.7 Hz, 2H), 7.44-7.45 (m, 3H), 7.25-7.28 (m, 2H), 6.98 (s, 1H), 6.28 (s, 1H), 5.17 (b d, J=16.5 Hz, 2H), 4.80 (s, 1H), 2.79-2.88 (m, 2H), 2.09-2.23 (m, 2H), 1.83-1.97 (m, 1H), 1.50 (s, 9H).

Mass: LC-MSD (ES+): m/z [416 (M+H)⁺, 100].

### Example 47

### Synthesis of 1-[4-(4-tert-butylamino-6-phenyl-pyrimidin-2-ylamino)-phenyl]-cyclobutanecarboxylic acid methoxy-methyl-amide

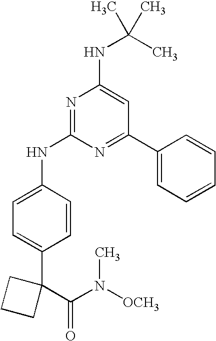

HATU (402 mg, 1.05 mmol) was added to 1-[4-(4-tert-butylamino-6-phenyl-pyrimidin-2-ylamino)-phenyl]-cyclobutanecarboxylic acid (420 mg, 1 mmol) that was dissolved in 5 mL DMF. To this mixture was added DIEA (0.18 mL, 1.05 mmol) and then stirred at about 20-35° C. for ˜1 h under a nitrogen atmosphere. To this mixture was added a solution of N,O-dimethylhydroxylamine hydrochloride (108 mg, 1.1 mmol) in 2 mL DMF and then stirred at about 20-35° C. for about 10-19 h. The mixture was diluted with EtOAc and washed 2 times with water followed by filtration though a plug of Celite. The organic layer was washed two times with brine, dried over anhydrous K₂CO₃, filter, and concentrated by rotary evaporation. Purification (Biotage Horizon HPFC system chromatography, SiO₂, 1:1 hexane:EtOAc) gave the title compound as a white solid (360 mg, 78% yield).

HPLC: Inertsil ODS-3V C18, 30:70 [KH₂PO₄ (0.01M, pH 3.2): CH₃CN], 264 nm, Rₜ 5.9 min, 98.5% purity.

¹H NMR (300 MHz, CDCl₃, TMS): δ 7.95-7.92 (m, 2H), 7.69 (d, J=8.7 Hz, 2H), 7.46-7.44 (m, 3H), 7.32 (apt d, J=8.7 Hz, 2H), 6.94 (s, 1H), 6.26 (s, 1H), 4.76 (s, 1H), 3.10 (s, 3H), 3.07 (br s, 3H), 2.83-2.73 (m, 2H), 2.43-2.34 (m, 2H), 1.99-1.81 (m, 2H), 1.49 (s, 9H).

Mass: LC-MSD (ES+): m/z [460 (M+H)⁺, 100].

### Example 48

### Synthesis of 1-{1-[4-(4-tert-butylamino-6-phenyl-pyrimidin-2-ylamino)-phenyl]-cyclobutyl}-ethanone

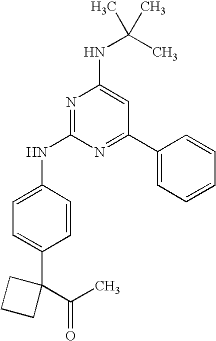

To 1-[4-(4-tert-butylamino-6-phenyl-pyrimidin-2-ylamino)-phenyl]-cyclobutane-carboxylic acid methoxy-methyl-amide (140 mg, 0.3 mmol), dissolved in 5 mL anhydrous THF, was added MeLi (0.94 mL, 1.5 mmol, 1.6 M in diethyl ether) and heated in an 60° C. oil bath for ˜1 h. The reaction was cooled to ˜0° C. with an ice-water bath and quenched slowly by adding saturated NH₄Cl (aq) followed by water. The mixture was extracted thee times with EtOAc. The organic layer was separated, washed with brine, dried over anhydrous MgSO₄, filtered, and concentrated by rotary evaporation. Purification (Biotage Horizon HPFC system chromatography, SiO₂, 1:1 hexane:EtOAc) gave a pale yellow solid (72 mg, 57.9% yield) as the title compound.

HPLC: Inertsil ODS-3V C18, [KH₂PO₄ (0.01M, pH 3.2): CH₃CN], gradient, 264 nm, Rₜ 16.8 min, 97.4% purity.

¹H NMR (300 MHz, CDCl₃, TMS): δ 7.95-7.92 (m, 2H), 7.68 (d, J=8.7 Hz, 2H), 7.46-7.44 (m, 3H), 7.19 (d, J=8.4 Hz, 2H), 6.94 (s, 1H), 4.78 (s, 1H), 2.77-2.68 (m, 2H), 2.46-2.37 (m, 2H), 1.94 (s, 3H), 1.87 (m, 2H), 1.49 (s, 9H).

Mass: LC-MSD (ES+): m/z [415 (M+H)⁺, 100].

### Example 49

### Synthesis of N⁴-tert-butyl-N²-[4-(1-oxazol-2-yl-cyclobutyl)-phenyl]-6-phenyl-pyrimidine-2,4-diamine

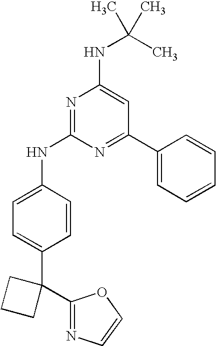

### Intermediate: Synthesis of 4-(1-oxazol-2-yl-cyclobutyl)-phenylamine

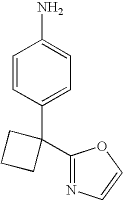

### Step (a): Synthesis of 1-(4-nitro-phenyl)-cyclobutanecarboxylic acid

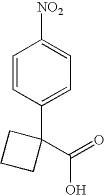

1-(4-Nitro-phenyl)-cyclobutanecarboxylic acid methyl ester (5.06 g, 21.2 mmol) was dissolved in methanol (30 mL) and THF (10 mL) followed by solution of NaOH (2.99 g, 74.2 mmol) in 10 mL water. The resulting mixture was stirred at reflux for about 10-19 h under nitrogen atmosphere. The mixture was concentrated by rotary evaporation to remove most of the methanol and THF, and adjusted to pH ˜1-2 with 6M HCl (aq). The precipitate was collected by vacuum filtration and was rinsed with water. The resulting solid was dried under vacuum which gave the title compound as a brown solid (3.7 g) and was used without further purification.

¹H NMR (300 MHz, CDCl₃, TMS): δ 8.21-8.17 (in, 2H), 7.47-7.42 (m, 2H), 2.95-2.87 (m, 6H), 2.58-2.51 (m, 2H), 2.22-1.87 (in, 2H).

LC-MSD (ES+): m/z [222 (M+H)⁺, 100].

### Step (b): Synthesis of 1-(4-nitro-phenyl)-cyclobutanecarboxylic acid (2,2-dimethyoxy-ethyl)-amide

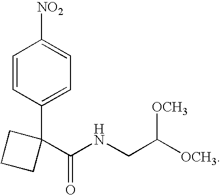

1-(4-Nitro-phenyl)-cyclobutanecarboxylic acid (500.2 mg, 2.26 mmol) was dissolved in dichloromethane (10 mL) and DMF (5 mL) followed by DIEA (0.6 mL, 3.39 mmol) addition. The resulting mixture was stirred for few min and followed by the addition of HATU (1.002 g, 2.71 mmol) and amino acetaldehyde dimethyl acetal (0.3 mL, 2.71 mmol). The resulting mixture was stirred for 3 h under nitrogen atmosphere. The mixture was diluted with dichloromethane. Then it was washed three times with water, one time with 1N HCl (aq) and one time with brine. The organic phase was dried over sodium sulfate and concentrated by rotary evaporation. The resulting solid was dried under vacuum which gave title compound as a gummy liquid (˜500 mg) that was used without purification.

¹H NMR (300 MHz, CDCl₃, TMS): δ 8.24-8.20 (m, 2H), 7.52-7.47 (m, 2H), 5.53 (b s, 1H), 4.26 (t, J=5.4 Hz, 1H), 3.35-3.32 (m, 2H), 3.29 (s, 6H), 2.87-2.82 (m, 2H), 2.55-2.45 (m, 2H), 2.15-1.89 (m, 2H).

### Step (c): Synthesis of 2-[1-(4-nitro-phenyl)-cyclobutyl]-oxazole

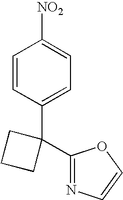

1-(4-Nitro-phenyl)-cyclobutanecarboxylic acid (2,2-dimethyoxy-ethyl)-amide (˜500 mg) was dissolved in Eaton's reagent (10 mL) and the resulting mixture was stirred at 135° C. for 5.5 h under nitrogen atmosphere. Solution was cooled to about 20-35° C. and poured over crushed ice. This mixture was diluted with dichloromethane and filtered though Celite. The Celite was rinsed with dichloromethane, and the filtrate was washed three times with water and one time with brine. The organic phase was dried over sodium sulfate and concentrated by rotary evaporation. The mixture was re-diluted with EtOAc and washed one time with saturated sodium bicarbonate (aq) and one time with brine. The organic phase was dried over sodium sulfate and concentrated by rotary evaporation. The resulting material was dried under vacuum and gave title compound as a gummy solid (460 mg) that was used without purification.

¹NMR (300 MHz, CDCl₃, TMS): δ 8.22-8.18 (m, 2H), 7.57 (d, J=0.9 Hz, 1H), 7.49-7.44 (m, 2H), 7.06 (d, J=0.9 Hz, 1H), 3.05-2.96 (m, 2H), 2.77-2.67 (m, 2H), 2.28-1.97 (m, 2H).

LC-MSD (ES+): m/z [245 (M+H)⁺, 100].

### Step (d): Synthesis of 4-(1-oxazol-2-yl-cyclobutyl)-phenylamine

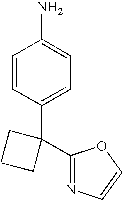

2-[1-(4-Nitro-phenyl)-cyclobutyl]-oxazole (0.38 g, 1.55 mmol) was dissolved in ethanol (10 mL) and followed by tin (II) chloride dihydrate (1.39 g, 6.2 mmol) addition. The resulting mixture was stirred and heated at reflux for 3 h under nitrogen atmosphere. The solution was cooled to about 20-35° C. and adjusted to pH 9-10 using 1N NaOH (aq). The mixture was diluted with EtOAc, and washed two times with water and one time with brine. The organic phase was dried over sodium sulfate and concentrated by rotary evaporation. Drying under vacuum gave title compound as a pale yellow solid (290 mg) that was used without purification.

¹H NMR (300 MHz, CDCl₃, TMS): δ 7.51(apt d, J=0.3 Hz, 1H), 7.12-7.05 (m, 2H), 7.01 (s, 1H), 6.70-6.62 (m, 2H), 3.59 (b s, 2H), 2.93-2.85 (m, 2H), 2.68-2.58 (m, 2H), 2.15-1.88 (m, 2H).

LC-MSD (ES+): m/z [215 (M+H)⁺, 100].

### Step (i): Synthesis of N⁴-tert-butyl-N²-[4-(1-oxazol-2-yl-cyclobutyl)-phenyl]-6-phenyl-pyrimidine-2,4-diamine

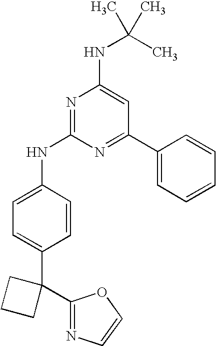

4-(1-Oxazol-2-yl-cyclobutyl)-phenylamine (0.317 g, 1.22 mmol) was dissolved in n-butanol (7 mL) followed by addition of tert-butyl-(2-chloro-6-phenyl-pyrimidin-4-yl)-amine (0.290 g, 1.35 mmol). The resulting mixture was stirred and heated at reflux for about 10-19 h under nitrogen atmosphere. The mixture was diluted with dichloromethane, and was washed two times with water and one time with brine. The organic phase was dried over sodium sulfate and concentrated by rotary evaporation. The resulting sample was dried for about 10-19 h under vacuum. Purification (flash column chromatography, SiO2, 50:50 hexane:EtOAc) gave a pale yellow solid (102 mg, 19% yield) as the title compound.

HPLC: Inertsil ODS-3V C18, 30:70 [KH₂PO₄ (0.01M, pH 3.2): CH₃CN], 264 nm, Rₜ 7.0 min, 97.7% purity.

¹H NMR (300 MHz, CDCl₃, TMS): δ 7.94-7.91 (m, 2H), 7.65(d, J=8.4 Hz, 2H), 7.53 (d, J=0.9 Hz, 1H), 7.45-7.43 (m, 3H), 7.27-7.24 (m, 3H), 7.03 (s, 2H), 6.25 (s, 1H), 2.98-2.90 (m, 2H), 2.75-2.65 (m, 2H), 2.15-1.95 (m, 2H), 1.49 (s, 9H).

LC-MSD (ES+): m/z [440 (M+H)⁺, 100].

### Example 50

### Synthesis of 1-[4-(4-tert-butylamino-6-phenyl-pyrimidin-2-ylamino)-phenyl]-cyclobutanecarboxylic acid (4-fluoro-phenyl)-amide

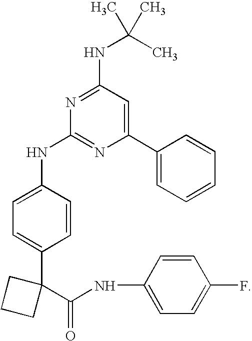

The title compound was prepared by a similar procedure described in Example 43 by using 4-fluoro-aniline to give the title compound as tan solid (80 mg, 21% yield) after purification using a Biotage Horizon HPFC chromatography system, SiO₂, 50:50 hexane:EtOAc).

HPLC: Betasil C-18, gradient, 30:70 [KH₂PO₄ (0.01M, pH 3.0): CH₃CN], 246 nm, Rₜ 11.9 min, 99% purity).

¹H NMR (300 MHz, CDCl₃, TMS): δ 7.94-7.90 (m, 2H), 7.77 (d, J=8.4 Hz, 2H), 7.48-7.44 (m, 3H), 7.35-7.27 (m, 6H), 6.90-6.97 (m, 2H), 6.79 (s, 1H), 6.28 (s, 1H), 4.86 (s, 1H), 2.96-2.87 (m, 2H), 2.57-2.48 (m, 2H), 2.50-2.14 (m, 1H), 2.08-1.90 (m 1H), 1.50 (s, 9H).

Mass: LC-MSD (ES+): m/z [510 (M+H)⁺, 100].

### Example 51

### Synthesis of (1-{4-[4-(3,5-dimethyl-isoxazol-4-yl)-6-morpholin-4-yl-pyrimidin-2-ylamino]-phenyl}-cyclobutyl)-methanol

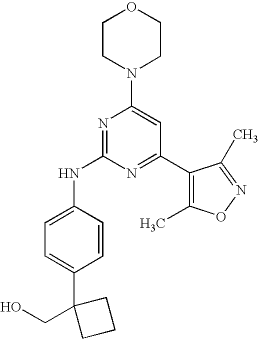

To 1-{4-[4-(3,5-dimethyl-isoxazol-4-yl)-6-morpholin-4-yl-pyrimidin-2-ylamino]-phenyl}-cyclobutanecarboxylic acid (1.50 g, 3.33 mmol) dissolved in dry THF (15 ml) and cooled to 0° C. with an ice bath is added lithium borohydride (218.3 mg, 1.0 mmol) and MeOH (0.81 mL, 20 mmol). The resulting solution is allowed to warm to about 20-35° C. and refluxed for about 10-19 h under a nitrogen atmosphere. The solution is cooled to 0° C. with an ice bath and is quenched with MeOH and water (50:50, 15 mL). The mixture was diluted with dichloromethane, and washed two times with water and one time with brine. The organic phase was dried over sodium sulfate, filtered, and concentrated by rotary evaporation. The resulting sample was dried under vacuum and purified. Flash column chromatography (SiO₂, 95:5 dichloromethane:MeOH) purification gave the title compound as an off white solid (321 mg, 22% yield).

HPLC: Betasil ODS-3V C18, 30:70 [KH₂PO₄ (0.01M, pH 3.2): CH₃CN], 264 nm, Rₜ 4.63 min, 96.9% purity.

¹H NMR (300 MHz, CDCl₃, TMS): δ 7.56-7.53 (m, 2H), 7.13 to 7.09 (m, 2H), 5.97 (s, 1H), 3.84-3.68 (m, 8H), 2.62 (s, 3H), 2.44 (s, 3H), 2.34-2.03 (m, 6H), 1.92-1.83 (m, 2H), 1.25 (b s, 2H).

LC-MSD (ES+): m/z [436 (M+H)⁺, 100].

### Example 52

### Synthesis of N⁴-tert-butyl-N²-[4-(1-methoxymethyl-cyclobutyl)-phenyl]-6-phenyl-pyrimidine-2,4-diamine

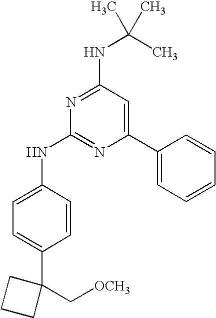

Under a nitrogen atmosphere, methanesulfonyl chloride (0.09 mL, 1.2 mmol) was added to {1-[4-(4-tert-butylamino-6-phenyl-pyrimidin-2-ylmethyl)-phenyl]-cyclobutyl}-methanol (430 mg, 1.06 mmol) that was dissolved in dry methylene chloride (10 mL) and was cooled in an ice-water bath. Then triethylamine (0.17 mL, 1.2 mmol) was added. The mixture was stirred at 0° C. for approximately 30 min and then was stirred at about 20-35° C. for 75 min under an nitrogen atmosphere. The reaction mixture was diluted with methylene chloride, washed with 10% NaHCO₃ (aq), and brine. The organic layer was dried over sodium sulfate, filtered, and concentrated under vacuum to give methanesulfonic acid 1-[4-(4-tert-butylamino-6-phenyl-pyrimidin-2-ylamino)-phenyl]-cyclobutylmethyl ester (mesylate) as an off-white solid (436 mg). The material was used without any purification. LC-MSD (ES+): m/z [481 (M+H)⁺, 100].

A portion of the mesylate (0.211 mg, 0.43 mmol) was dissolved in dry MeOH (2 mL) and sodium methoxide (235.9 mg, 4.4 mmol) was added. The mixture was stirred for approximately four days at about 20-35° C. under a nitrogen atmosphere. Water was added, and then extracted two times with EtOAc. The EtOAc layer was with brine, dried over anhydrous sodium sulfate, filtered, and concentrated under vacuum to give the product. Purification using a Biotage Horizon HPFC chromatography system, SiO₂, 20:80 hexane:EtOAc) gave the title compound as an off-white solid (75 mg, 42% yield).

HPLC: Betasil C-18, 30:70 [KH₂PO₄ (0.01M, pH 3.0): CH₃CN], 246 nm, Rₜ 18.4 min, 98% purity.

¹H NMR (300 MHz, CDCl₃, TMS): δ 7.95-7.92 (m, 2H), 7.60 (d, J=8.4 Hz, 2H), 7.48-7.43 (m 3H), 7.20 (d, J=8.4 Hz, 2H), 6.86 (s, 1H), 6.25 (s, 1H), 4.75 (s, 1H), 3.26 (s, 3H), 2.89 (s, 2H), 2.15-1.92 (m, 4H), 1.76-1.66 (m, 1H), 1.58-1.44 (m, 10H).

LC-MSD (ES+): m/z [417 (M+H)⁺, 100].

### Example 53

### Synthesis of N⁴-tert-butyl-N²-[4-(1-methyl-cyclobutyl)-phenyl]-6-phenyl-pyrimidine-2,4-diamine

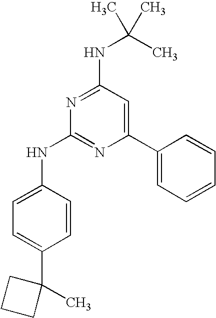

Unpurified methanesulfonic acid 1-[4-(4-tert-butylamino-6-phenyl-pyrimidin-2-ylamino)-phenyl]-cyclobutylmethyl ester (184.3 mg, 0.45 mmol), prepared as described in Example 52, was dissolved in dry THF (4 mL) and cooled in an ice-water bath. Lithium aluminum hydride (0.77 mL, 1.5 mmol, 2M in THF) was added and then stirred for approximately 18 h under a nitrogen atmosphere, gradually warming to about 20-35° C. The reaction as cooled in an ice-bath and then it was quenched with 1 mL water and 2 mL 10% NaOH. The mixture was filtered through Celite with an EtOAc rinse. The organic phase was separated, washed with brine, dried over anhydrous potassium carbonate, filtered, and concentrated under vacuum to give the product. Purification using a Biotage Horizon HPFC chromatography system, SiO₂, (20:80 hexane:EtOAc) gave the title compound as a pale yellow solid (79 mg, 45% yield).

HPLC: Betasil C-18, 30:70 [KH₂PO₄ (0.01M, pH 3.0): CH₃CN], 246 nm, Rₜ 11.9 min, 99% purity.

¹H NMR (300 MHz, CDCl₃, TMS): δ 7.95-7.92 (m, 2H), 7.60 (d, J=8.4 Hz, 2H), 7.47-7.43 (m, 3H), 7.14-7.11, m, 2H), 6.85 (s, 1H), 6.25 (s, 1H), 4.75 (s, 1H), 2.43-2.35 (m, 2H), 2.14-2.00 (m, 3H), 1.88-1.79 (m, 1H), 1.49 (s, 9H), 1.45 (s, 3H).

LC-MSD (ES+): m/z [387 (M+H)⁺, 100].

### Example 54

### Synthesis of [4-(3,5-dimethyl-isoxazol-4-yl)-6-morpholin-4-yl-pyrimidin-2-yl]-[4-(1-methanesulfonylmethyl-cyclobutyl)-phenyl]-amine

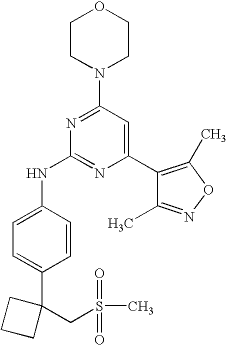

### Step (i): Synthesis of intermediate 4-(1-methanesulfonylmethyl-cyclobutyl)-phenylamine

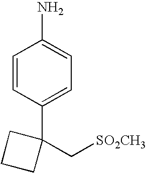

### (a) Synthesis of [1-(4-nitro-phenyl)-cyclobutyl]-methanol

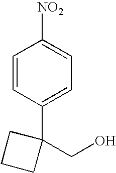

A solution of 1-(4-nitro-phenyl)-cyclobutanecarboxylic acid methyl ester (4.02 g, 17 mmol) dissolved in dry THF (100 mL) under a nitrogen atmosphere, was cooled to 0° C., then diisobutylaluminium hydride (54 mL, 54 mmol, 1M) was added slowly. The resulting solution was allowed to stir at 0° C. for few min and then was stirred at about 20-35° C. for 1 h. The solution was cooled to 0° C., treated with potassium sodium tartrate tetrahydrate (150 mL, 1 M), stirred at 0° C. for few min, and then was stirred vigorously at about 20-35° C. for 30 min. The mixture was diluted with ethyl acetate, and filtered through plug of celite, and extracted two times with water and one time with brine. The organic phase was dried over sodium sulfate, filtered, and concentrated by rotary evaporation. The resulting sample was dried under vacuum and purified.

Purification (Biotage Horizon HPFC chromatography system, SiO₂, 50:50 hexane:ethyl acetate) gave a pale yellow solid (2.73 g, 77% yield) as the title compound.

¹H NMR (300 MHz, CDCl₃, TMS): δ 8.16-8.11 (m, 2H), 7.31-7.26 (m, 2H), 3.79 (d, J=6 Hz, 2H), 2.36-2.28 (m, 4H), 2.85-2.01 (m, 1H), 1.98-1.89 (m, 1H), 1.70 (t, d, J=6 Hz, 1H).

LC-MSD (ES+): m/z [208 (M+H)⁺, 100].

### (b): Synthesis of 1-(1-iodomethyl-cyclobutyl)-4-nitro-benzene

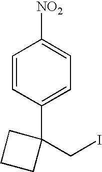

[1-(4-Nitro-phenyl)-cyclobutyl]-methanol (1.905 g, 9.2 mmol) was dissolved in THF (60 mL) then triphenyl phosphine (2.891 g, 11.04 mmol), imidazole (0.9712 g, 14.25 mmol), and iodine (2.815 g, 11.04 mmol) were added sequentially. The resulting mixture was stirred at about 20-35° C. under nitrogen atmosphere for about 10-19 h. The mixture was quenched with ammonium chloride and diluted with dichloromethane. The organic layer was washed one time with sodium thiosulfate (10%), one time with water, and one time with brine. The organic phase was dried over sodium sulfate, filtered, and concentrated by rotary evaporation. The resulting sample was dried under vacuum and purified. Purification (Biotage Horizon HPFC chromatography system, SiO₂, 70:30 hexane:EtOAc) gave a yellow solid (2.5 g, 85% yield).

¹H NMR (300 MHz, CDCl₃, TMS): δ 8.17-8.14 (m, 2H), 7.31-7.28 (m, 2H), 3.63 (s,1H), 2.54-2.44 (m, 2H), 2.34-2.25 (m, 2H), 2.17-2.01 (m, 1H), 1.93-1.81 (m, 1H).

LC-MSD (ES+): m/z [318 (M+H)⁺, 100].

### (c): Synthesis of 1-(1-methanesulfonylmethyl-cyclobutyl)-4-nitro-benzene

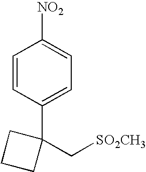

1-(1-Iodomethyl-cyclobutyl)-4-nitro-benzene (1.004 g, 3.14 mmol) was dissolved in ethanol (8 mL, 0.4 M) and sodium methane sulfinic acid (0.802 g, 7.85 mmol) was added. The resulting mixture was refluxed for 48 h under nitrogen atmosphere. The reaction mixture was cooled. The solid was filtered, rinsed with ethanol, and dried under vacuum to give title compound a white solid (0.530 g, 63% yield).

¹H NMR (300 MHz, CDCl₃, TMS): δ 8.26-8.21 (m, 2H), 752-7.47 (m, 2H), 3.60 (s, 2H), 2.65-2.52 (m, 2H), 2.27 (s, 3H), 2.24-2.12 (m, 1H), 2.05-1.91 (m, 1H), LC-MSD (ES+): m/z [270 (M+H)⁺, 100].

### (d): Synthesis of 4-(1-methanesulfonylmethyl-cyclobutyl)-phenylamine

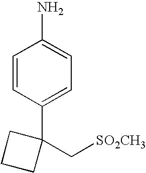

A similar method was used as in Example 1, step i(c) by using 1-(1-methanesulfonyl-methyl-cyclobutyl)-4-nitro-benzene (0.511 g, 1.9 mmol), ethanol (10 mL) and tin (II) chloride dihydrate (1.717 g, 7.6 mmol). The title compound, a pale yellow solid, was dried under vacuum for about 10-19 h and was used without further purification (0.23 g, 45% yield).

¹H NMR (300 MHz, CDCl₃, TMS): δ 7.10 (d, J=8.4 Hz, 2H), 6.69 (d, J=8.4 Hz, 2H), 3.69 (s, 2H), 3.47 (s, 2H), 2.54-2.40 (m, 4H), 2.16-1.87 (m, 5H).

LC-MSD (ES+): m/z [240 (M+H)⁺, 100].

### Step (ii): Synthesis of [4-(3,5-dimethyl-isoxazol-4-yl)-6-morpholin-4-yl-pyrimidin-2-yl]-[4-(1-methanesulfonylmethyl-cyclobutyl)-phenyl]-amine

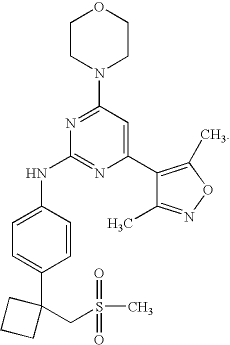

A similar method was used as in Example 1, step (iv) by using 4-[2-chloro-6-(3,5-dimethyl-isoxazol-4-yl)-pyrimidin-4-yl]-morpholine and 4-(1-methanesulfonylmethyl-cyclobutyl)-phenylamine instead of tert-butyl-(2-chloro-6-phenyl-pyrimidine-4-yl)-amine and 1-(4-aminophenyl)-cyclobutane-carboxylic acid methyl ester. Purification (Biotage Horizon HPFC chromatography system, SiO₂, 95:5 dichloromethane:MeOH) gave a light brown solid (0.32 mg, 38% yield) as the title compound.

HPLC: Betasil ODS-3V C18, 30:70 [KH₂PO₄ (0.01M, pH 3.2): CH₃CN], 264 nm, Rₜ 4.6 min, 96% purity.

¹H NMR (300 MHz, DMSO-d₆): δ 9.03 (s , 1H), 7.71 (d, J=8.1 Hz, 2H), 7.21 (d, J=8.4 Hz, 2H), 6.27 (s, 1H), 3.71-3.65 (m, 10H), 2.59-2.39 (m, 10H), 2.11 (s, 4H), 1.81 (s, 1H).

LC-MSD (ES+): m/z [498 (M+H)⁺, 100].

### Example 55

### Synthesis of 1-[4-(2-tert-butylamino-6-phenyl-pyrimidin-4-ylamino)-phenyl]-cyclobutane-carboxylic acid

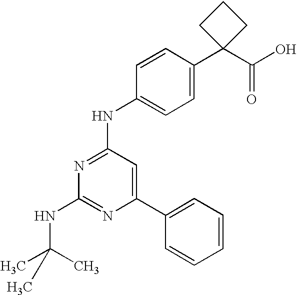

### Step (i): Synthesis of tert-butyl-(4-chloro-6-phenyl-pyrimidin-2yl)-amine

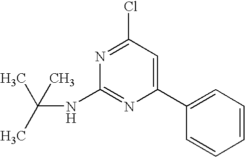

2,4-Dichloro-6-phenyl pyrimidine (5.03 g, 22.2 mmol) was dissolved in tert-butylamine (19.5 mL, 184.26 mmol). The resulting mixture was stirred at reflux for 1.5 h under nitrogen atmosphere. The mixture was concentrated by rotary evaporation. The resulting solid was recrystallized using ether and hexane. The mother liquor was concentrated and purified (flash chromatography system, SiO₂, 80:20 hexane:ethyl acetate) to give the title compound as gummy material (2.3 g, 40% yield).

HPLC: Inertsil ODS-3V C18, 30:70 [KH₂PO₄ (0.01M, pH 3.2): CH₃CN], 264 nm, Rₜ 26.7 min, 88.5% purity.

¹H NMR (300 MHz, CDCl₃, TMS): δ 8.00-7.97 (m, 2H), 7.49-7.45 (m, 3H), 6.95 (s, 1H), 1.49 (s, 9H).

Mass: LC-MSD (ES+): m/z 262 (M+H, 100).

### Step (ii): Synthesis of 1-[4-(2-tert-butylamino-6-phenyl-pyrimidin-4-ylamino)-phenyl]-cyclobutanecarboxylic acid methyl ester

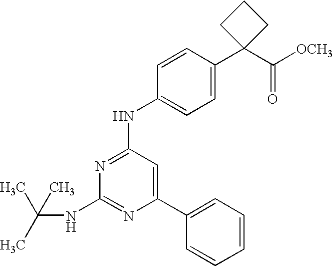

tert-Butyl-(4-chloro-6-phenyl-pyrimidin-2yl)-amine (1.505 g, 5.74 mmol) was dissolved in n-butanol (15 mL) followed by addition of 1-(4-aminophenyl)-cyclobutanecarboxylic acid methyl ester (1.392 g, 6.8 mmol). The resulting mixture was stirred and refluxed for about 10-19 h under nitrogen atmosphere. The mixture was diluted with dichloromethane, and was washed two times with water and one time with brine. The organic phase was dried over sodium sulfate and concentrated by rotary evaporation. The resulting sample was dried for about 10-19 h under vacuum. Purification (flash column chromatography, SiO₂, 70:30 hexane:EtOAc) gave a pale yellow solid as the title compound (200 mg, 8% yield).

HPLC: Inertsil ODS-3V C18, 30:70 [KH₂PO₄ (0.01M, pH 3.2): CH₃CN], 264 nm, Rₜ 6.5 min, 90% purity.

¹H NMR (300 MHz, CDCl₃, TMS): δ 7.94-7.92 (m, 2H), 7.47-7.24 (m, 7H), 6.03 (s, 1H), 6.47 (s, 1H), 5.03 (s, 1H), 3.61 (s, 3H), 2.87-2.79 (m, 2H), 2.55-2.46 (m, 2H), 2.07-1.85 (m, 2H), 1.49 (s, 9H).

LC-MSD (ES+): m/z [431 (M+H)⁺, 100].

### Step (iii): Synthesis of 1-[4-(2-tert-butylamino-6-phenyl-pyrimidin-4-ylamino)-phenyl]-cyclobutanecarboxylic acid

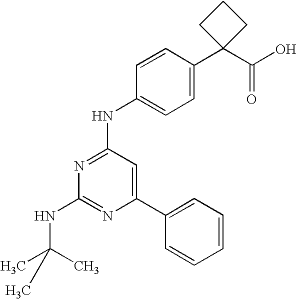

1-[4-(2-tert-Butylamino-6-phenyl-pyrimidin-4-ylamino)-phenyl]-cyclobutane-carboxylic acid methyl ester (200 g, 0.46 mmol) was dissolved in 2-propanol (6 mL) followed by solution of KOH in 5 mL water (0.103 g, 1.84 mmol). The resulting mixture was stirred at reflux for 4 h under nitrogen atmosphere. The mixture was concentrated by rotary evaporation to remove most of the isopropanol then adjusted to pH 3-4 with dilute HCl. The precipitate was then filtered under vacuum and rinsed the precipitate with pentane. The resulting solid was dried under vacuum. Purification (flash chromatography system, SiO₂, 95:5 dichloromethane:methanol) gave the title compound as a pale yellow solid (80 mg, 41% yield).

HPLC: Inertsil ODS-3V C18, [KH₂PO₄ (0.01M, pH 3.2): CH₃CN], gradient, 264 nm, Rₜ 15.3 min, 96.3% purity.

¹H NMR (300 MHz, DMSO-d₆): δ 12.27 (b s, 1H), 9.19 (s, 1H), 7.96 (d, J=8.1 Hz, 2H), 7.73(d, J=8.4 Hz, 2H), 7.52-7.45 (m, 3H), 7.194 (d, J=8.7 Hz, 2H), 6.51 (s, 1H), 6.27 (s, 1H), 2.73-2.65 (m, 2H), 2.42-2.33 (m, 2H), 1.94-1.78 (m, 2H), 1.46 (s, 9H)

LC-MSD (ES+): m/z [417 (M+H)⁺, 100].

### Example 56

### Synthesis of 1-{4-[2-(tert-butyl-methyl-amino)-6-phenyl-pyrimidin-4-ylamino]-phenyl}-cyclobutanecarboxylic acid

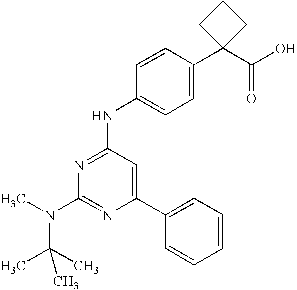

Step (i): Synthesis of 1-{4-[4-(tert-butyl-methyl-amino)-6-phenyl-pyrimidin-2-ylamino]-phenyl}-cyclobutanecarboxylic acid methyl ester

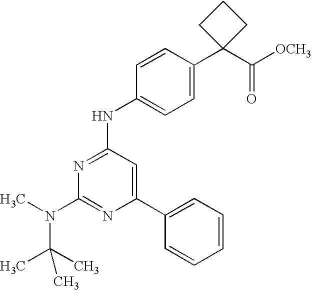

tert-Butyl-(4-chloro-6-phenyl-pyrimidin-2yl)-methylamine (550 mg, 2 mmol) was dissolved in dry toluene (10 mL) and followed by adding 1-(4-aminophenyl)-cyclobutanecarboxylic acid methyl ester (0.619 g, 3 mmol), tris(dibenzylideneacetone) dipalladium (0) (0.0371 g, 0.04 mmol), 1,3-bis(diphenylphosphino)propane (0.0335 g, 0.08 mmol), sodium-tert-butoxide (0.2695 g, 2.8 mmol). The reaction mixture was stirred at reflux for about 10-19 h under nitrogen atmosphere. The reaction mixture was diluted with dichloromethane, and filtered though Celite, and rinsed with dichloromethane. The filtrate was washed two times with water and one time with brine. The organic phase was dried over sodium sulfate and concentrated by rotary evaporation. The resulting sample was dried under vacuum. Purification (Biotage Horizon HPFC system chromatography, SiO₂, 90:10 hexane:EtOAc) gave a pale yellow solid (560 mg, 62% yield) as the title compound.

HPLC: Betasil ODS-3V C18, 30:70 [KH₂PO₄ (0.01M, pH 3.2): CH₃CN], 264 nm, Rₜ 38.3 min, purity 99.1%.

¹H NMR (300 MHz, CDCl₃, TMS): δ 7.98-7.95 (m, 2H), 7.41-7.23 (m, 7H), 6.46 (s, 1H), 6.44 (s, 1H), 3.64 (s, 3H), 3.28 (s, 3H), 2.86-2.77 (m, 2H), 2.54-2.44 (m, 2H), 2.06-1.80 (m, 2H), 1.59 (s, 9H).

LC-MSD (ES+): m/z [445 (M+H)⁺, 100].

### Step (ii): Synthesis of 1-{4-[4-(tert-butyl-methyl-amino)-6-phenyl-pyrimidin-2-ylamino]-phenyl}-cyclobutanecarboxylic acid

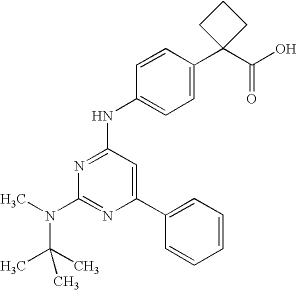

1-{4-[4-(tert-Butyl-methyl-amino)-6-phenyl-pyrimidin-2-ylamino]-phenyl}-cyclobutanecarboxylic acid methyl ester (530 mg, 1.19 mmol) was dissolved in methanol (7 mL) and THF (4 mL) followed by solution of NaOH (0.1921 g, 4.76 mmol) in 10 mL water. The resulting mixture was stirred and refluxed for about 10-19 h under nitrogen atmosphere. The mixture was concentrated by rotary evaporation to remove most of the methanol then adjusted to pH 3-4 with 10% HCl (aq). The precipitate was then filtered with ether. The resulting solid was dried under vacuum which gave the title compound as a pale yellow solid (420 mg, 82% yield).

HPLC: Betasil ODS-3V C18, 30:70 [KH₂PO₄ (0.01M, pH 3.2): CH₃CN], 264 nm, Rₜ 6.4 min, 96.1% purity.

¹H NMR (300 MHz, DMSO-d₆): δ 12.27 (b s, 1H), 9.32 (b s, 1H), 7.90 (s, 2H), 7.63 to 7.50 (m, 5H), 7.22 (d, J=7.5 Hz, 2H), 6.58 (s, 1H), 3.21 (s, 3H), 2.76-2.62 (m, 2H), 2.41-2.32 (m, 2H), 1.98-1.72 (m, 2H), 1.53 (s, 9H).

LC-MSD (ES+): m/z [431 (M+H)⁺, 100].

### Example 57

### Synthesis of 1-[4-(6-tert-butylamino-2-phenyl-pyrimidin-4-ylamino)-phenyl]-cyclobutanecarboxylic acid

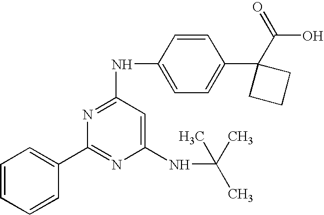

### Step (i): Synthesis of tert-butyl-(2,6-dichloro-pyrimidin-4-yl)-amine

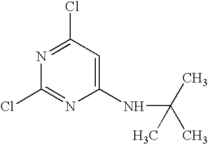

To 2,4,6-trichloropyrimidine (2.08 g, 10.9 mmol), dissolved in DMF (10 mL), was added tert-butyl amine (1.4 mL, 13.08 mmol) and potassium carbonate (4.56 g, 32.7). The resulting mixture was stirred for about 10-19 h at about 20-35° C. under a nitrogen atmosphere. The reaction mixture was extracted 2 times with dichloromethane. The combined organic layers were washed with water and brine, dried over sodium sulfate, filtered, and then concentrated under vacuum to give product. The solid was triturated with ether and hexane, filtered, and dried under vacuum to give title compound as white solid (1.4 g, 56% yield). HPLC: Betasil ODS-3V C18, 30:70 [KH₂PO₄ (0.01M, pH 3.2): CH₃CN], 240 nm, Rₜ 8.1 min, 95% purity.

¹H NMR (300 MHz, CDCl₃, TMS): δ 6.29 (s, 1H), 1.44 (s, 9H).

LC-MSD (ES+): m/z [220 (M+H)⁺, 100].

### Step (ii): Synthesis of 1-[4-(6-tert-butylamino-2-chloro-pyrimidin-4-ylamino)-phenyl]-cyclobutanecarboxylic acid methyl ester

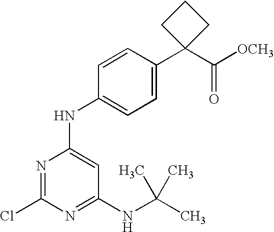

To tert-butyl-(2,6-dichloro-pyrimidin-4-yl)-amine (1.4 g, 6.4 mmol), dissolved in Toluene (15 mL), was added 1-(4-aminophenyl)-cyclobutane-carboxylic acid methyl ester (1.305 g, 6.4 mmol), tri(dibenylidene acetone)dipalladium (0.1996 g, 0.217 mmol), 1,3-bis(diphenyl phosphino)propane (0.1572 g, 0.38 mmol) and sodium-tert-butoxide (0.8615 g, 8.96 mmol) sequentially. The resulting mixture was stirred for 48 h at reflux under nitrogen atmosphere. The reaction mixture was diluted by dichloromethane and filtered through Celite with a dichloromethane wash. The filtrate was washed two times with water and one time with brine. The organic phase was dried over sodium sulfate, filtered, and concentrated by rotary evaporation. Purification (Biotage Horizon HPFC chromatography system, SiO₂, 80:20 hexane:EtOAc) gave the title compound as a solid (250 mg, 10% yield).

¹H NMR (300 MHz, CDCl₃, TMS): δ 7.22-7.11 (m, 5H), 5.61 (s, 1H), 4.93 (s, 1H), 3.57 (s, 3H), 2.75 (s, 2H), 2.45-2.36 (m, 2H), 2.10-1.71 (m, 2H), 1.27 (s, 9H).

LC-MSD (ES+): m/z [389 (M+H)⁺, 100].

### Step (iii): Synthesis of 1-[4-(6-tert-butylamino-2-phenyl-pyrimidin-4-ylamino)-phenyl]-cyclobutanecarboxylic acid methyl ester

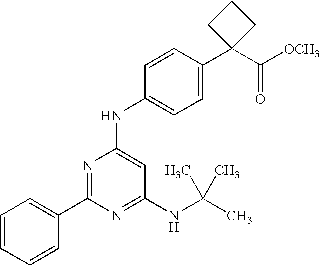

1-[4-(6-tert-butylamino-2-chloro-pyrimidin-4-ylamino)-phenyl]-cyclobutane-carboxylic acid methyl ester (220 mg, 0.56 mmol) was dissolved in diethylene glycol dimethyl ether (7 mL) and phenyl boronic acid (0.153 g, 1.26 mmol), palladium tetrakis (triphenylphospine) (0.101 g, 0.087 mmol), sodium carbonate (0.85 mL, 1.68 mmol, 2M) were added sequentially. The resulting mixture was stirred at reflux for about 10-19 h under nitrogen atmosphere. The mixture was diluted with dichloromethane, filtered through plug of celite, and washed two times with water and one time with brine. The organic phase was dried over sodium sulfate, filtered, and concentrated by rotary evaporation. The resulting sample was dried under vacuum and purified. Purification (Biotage Horizon HPFC chromatography system, SiO₂, 80:20 hexane:EtOAc) gave the title compound as a solid (150 mg, 62% yield).

HPLC: Inertsil ODS-3V C18, 30:70 [KH₂PO₄ (0.01M, pH 3.2): CH₃CN], 264 nm, Rₜ 27.4 min, 95% purity.

¹H NMR (300 MHz, CDCl₃, TMS): δ 8.35-8.32 (m, 2H), 7.43 to 7.21 (m, 8H), 6.89 (s, 1H), 5.74 (s, 1H), 4.80 (s, 1H), 3.86 (s, 3H), 2.87-2.78 (m, 2H), 2.54-2.44 (m, 2H), 2.07-1.81 (m, 2H), 1.44 (s, 9H).

LC-MSD (ES+): m/z [431 (M+H)⁺, 100].

### Step (iv): Synthesis of 1-[4-(6-tert-butylamino-2-phenyl-pyrimidin-4-ylamino)-phenyl]-cyclobutanecarboxylic acid

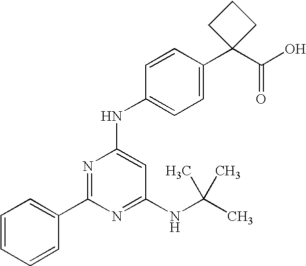

A similar method was used as in Example 1, step (v) by using 1-[4-(6-tert-butylamino-2-phenyl-pyrimidin-4-ylamino)-phenyl]-cyclobutane carboxylic acid methyl instead of 1-[4-(4-tert-butylamino-6-phenyl-pyrimidin-2-ylamino)-phenyl]-cyclobutanecarboxylic acid methyl ester. The resulting pale yellow solid was dried under vacuum to give the title compound (90 mg, 62% yield).

HPLC: Betasil ODS-3V C18, [KH₂PO₄ (0.01M, pH 3.2): CH₃CN], gradient, 280 nm, Rₜ 11.8 min, 97% purity.

¹H NMR (300 MHz, CDCl₃, TMS): δ 9.60 (b s, 1H), 8.28 (d, J=7.5 Hz, 2H), 7.55-7.42 (m, 3H), 7.17-7.15 (m, 5 H), 5.83 (b s, 1H), 2.81-2.71 (m, 2H), 2.41-2.31 (m, 2H), 2.02-1.70 (m, 2H), 1.40 (s, 9H).

LC-MSD (ES+): m/z [417 (M+H)⁺, 100].

### Example 58

### Synthesis of 1-{4-[6-(3,5-dimethyl-isoxazol-4-yl)-2-methyl-pyrimidin-4-ylamino]-phenyl}-cyclobutanecarboxylic acid

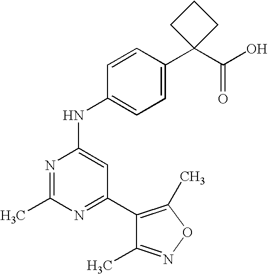

### Step (i): Synthesis of 4-chloro-6-(3,5-dimethyl-isoxazol-4yl)-2-methyl-pyrimidine

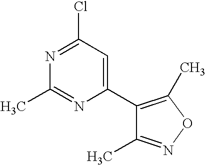

A similar method was used as in Example 1, step (ii) by using 4,6 dichloro-2-methyl pyrimidine (2.01 g, 12.3 mmol), THF (20 mL), 3,5 dimethyl isoxazole-4-boronic acid (1.439 g, 10.2 mmol), palladium acetate (II) (0.0471 g, 0.208 mmol), triphenylphosphine (0.107 g, 0.405 mmol) and sodium carbonate (9.8 mL, 19.68 mmol, 2 M). The resulting mixture was stirred at reflux in a 60° C. oil bath under a nitrogen atmosphere for 6 h. Flash column chromatography, (SiO₂, 70:30 Hexane:EtOAc) purification gave a pale yellow solid (0.780 g, 72% yield) as the title compound.

HPLC: Betasil ODS-3V C18, 30:70 [KH₂PO₄ (0.01M, pH 3.2): CH₃CN], 264 nm, Rₜ 6.9 min, 99% purity.

¹H NMR (300 MHz, CDCl₃, TMS) δ 7.18 (s, 1H), 2.72 (s, 3H), 2.68 (s, 3H), 2.49 (s, 3H).

LC-MSD (ES+): (m/z) [224 (M+H)⁺, 100].

### Step (ii): Synthesis of 1-{4-[6-(3,5-dimethyl-isoxazol-4-yl)-2-methyl-pyrimidin-4-ylamino]-phenyl}-cyclobutanecarboxylic acid methyl ester

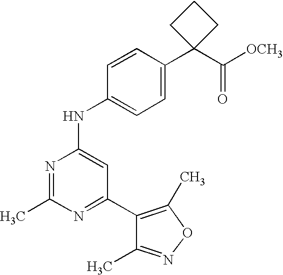

A similar method was used as in Example 1, step (iv) by using 4-chloro-6-(3,5-dimethyl-isoxazol-4yl)-2-methyl-pyrimidine instead of tert-butyl-(2-chloro-6-phenyl-pyrimidine-4-yl)-amine. Purification (Biotage Horizon HPFC chromatography system, SiO₂, 50:50 hexane:EtOAc) gave a pale yellow solid [1.3 g (wet with solvent), 100% yield].

HPLC: Betasil ODS-3V C18, 30:70 [KH₂PO₄ (0.01M, pH 3.2): CH₃CN], 264 nm, Rₜ 9.6 min, 96% purity.

¹H NMR (300 MHz, CDCl₃, TMS) δ 7.32 (s, 4H), 7.12 (s, 1H), 6.53 (s, 1H), 3.68 (s, 3H), 2.89-2.79 (m, 2H), 2.57 (d, J=1.2 Hz, 6H), 2.56-2.44 (m, 2H), 2.38 (s, 3H), 2.11-1.82 (m, 2H).

LC-MSD (ES+): (m/z) [393 (M+H)⁺, 100].

### Step (iii): Synthesis of 1-{4-[6-(3,5-dimethyl-isoxazol-4-yl)-2-methyl-pyrimidin-4-ylamino]-phenyl}-cyclobutanecarboxylic acid

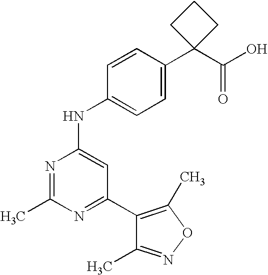

A similar method was used as in Example 1, step (v) by using 1-{4-[6-(3,5-dimethyl-isoxazol-4-yl)-2-methyl-pyrimidin-4-ylamino]-phenyl}-cyclobutane carboxylic acid methyl ester instead of 1-[4-(4-tert-butylamino-6-phenyl-pyrimidin-2-ylamino)-phenyl]-cyclobutanecarboxylic acid methyl ester. The resulting light yellow solid was dried under vacuum to give the title compound (1.1 g, 88% yield).

HPLC: Betasil ODS-3V C18, 30:70 [KH₂PO₄ (0.01M, pH 3.2): CH₃CN], 264 nm, Rₜ 4.69 min, 95% purity.

¹H NMR (300 MHz, DMSO-d₆) δ 12.41 (br s,1H), 10.61 (br s,1H), 7.68 (d, J=7.5 Hz, 2H), 7.28 (d, J=8.4 Hz, 2H), 6.92 (s, 1H), 2.71-2.38 (m, 13H), 1.96-1.79 (m, 2H).

LC-MSD (ES+): (m/z) [379 (M+H)⁺, 100].

### Example 59

### Synthesis of 1-{4-[2-(4-fluoro-phenyl)-6-phenyl-pyrimidin-4-ylamino]-phenyl}-cyclobutanecarboxylic acid

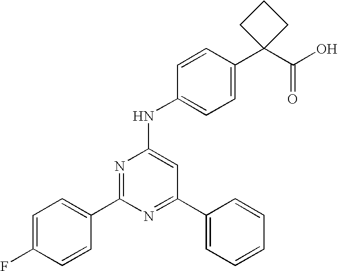

### Step (i): Synthesis of 1-[4-(2-chloro-6-phenyl-pyrimidin-4-ylamino)-phenyl]-cyclobutanecarboxylic acid methyl ester

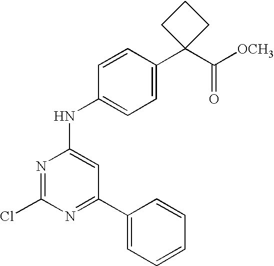

A similar procedure was used as in Example 5, step (i) by using 1-(4-aminophenyl)cyclobutane carboxylic acid methyl ester instead of 2-(aminomethyl)-1-ethylpyrrolidine. Purification (Biotage Horizon HPFC chromatography system, SiO₂, 80:20 hexane:EtOAc) gave the title compound as a pale yellow solid (2.7 g, 68% yield).

HPLC: Betasil ODS-3V C18, 30:70 [KH₂PO₄ (0.01M, pH 3.2): CH₃CN], 264 nm, Rₜ 17.3 min, 97% purity.

¹H NMR (300 MHz, CDCl₃, TMS): δ 7.92-7.87 (m, 2H), 7.44-7.28 (m, 8H), 6.95 (s, 1H), 3.65 (s, 3H), 2.88-2.79 (m, 2H), 2.54-2.44 (m, 2H), 2.08-1.84 (m, 2H),

LC-MSD (ES+): m/z [394 (M+H)⁺, 100].

### Step (ii): Synthesis of 1-{4-[2-(4-fluoro-phenyl)-6-phenyl-pyrimidin-4-ylamino]-phenyl}-cyclobutanecarboxylic acid methyl ester

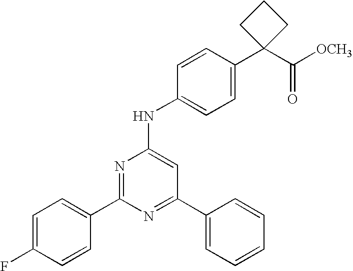

A similar method was used as in Example 57, step (iii) by using 1-[4-(2-chloro-6-phenyl-pyrimidin-4-ylamino)-phenyl]-cyclobutanecarboxylic acid methyl ester and 4-fluoro phenyl boronic acid instead of 1-[4-(6-tert-butylamino-2-pyrimidin-4-ylamino)-phenyl]-cyclobutane-carboxylic acid methyl ester and phenyl boronic acid. Purification (Biotage Horizon HPFC chromatography system, SiO₂, 80:20 hexane:EtOAc) gave the title compound as a yellow solid (450 mg, 58% yield).

HPLC: Betasil ODS-3V C18, 30:70 [KH₂PO₄ (0.01M, pH 3.2): CH₃CN], 264 nm, Rₜ 54.2 min, 99% purity.

¹H NMR (300 MHz, CDCl₃, TMS): δ 8.57-8.51 (m, 2H), 8.11-8.08 (m, 2H), 7.51-7.42 (m, 5H), 7.37-7.34 (m, 2H), 7.20-7.12 (m, 2H), 7.01 (s, 1H), 6.92-6.86 (m, 1H), 3.69 (s, 3H), 2.91-2.82 (m, 2H), 2.59-2.49 (m, 2H), 2.14-1.86 (m, 2H).

LC-MSD (ES+): m/z [454 (M+H)⁺, 100].

### Step (iii): Synthesis of 1-{4-[2-(4-fluoro-phenyl)-6-phenyl-pyrimidin-4-ylamino]-phenyl}-cyclobutanecarboxylic acid

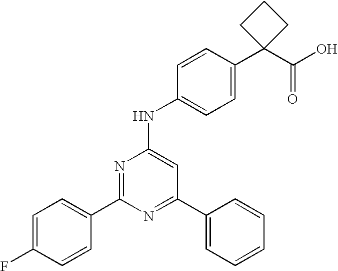

1-{4-[2-(4-Fluoro-phenyl)-6-phenyl-pyrimidin-4-ylamino]-phenyl}-cyclobutane-carboxylic acid methyl ester (0.407 g, 0.9 mmol) was dissolved in ethylene glycol (15 mL) then a solution of NaOH (0.289 g, 7.2 mmol) in 25 mL water was added. The resulting mixture was stirred at reflux for 48 h at 120° C. under nitrogen atmosphere. The mixture cooled and was adjusted to pH 3-4 with dilute HCl (aq). The precipitate was then filtered and washed with water and ether. Purification (Biotage Horizon HPFC chromatography system, SiO₂, 95:5 dichloromethane:methanol) gave the title compound as a pale yellow solid (275 mg, 70% yield).

HPLC: Betasil ODS-3V C18, 30:70 [KH₂PO₄ (0.01M, pH 3.2): CH₃CN], 264 nm, Rₜ 21.76 min, 97% purity.

¹H NMR (300 MHz, CDCl₃, TMS): δ 8.53-8.47 (m, 2H), 8.10-8.05 (m, 2H), 7.49-7.35 (m, 7H), 7.18-7.12 (m, 314), 7.09 (s, 1H), 2.93-2.84 (m, 2H), 2.61-2.51 (m, 2H), 2.18-1.87 (m, 2H).

LC-MSD (ES+): m/z [440 (M+H)⁺, 100].

### Example 60

### Synthesis of 1-{4-[2-(4-acetyl-phenyl)-6-phenyl-pyrimidin-4-ylamino]-phenyl}-cyclobutanecarboxylic acid

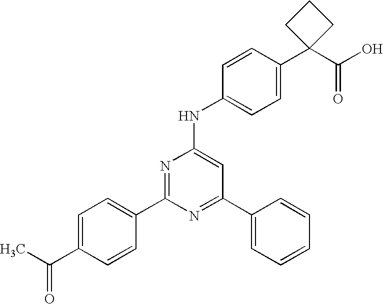

### Step (i): Synthesis of 1-{4-[2-(4-acetyl-phenyl)-6-phenyl-pyrimidin-4-ylamino]-phenyl}-cyclobutanecarboxylic acid methyl ester

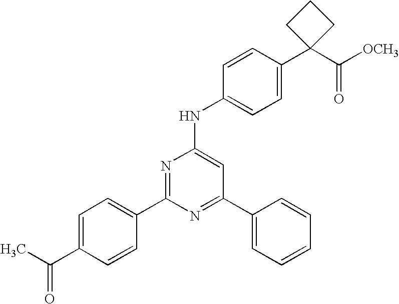

A similar method was used as in Example 57, step (iii) by using 1-[4-(2-chloro-6-phenyl-pyrimidin-4-ylamino)-phenyl]-cyclobutanecarboxylic acid methyl ester and 4-acetyl benzene boronic acid instead of 1-[4-(6-tert-butylamino-2-pyrimidin-4-ylamino)-phenyl]-cyclobutane-carboxylic acid methyl ester and phenyl boronic acid. Purification (Biotage Horizon HPFC chromatography system, SiO₂, 70:30 hexane:EtOAc) gave the title compound as a pale yellow solid (160 mg, 44% yield).

HPLC: Betasil ODS-3V C18, 30:70 [KH₂PO₄ (0.01M, pH 3.2): CH₃CN], 264 nm, Rₜ 30.6 min, 93% purity.

¹H NMR (300 MHz, CDCl₃, TMS): δ 8.61 (d, J=8.7 Hz, 2H), 8.10-8.04 (m, 4H), 7.48-7.45 (m, 5H), 7.35 (d, J=8.4 Hz, 2H), 7.23 (b s, 1H), 7.04 (s, 1H), 3.69 (s, 3H), 2.91-2.82 (m, 2H), 2.65 (s, 3H), 2.58-2.48 (m, 2H), 2.11-1.85 (m, 2H).

LC-MSD (ES+): m/z [478 (M+H)⁺, 100].

### Step (ii): Synthesis of 1-{4-[2-(4-acetyl-phenyl)-6-phenyl-pyrimidin-4-ylamino]-phenyl}-cyclobutanecarboxylic acid

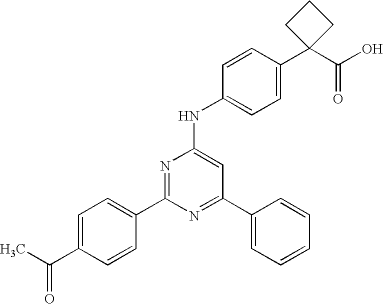

1-{4-[2-(4-Acetyl-phenyl)-6-phenyl-pyrimidin-4-ylamino]-phenyl}-cyclo-butane carboxylic acid methyl ester (0.100 g, 0.21 mmol) was dissolved in ethylene glycol (10 mL) then a solution of NaOH (0.069 g, 1.68 mmol) in 10 mL water was added. The resulting mixture was stirred at reflux for 5 h under nitrogen atmosphere. The mixture cooled and was adjusted to pH 3-4 with dilute HCl (aq). The precipitate was then filtered and washed with water. Purification (Biotage Horizon HPFC chromatography system, SiO₂, 95:5 dichloromethane:MeOH) gave the title compound as a pale yellow solid (50 mg, 51% yield).

HPLC: Betasil ODS-3V C18, 30:70 [KH₂PO₄ (0.01M, pH 3.2): CH₃CN], 264 nm, Rₜ 13.3 min, 97% purity.

¹H NMR (300 MHz, DMSO-d₆): δ 12.31 (br s, 1H), 9.88 (s, 1H), 8.60 (d, J=7.8 Hz, 2H), 8.17-8.12 (m, 4H), 7.81 (d, J=8.4 Hz, 2H), 7.57 (d, J=6 Hz, 3H), 7.33 (d, J=8.1 Hz, 2H), 7.23 (s, 1H), 2.71-2.65 (m, 5H), 2.50-2.40 (m, 2H), 1.98-1.80 (m, 2H).

LC-MSD (ES+): m/z [464(M+H)⁺, 100].

### Example 61

### Synthesis of 1-{4-[6-3,5-dimethyl-isoxazol-4-yl)-2-(4-fluoro-phenyl)-pyrimidin-4-ylamino]-phenyl}-cyclobutanecarboxylic acid

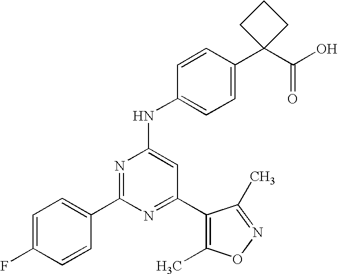

### Step (i): Synthesis of 1-{4-[2-chloro-6-(3,5-dimethyl-isoxazol-4-yl)-pyrimidin-4-ylamino]-phenyl}-cyclobutanecarboxylic acid methyl ester

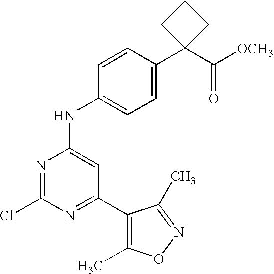

The title compound was afforded as a light yellow solid following the procedure as described in Example 59 step (i) by using 3,5-dimethylisoxazole boronic acid instead of phenyl boronic acid. Purification (Biotage Horizon HPFC chromatography system, SiO₂, 70:30 hexane:EtOAc) purification gave the title compound (53% yield).

HPLC: Betasil ODS-3V C18, 30:70 [KH₂PO₄ (0.01M, pH 3.2): CH₃CN], 264 nm, Rₜ 13.24 min, 98.9% purity.

¹H NMR (300 MHz, CDCl₃, TMS): δ 7.37-7.26 (m, 4H), 7.04 (s, 1H), 6.56 (s, 1H), 3.66 (s, 3H), 2.90-2.81 (m, 1H), 2.61 (s, 3H), 2.55-2.45 (m, 1H), 2.38 (s, 3H), 2.12-1.85 (m, 2H), 1.59 (b s, 2H).

LC-MSD (ES+): m/z [413 (M+H)⁺, 100].

### Step (ii): Synthesis of 1-{4-[6-(3,5-dimethyl-isoxazol-4-yl)-2-(4-fluoro-phenyl)-pyrimidin-4-ylamino]-phenyl}-cyclobutanecarboxylic acid methyl ester

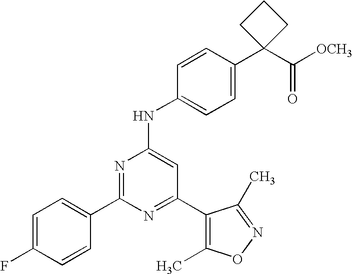

The title compound was afforded as a light yellow solid following the procedure as described in Example 59 step (ii) by using 3,5-dimethylisoxazole boronic acid instead of phenyl boronic acid. Purification (Biotage Horizon HPFC chromatography system, SiO₂, 50:50 hexane:EtOAc) purification gave the title compound (59% yield).

HPLC: Betasil ODS-3V C18, 30:70 [KH₂PO₄ (0.01M, pH 3.2): CH₃CN], 264 nm, Rₜ 31.3 min, 99.1% purity.

¹H NMR (300 MHz, CDCl₃, TMS): δ 8.46-8.41 (m, 2H), 7.44-7.34 (m, 4H), 7.20-7.11 (m, 2H), 6.88 (s, 1H), 6.61 (s, 1H), 3.68 (s, 3H), 2.89-2.82 (m, 1H), 2.69 (s, 3H), 2.58-2.50 (m, 1H), 2.48 (s, 3H), 2.09-1.87 (m, 2H), 1.58 (s, 2H).

LC-MSD (ES+): m/z [473 (M+H)⁺, 100].

### Step (iii): Synthesis of 1-{4-[6-(3,5-dimethyl-isoxazol-4-yl)-2-(4-fluoro-phenyl)-pyrimidin-4-ylamino]-phenyl}-cyclobutanecarboxylic acid

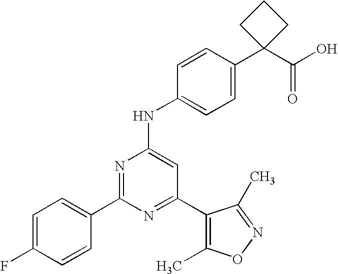

The title compound was afforded as a yellow solid following the procedure as described in Example 1, step (v) (82% yield).

HPLC: Betasil ODS-3V C18, 30:70 [KH₂PO₄ (0.01M, pH 3.2): CH₃CN], 264 nm, Rₜ 31.3 min, 99.2% purity.

¹H NMR (300 MHz, CDCl₃, TMS): δ 8.43-8.37 (m, 2H), 7.43 to 7.35 (m, 4H), 7.19-7.11 (m, 4H), 6.60 (s, 1H), 2.90-2.84 (m, 1H), 2.67 (s, 3H), 2.68-2.60 (m, 1H), 2.47(s, 3H), 2.16-2.07 (m, 2H), 1.95-1.87 (m, 2H).

LC-MSD (ES+): m/z [459 (M+H)⁺, 100].

### Example 62

### Synthesis of 1-{4-[2-(6-methoxy-pyridin-3-yl)-6-phenyl-pyrimidin-4-ylamino]-phenyl}-cyclobutanecarboxylic acid

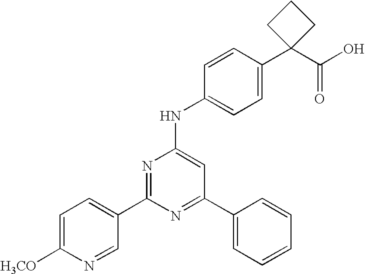

The title compound was afforded as a yellow solid following the procedure as described in Example 59, step iii by using methanol as solvent and using 2-methoxy-5-pyridine boronic acid (59% yield).

HPLC: Betasil ODS-3V C18, 30:70 [KH₂PO₄ (0.01M, pH 3.2): CH₃CN], 264 nm, Rₜ 15.4 min, 94.8% purity.

¹H NMR (300 MHz, CDCl₃, TMS): δ 9.33-9.32 (m, 1H), 8.67 (dd, J=8.7, 2.4 Hz, 1H), 8.11-8.08 (m, 2H), 7.55-7.36 (m, 9H), 7.00 (s, 1H), 4.04 (s, 3H), 2.92-2.86 (m, 2H), 2.59-2.52 (m, 2H), 2.13-1.88 (m, 2H).

LC-MSD (ES+): m/z [453 (M+H)⁺, 100].

### Example 63

### Synthesis of 1-{4-[2-(4-methanesulfonyl-phenyl)-6-phenyl-pyrimidin-4-ylamino]-phenyl}-cyclobutanecarboxylic acid

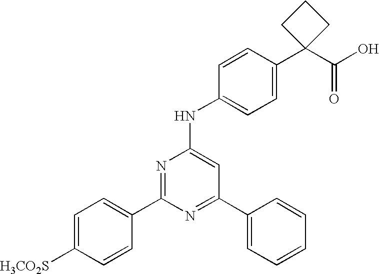

The title compound was afforded as a yellow solid following the procedure as described in Example 59 steps (ii) and (iii) using methanol as solvent and using 4-methanesulfonyl boronic acid (16% yield).

HPLC: Betasil ODS-3V C18, 30:70 [KH₂PO₄ (0.01M, pH 3.2): CH₃CN], 264 nm, Rₜ 7.79 min, 94.01% purity.

¹H NMR (300 MHz, DMSO-d₆): δ 9.96 (br s, 1H), 8.67 (d, J=8.4 Hz, 1H), 8.16-8.07 (m, 4H), 7.73 to 7.54 (m, 5H), 7.30-7.26 (m, 4H), 3.32 (s, 3H), 2.70 (br s, 1H), 2.34-2.26 (m, 2H), 1.92-1.65 (m, 4H).

LC-MSD (ES+): m/z [500 (M+H)⁺, 100].

### Example 64

### Synthesis of 1-{4-[2-(6-methyl-pyridin-3-yl)-6-phenyl-pyrimidin-4-ylamino]-phenyl}-cyclobutanecarboxylic acid

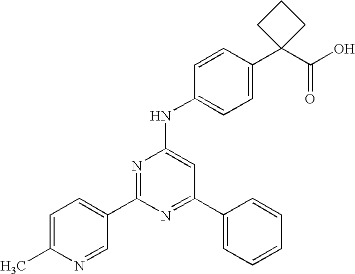

### Step (i): Synthesis of 1-{4-[2-(6-methyl-pyridin-3-yl)-6-phenyl-pyrimidin-4-ylamino]-phenyl}-cyclobutanecarboxylic acid methyl ester

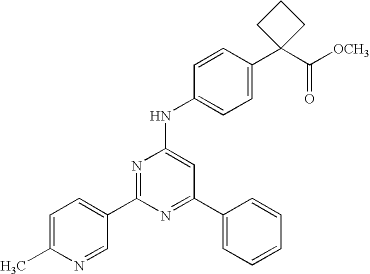

The title compound was afforded as a light yellow solid following the procedure as described in Example 59, step (ii) by using 2-methylpyridine-5-boronic acid. Purification (Biotage Horizon HPFC chromatography system, SiO₂, 50:50 hexane:EtOAc) purification gave the title compound (73% yield).

HPLC: Betasil ODS-3V C18, 30:70 [KH₂PO₄ (0.01M, pH 3.2): CH₃CN], 264 nm, Rₜ 15.66 min, 99.2% purity.

¹H NMR (300 MHz, CDCl₃, TMS): δ 9.62 (d, J=2.1 Hz, 1H), 8.68 (dd, J=2.1, 8.1 Hz, 1H), 8.13-8.07 (m, 2H), 7.53 to 7.45 (m, 5H), 7.38-7.31 (m, 4H), 7.03 (s, 1H), 3.67 (s, 3H), 2.91-2.82 (m, 2H), 2.65 (s, 3H), 2.14-1.87 (m, 4H).

LC-MSD (ES+): m/z [451 (M+H)⁺, 100].

### Step (ii): Synthesis of 1-{4-[2-(6-methyl-pyridin-3-yl)-6-phenyl-pyrimidin-4-ylamino]-phenyl}-cyclobutanecarboxylic acid

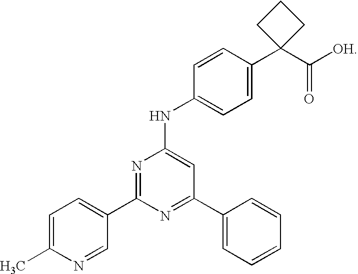

The title compound was afforded as a light yellow solid following the procedure as described in Example 59, step (iii) using methanol as solvent (93% yield).

HPLC: Betasil ODS-3V C18, 30:70 [KH₂PO₄ (0.01M, pH 3.2): CH₃CN], 264 nm, Rₜ 7.57 min, 98.4% purity.

¹H NMR (300 MHz, DMSO-d₆): δ 10.01 (s, 1H), 9.45 (s, 1H), 8.17-8.14 (m, 2H), 7.79-7.50 (m, 8H), 7.32-7.27 (m, 3H), 2.65 (s, 3H), 2.42-2.36 (m, 2H), 1.92-1.76 (m, 4H).

LC-MSD (ES+): m/z [437 (M+H)⁺, 100].

### Example 65

### Synthesis of 1-{4-[2-(1,1-dioxo-1λ⁶-thiomorpholin-4-yl)-6-phenyl-pyrimidin-4-ylamino]-phenyl}-cyclobutanecarboxylic acid

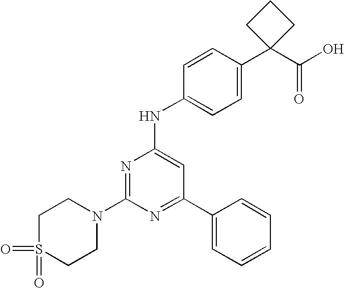

### Step (i): Synthesis of 1-[4-(6-phenyl-2-thiomorpholin-4-yl-pyrimidin-4-ylamino)-phenyl]-cyclobutanecarboxylic acid methyl ester

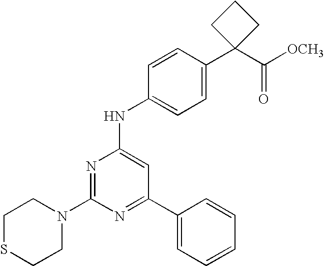

A similar method was used as in Example 1, step (iv) by using 1-[4-(2-chloro-6-phenyl-pyrimidin-4-ylamino)-phenyl]-cyclobutanecarboxylic acid methyl ester and thiomorpholine instead of tert-butyl-(2-chloro-6-phenyl-pyrimidine-4-yl)-amine and 1-(4-aminophenyl)-cyclobutane-carboxylic acid methyl ester. Purification (Biotage Horizon HPFC chromatography system, SiO₂, 80:20 hexanes:EtOAc) gave the title compound as a white solid (212 mg, 90% yield).

HPLC: Betasil ODS-3V C18, 30:70 [KH₂PO₄ (0.01M, pH 3.2): CH₃CN], 264 nm, Rₜ 34.4 min, 96% purity.

¹H NMR (300 MHz, CDCl₃, TMS): δ 7.97-7.93 (m, 2H), 7.44-7.26 (m, 7H), 6.54 (s, 1H), 6.45 (s, 1H), 4.25-4.22 (m, 4H), 3.67 (s, 3H), 2.89-2.80 (m, 2H), 2.72-2.69 (m, 4H), 2.56-2.47 (m, 2H), 2.10-1.84 (m, 2H).

LC-MSD (ES+): m/z [461 (M+H)⁺, 100].

### Step (ii): Synthesis of 1-{4-[2-(1,1-dioxo-1λ⁶-thiomorpholin-4-yl)-6-phenyl-pyrimidin-4-ylamino]-phenyl}-cyclobutanecarboxylic acid methyl ester

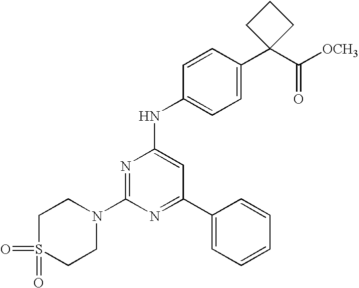

1-[4-(6-Phenyl-2-thiomorpholin-4-yl-pyrimidin-4-ylamino)-phenyl]-cyclobutane-carboxylic acid methyl ester (0.200 g, 0.43 mmol) was dissolved in methanol (6 mL) and oxone (0.925 g, 1.5 mmol) was added. The resulting mixture was stirred at about 20-35° C. under nitrogen atmosphere for 2 h. The mixture was diluted with EtOAc, washed two times with water and one time with brine. The organic phase was dried over sodium sulfate, filtered, and concentrated by rotary evaporation. The resulting sample was dried under vacuum and purified. Purification (Biotage Horizon HPFC chromatography system, SiO₂, 95:5 dichloromethane:methanol) gave a light brown solid (120 mg, 56% yield) as the title compound.

HPLC: Betasil ODS-3V C18, 30:70 [KH₂PO₄ (0.01M, pH 3.2): CH₃CN], 264 nm, Rₜ 9.7 min, 98% purity.

¹H NMR (300 MHz, CDCl₃, TMS): δ 7.94-7.91 (m, 2H), 7.45-7.29 (m, 7H), 6.81 (s, 1H), 6.56 (s, 1H), 4.42 (s, 4H), 3.67(s, 3H), 3.09 (s, 4H), 2.89-2.81 (m, 2H), 2.56-2.49 (m, 2H), 2.12-1.81 (m, 2H).

LC-MSD (ES+): m/z [493 (M+H)⁺, 100].

### Step (iii): Synthesis of 1-{4-[2-(1,1-dioxo-1λ⁶-thiomorpholin-4-yl)-6-phenyl-pyrimidin-4-ylamino]-phenyl}-cyclobutanecarboxylic acid

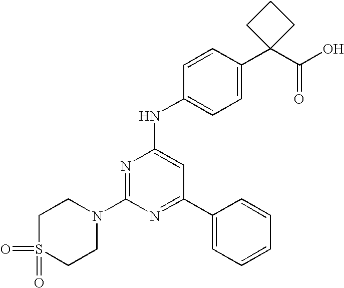

A similar method was used as in Example 1, step (v) by using 1-{4-[2-(1,1-dioxo-1λ⁶-thiomorpholin-4-yl)-6-phenyl-pyrimidin-4-ylamino]-phenyl}-cyclobutane carboxylic acid methyl ester instead of 1-[4-(4-tert-butylamino-6-phenyl-pyrimidin-2-ylamino)-phenyl]-cyclobutanecarboxylic acid methyl ester. The resulting pale yellow solid was dried under vacuum to give the title compound (90.0 mg, 97% yield).

HPLC: Betasil ODS-3V C18, [KH₂PO₄ (0.01M, pH 3.2): CH₃CN], gradient, 264 nm, Rₜ 17.1 min, 98% purity.

¹H NMR (300 MHz, DMSO-d₆): δ 12.06 (br s, 1H), 9.47 (s, 1H), 7.99-7.96 (m, 2H), 7.62-7.46 (m, 5H), 7.24 (d, J=8.7 Hz, 2H), 6.64 (s, 1H), 4.32 (s, 4H), 3.18 (s, 4H), 2.79-2.68 (m, 2H), 2.45-2.36 (m, 2H), 2.00-1.79 (m, 2H).

LC-MSD (ES+): m/z [479 (M+H)⁺, 100].

### Example 66

### Synthesis of 1-{4-[6-(3,5-dimethyl-isoxazol-4-yl)-2-morpholin-4-yl-pyrimidin-4-ylamino]-phenyl}-cyclobutanecarboxylic acid

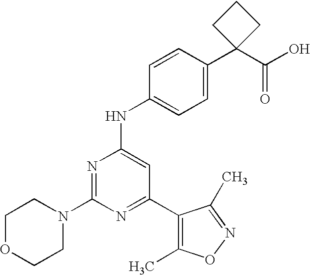

1-{4-[2-Chloro-6-(3-methyl-isoxazol-4-yl)-pyrimidin-4-ylamino]-phenyl}-cyclo-butanecarboxylic acid methyl ester (400 mg, 0.97 mmol) was dissolved in morpholine (10 mL). The resulting solution was allowed to stir at reflux for about 10-19 h. The mixture was diluted with dichloromethane, washed two times with water and one time with brine. The organic phase was dried over sodium sulfate, filtered, and concentrated by rotary evaporation. The resulting sample was dried under vacuum and purified. Flash column chromatography (SiO₂, 95:5 dichloromethane:methanol) purification gave the title compound as a light brown solid (424 mg, 94% yield).

HPLC: Betasil ODS-3V C18, 30:70 [KH₂PO₄ (0.01M, pH 3.2): CH₃CN], 264 nm, Rₜ 6.39 min, 99.6% purity.

¹H NMR (300 MHz, DMSO-d₆): δ 12.24 (s, 1H), 9.40 (s, 1H), 7.62 (d, J=8.7 Hz, 2H), 7.20 (d, J=8.7 Hz, 2H), 6.25 (s, 1H), 3.67 (s, 8H), 3.32 (s, 2H), 2.70-2.35 (m, 8H, including DMSO), 1.93-1.76 (m, 2H).

LC-MSD (ES+): m/z [450 (M+H)⁺, 100].

### Example 67

### Synthesis of 1-[4-(2-morpholin-4-yl-6-phenyl-pyrimidin-4-ylamino)-phenyl]-cyclobutanecarboxylic acid

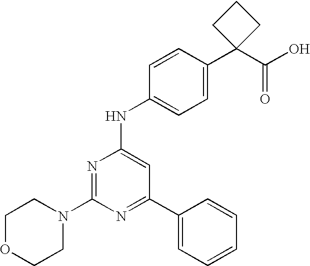

### Step (i): Synthesis of 1-[4-(2-morpholin-4-yl-6-phenyl-pyrimidin-4-ylamino)-phenyl]-cyclobutanecarboxylic acid methyl ester (A) and 1-[4-(2-morpholin-4-yl-6-phenyl-pyrimidin-4-ylamino)-phenyl}-cyclobutanecarboxylic acid (B)

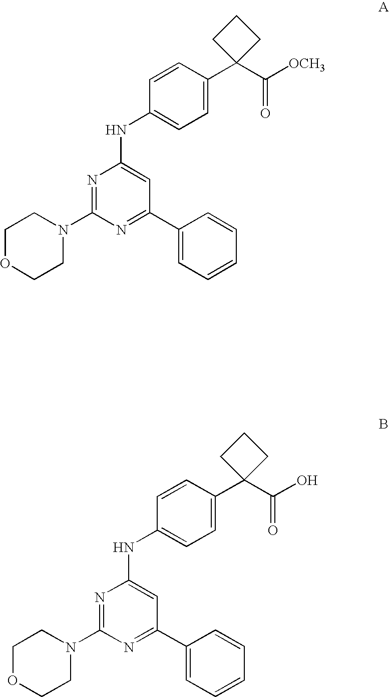

1-[4-(2-Chloro-6-phenyl-pyrimidin-4-ylamino)-phenyl]-cyclobutane-carboxylic acid methyl ester (0.270 g, 0.69 mmol) was dissolved in morpholine (5 mL). The resulting mixture was stirred at reflux for about 10-19 h under nitrogen atmosphere. The mixture was diluted with dichloromethane, and washed two times with water. The organic phase was dried over sodium sulfate, filtered, and concentrated by rotary evaporation. The resulting sample was dried under vacuum and purified. Purification (Biotage Horizon HPFC chromatography system, SiO₂, 95:5 dichloromethane: methanol) afforded both the ester (A) and acid (B).

Compound A: 1-[4-(2-morpholin-4-yl-6-phenyl-pyrimidin-4-ylamino)-phenyl]-cyclo-butanecarboxylic acid methyl ester (pale yellow solid, 70 mg, 22% yield).

HPLC: Betasil ODS-3V C18, 30:70 [KH₂PO₄ (0.01M, pH 3.2): CH₃CN], 264 nm, Rₜ 18.1 min, 95% purity.

¹H NMR (300 MHz, CDCl₃, TMS): δ 7.99-7.96 (m, 2H), 7.45-7.27 (m, 7H), 6.60 (br s, 1H), 6.49 (s, 1H), 3.92-3.79 (m, 8H), 3.68 (s, 3H), 2.89-2.81 (m, 2H), 2.57-2.48 (m, 2H), 2.11-1.80 (m, 2H).

LC-MSD (ES+): m/z [445 (M+H)⁺, 100].

Compound B: 1-[4-(2-morpholin-4-yl-6-phenyl-pyrimidin-4-ylamino)-phenyl]-cyclo-butanecarboxylic acid (white solid, 40 mg, 13% yield).

HPLC: Betasil ODS-3V C18, 30:70 [KH₂PO₄ (0.01M, pH 3.2): CH₃CN], 264 nm, Rₜ 9.0 min, 99% purity.

¹H NMR (300 MHz, DMSO-d₆): δ 9.13 (s, 1H), 8.19 (s, 1H), 8.00-7.96 (m, 2H), 7.52-7.44 (m, 5H), 7.26 (d, J=8.7 Hz, 2H), 6.56 (s, 1H), 3.81-3.73 (m, 8H), 2.82-2.71 (m, 2H), 2.42-2.20 (m, 2H), 1.89-1.61 (m, 2H).

LC-MSD (ES+): m/z [431 (M+H)⁺, 100].

### Example 68

### Synthesis of 1-[4-(4-isopropoxy-6-phenyl-pyrimidin-2-ylamino)-phenyl]-cyclobutane-carboxylic acid

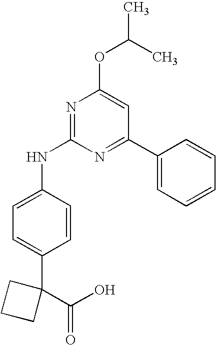

### Step (i): Synthesis of 2-chloro-4-isopropoxy-6-phenyl-pyrimidine

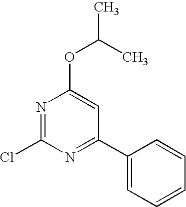

The title compound was prepared following a method used by Tamayo, N. et al. J. Med. Chem. 2008, 51, 2744-2757.

¹H NMR (400 MHz, CDCl₃) δ 8.15-7.96 (m, 2H), 7.55-7.46 (m, 3H), 6.94 (s, 1H), 5.56-5.46 (m, 1H), 1.40 (d, J=6.2 Hz, 6H).

LC-MS (ES+): m/z [273 (M+Na)⁺, 100)].

### Step (ii): Synthesis of 1-[4-(4-isopropoxy-6-phenyl-pyrimidin-2-ylamino)-phenyl]-cyclobutanecarboxylic acid methyl ester

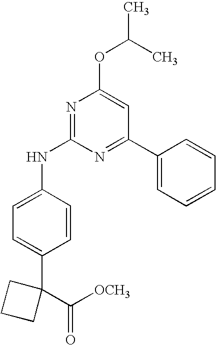

The title compound was prepared following the procedure as described in Example 9 step (ii), by using 2-chloro-4-isopropoxy-6-phenyl-pyrimidine instead of 2-chloro-6-(4-fluoro-phenyl)-pyrimidin-4-yl]-cyclopropylmethyl-amine.

HPLC: Inertsil C18, gradient, [KH₂PO₄ (0.01M, pH 3.2): CH₃CN], 271 nm, Rₜ 11.9 min, 94.46% purity.

¹H NMR (400 MHz, CDCl₃) δ 8.15-7.96 (m, 2H), 7.68 (d, J=8.2 Hz, 2H), 7.54-7.48 (m, 3H), 7.28 (d, J=8.2 Hz, 2H), 7.14 (b s, 1H), 6.58 (s, 1H), 5.42-5.38 (m, 1H), 3.62 (s, 3H), 2.91-2.81 (m, 2H), 2.58-2.46 (m, 2H), 2.16-1.86 (m, 2H), 1.41 (d, J=6.2 Hz, 6H).

LC-MSD (ES+): m/z [418 (M+H)⁺, 100].

### Step (iii): Synthesis of 1-[4-(4-isopropoxy-6-phenyl-pyrimidin-2-ylamino)-phenyl]-cyclobutane-carboxylic acid

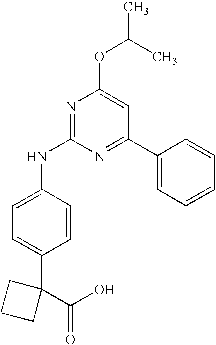

The title compound was afforded as a white powder following the method described in Example 9, step (iii) by using an appropriate starting material.

HPLC: Inertsil C18, gradient, [KH₂PO₄ (0.01M, pH 3.2): CH₃CN], 210 nm, Rₜ 12.04 min, 96.88% purity.

¹H NMR (400 MHz, DMSO-d₆) δ 12.20 (b s, 1H), 9.50 (b s, 1H), 8.18 (d, J=7.8 Hz, 2H), 7.80 (d, J=7.8 Hz, 2H), 7.52-7.44 (m, 3H), 7.24-7.19 (m, 2H), 6.68 (s, 1H), 5.49-5.37 (m, 1H), 2.71-2.64 (m, 2H), 2.32-2.45 (m, 2H), 1.98-1.67 (m, 2H), 1.36 (d, J=6.2 Hz, 6H).

LC-MSD (ES+): m/z [404 (M+H)⁺, 100].

### Example 69

### GATA Assay

Human HepG2 (ATCC) cells were grown in Eagle's Minimum Essential Media (MEM) containing 10% heat inactivated Fetal Bovine Serum (FBS) (Invitrogen) and 5 mL Penicillin-Streptomycin (Invitrogen) in T75 flasks (Corning) in a 37° C./5% CO₂ tissue culture incubator (Thermo Electron Corporation) prior to the study. 96-well plates (Corning) were seeded in MEM containing 10% FBS to yield ˜50% confluency. The following day, cells were transfected using FuGENE 6 transfection reagent (Roche) and 550 ng/μL GATA Translucent Reporter Vector (Panomics) according the manufacturer's (Roche) protocol and incubated for about 10-19 h. The next day, the cells were treated with equal volumes of vehicle, dimethyl sulfoxide (DMSO) (Hybri-Max Sterile Filtered Sigma Aldrich) or compounds of formula (I) (in DMSO) diluted in MEM containing 1% FBS. The treated cells were incubated about 8-19 h. The next day the treatment media was removed and the cells were lysed and assayed for luciferase activity using the luciferase assay system (Promega) and using the Envision plate reader (Perkin Elmer). The lysates were also used to measure total protein content with the MicroBCA protein assay kit (Pierce) using the Multiskan Ascent plate reader at 570 nm. Luciferase values were normalized to total protein and fold induction was calculated.

Compounds that show an increase of ≧40% in luciferase levels at ≦20 μm concentration were considered as GATA activators.

| | |
|---|---|
| | GATA1 Activation (Fold |
| Example | Change Vs Control) |

| No. | 5μ | 10μ |
|---|---|---|
| 16 | 2.44 | 2.13 |
| 25 | 3.69 | 3.32 |
| 30 | 1.64 | 1.74 |
| 55 | 2.86 | 2.40 |
| 68 | 2.57 | 2.44 |

### Example 70

### Enzyme-Linked Immunosorbant Assay (ELISA)

Human HepG2 (ATCC) cells were grown in Eagle's Minimum Essential Media (MEM) Modified containing 10% heat inactivated Fetal Bovine Serum (FBS) (Invitrogen) and 5 mL Penicillin-Streptomycin (Invitrogen) in T75 flasks (Corning) in a 5% CO₂/37° C. the tissue culture incubator (Thermo Electron Corporation) prior to the study. 96-well plates (Corning) were seeded with 25,000 cells per well in MEM+10% FBS. The third day the media was changed to treatment media (MEM+1% FBS). Treatments were done with enough replicates for there to be triplicate samples for both the ELISA procedures as well as the MicroBCA procedure. The cells were treated with equal volumes of vehicle DMSO Hybri-Max Sterile Filtered (Sigma Aldrich) or compounds of formula (I) (prepared in DMSO) prepared in MEM containing 1% FBS at different concentrations for 24 & 48 hours. Cells from each time point were washed with 1×PBS and fixed with 100% methanol (Sigma Aldrich) for 10 minutes at about 20-35° C. or lysed with T-PER Tissue Protein Extraction Reagent (Pierce).

For the ELISAs, the methanol was removed and the plates were air dried and ready for use. The wells were blocked with 1×PBS containing 0.5% Bovine Serum Albumin fraction V (Fisher Scientific) with 200 μL/well for one hour at about 20-35° C. The block was removed and the wells were incubated with primary antibody (100 μL/well) for two hours at about 20-35° C. The primary antibodies are either ABCA1 (Santa Cruz Biotechnology) diluted 1:200 in 1×PBS+0.5% BSA or Anti-SRB1 (Novus Biologicals) diluted 1:2000 in 1×PBS+0.5% BSA. After the incubation the wells were washed two times. Wells were washed with 1×PBS+0.5% BSA (200 μL/well) at about 20-35° C. for 5 minutes on an orbital shaker. After the washes wells were incubated in the secondary antibody, Goat α Rabbit IgG-HRP (Zymed, Invitrogen) (100 μL/well) diluted 1:5000 in 1×PBS+0.5% BSA, for one hour at about 20-35° C. Wells were washed as described above. For detection, 100 μL/well of color reagent (R&D systems) was added to the wells and the plates were incubated in the dark for 20 minutes. The color reaction was stopped with 50 μL/well 0.2N sulfuric acid (Fisher Scientific). The plates were read on the Multiskan Ascent plate reader at both 450 and 570 nm.

Cells for normalization with MicroBCA Protein assay were lysed by adding T-PER. The lysates were used to measure total protein content with the MicroBCA protein assay kit (Pierce) using the Multiskan Ascent plate reader at 570 nm.

The ELISA values were normalized total protein.

Compounds that showed an increase of ≧30% in ABCA1 and/or SR-B1 at ≦20 μm concentration are considered active compounds for RCT activity.

### Example 71

### Cholesterol Efflux Assay

Human THP-1 cells, monocytes (macrophages), from ATCC are plated in 24 well tissue culture plates (Corning) at a density of 2.2E⁵cells per well in RMPI 1640 (ATCC) media containing 10% heat inactivated Fetal Bovine Serum (Invitrogen), 48.96 μM β-Mercaptoethanol (Sigma Aldrich), and 200 ng/mL Phorbol 12-myristate 13-acetate (PMA) (EMD Biosciences Inc). The macrophages were allowed to attach and adhere to the wells for 72 hours in a 37° C./5% CO₂ tissue culture incubator (Thermo Electron Corporation). The next day the cells were labeled with RPMI 1640 containing 5% Lipid Reduced Fetal Bovine Serum (LRFBS) (Hyclone), 25 μg/mL human Acetylated Low Density Lipoprotein (Biomedical Technologies), and 1 μCI/mL 3H-Cholesterol (Perkin Elmer) for 24 hours. The following day, the wells were washed with 700 μL per well PBS, pH 7.4, and then 500 μL of RPMI 1640 media containing 0.2% Bovine Serum Albumin fraction V (Fisher Scientific) and either 20 μM of TO901317 (Cayman Chemical) or 20 μM compound of formula (I) or an equivalent volume of DMSO Hybri-Max Sterile Filtered (Sigma Aldrich) was added to the wells. The plates were incubated with compound treatments for 24 hours. The following day, the compound treatments were removed and 500 μL of RPMI 1640 media containing 0.2% BSA +/−25 μg/mL Apolipoprotein A-1, human (ApoA1) (Biomedical Technologies) was added to the wells. The plates were incubated with efflux media treatments for 24 hours. On the final day, the media was transferred to vials containing Ecocint A (National Diagnostics USA) scintillation fluid; the cells were washed once in 1×PBS and then lysed with 500 μL lysis buffer containing 0.1 N NaOH (Fisher scientific) and 0.1% sodium lauryl sulfate (Sigma Aldrich). The lysates were also transferred to vials containing scintillation fluid. The samples were run on the TriCarb 2900 liquid scintillation counter (Perkin Elmer). Data was generated as counts per minute (CPM). The percentage cholesterol efflux was calculated by dividing media derived radioactivity by the sum of the radioactivity in the media and cells. The % efflux into BSA media was subtracted from the % efflux into the ApoA1 media for each treatment condition. The data are expressed as a percentage of the DMSO control.

Compounds that showed an increase of ≧30% at ≦30 μm concentration in cholesterol efflux are considered active RCT enhancing compounds.

Although several embodiments of the invention have been described using specific terms, devices, and methods, such description is for illustrative purposes only. The words used are words of description rather than of limitation. It is to be understood that changes and variations may be made by those of ordinary skill in the art without departing from the spirit or scope of the present invention, which is set forth in the following claims. In addition, it should be understood that aspects of the various embodiments may be exchanged in whole or in part.

All references cited in this specification are hereby incorporated by reference into this specification in their entireties.

The discussion of references herein is intended merely to summarize the assertions made by their authors and no admission is made that any reference constitutes prior art. Applicants reserve the right to challenge the accuracy and pertinence of the cited references.

We claim:

1. A compound of formula (I), 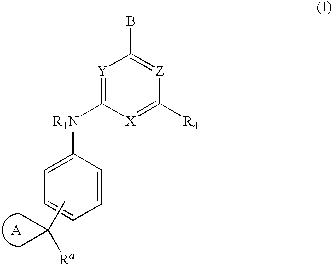
stereoisomers thereof and/or pharmaceutically acceptable salts of compounds of formula (I) and/or pharmaceutically acceptable salts of the stereoisomers of compounds of formula (I); wherein,
X, Y and Z are each independently selected from N or CR^{b}, wherein R^{b} is selected from hydrogen, halogen or alkyl; with a proviso that when X and Y are each independently N, Z is —CR^{b} and when Y and Z are each independently N, X is —CR^{b};
A is a 3 to 7 membered cycloalkyl ring;
B is an optionally substituted group selected from alkyl, alkoxy, cycloalkyl, aryl, heteroaryl or —NR₂R₃, wherein one or more optional substituents on B are selected from halogen, hydroxy, cyano, alkyl, haloalkyl, alkoxy, cycloalkyl, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl;
R₁ is selected from hydrogen or alkyl;
R₂ and R₃ are each independently selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl, wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl; or
R₂ and R₃ optionally combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring are selected from halogen, hydroxy, alkoxy, alkyl, cycloalkyl or —CO-alkyl;
R₄ is hydrogen or an optionally substituted group selected from amino, alkyl, cycloalkyl, aryl, heterocyclyl or heteroaryl, wherein one or more optional substituents on R₄ are selected from halogen, hydroxy, cyano, alkyl, —(CH₂)ₙ—OH, cycloalkyl, alkoxy or —CO-alkyl;
R^{a} is selected from cyano, —(CH₂)ₙ—OH, —(CH₂)ₙ-alkoxy, alkyl, aryl, —NH-aryl, heteroaryl, —(CH₂)ₙCO-alkyl, —(CH₂)ₙCOOR^{c}, —(CH₂)ₙCONR^{d}R^{e}, —(CH₂)ₙSO₂R^{f}, or —(CH₂)ₙSO₂NR^{c}R^{d};
R^{c} is selected from hydrogen, alkyl, cycloalkyl, aryl, heterocyclyl or heteroaryl;
R^{d} and R^{e} are each independently selected from hydrogen or an optionally substituted group selected from alkyl, cycloalkyl, alkoxy, aryl, heteroaryl or heterocyclyl, wherein one or more optional substituents on R^{d} and R^{e} are each independently selected from halogen, hydroxy or alkyl;
R^{f} is selected from alkyl, cycloalkyl, heterocyclyl, aryl or heteroaryl; and
n is an integer from 0 to 2.

2. The compound as claimed in claim 1, having the formula (II) 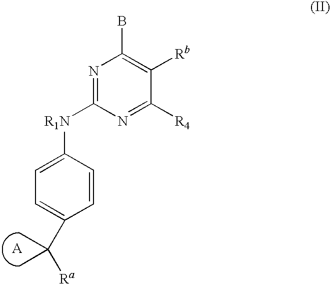
stereoisomers thereof and/or pharmaceutically acceptable salts of compounds of formula (II) and/or pharmaceutically acceptable salts of the stereoisomers of compounds of formula (II)

3. The compound as claimed in claim 1, having the formula (III) 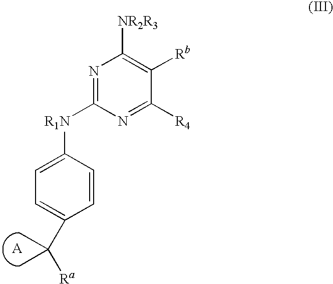
stereoisomers thereof and/or pharmaceutically acceptable salts of compounds of formula (III) and/or pharmaceutically acceptable salts of the stereoisomers of compounds of formula (III).

4. The compound as claimed in claim 3, wherein
A is selected from cyclopropyl, cyclobutyl or cyclopentyl;
R₁ is hydrogen;
R₂ is selected from hydrogen or —(CH₂)ₙ-cycloalkyl;
R₃ is selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl; wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl;
R₄ is an optionally substituted group selected from amino, alkyl, cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;
R^{a} is selected from cyano, alkyl, —(CH₂)ₙ—OH, —(CH₂)ₙ-alkoxy, —CO-alkyl, heteroaryl, —(CH₂)ₙCOOR^{c}, —(CH₂)ₙCONR^{d}R^{e}, —(CH₂)ₙSO₂R^{f} or —(CH₂)ₙSO₂NR^{c}R^{d};
R^{b} is selected from hydrogen or alkyl;
R^{c} is selected from hydrogen or alkyl;
R^{d} and R^{e} are each independently selected from hydrogen, alkyl, alkoxy or aryl, which is optionally substituted with halogen;
R^{f} is selected from alkyl, cycloalkyl, heterocyclyl, aryl or heteroaryl; and
n is an integer from 0 to 2.

5. The compound as claimed in claim 4, wherein
R₃ is selected from hydrogen, alkyl, —(CH₂)ₙ-cycloalkyl, and optionally substituted groups selected from —(CH₂)ₙ—OH, —(CH₂)ₙ-heterocyclyl, —(CH₂)ₙ-aryl, —(CH₂)ₙ-heteroaryl, wherein the optional substituents on R₃ are selected from halogen, —CH₂—OH, alkyl, aryl, —(CH₂)ₙSO₂NR^{c}R^{d} or —(CH₂)ₙSO₂R^{f};
R₄ is selected from an optionally substituted group selected from amino, alkyl, cycloalkyl, aryl, heterocyclyl or heteroaryl, wherein one or more optional substituents on R₄ are selected from halogen, alkyl or alkoxy;
R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂)ₙSO₂NR^{c}R^{d};
R^{c} is selected from hydrogen or alkyl, and
R^{d} and R^{e} are each independently selected from hydrogen, alkyl, alkoxy or aryl, which is optionally substituted with halogen.

6. The compound as claimed in claim 3, wherein
R₂ and R₃ combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring fowled may be selected from halogen, hydroxyl, alkoxy, alkyl, cycloalkyl or —CO-alkyl;
R₄ is an optionally substituted group selected from cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;
R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂)ₙSO₂NR^{c}R^{d};
R^{c} is selected from hydrogen or alkyl;
R^{d} and R^{e} are each independently selected from hydrogen, alkyl or alkoxy;
R^{f} is selected from alkyl, cycloalkyl, heterocyclyl, aryl or heteroaryl; and
n is an integer from 0 to 2.

7. The compound as claimed in claim 6, wherein
R₂ and R₃ combine with the nitrogen atom, to which they are attached, to form an optionally substituted morpholinyl, piperidinyl, pyrrolidinyl, piperazinyl, thiomorpholinyl, dioxo-thiomorpholinyl, wherein one or more optional substituents on the heterocyclic ring formed may be selected from halogen, hydroxy, alkoxy, alkyl or —CO-alkyl;
R₄ is an optionally substituted group selected from amino, alkyl, cycloalkyl, aryl, heterocyclyl or heteroaryl, wherein one or more optional substituents on R₄ are selected from halogen, alkyl or alkoxy;
R^{a} is selected from —COOR^{c} or —CONR^{d}R^{e};
R^{c} is selected from hydrogen or alkyl; and
R^{d} and R^{e} are independently selected from hydrogen, alkyl, alkoxy or aryl, which is optionally substituted with halogen.

8. The compound as claimed in claim 1, having the formula (IV), 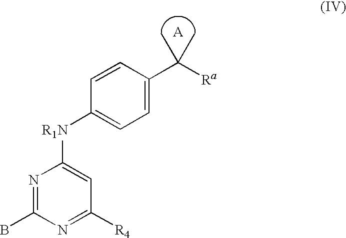
stereoisomers thereof and/or pharmaceutically acceptable salts of compounds of formula (IV) and/or pharmaceutically acceptable salts of the stereoisomers of compounds of formula (IV).

9. The compound as claimed in claim 8, wherein
R₁ is hydrogen;
A is selected from cyclopropyl, cyclobutyl or cyclopentyl;
B is an optionally substituted group selected from alkyl, cycloalkyl, aryl, heteroaryl or —NR₂R₃, wherein one or more optional substituents on B are selected from halogen, hydroxy, alkyl, haloalkyl, alkoxy, —CO-alkyl, —(CH₂)ₙSO₂R^{f} or —(CH₂)ₙSO₂NR^{c}R^{d};
R₃ is selected from hydrogen, —(CH₂)ₙ—OH, —(CH₂)ₙ-cycloalkyl, an optionally substituted groups selected from alkyl, —(CH₂)ₙ-heterocyclyl, —(CH₂)ₙ-aryl, —(CH₂)ₙ-heteroaryl, wherein one or more optional substituents on R₃ are selected from halogen, —CH₂—OH, alkyl, aryl, —(CH₂)ₙSO₂NR^{c}R^{d} or —(CH₂)ₙSO₂R^{f};
R₄ is an optionally substituted group selected from alkyl, cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;
R^{a} is selected from cyano, alkyl, —(CH₂)ₙ—OH, —(CH₂)ₙ-alkoxy, —CO-alkyl, heteroaryl, —COOR^{c}, —CONR^{d}R^{e}, —(CH₂)ₙSO₂R^{f} or —(CH₂)—SO₂NR^{c}R^{d};
R^{c} is hydrogen;
R^{d} and R^{e} are each independently selected from hydrogen, alkyl, alkoxy or aryl, which is optionally substituted with halogen;
R^{f} is selected from alkyl, cycloalkyl, heterocyclyl, aryl or heteroaryl; and
n is an integer from 0 to 2.

10. The compound as claimed in claim 8, wherein 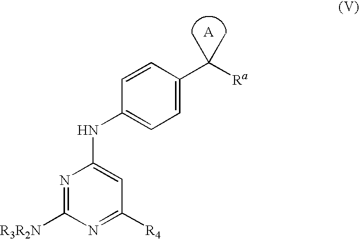
stereoisomers thereof and/or pharmaceutically acceptable salts of compounds of formula (V) and/or pharmaceutically acceptable salts of the stereoisomers of compounds of formula (V); wherein
R₂ and R₃ combine with the nitrogen atom, to which they are attached, to form an optionally substituted 5- to 6-membered cyclic ring optionally having 1 to 3 additional hetero atoms or groups selected from nitrogen, oxygen, sulfur, SO₂ or CO; wherein one or more optional substituents on the heterocyclic ring formed may be selected from halogen, hydroxy, alkoxy, alkyl, cycloalkyl or —CO-alkyl;
R₄ is an optionally substituted group selected from cycloalkyl, aryl, heterocyclyl or heteroaryl, wherein one or more optional substituents on R₄ are selected from halogen, hydroxy, cyano, alkyl, haloalkyl, cycloalkyl, alkoxy or —CO-alkyl;
R^{a} is selected from —(CH₂)ₙ—OH, —(CH₂)ₙCOOR^{c}, —(CH₂)ₙCONR^{d}R^{e}, —(CH₂)ₙSO₂R^{f} or —(CH₂)ₙSO₂NR^{c}R^{d};
R^{c} is hydrogen;
R^{d} and R^{e} are each independently selected from hydrogen, alkyl or alkoxy;
R^{f} is selected from alkyl, cycloalkyl, heterocyclyl, aryl or heteroaryl; and
n in an integer from 0 to 2.

11. A composition comprising one or more compounds selected from:
1-[4-(4-tert-Butylamino-6-phenyl-pyrimidin-2-ylamino)-phenyl]-cyclobutane carboxylic acid,
1-[4-(4-tert-Butylamino-6-phenyl-pyrimidin-2-ylamino)-phenyl]-cyclopropane carboxylic acid,
1-[4-(4-tert-Butylamino-6-phenyl-pyrimidin-2-ylamino)-phenyl]-cyclopentane carboxylic acid,
1-[4-(4-tert-Butyl-6-tert-butylamino-pyrimidin-2-ylamino)-phenyl]-cyclobutane carboxylic acid,
1-(4-{4-[(1-Ethyl-pyrrolidin-2-ylmethyl)-amino]-6-phenyl-pyrimidin-2-ylamino}-phenyl)-cyclobutanecarboxylic acid methyl ester,
1-[4-(4-tert-Butylamino-5-fluoro-6-phenyl-pyrimidin-2-ylamino)-phenyl]-cyclobutanecarboxylic acid,
1-{4-[4-(Bis-cyclopropylmethyl-amino)-6-(4-fluoro-phenyl)-pyrimidin-2-ylamino]-phenyl}-cyclobutanecarboxylic acid methyl ester,
1-{4-[4-(Cyclopropylmethyl-amino)-6-(4-fluoro-phenyl)-pyrimidin-2-ylamino]-phenyl}-cyclopentanecarboxylic acid,
1-{4-[4-(Cyclopropylmethyl-amino)-6-(4-fluoro-phenyl)-pyrimidin-2-ylamino]-phenyl}-cyclobutanecarboxylic acid,
1-[4-(4-Methylamino-6-phenyl-pyrimidin-2-ylamino)-phenyl]-cyclobutane carboxylic acid,
1-{4-[4-(tert-Butyl-methyl-amino)-6-phenyl-pyrimidin-2-ylamino]-phenyl}-cyclobutanecarboxylic acid,
1-{4-[4-(Bicyclo[2.2.1]hept-2-ylamino)-6-phenyl-pyrimidin-2-ylamino]-phenyl}-cyclobutanecarboxylic acid,
1-{4-[4-(4-Fluoro-phenyl)-6-morpholin-4-yl-pyrimidin-2-ylamino]-phenyl}-cyclobutanecarboxylic acid,
1-{4-[4-(4-Fluoro-phenyl)-6-(4-hydroxy-piperidin-1-yl)-pyrimidin-2-ylamino]-phenyl}-cyclobutanecarboxylic acid,
1-[4-(4-Cyclobutylamino-6-phenyl-pyrimidin-2-ylamino)-phenyl]-cyclobutane carboxylic acid,
1-[4-(4-Cyclopentylamino-6-phenyl-pyrimidin-2-ylamino)-phenyl]-cyclobutane carboxylic acid,
1-{4-[4-(1,1-Dimethyl-propylamino)-6-phenyl-pyrimidin-2-ylamino]-phenyl}-cyclobutanecarboxylic acid,
1-{4-[4-(4-Fluoro-phenylamino)-6-phenyl-pyrimidin-2-ylamino]-phenyl}-cyclobutanecarboxylic acid,
1-{4-[4-(4-Methanesulfonyl-phenylamino)-6-phenyl-pyrimidin-2-ylamino]-phenyl}-cyclobutanecarboxylic acid,
1-{4-[4-Phenyl-6-(1-phenyl-ethylamino)-pyrimidin-2-ylamino]-phenyl}-cyclobutanecarboxylic acid,
1-{4-[4-Phenyl-6-(1-phenyl-ethylamino)-pyrimidin-2-ylamino]-phenyl}-cyclobutanecarboxylic acid,
1-{4-[4-(2-Hydroxy-1-phenyl-ethyl amino)-6-phenyl-pyrimidin-2-ylamino]-phenyl}-cyclobutanecarboxylic acid,
1-[4-(4-Amino-6-phenyl-pyrimidin-2-ylamino)-phenyl]-cyclobutanecarboxylic acid,
1-{4-[4-(5-Methyl-isoxazol-3-ylamino)-6-phenyl-pyrimidin-2-ylamino]-phenyl}-cyclobutanecarboxylic acid,
1-[4-(4-Cyclopentyl-6-cyclopentylamino-pyrimidin-2-ylamino)-phenyl]-cyclobutanecarboxylic acid,
1-[4-(4-tert-Butylamino-6-cyclopentyl-pyrimidin-2-ylamino)-phenyl]-cyclobutane carboxylic acid,
1-{4-[4-(1-Methyl-1H-pyrazol-4-yl)-6-morpholin-4-yl-pyrimidin-2-ylamino]-phenyl}-cyclobutanecarboxylic acid,
1-{4-[4-(1-Methyl-1H-pyrazol-4-yl)-6-pyrrolidin-1-yl-pyrimidin-2-ylamino]-phenyl}-cyclobutanecarboxylic acid,
1-{4-[4-(4-Hydroxy-piperidin-1-yl)-6-(1-methyl-1H-pyrazol-4-yl)-pyrimidin-2-ylamino]-phenyl}-cyclobutanecarboxylic acid,
1-{4-[4-(3,5-Dimethyl-isoxazol-4-yl)-6-morpholin-4-yl-pyrimidin-2-ylamino]-phenyl}-cyclobutanecarboxylic acid,
1-{3-[4-(3,5-Dimethyl-isoxazol-4-yl)-6-morpholin-4-yl-pyrimidin-2-ylamino]-phenyl}-cyclobutanecarboxylic acid,
1-{4-[4-(3,5-Dimethyl-isoxazol-4-yl)-5-methyl-6-morpholin-4-yl-pyrimidin-2-ylamino]-phenyl}-cyclobutanecarboxylic acid,
1-{4-[4-(3,5-Dimethyl-isoxazol-4-yl)-6-piperazin-1-yl-pyrimidin-2-ylamino]-phenyl}-cyclobutanecarboxylic acid,
1-{4-[4-(4-Acetyl-piperazin-1-yl)-6-(3,5-dimethyl-isoxazol-4-yl)-pyrimidin-2-ylamino]-phenyl}-cyclobutanecarboxylic acid,
1-{4-[4-(6-Methoxy-pyridin-3-yl)-6-morpholin-4-yl-pyrimidin-2-ylamino]-phenyl}-cyclobutanecarboxylic acid,
1-{4-[4-(4,4-Difluoro-piperidin-1-yl)-6-(3,5-dimethyl-isoxazol-4-yl)-pyrimidin-2-ylamino]-phenyl}-cyclobutanecarboxylic acid,
1-{4-[4-Butylamino-6-(3,5-dimethyl-isoxazol-4-yl)-pyrimidin-2-ylamino]-phenyl}-cyclobutanecarboxylic acid,
1-{4-[4-tert-Butylamino-6-(3,5-dimethyl-isoxazol-4-yl)-pyrimidin-2-ylamino]-phenyl}-cyclobutanecarboxylic acid,
1-{4-[4-Cyclopentylamino-6-(3,5-dimethyl-isoxazol-4-yl)-pyrimidin-2-ylamino]-phenyl}-cyclobutanecarboxylic acid,
1-[4-(4-Cyclopentylamino-6-phenyl-pyrimidin-2-ylamino)-phenyl]-cyclobutanecarbonitrile,
Sodium 1-[4-(4-tert-butylamino-6-phenyl-pyrimidin-2-ylamino)-phenyl]-cyclobutane carboxylate,
{1-[4-(4-tert-Butylamino-6-phenyl-pyrimidin-2-ylamino)-phenyl}-cyclobutyl}-methanol,
1-[4-(4-tert-Butylamino-6-phenyl-pyrimidin-2-ylamino)-phenyl]-cyclobutane carboxylic acid ethylamide,
1-[4-(4-tert-Butylamino-6-phenyl-pyrimidin-2-ylamino)-phenyl]-cyclobutane carboxylic acid dimethylamide,
1-[4-(4-tert-Butylamino-6-phenyl-pyrimidin-2-ylamino)-phenyl]-cyclobutane carboxylic acid tert-butylamide,
1-[4-(4-tert-Butylamino-6-phenyl-pyrimidin-2-ylamino)-phenyl]-cyclobutane carboxylic acid amide,
1-[4-(4-tert-Butylamino-6-phenyl-pyrimidin-2-ylamino)-phenyl]-cyclobutane carboxylic acid methoxy-methyl-amide,
1-{1-[4-(4-tert-Butylamino-6-phenyl-pyrimidin-2-ylamino)-phenyl]-cyclobutyl}-ethanone,
N⁴-tert-Butyl-N²-[4-(1-oxazol-2-yl-cyclobutyl)-phenyl]-6-phenyl-pyrimidine-2,4-diamine,
1-[4-(4-tert-Butylamino-6-phenyl-pyrimidin-2-ylamino)-phenyl}-cyclobutane carboxylic acid (4-fluoro-phenyl)-amide,
(1-{4-[4-(3,5-Dimethyl-isoxazol-4-yl)-6-morpholin-4-yl-pyrimidin-2-ylamino]-phenyl}-cyclobutyl)-methanol,
N⁴-tert-Butyl-N²-[4-(1-methoxymethyl-cyclobutyl)-phenyl]-6-phenyl-pyrimidine-2,4-diamine,
N⁴-tert-Butyl-N²-[4-(1-methyl-cyclobutyl)-phenyl]-6-phenyl-pyrimidine-2,4-diamine,
[4-(3,5-Dimethyl-isoxazol-4-yl)-6-morpholin-4-yl-pyrimidin-2-yl]-[4-(1-methanesulfonylmethyl-cyclobutyl)-phenyl]-amine,
1-[4-(2-tert-Butylamino-6-phenyl-pyrimidin-4-ylamino)-phenyl]-cyclobutane carboxylic acid,
1-{4-[2-(tert-Butyl-methyl-amino)-6-phenyl-pyrimidin-4-ylamino]-phenyl}-cyclobutanecarboxylic acid,
1-[4-(6-tert-Butylamino-2-phenyl-pyrimidin-4-ylamino)-phenyl)-cyclobutane carboxylic acid,
1-{4-[6-(3,5-Dimethyl-isoxazol-4-yl)-2-methyl-pyrimidin-4-ylamino]-phenyl}-cyclobutanecarboxylic acid,
1-{4-[2-(4-Fluoro-phenyl)-6-phenyl-pyrimidin-4-ylamino]-phenyl}-cyclobutanecarboxylic acid,
1-{4-[2-(4-Acetyl-phenyl)-6-phenyl-pyrimidin-4-ylamino]-phenyl}-cyclobutanecarboxylic acid,
1-{4-[6-(3,5-Dimethyl-isoxazol-4-yl)-2-(4-fluoro-phenyl)-pyrimidin-4-ylamino]-phenyl}-cyclobutanecarboxylic acid,
1-{4-[2-(6-Methoxy-pyridin-3-yl)-6-phenyl-pyrimidin-4-ylamino]-phenyl}-cyclobutanecarboxylic acid,
1-{4-[2-(4-Methanesulfonyl-phenyl)-6-phenyl-pyrimidin-4-ylamino]-phenyl}-cyclobutanecarboxylic acid,
1-{4-[2-(6-Methyl-pyridin-3-yl)-6-phenyl-pyrimidin-4-ylamino]-phenyl}-cyclobutanecarboxylic acid,
1-}4-[2-(1,1-Dioxo-1λ⁶-thiomorpholin-4-yl)-6-phenyl-pyrimidin-4-ylamino]-phenyl}-cyclobutanecarboxylic acid,
1-{4-[6-(3,5-Dimethyl-isoxazol-4-yl)-2-morpholin-4-yl-pyrimidin-4-ylamino]-phenyl}-cyclobutanecarboxylic acid,
1-[4-(2-Morpholin-4-yl-6-phenyl-pyrimidin-4-ylamino)-phenyl]-cyclobutane carboxylic acid,
1-[4-(4-Isopropoxy-6-phenyl-pyrimidin-2-ylamino)-phenyl]-cyclobutane carboxylic acid,
stereoisomers thereof and/or pharmaceutically acceptable salts of the compounds and/or pharmaceutically acceptable salts of the stereoisomers of the compounds.

12. The compound as claimed in claim 11, wherein the compound is:
1-[4-(4-tert-Butylamino-6-phenyl-pyrimidin-2-ylamino)-phenyl]-cyclobutanecarboxylic acid,
1-[4-(4-tert-Butylamino-6-phenyl-pyrimidin-2-yl-amino)-phenyl]-cyclopropanecarboxylic acid,
1-[4-(4-tert-Butylamino-6-phenyl-pyrimidin-2-ylamino)-phenyl]-cyclopentanecarboxylic acid,
1-[4-(4-tert-Butyl-6-tert-butylamino-pyrimidin-2-ylamino]-phenyl]-cyclobutanecarboxylic acid,
1-(4-{4-[(1-Ethyl-pyrrolidin-2-ylmethyl)-amino]-6-phenyl-pyrimidin-2-ylamino}-phenyl)-cyclobutanecarboxylic acid methyl ester,
1-[4-(4-tert-Butylamino-5-fluoro-6-phenyl-pyrimidin-2-ylamino)-phenyl]-cyclobutanecarboxylic acid,
1-{4-[4-(bis-Cyclopropylmethyl-amino)-6-(4-fluoro-phenyl)-pyrimidin-2-ylamino]-phenyl}-cyclobutanecarboxylic acid methyl ester,
1-{4-[4-(Cyclopropylmethyl-amino)-6-(4-fluoro-phenyl)-pyrimidin-2-ylamino]-phenyl}-cyclopentane-carboxylic acid,
1-{4-[4-(Cyclopropylmethyl-amino)-6-(4-fluoro-phenyl)-pyrimidin-2-ylamino]-phenyl}-cyclobutane-carboxylic acid,
1-[4-(4-Methylamino-6-phenyl-pyrimidin-2-ylamino)-phenyl]-cyclobutanecarboxylic acid,
1-{4-[4-(tert-Butyl-methyl-amino)-6-phenyl-pyrimidin-2-ylamino]-phenyl}-cyclobutane carboxylic acid,
1-{4-[4-(Bicyclo[2.2.1]hept-2-ylamino)-6-phenyl-pyrimidin-2-ylamino}-phenyl}-cyclobutane carboxylic acid,
1-[4-(4-Cyclobutylamino-6-phenyl-pyrimidin-2-ylamino)-phenyl]-cyclobutanecarboxylic acid,
1-[4-(4-Cyclopentylamino-6-phenyl-pyrimidin-2-ylamino)-phenyl]-cyclobutane-carboxylic acid,
1-{4-[4-(1,1-Dimethyl-propylamino)-6-phenyl-pyrimidin-2-ylamino]-phenyl}-cyclobutanecarboxylic acid,
1-{4-[4-(4-Fluoro-phenylamino)-6-phenyl-pyrimidin-2-ylamino]-phenyl}-cyclobutanecarboxylic acid,
1-{4-[4-(4-Methanesulfonyl-phenylamino)-6-phenyl-pyrimidin-2-ylamino]-phenyl}-cyclobutanecarboxylic acid,
(1-{4-[4-Phenyl-6-((R)-1-phenyl-ethylamino)-pyrimidin-2-ylamino]-phenyl}-cyclobutyl)-acetic acid,
(1-{4-[4-Phenyl-6-((S)-1-phenyl-ethylamino)-pyrimidin-2-ylamino]-phenyl}-cyclobutyl)-acetic acid,
1-{4-[4-((S)-2-Hydroxy-1-phenyl-ethylamino)-6-phenyl-pyrimidin-2-ylamino]-phenyl}-cyclobutanecarboxylic acid,
1-[4-(4-Amino-6-phenyl-pyrimidin-2-ylamino)-phenyl}-cyclobutane-carboxylic acid,
1-{4-[4-(5-Methyl-isoxazol-3-ylamino)-6-phenyl-pyrimidin-2-ylamino]-phenyl}-cyclobutanecarboxylic acid,
1-[4-(4-Cyclopentyl-6-cyclopentylamino-pyrimidin-2-ylamino)-phenyl]-cyclobutanecarboxylic acid,
1-[4-(4-tert-Butylamino-6-cyclopentyl-pyrimidin-2-ylamino)-phenyl]-cyclobutanecarboxylic acid,
1-{4-[4-Butylamino-6-(3,5-dimethyl-isoxazol-4-yl)-pyrimidin-2-ylamino]-phenyl}-cyclobutane carboxylic acid,
1-{4-[4-tert-Butylamino-6-(3,5-dimethyl-isoxazol-4-yl)-pyrimidin-2-ylamino]-phenyl}-cyclobutanecarboxylic acid,
1-{4-[4-Cyclopentylamino-6-(3,5-dimethyl-isoxazol-4-yl)-pyrimidin-2-yl-amino]-phenyl}-cyclobutanecarboxylic acid,
1-[4-(4-Cyclopentylamino-6-phenyl-pyrimidin-2-ylamino)-phenyl]-cyclobutanecarbonitrile,
Sodium1-[4-(4-tert-butylamino-6-phenyl-pyrimidin-2-ylamino)-phenyl]-cyclobutane carboxylate,
{1-[4-(4-tert-Butylamino-6-phenyl-pyrimidin-2-ylmethyl)-phenyl]-cyclobutyl}-methanol,
1-[4-(4-tert-Butylamino-6-phenyl-pyrimidin-2-ylamino)-phenyl]-cyclobutanecarboxylic acid ethylamide,
1-[4-(4-tert-Butylamino-6-phenyl-pyrimidin-2-ylamino)-phenyl]-cyclobutanecarboxylic acid dimethylamide,
1-[4-(4-tert-Butylamino-6-phenyl-pyrimidin-2-ylamino)-phenyl]-cyclobutanecarboxylic acid tert-butylamide,
1-[4-(4-tert-Butylamino-6-phenyl-pyrimidin-2-ylamino)-phenyl]-cyclobutanecarboxylic acid methoxy-methyl-amide,
1-{1-[4-(4-tert-Butylamino-6-phenyl-pyrimidin-2-ylamino)-phenyl]-cyclobutyl}-ethanone,
N⁴-tert-Butyl-N²-[4-(1-oxazol-2-yl-cyclobutyl)-phenyl]-6-phenyl-pyrimidine-2,4-diamine,
1-[4-(4-tert-Butylamino-6-phenyl-pyrimidin-2-ylamino)-phenyl]-cyclobutanecarboxylic acid (4-fluoro-phenyl)-amide,
N⁴-tert-Butyl-N²-[4-(1-methoxymethyl-cyclobutyl)-phenyl]-6-phenyl-pyrimidine-2,4-diamine,
N⁴-tert-Butyl-N²-[4-(1-methyl-cyclobutyl)-phenyl]-6-phenyl-pyrimidine-2,4-diamine;
stereoisomers thereof and/or pharmaceutically acceptable salts of the compounds and/or pharmaceutically acceptable salts of the stereoisomers of the compounds.

13. The compound as claimed in claim 11, wherein the compound is
1-{4-[4-(4-fluoro-phenyl)-6-morpholin-4-yl-pyrimidin-2-ylamino]-phenyl}-cyclobutane-carboxylic acid,
1-{4-[4-(4-fluoro-phenyl)-6-(4-hydroxy-piperidin-1-yl)-pyrimidin-2-ylamino]-phenyl}-cyclobutane-carboxylic acid,
1-{4-[4-(1-methyl-1H-pyrazol-4-yl)-6-morpholin-4-yl-pyrimidin-2-ylamino]-phenyl}-cyclobutanecarboxylic acid,
1-{4-[4-(1-methyl-1H-pyrazol-4-yl)-6-pyrrolidin-1-yl-pyrimidin-2-ylamino]-phenyl}-cyclobutanecarboxylic acid,
1-{4-[4-(4-hydroxy-piperidin-1-yl)-6-(1-methyl-1H-pyrazol-4-yl)-pyrimidin-2-ylamino]-phenyl}-cyclobutanecarboxylic acid,
1-{4-[4-(3,5-dimethyl-isoxazol-4-yl)-6-morpholin-4-yl-pyrimidin-2-ylamino]-phenyl}-cyclobutanecarboxylic acid,
1-{3-[4-(3,5-dimethyl-isoxazol-4-yl)-6-morpholin-4-yl-pyrimidin-2-ylamino]-phenyl}-cyclobutanecarboxylic acid,
1-{4-[4-(3,5-dimethyl-isoxazol-4-yl)-5-methyl-6-morpholin-4-yl-pyrimidin-2-ylamino]-phenyl}-cyclobutanecarboxylic acid,
1-{4-[4-(3,5-dimethyl-isoxazol-4-yl)-6-piperazin-1-yl-pyrimidin-2-ylamino]-phenyl}-cyclobutanecarboxylic acid,
1-{4-[4-(4-acetyl-piperazin-1-yl)-6-(3,5-dimethyl-isoxazol-4-yl)-pyrimidin-2-ylamino]-phenyl}-cyclobutanecarboxylic acid,
1-{4-[4-(6-methoxy-pyridin-3-yl)-6-morpholin-4-yl-pyrimidin-2-ylamino]-phenyl}-cyclobutanecarboxylic acid,
1-{4-[4-(4,4-difluoro-piperidin-1-yl)-6-(3,5-dimethyl-isoxazol-4-yl)-pyrimidin-2-ylamino]-phenyl}-cyclobutanecarboxylic acid,
(1-{4-[4-(3,5-dimethyl-isoxazol-4-yl)-6-morpholin-4-yl-pyrimidin-2-ylamino]-phenyl}-cyclobutyl)-methanol,
[4-(3,5-dimethyl-isoxazol-4-yl)-6-morpholin-4-yl-pyrimidin-2-yl]-[4-(1-methanesulfonyl methyl-cyclobutyl)-phenyl]-amine,
stereoisomers thereof and/or pharmaceutically acceptable salts of the compounds and/or pharmaceutically acceptable salts of the stereoisomers of the compounds.

14. The compound as claimed in claim 11, wherein the compound is
1-[4-(2-tert-butylamino-6-phenyl-pyrimidin-4-ylamino)-phenyl}-cyclobutane-carboxylic acid,
1-{4-[2-(tert-butyl-methyl-amino)-6-phenyl-pyrimidin-4-ylamino]-phenyl}-cyclobutane carboxylic acid,
1-[4-(6-tert-butylamino-2-phenyl-pyrimidin-4-ylamino)-phenyl]-cyclobutanecarboxylic acid,
1-{4-[6-(3,5-dimethyl-isoxazol-4-yl)-2-methyl-pyrimidin-4-ylamino]-phenyl}-cyclobutane carboxylic acid,
1-{4-[2-(4-fluoro-phenyl)-6-phenyl-pyrimidin-4-ylamino]-phenyl}-cyclobutanecarboxylic acid,
1-{4-[2-(4-acetyl-phenyl)-6-phenyl-pyrimidin-4-ylamino]-phenyl}-cyclobutanecarboxylic acid,
1-{4-[6-3,5-dimethyl-isoxazol-4-yl)-2-(4-fluoro-phenyl)-pyrimidin-4-ylamino]-phenyl}-cyclobutanecarboxylic acid,
1-{4-[2-(6-methoxy-pyridin-3-yl)-6-phenyl-pyrimidin-4-ylamino]-phenyl}-cyclobutane carboxylic acid,
1-{4-[2-(4-methanesulfonyl-phenyl)-6-phenyl-pyrimidin-4-ylamino]-phenyl}-cyclobutane carboxylic acid,
1-{4-[2-(6-methyl-pyridin-3-yl)-6-phenyl-pyrimidin-4-ylamino]-phenyl}-cyclobutanecarboxylic acid,
1-{4-[2-(1,1-dioxo-1λ⁶-thiomorpholin-4-yl)-6-phenyl-pyrimidin-4-ylamino]-phenyl}-cyclobutanecarboxylic acid,
1-{4-[6-(3,5-dimethyl-isoxazol-4-yl)-2-morpholin-4-yl-pyrimidin-4-ylamino]-phenyl}-cyclobutanecarboxylic acid,
1-[4-(2-morpholin-4-yl-6-phenyl-pyrimidin-4-ylamino)-phenyl]-cyclobutanecarboxylic acid,
stereoisomers thereof and/or pharmaceutically acceptable salts of the compounds and/or pharmaceutically acceptable salts of the stereoisomers of the compounds.

15. A pharmaceutical composition comprising a pharmaceutically acceptable carrier and at least one compound according to claim 1.

16. A compound as claimed in claim 1, wherein the compound is a GATA modulator.

17. A method for the treatment of atherosclerosis and/or diabetes in a subject, which comprises administering to the subject a therapeutically effective amount of a compound comprising: 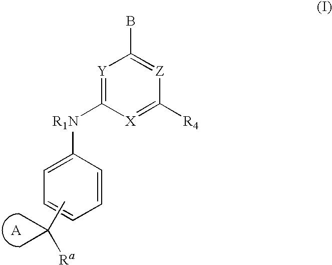
stereoisomers thereof and/or pharmaceutically acceptable salts of compounds of formula (I) and/or pharmaceutically acceptable salts of the stereoisomers of compounds of formula (I); wherein,
X, Y and Z are each independently selected from N or CR^{b}, wherein R^{b} is selected from hydrogen, halogen or alkyl; with a proviso that when X and Y are each independently N, Z is —CR^{b} and when Y and Z are each independently N, X is —CR^{b};
A is a 3 to 7 membered cycloalkyl ring;
B is an optionally substituted group selected from alkyl, alkoxy, cycloalkyl, aryl, heteroaryl or —NR₂R₃, wherein one or more optional substituents on B are selected from halogen, hydroxy, cyano, alkyl, haloalkyl, alkoxy, cycloalkyl, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl;
R₁ is selected from hydrogen or alkyl;
R₂ and R₃ are each independently selected from hydrogen, —(CH₂)ₙ-hydroxy, —(CH₂)ₙ-halogen, and an optionally substituted group selected from alkyl, —(CH₂)ₙ-cycloalkyl, —(CH₂)ₙ-heterocyclyl, —(CH₂)ₙ-aryl or —(CH₂)ₙ-heteroaryl, wherein one or more optional substituents on R₂ and R₃ are each independently selected from halogen, cyano, hydroxy, alkyl, cycloalkyl, alkoxy, —CO-alkyl, —COOR^{c}, —CONR^{d}R^{e}, —(CH₂)ₙSO₂R^{f}, —(CH₂)ₙSO₂NR^{c}R^{d}, aryl, heteroaryl or heterocyclyl; or
R₂ and R₃ optionally combine with the nitrogen atom, to which they are attached, to form an optionally substituted 5- to 6-membered heterocyclic ring optionally having 1 to 3 additional hetero atoms or groups selected from nitrogen, oxygen, sulfur, SO₂ or CO, wherein one or more optional substituents on the heterocyclic ring are selected from halogen, hydroxy, alkoxy, alkyl, cycloalkyl, or —CO-alkyl;
R₄ is hydrogen or an optionally substituted group selected from amino, alkyl, cycloalkyl, aryl, heterocyclyl or heteroaryl, wherein one or more optional substituents on R₄ are selected from halogen, hydroxy, cyano, alkyl, —(CH₂)ₙ—OH, cycloalkyl, alkoxy or —CO-alkyl;
R^{a} is selected from cyano, —(CH₂)ₙ—OH, —(CH₂)ₙ-alkoxy, alkyl, aryl, —NH-aryl, heteroaryl, —(CH₂)ₙCO-alkyl, —(CH₂)ₙCOOR^{c}, —(CH₂)ₙCONR^{d}R^{e}, —(CH₂)ₙSO₂R^{f}, or —(CH₂)ₙSO₂NR^{c}R^{d};
R^{c} is selected from hydrogen, alkyl, cycloalkyl, aryl, heterocyclyl or heteroaryl;
R^{d} and R^{e} are each independently selected from hydrogen or an optionally substituted group selected from alkyl, cycloalkyl, alkoxy, aryl, heteroaryl or heterocyclyl, wherein one or more optional substituents on R^{d} and R^{e} are each independently selected from halogen, hydroxy or alkyl;
R^{f} is selected from alkyl, cycloalkyl, heterocyclyl, aryl or heteroaryl; and
n is an integer from 0 to 2.

18. The method as claimed in claim 17, comprising treating a subject having diabetes.

19. The method as claimed in claim 17, comprising treating a subject having atherosclerosis.

20. A method for the treatment of atherosclerosis and/or diabetes in a subject, which comprises administering to the subject a therapeutically effective amount of at least one compound selected from one or more of:
1-[4-(4-tert-Butylamino-6-phenyl-pyrimidin-2-ylamino)-phenyl}-cyclobutane carboxylic acid,
1-[4-(4-tert-Butylamino-6-phenyl-pyrimidin-2-ylamino)-phenyl}-cyclopropane carboxylic acid,
1-[4-(4-tert-Butylamino-6-phenyl-pyrimidin-2-ylamino)-phenyl]-cyclopentane carboxylic acid,
1-[4-(4-tert-Butyl-6-tert-butylamino-pyrimidin-2-ylamino)-phenyl}-cyclobutane carboxylic acid,
1-(4-{4-[(1-Ethyl-pyrrolidin-2-ylmethyl)-amino]-6-phenyl-pyrimidin-2-ylamino}-phenyl)-cyclobutanecarboxylic acid methyl ester,
1-[4-(4-tert-Butylamino-5-fluoro-6-phenyl-pyrimidin-2-ylamino)-phenyl]-cyclobutanecarboxylic acid,
1-{4-[4-(Bis-cyclopropylmethyl-amino)-6-(4-fluoro-phenyl)-pyrimidin-2-ylamino]-phenyl}-cyclobutanecarboxylic acid methyl ester,
1-{4-[4-(Cyclopropylmethyl-amino)-6-(4-fluoro-phenyl)-pyrimidin-2-ylamino]-phenyl}-cyclopentanecarboxylic acid,
1-{4-[4-(Cyclopropylmethyl-amino)-6-(4-fluoro-phenyl)-pyrimidin-2-ylamino]-phenyl}-cyclobutanecarboxylic acid,
1-[4-(4-Methylamino-6-phenyl-pyrimidin-2-ylamino)-phenyl}-cyclobutane carboxylic acid,
1-{4-[4-(tert-Butyl-methyl-amino)-6-phenyl-pyrimidin-2-ylamino]-phenyl}-cyclobutanecarboxylic acid,
1-{4-[4-(Bicyclo[2.2.1]hept-2-ylamino)-6-phenyl-pyrimidin-2-ylamino]-phenyl}-cyclobutanecarboxylic acid,
1-{4-[4-(4-Fluoro-phenyl)-6-morpholin-4-yl-pyrimidin-2-ylamino]-phenyl}-cyclobutanecarboxylic acid,
1-{4-[4-(4-Fluoro-phenyl)-6-(4-hydroxy-piperidin-1-yl)-pyrimidin-2-ylamino]-phenyl}-cyclobutanecarboxylic acid,
1-[4-(4-Cyclobutylamino-6-phenyl-pyrimidin-2-ylamino)-phenyl]-cyclobutane carboxylic acid,
1-[4-(4-Cyclopentylamino-6-phenyl-pyrimidin-2-ylamino)-phenyl}-cyclobutane carboxylic acid,
1-{4-[4-(1,1-Dimethyl-propylamino)-6-phenyl-pyrimidin-2-ylamino]-phenyl}-cyclobutanecarboxylic acid,
1-{4-[4-(4-Fluoro-phenylamino)-6-phenyl-pyrimidin-2-ylamino]-phenyl}-cyclobutanecarboxylic acid,
1-{4-[4-(4-Methanesulfonyl-phenylamino)-6-phenyl-pyrimidin-2-ylamino]-phenyl}-cyclobutanecarboxylic acid,
1-{4-[4-Phenyl-6-(1-phenyl-ethylamino)-pyrimidin-2-ylamino]-phenyl}-cyclobutanecarboxylic acid,
1-{4-8 4-Phenyl-6-(1-phenyl-ethylamino)-pyrimidin-2-ylamino]-phenyl}-cyclobutanecarboxylic acid,
1-{4-[4-(2-Hydroxy-1-phenyl-ethylamino)-6-phenyl-pyrimidin-2-ylamino]-phenyl}-cyclobutanecarboxylic acid,
1-[4-(4-Amino-6-phenyl-pyrimidin-2-ylamino)-phenyl]-cyclobutanecarboxylic acid,
1-{4-[4-(5-Methyl-isoxazol-3-ylamino)-6-phenyl-pyrimidin-2-ylamino]-phenyl}-cyclobutanecarboxylic acid,
1-[4-(4-Cyclopentyl-6-cyclopentylamino-pyrimidin-2-ylamino)-phenyl]-cyclobutanecarboxylic acid,
1-[4-(4-tert-Butylamino-6-cyclopentyl-pyrimidin-2-ylamino)-phenyl]-cyclobutane carboxylic acid,
1-{4-[4-(1-Methyl-1H-pyrazol-4-yl)-6-morpholin-4-yl-pyrimidin-2-ylamino]-phenyl}-cyclobutanecarboxylic acid,
1-{4-[4-(1-Methyl-1H-pyrazol-4-yl)-6-pyrrolidin-1-yl-pyrimidin-2-ylamino]-phenyl}-cyclobutanecarboxylic acid,
1-{4-[4-(4-Hydroxy-piperidin-1-yl)-6-(1-methyl-1H-pyrazol-4-yl)-pyrimidin-2-ylamino]-phenyl}-cyclobutanecarboxylic acid,
1-{4-[4-(3,5-Dimethyl-isoxazol-4-yl)-6-morpholin-4-yl-pyrimidin-2-ylamino]-phenyl}-cyclobutanecarboxylic acid,
1-{3-[4-(3,5-Dimethyl-isoxazol-4-yl)-6-morpholin-4-yl-pyrimidin-2-ylamino]-phenyl}-cyclobutanecarboxylic acid,
1-{4-[4-(3,5-Dimethyl-isoxazol-4-yl)-5-methyl-6-morpholin-4-yl-pyrimidin-2-ylamino]-phenyl}-cyclobutanecarboxylic acid,
1-{4-[4-(3,5-Dimethyl-isoxazol-4-yl)-6-piperazin-1-yl-pyrimidin-2-ylamino]-phenyl}-cyclobutanecarboxylic acid,
1-{4-[4-(4-Acetyl-piperazin-1-yl)-6-(3,5-dimethyl-isoxazol-4-yl)-pyrimidin-2-ylamino]-phenyl}-cyclobutanecarboxylic acid,
1-{4-[4-(6-Methoxy-pyridin-3-yl)-6-morpholin-4-yl-pyrimidin-2-ylamino]-phenyl}-cyclobutanecarboxylic acid,
1-{4-[4-(4,4-Difluoro-piperidin-1-yl)-6-(3,5-dimethyl-isoxazol-4-yl)-pyrimidin-2-ylamino]-phenyl}-cyclobutanecarboxylic acid,
1-{4-[4-Butylamino-6-(3,5-dimethyl-isoxazol-4-yl)-pyrimidin-2-ylamino]-phenyl}-cyclobutanecarboxylic acid,
1-{4-[4-tert-Butylamino-6-(3,5-dimethyl-isoxazol-4-yl)-pyrimidin-2-ylamino]-phenyl}-cyclobutanecarboxylic acid,
1-{4-[4-Cyclopentylamino-6-(3,5-dimethyl-isoxazol-4-yl)-pyrimidin-2-ylamino]-phenyl}-cyclobutanecarboxylic acid,
1-[4-(4-Cyclopentylamino-6-phenyl-pyrimidin-2-ylamino)-phenyl]-cyclobutanecarbonitrile,
Sodium 1-[4-(4-tert-butylamino-6-phenyl-pyrimidin-2-ylamino)-phenyl}-cyclobutane carboxylate,
{1-[4-(4-tert-Butylamino-6-phenyl-pyrimidin-2-ylamino)-phenyl}-cyclobutyl}-methanol,
1-[4-(4-tert-Butylamino-6-phenyl-pyrimidin-2-ylamino)-phenyl}-cyclobutane carboxylic acid ethylamide,
1-[4-(4-tert-Butylamino-6-phenyl-pyrimidin-2-ylamino)-phenyl]-cyclobutane carboxylic acid dimethylamide,
1-[4-(4-tert-Butylamino-6-phenyl-pyrimidin-2-ylamino)-phenyl}-cyclobutane carboxylic acid tert-butylamide,
1-[4-(4-tert-Butylamino-6-phenyl-pyrimidin-2-ylamino)-phenyl]-cyclobutane carboxylic acid amide,
1-[4-(4-tert-Butylamino-6-phenyl-pyrimidin-2-ylamino)-phenyl]-cyclobutane carboxylic acid methoxy-methyl-amide,
1-{1-[4-(4-tert-Butylamino-6-phenyl-pyrimidin-2-ylamino)-phenyl]-cyclobutyl}-ethanone,
N⁴-tert-Butyl-N²-[4-(1-oxazol-2-yl-cyclobutyl)-phenyl]-6-phenyl-pyrimidine-2,4-diamine,
1-[4-(4-tert-Butylamino-6-phenyl-pyrimidin-2-ylamino)-phenyl]-cyclobutane carboxylic acid (4-fluoro-phenyl)-amide,
(1-{4-[4-(3,5-Dimethyl-isoxazol-4-yl)-6-morpholin-4-yl-pyrimidin-2-ylamino]-phenyl}-cyclobutyl)-methanol,
N⁴-tert-Butyl-N²-[4-(1-methoxymethyl-cyclobutyl)-phenyl]-6-phenyl-pyrimidine-2,4-diamine,
N⁴-tert-Butyl-N²-[4-(1-methyl-cyclobutyl)-phenyl]-6-phenyl-pyrimidine-2,4-diamine,
[4-(3,5-Dimethyl-isoxazol-4-yl)-6-morpholin-4-yl-pyrimidin-2-yl]-[4-(1-methanesulfonylmethyl-cyclobutyl)-phenyl]-amine,
1-[4-(2-tert-Butylamino-6-phenyl-pyrimidin-4-ylamino)-phenyl]-cyclobutane carboxylic acid,
1-{4-[2-(tert-Butyl-methyl-amino)-6-phenyl-pyrimidin-4-ylamino]-phenyl}-cyclobutanecarboxylic acid,
1-[4-(6-tert-Butylamino-2-phenyl-pyrimidin-4-ylamino)-phenyl}-cyclobutane carboxylic acid,
1-{4-[6-(3,5-Dimethyl-isoxazol-4-yl)-2-methyl-pyrimidin-4-ylamino]-phenyl}-cyclobutanecarboxylic acid,
1-{4-[2-(4-Fluoro-phenyl)-6-phenyl-pyrimidin-4-ylamino]-phenyl}-cyclobutanecarboxylic acid,
1-{4-]2-(4-Acetyl-phenyl)-6-phenyl-pyrimidin-4-ylamino]-phenyl}-cyclobutanecarboxylic acid,
1-{4-[6-(3,5-Dimethyl-isoxazol-4-yl)-2-(4-fluoro-phenyl)-pyrimidin-4-ylamino]-phenyl}-cyclobutanecarboxylic acid,
1-{4-[2-(6-Methoxy-pyridin-3-yl)-6-phenyl-pyrimidin-4-ylamino]-phenyl}-cyclobutanecarboxylic acid,
1-{4-[2-(4-Methanesulfonyl-phenyl)-6-phenyl-pyrimidin-4-ylamino]-phenyl}-cyclobutanecarboxylic acid,
1-{4-[2-(6-Methyl-pyridin-3-yl)-6-phenyl-pyrimidin-4-ylamino]-phenyl}-cyclobutanecarboxylic acid,
1-{4-[2-(1,1-Dioxo-1λ⁶-thiomorpholin-4-yl)-6-phenyl-pyrimidin-4-ylamino]-phenyl}-cyclobutanecarboxylic acid,
1-{4-[6-(3,5-Dimethyl-isoxazol-4-yl)-2-morpholin-4-yl-pyrimidin-4-ylamino]-phenyl}-cyclobutanecarboxylic acid,
1-[4-(2-Morpholin-4-yl-6-phenyl-pyrimidin-4-ylamino)-phenyl]-cyclobutane carboxylic acid,
1-[4-(4-Isopropoxy-6-phenyl-pyrimidin-2-ylamino)-phenyl}-cyclobutane carboxylic acid,
stereoisomers thereof and/or pharmaceutically acceptable salts of the compounds and/or pharmaceutically acceptable salts of the stereoisomers of the compounds.

21. The method as claimed in claim 20, comprising treating a subject having diabetes.

22. The method as claimed in claim 20, comprising treating a subject having atherosclerosis.
